(19) United States
(12) Patent Application Publication
(10) Pub. No.: US 2024/0325558 A1
(43) Pub. Date: Oct. 3, 2024

(54) MUSCLE TARGETING COMPLEXES AND USES THEREOF FOR TREATING MYOTONIC DYSTROPHY

(71) Applicant: Dyne Therapeutics, Inc., Waltham, MA (US)

(72) Inventors: Stefano Zanotti, Waltham, MA (US); Tyler Picariello, Waltham, MA (US); Timothy Weeden, Waltham, MA (US); Cody A. Desjardins, Waltham, MA (US); Romesh R. Subramanian, Framingham, MA (US); Mohammed T. Qatanani, Waltham, MA (US); Brendan Quinn, Boston, MA (US); John Najim, Waltham, MA (US)

(73) Assignee: Dyne Therapeutics, Inc., Waltham, MA (US)

(21) Appl. No.: 18/577,468

(22) PCT Filed: Jul. 8, 2022
(86) PCT No.: PCT/US2022/073536

§ 371 Date: Jan. 8, 2024

Related U.S. Application Data
(60) Provisional application No. 63/220,000, filed on Jul. 9, 2021.
(60) Provisional application No. 63/316,905, filed on Mar. 4, 2022.

(51) Int. Cl. A61K 47/68; A61P 21/00

(52) U.S. Cl. CPC A61K 47/6807; A61K 47/6849; A61K 47/6889; A61P 21/00

(57) ABSTRACT

The present application relates to oligonucleotides (e.g., antisense oligonucleotides such as gapmers) designed to target DMPK RNAs and targeting complexes for delivering the oligonucleotides to cells (e.g., muscle cells) and uses thereof, particularly uses relating to treatment of disease. In some embodiments, the muscle-targeting agent specifically binds to an internalizing cell surface receptor on muscle cells. In some embodiments, the molecular payload inhibits expression or activity of DMPK.

### RELATED APPLICATIONS

This application claims priority under 35 U.S.C. § 119(e) to U.S. Provisional Application Ser. No. 63/220,000, entitled “MUSCLE TARGETING COMPLEXES AND USES THEREOF FOR TREATING MYOTONIC DYSTROPHY”, filed on Jul. 9, 2021, and to U.S. Provisional Application Ser. No. 63/316,905, entitled “MUSCLE TARGETING COMPLEXES AND USES THEREOF FOR TREATING MYOTONIC DYSTROPHY”, filed on Mar. 4, 2022; the contents of each of which are incorporated herein by reference in their entirety.

### FIELD OF THE INVENTION

The present application relates to oligonucleotides designed to target DMPK RNAs and targeting complexes for delivering the oligonucleotides to cells (e.g., muscle cells) and uses thereof, particularly uses relating to treatment of disease.

### REFERENCE TO AN ELECTRONIC SEQUENCE LISTING

The contents of the electronic sequence listing (D082470054WO00-SEQ-COB.xml; Size: 574,699 bytes; and Date of Creation: Jul. 7, 2022) are herein incorporated by reference in their entirety.

### BACKGROUND OF INVENTION

Myotonic dystrophy (DM) is a dominantly inherited genetic disease that is characterized by myotonia, muscle loss or degeneration, diminished muscle function, insulin resistance, cardiac arrhythmia, smooth muscle dysfunction, and neurological abnormalities. DM is the most common form of adult-onset muscular dystrophy, with a worldwide incidence of about 1 in 8000 people worldwide. Two types of the disease, myotonic dystrophy type 1 (DM1) and myotonic dystrophy type 2 (DM2), have been described. DM1, the more common form of the disease, results from a repeat expansion of a CTG trinucleotide repeat in the 3′ non-coding region of DMPK on chromosome 19; DM2 results from a repeat expansion of a CCTG tetranucleotide repeat in the first intron of ZNF9 on chromosome 3. In DM1 patients, the repeat expansion of a CTG trinucleotide repeat, which may comprise greater than about 50 to about 3,000 or more total repeats, leads to generation of toxic RNA repeats capable of forming hairpin structures that bind essential intracellular proteins, e.g., muscleblind-like proteins, with high affinity resulting in protein sequestration and the loss-of-function phenotypes that are characteristic of the disease. Apart from supportive care and treatments to address the symptoms of the disease, no effective therapeutic for DM1 is currently available.

### SUMMARY OF INVENTION

In some aspects, the disclosure provides oligonucleotides designed to target DMPK RNAs. In some embodiments, the disclosure provides oligonucleotides complementary with DMPK RNA that are useful for reducing levels of toxic DMPK having disease-associated repeat expansions, e.g., in a subject having or suspected of having myotonic dystrophy. In some embodiments, the oligonucleotides are designed to direct RNAse H mediated degradation of the target DMPK RNA. In some embodiments, the oligonucleotides are designed to direct RNAse H mediated degradation of the target DMPK RNA residing in the nucleus of cells, e.g., muscle cells (e.g., myotubes) or cells of the nervous system (e.g., central nervous system (CNS) cells). In some embodiments, the oligonucleotides are designed to have desirable bioavailability and/or serum-stability properties. In some embodiments, the oligonucleotides are designed to have desirable binding affinity properties. In some embodiments, the oligonucleotides are designed to have desirable toxicity profiles. In some embodiments, the oligonucleotides are designed to have low-complement activation and/or cytokine induction properties.

In some embodiments, oligonucleotides provided herein are designed to facilitate conjugation to other molecules, e.g., targeting agents, e.g., muscle targeting agents. Accordingly, in some aspects, the disclosure provides complexes that target specific cell types for purposes of delivering the oligonucleotides to those cells. For example, in some embodiments, the disclosure provides complexes that target muscle cells for purposes of delivering oligonucleotides to those cells. In some embodiments, complexes provided herein are particularly useful for delivering molecular payloads that inhibit the expression or activity of a DMPK allele comprising an expanded disease-associated-repeat, e.g., in a subject having or suspected of having myotonic dystrophy. Accordingly, in some embodiments, complexes provided herein comprise muscle-targeting agents (e.g., muscle targeting antibodies) that specifically bind to receptors on the surface of muscle cells for purposes of delivering molecular payloads to the muscle cells. In some embodiments, the complexes are taken up into the cells via a receptor mediated internalization, following which the molecular payload may be released to perform a function inside the cells. For example, complexes engineered to deliver oligonucleotides may release the oligonucleotides such that the oligonucleotides can inhibit mutant DMPK expression in the muscle cells. In some embodiments, the oligonucleotides are released by endosomal cleavage of covalent linkers connecting oligonucleotides and muscle-targeting agents of the complexes. It should be understood that the oligonucleotides and/or complexes provided herein can be useful in multiple tissue and cell types, such as within muscle tissues (e.g., in muscle cells) and in the central nervous system (e.g., in CNS cells such as neurons).

Some aspects of the present disclosure provide oligonucleotides that target a DMPK RNA.

According to some aspects, complexes comprising an anti-transferrin receptor 1 (TfR1) antibody covalently linked to an oligonucleotide configured for reducing expression or activity of DMPK are provided, wherein the anti-TfR1 antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR1 antibodies listed in Tables 2-7,
and wherein the oligonucleotide comprises a 5′-X-Y-Z-3′ configuration, wherein
X comprises 3-7 linked nucleosides, wherein at least one of the nucleosides in X is a 2′-modified nucleoside;
Y comprises 6-15 linked 2′-deoxyribonucleosides, wherein each cytosine in Y is optionally and independently a 5-methyl-cytosine; and
Z comprises 3-7 linked nucleosides, wherein at least one of the nucleosides in Z is a 2′-modified nucleoside; and
wherein the oligonucleotide comprises a region of complementarity to at least 15 consecutive nucleosides of any one of SEQ ID NOs: 205, 214, 222, 217, 211, 215, 220, 225, 160-204, 206-210, 212, 213, 216, 218, 219, 221, 223, 224, and 226-230.

In some embodiments, X comprises 3-5 linked nucleosides, wherein at least one of the nucleosides in X is a 2′-modified nucleoside;
Y comprises 6-10 linked 2′-deoxyribonucleosides, wherein each cytosine in Y is optionally and independently a 5-methyl-cytosine; and
Z comprises 3-5 linked nucleosides, wherein at least one of the nucleosides in Z is a 2′-modified nucleoside.

In some embodiments, the anti-TfR1 antibody comprises a heavy chain variable region (VH) comprising an amino acid sequence at least 95% identical to SEQ ID NO: 76 and/or a light chain variable region (VL) comprising an amino acid sequence at least 95% identical to SEQ ID NO: 75,
optionally wherein the anti-TfR1 antibody comprises a VH comprising the amino acid sequence of SEQ ID NO: 76 and a VL comprising the amino acid sequence of SEQ ID NO: 75.

In some embodiments, the anti-TfR1 antibody is a Fab, wherein the Fab comprises a heavy chain comprising an amino acid sequence at least 85% identical to SEQ ID NO: 101 and/or a light chain comprising an amino acid sequence at least 85% identical to SEQ ID NO: 90,
optionally wherein the Fab comprises a heavy chain comprising the amino acid sequence of SEQ ID NO: 101 and a light chain comprising the amino acid sequence of SEQ ID NO: 90.

In some embodiments, the antibody and the oligonucleotide are covalently linked via a cleavable linker, wherein the cleavable linker optionally comprises a valine-citrulline sequence.

In some embodiments, the oligonucleotide is 15 to 25 nucleosides in length, optionally wherein the oligonucleotide is 15 to 20 nucleosides in length.

In some embodiments, the oligonucleotide comprises at least 15 consecutive nucleosides of any one of SEQ ID NOs: 276, 348, 354, 350, 345, 286, 352, 357, 231-275, 277-285, 287-344, 346, 347, 349, 351, 353, 355, 356, and 358-362, wherein each thymine base (T) may independently and optionally be replaced with a uracil base (U), and each U may independently and optionally be replaced with a T.

In some embodiments, each nucleoside in X is a 2′-modified nucleoside and/or each nucleoside in Z is a 2′-modified nucleoside, optionally wherein each 2′-modified nucleoside is independently a 2′-4′ bicyclic nucleoside or a non-bicyclic 2′-modified nucleoside.

In some embodiments, the oligonucleotide comprises a 5′-X-Y-Z-3′ configuration of:

| X | Y | Z |
|---|---|---|
| EEEEE | (D)₁₀ | EEEEE, |
| EEE | (D)₁₀ | EEE, |
| EEEEE | (D)₁₀ | EEEE, |
| EEEEE | (D)₁₀ | EE, |
| LLL | (D)₁₀ | LLL, |
| EELL | (D)₈ | LLEE, |
| LLEE | (D)₈ | EELL, or |
| LLEEE | (D)₁₀ | EEELL, |

wherein “E” is a 2′-MOE modified ribonucleoside; “L” is LNA; “D” is 2′-deoxyribonucleoside; and “10” or “8” is the number of the 2′-deoxyribonucleosides in Y.

In some embodiments, the oligonucleotide comprises one or more phosphorothioate internucleoside linkages.

In some embodiments, each internucleoside linkage in the oligonucleotide is a phosphorothioate internucleoside linkage.

In some embodiments, the oligonucleotide comprises one or more phosphodiester internucleoside linkages, optionally wherein the one or more phosphodiester internucleoside linkages are in X and/or Z.

In some embodiments, the oligonucleotide comprises a structure selected from:

(SEQ ID NO: 302)
oC*oA*oU*oG*xG*dC*dA*dT*dA*dC*dA*
dC*dC*dT*dG*oG*C*oC*oC*oG,
(SEQ ID NO: 303)
oC*oA*oC*oC*xA*dA*dC*dA*xdC*dG*dT
*dC*dC*dC*dT*oC*oU*oC*oC*oU,
(SEQ ID NO: 304)
oU*oC*oA*oC*xC*dA*dA*dC*dA*xdC*dG
*dT*dC*dC*dC*oU*oC*oU*oC*oC,
(SEQ ID NO: 305)
oC*oC*oA*oU*oU*dC*dA*dC*dC*dA*dA*
dC*dA*xdC*dG*oU*oC*oC*oC*oU,
(SEQ ID NO: 306)
oU*oA*xC*xA*oG*dG*dT*dA*dG*dT*dT*
dC*dT*dC*dA*oU*oC*oC*oU*oG,
(SEQ ID NO: 307)
oG*oU*oA*oC*xA*dG*dG*dT*dA*dG*dT*
dT*dC*dT*dC*oA*oU*oC*oC*oU,
(SEQ ID NO: 308)
oA*oC*xC*xA*oG*dG*dT*dA*dC*dA*dG*
dG*dT*dA*dG*oU*oU*oC*oU*oC,
(SEQ ID NO: 309)
oG*oA*oC*xC*xA*dG*dG*dT*dA*dC*dA*
dG*dG*dT*dA*oG*oU*oU*oC*oU,
(SEQ ID NO: 310)
oU*oG*oA*oC*oC*dA*dG*dG*dT*dA*dC*
dA*dG*dG*dT*oA*xoG*oU*oU*oC,
(SEQ ID NO: 311)
oC*oC*C*oA*xA*dA*dC*dT*dT*dG*dC*d
T*dC*dA*dG*oC*oA*oG*oU*oG,
(SEQ ID NO: 312)
oU*oG*xA*xC*xA*dA*dT*dC*dT*dC*xdC
*dG*dC*dC*dA*oG*oG*oU*oA*oG,
(SEQ ID NO: 313)
oA*oU*oG*xA*xC*dA*dA*dT*dC*dT*dC*
xdC*dG*dC*dC*oA*oG*G*oU*oA,
(SEQ ID NO: 314)
oC*xA*oU*oG*A*dC*dA*dA*dT*dC*dT*d
C*xdC*dG*dC*oC*oA*oG*oG*oU,
(SEQ ID NO: 315)
oC*oC*oA*oU*G*dA*dC*dA*dA*dT*dC*d
T*dC*xdC*dG*oC*oC*oA*oG*oG,
(SEQ ID NO: 316)
oG*oC*oC*xA*oU*dG*dA*dC*dA*dA*dT*
dC*dT*dC*xdC*oG*oC*oC*xA*oG,
(SEQ ID NO: 246)
oG*oG*oC*xC*xA*dT*G*dA*dC*dA*dA*d
T*dC*dT*dC*oC*oG*oC*oC*oA,
(SEQ ID NO: 317)
oU*oG*oG*oC*oC*dA*dT*dG*dA*dC*dA*
dA*dT*dC*dT*oC*oC*oG*oC*oC,
(SEQ ID NO: 318)
oU*oG*oU*oG*oC*dA*xdC*dG*dT*dA*dG
*dC*dC*dA*dA*G*oC*oC*oG*oG,
(SEQ ID NO: 319)
oC*oU*oG*oU*oG*dC*dA*xdC*dG*dT*dA
*dG*dC*dC*dA*oA*oG*oC*C*oG,
(SEQ ID NO: 320)
oC*oA*oC*A*oG*xdC*dG*dG*dT*dC*dC*
dA*dG*dC*dA*oG*oG*xA*oU*oG,
(SEQ ID NO: 321)
oU*oG*oG*oC*oC*dA*dC*dA*dG*xdC*dG
*dG*dT*dC*dC*A*oG*oC*oA*oG,
(SEQ ID NO: 322)
oA*oG*oC*oG*oC*dC*dC*dA*dC*dC*dA*
dG*dT*dC*dA*oC*xA*oC*oU*oC,
(SEQ ID NO: 323)
oC*xA*oG*oC*xG*dC*dC*dC*dA*dC*dC*
dA*dG*dT*dC*oA*oC*xA*C*oU,
(SEQ ID NO: 254)
oC*oC*oA*oG*oC*dG*dC*dC*dC*dA*dC*
dC*dA*dG*dT*oC*oA*oC*xA*oC,
(SEQ ID NO: 255)
oG*oC*G*oA*xA*dT*dA*dC*dA*dC*dC*d
C*dA*dG*xdC*oG*oC*oC*oC*oA,
(SEQ ID NO: 256)
oG*oG*C*oG*oA*dA*dT*dA*dC*dA*dC*d
C*dC*dA*dG*oC*oG*oC*oC*oC,
(SEQ ID NO: 324)
oU*oU*oG*oU*oA*dG*dT*dG*dG*dA*xdC
*dG*dA*dT*dC*oU*oU*oG*oC*oC,
(SEQ ID NO: 325)
oC*oU*oU*oG*oU*dA*dG*dT*dG*dG*dA*
xdC*dG*dA*dT*oC*oU*oU*oG*oC,
(SEQ ID NO: 326)
oC*oC*oU*oU*xG*dT*dA*G*dT*dG*dG*d
A*xdC*dG*dA*oU*oC*oU*oU*oG,
(SEQ ID NO: 327)
oC*oG*oG*oA*oG*dA*dC*dC*dA*dT*dC*
dC*dC*dA*dG*oU*oC*oG*oA*oG,
(SEQ ID NO: 328)
oG*oA*xA*oU*G*dT*dC*xdC*dG*dA*dC*
dA*dG*dT*dG*oU*oC*oU*oC*oC,
(SEQ ID NO: 329)
oC*oG*oA*xA*oU*dG*dT*dC*xdC*dG*dA
*dC*dA*dG*dT*oG*oU*oC*oU*oC,
(SEQ ID NO: 330)
oG*oG*oG*oC*oC*dT*dG*dG*dG*dA*dC*
dC*dT*dC*dA*oC*oU*oG*oU*oC,
(SEQ ID NO: 331)
oU*G*oC*xA*oC*dG*dT*dG*dT*dG*dG*d
C*dT*dC*dA*oA*G*oC*oA*oG,
(SEQ ID NO: 332)
oC*oC*A*oC*oU*T*dC*dA*dG*dC*dT*dG
*dT*dT*dT*oC*oA*oU*oC*oC,
(SEQ ID NO: 333)
oG*oC*oG*oU*oC*dA*dC*dC*dT*xdC*dG
*dG*dC*dC*dT*oC*A*oG*oC*oC,
(SEQ ID NO: 334)
oA*oG*C*oG*oU*dC*dA*dC*dC*dT*xdC*
dG*dG*dC*dC*oU*oC*oA*oG*oC,
(SEQ ID NO: 335)
oC*oG*oU*oA*oG*dT*dT*dG*dA*dC*dT*
dG*dG*xdC*dG*xA*oA*oG*oU*oU,
(SEQ ID NO: 336)
oG*oG*oG*oC*xC*xdC*dG*dG*dA*dT*dC
*dA*dC*dA*dG*oG*oA*oC*oU*oG,
(SEQ ID NO: 337)
oU*oU*oG*oC*oC*dC*dA*dT*dC*dC*dA*
xdC*dG*dT*dC*oA*oG*oG*oG*oC,
(SEQ ID NO: 338)
oG*oG*xA*oC*oG*dG*dC*dC*xdC*dG*dG
*dC*dT*dT*dG*oC*oU*oG*oC*oC,
(SEQ ID NO: 339)
oU*oG*oG*xA*oA*dC*dA*xdC*dG*dG*dA
*xdC*dG*dG*dC*oC*oC*oG*oG*oC,
(SEQ ID NO: 340)
oC*oA*oU*oC*oC*dA*dA*dA*dA*xdC*dG
*dT*dG*dG*dA*oU*oU*oG*oG*oG,
and
(SEQ ID NO: 341)
oG*C*oA*oU*oC*dC*dA*dA*dA*dA*xdC*
dG*dT*dG*dG*oA*oU*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

In some embodiments, the oligonucleotide is conjugated to an amine group at its 5′-end and comprises a structure selected from:

(SEQ ID NO: 302)
NH₂-(CH₂)₆-oC*oA*oU*oG*G*dC*dA*dT*dA*
dC*dA*dC*dC*dT*dG*oG*oC*oC*oC*oG,
(SEQ ID NO: 303)
NH₂-(CH₂)₆-oC*oA*oC*oC*oA*dA*dC*dA*xd
C*dG*dT*dC*dC*dC*dT*oC*oU*oC*oC*oU,
(SEQ ID NO: 304)
NH₂-(CH₂)₆-oU*oC*oA*oC*oC*dA*dA*dC*dA
*xdC*dG*dT*dC*dC*dC*oU*oC*oU*oC*oC,
(SEQ ID NO: 305)
NH₂-(CH₂)₆-oC*oC*oA*oU*U*dC*dA*dC*dC*
dA*dA*dC*dA*xdC*dG*oU*oC*oC*oC*oU,
(SEQ ID NO: 306)
NH₂-(CH₂)₆-oU*A*oC*oA*oG*dG*dT*dA*dG*
dT*dT*dC*dT*dC*dA*oU*oC*oC*oU*oG,
(SEQ ID NO: 307)
NH₂-(CH₂)₆-oG*oU*oA*oC*oA*dG*dG*dT*dA
*dG*dT*dT*dC*dT*dC*oA*oU*oC*oC*oU,
(SEQ ID NO: 308)
NH₂-(CH₂)₆-oA*oC*oC*oA*oG*G*dT*dA*dC*
dA*dG*dG*dT*dA*dG*oU*oU*oC*oU*oC,
(SEQ ID NO: 309)
NH₂-(CH₂)₆-oG*oA*oC*oC*oA*dG*dG*dT*dA
*dC*dA*dG*dG*dT*dA*oG*oU*oU*oC*oU,
(SEQ ID NO: 310)
NH₂-(CH₂)₆-oU*oG*oA*C*C*dA*dG*dG*dT*d
A*dC*dA*dG*dG*dT*oA*xoG*oU*oU*oC,
(SEQ ID NO: 311)
NH₂-(CH₂)₆-oC*C*C*A*oA*dA*dC*dT*dT*dG
*dC*dT*dC*dA*dG*oC*oA*oG*oU*oG,
(SEQ ID NO: 312)
NH₂-(CH₂)₆-oU*oG*oA*oC*xA*dA*dT*dC*dT
*dC*xdC*dG*dC*dC*dA*oG*oG*oU*oA*oG,
(SEQ ID NO: 313)
NH₂-(CH₂)₆-oA*oU*oG*oA*oC*dA*dA*dT*dC
*dT*dC*xdC*dG*dC*dC*oA*oG*oG*oU*oA,
(SEQ ID NO: 314)
NH₂-(CH₂)₆-oC*oA*oU*oG*oA*dC*dA*dA*dT
*dC*dT*dC*xdC*dG*dC*oC*oA*oG*oG*oU,
(SEQ ID NO: 315)
NH₂-(CH₂)₆-oC*oC*oA*oU*G*dA*dC*dA*dA*
dT*dC*dT*dC*xdC*dG*oC*oC*oA*oG*oG,
(SEQ ID NO: 316)
NH₂-(CH₂)₆-oG*oC*oC*oA*U*dG*dA*dC*dA*
dA*dT*dC*dT*dC*xdC*oG*oC*oC*A*oG,
(SEQ ID NO: 246)
NH₂-(CH₂)₆-oG*oG*oC*C*oA*dT*dG*dA*dC*
dA*dA*dT*dC*dT*dC*oC*oG*oC*oC*oA,
(SEQ ID NO: 317)
NH₂-(CH₂)₆-oU*oG*oG*oC*oC*dA*dT*dG*dA
*dC*dA*dA*dT*dC*dT*oC*oC*G*oC*oC,
(SEQ ID NO: 318)
NH₂-(CH₂)₆-oU*oG*oU*oG*oC*dA*xdC*dG*d
T*dA*dG*dC*dC*dA*dA*oG*oC*oC*G*oG,
(SEQ ID NO: 319)
NH₂-(CH₂)₆-oC*oU*oG*U*oG*dC*dA*xdC*dG
*dT*dA*dG*dC*dC*dA*oA*oG*oC*oC*oG,
(SEQ ID NO: 320)
NH₂-(CH₂)₆-oC*oA*oC*oA*oG*xdC*dG*dG*d
T*dC*dC*dA*dG*dC*dA*oG*G*oA*oU*oG,
(SEQ ID NO: 321)
NH₂-(CH₂)₆-oU*oG*oG*oC*oC*dA*dC*dA*dG
*xdC*dG*dG*dT*dC*dC*oA*oG*oC*oA*oG,
(SEQ ID NO: 322)
NH₂-(CH₂)₆-oA*G*C*oG*oC*dC*dC*dA*dC*d
C*dA*dG*dT*dC*dA*oC*oA*oC*oU*oC,
(SEQ ID NO: 323)
NH₂-(CH₂)₆-oC*oA*oG*oC*oG*dC*dC*dC*dA
*dC*dC*dA*dG*dT*dC*oA*oC*oA*xC*oU,
(SEQ ID NO: 254)
NH₂-(CH₂)₆-oC*oC*oA*oG*oC*dG*dC*dC*dC
*dA*dC*dC*dA*dG*dT*oC*oA*oC*oA*oC,
(SEQ ID NO: 255)
NH₂-(CH₂)₆-oG*oC*oG*oA*oA*dT*dA*dC*dA
*dC*dC*dC*dA*dG*xdC*G*oC*oC*oC*oA,
(SEQ ID NO: 256)
NH₂-(CH₂)₆-oG*oG*oC*oG*oA*dA*dT*dA*dC
*dA*dC*dC*dC*dA*dG*oC*oG*oC*oC*oC,
(SEQ ID NO: 324)
NH₂-(CH₂)₆-oU*oU*oG*oU*xA*dG*dT*dG*dG
*dA*xdC*dG*dA*dT*dC*oU*oU*oG*oC*oC,
(SEQ ID NO: 325)
NH₂-(CH₂)₆-oC*oU*oU*oG*oU*dA*dG*dT*dG
*dG*dA*xdC*dG*dA*dT*oC*oU*oU*oG*oC,
(SEQ ID NO: 326)
NH₂-(CH₂)₆-oC*oC*oU*oU*G*dT*dA*dG*dT*
dG*dG*dA*xdC*dG*dA*oU*oC*oU*oU*oG,
(SEQ ID NO: 327)
NH₂-(CH₂)₆-oC*oG*oG*oA*oG*dA*dC*dC*dA
*dT*dC*dC*dC*dA*dG*oU*oC*oG*oA*oG,
(SEQ ID NO: 328)
NH₂-(CH₂)₆-oG*oA*oA*oU*G*dT*dC*xdC*dG
*dA*dC*dA*dG*dT*dG*oU*oC*oU*oC*oC,
(SEQ ID NO: 329)
NH₂-(CH₂)₆-oC*oG*oA*oA*oU*dG*dT*dC*xd
C*dG*dA*dC*dA*dG*dT*oG*oU*oC*oU*oC,
(SEQ ID NO: 330)
NH₂-(CH₂)₆-oG*oG*oG*oC*oC*dT*dG*dG*dG
*dA*dC*dC*dT*dC*dA*oC*oU*oG*oU*oC,
(SEQ ID NO: 331)
NH₂-(CH₂)₆-oU*oG*oC*oA*oC*dG*dT*dG*dT
*dG*dG*dC*dT*dC*dA*oA*oG*oC*oA*oG,
(SEQ ID NO: 332)
NH₂-(CH₂)₆-oC*oC*A*oC*oU*dT*dC*dA*G*d
C*dT*dG*dT*dT*dT*oC*oA*oU*oC*oC,
(SEQ ID NO: 333)
NH₂-(CH₂)₆-oG*oC*oG*oU*oC*dA*dC*dC*dT
*xdC*dG*dG*dC*dC*dT*oC*oA*oG*oC*oC,
(SEQ ID NO: 334)
NH₂-(CH₂)₆-oA*oG*oC*G*oU*dC*dA*dC*dC*
dT*xdC*dG*dG*dC*dC*oU*oC*oA*oG*oC,
(SEQ ID NO: 335)
NH₂-(CH₂)₆-oC*oG*oU*oA*oG*dT*dT*dG*dA
*dC*dT*dG*dG*xdC*dG*oA*oA*oG*U*oU,
(SEQ ID NO: 336)
NH₂-(CH₂)₆-oG*oG*oG*oC*oC*xdC*dG*dG*d
A*dT*dC*dA*dC*dA*dG*oG*oA*C*oU*oG,
(SEQ ID NO: 337)
NH₂-(CH₂)₆-oU*oU*G*oC*oC*dC*dA*dT*dC*
dC*dA*xdC*dG*dT*dC*oA*oG*oG*oG*oC,
(SEQ ID NO: 338)
NH₂-(CH₂)₆-oG*oG*oA*oC*G*dG*dC*dC*xdC
*dG*dG*dC*dT*dT*dG*oC*oU*oG*oC*oC,
(SEQ ID NO: 339)
NH₂-(CH₂)₆-oU*oG*oG*oA*oA*dC*dA*xdC*d
G*dG*dA*xdC*dG*dG*dC*oC*oC*oG*G*oC,
(SEQ ID NO: 340)
NH₂-(CH₂)₆-oC*oA*oU*oC*oC*dA*dA*dA*dA
*xdC*dG*dT*dG*dG*dA*oU*oU*oG*G*oG,
and
(SEQ ID NO: 341)
NH₂-(CH₂)₆-oG*oC*oA*oU*oC*dC*dA*dA*dA
*dA*xdC*dG*dT*dG*dG*oA*oU*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage,
and optionally wherein a phosphodiester linkage or other moiety is present between the 5′-NH₂—(CH₂)₆— and the oligonucleotide.

In some embodiments, the oligonucleotide comprises a structure selected from:

(SEQ ID NO: 276)
+G*x+C*xA*xoC*dG*dT*dG*dT*dG*dG*xdC*
dT*xoC*oA*+A*+G,
(SEQ ID NO: 348)
+A*x+C*xoC*oA*dA*xdC*dA*xdC*dG*dT*xd
C*xdC*xoC*oU*x+C*+U,
(SEQ ID NO: 354)
x+C*x+C*xoC*oG*dG*dA*dT*xdC*dA*xdC*d
A*dG*oG*oA*x+C*+U,
(SEQ ID NO: 350)
+G*+U*xA*oG*dT*dT*dG*dA*xdC*dT*dG*dG
*xoC*oG*+A*+A,
(SEQ ID NO: 345)
x+C*+A*oU*oG*dA*xdC*dA*dA*dT*xdC*dT*
xdC*xoC*oG*x+C*x+C,
(SEQ ID NO: 286)
xoC*xoC*+A*+A*dA*xdC*dT*dT*dG*xdC*dT
*xdC*+A*+G*xoC*oA,
(SEQ ID NO: 352)
xoC*oU*+U*x+C*dA*dG*xdC*dT*dG*dT*dT*
dT*x+C*+A*oU*xoC,
(SEQ ID NO: 357)
x+C*+G*oU*xoC*dA*xdC*xdC*dT*xdC*dG*d
G*xdC*xoC*oU*x+C*+A,
(SEQ ID NO: 275)
x+C*+A*xoC*G*dT*dG*dT*dG*dG*xdC*dT*x
dC*oA*oA*+G*x+C,
(SEQ ID NO: 275)
xoC*oA*x+C*+G*dT*dG*dT*dG*dG*xdC*dT*
xdC*+A*+A*oG*xoC,
(SEQ ID NO: 276)
oG*xoC*+A*x+C*dG*dT*dG*dT*dG*dG*xdC*
dT*x+C*+A*oA*oG,
(SEQ ID NO: 342)
+A*x+C*oG*oU*dG*dT*dG*dG*xdC*dT*xdC*
dA*oA*oG*x+C*+A,
(SEQ ID NO: 342)
oA*xoC*+G*+U*dG*dT*dG*dG*xdC*dT*xdC*
dA*+A*+G*xoC*oA,
(SEQ ID NO: 278)
x+C*+A*oA*oA*xdC*dT*dT*dG*xdC*dT*xdC
*dA*oG*xoC*+A*+G,
(SEQ ID NO: 343)
+A*x+C*oU*oU*xdC*dA*dG*xdC*dT*dG*dT*
dT*oU*xoC*+A*+U,
(SEQ ID NO: 344)
+U*+A*oG*oU*dT*dG*dA*xdC*dT*dG*dG*xd
C*G*oA*+A*+G,
(SEQ ID NO: 281)
+G*x+C*xoC*xoC*dG*dG*dA*dT*dC*dA*xdC
*dA*oG*oG*+A*x+C,
(SEQ ID NO: 346)
+G*+U*xoC*xA*xdC*xdC*dT*xdC*dG*dG*xd
C*dC*oU*xoC*+A*+G,
(SEQ ID NO: 347)
x+C*x+C*xA*oG*dG*dT*dA*xdC*dA*dG*dG*
dT*oA*oG*+U*+U,
(SEQ ID NO: 286)
x+C*x+C*oA*oA*dA*xdC*dT*dT*dG*xdC*dT
*xdC*oA*oG*x+C*+A,
(SEQ ID NO: 349)
x+C*+A*xoC*oU*dT*xdC*dA*dG*xdC*dT*dG
*dT*oU*oU*x+C*+A,
(SEQ ID NO: 289)
+G*+G*xoC*xoC*xdC*dG*dG*dA*dT*xdC*dA
*xdC*oA*oG*+G*+A,
(SEQ ID NO: 351)
+A*+A*oA*xoC*dT*dT*dG*xdC*dT*xdC*dA*
dG*xoC*oA*+G*+U,
(SEQ ID NO: 352)
x+C*+U*oU*xoC*dA*dG*xdC*dT*dG*dT*dT*
dT*xoC*oA*+U*x+C,
(SEQ ID NO: 353)
+A*+G*oU*oU*dG*dA*xdC*dT*dG*dG*xdC*d
G*oA*oA*+G*+U,
(SEQ ID NO: 278)
xoC*oA*+A*+A*xdC*dT*dT*dG*xdC*dT*xdC
*dA*+G*x+C*oA*oG,
(SEQ ID NO: 343)
oA*xoC*+U*+U*xdC*dA*dG*xdC*dT*dG*dT*
dT*+U*x+C*oA*oU,
(SEQ ID NO: 344)
oU*xA*+G*+U*dT*dG*dA*xdC*dT*dG*dG*xd
C*+G*+A*oA*oG,
(SEQ ID NO: 281)
oG*xoC*x+C*x+C*dG*dG*dA*dT*dC*dA*dC*
dA*+G*+G*oA*xoC,
(SEQ ID NO: 349)
xoC*oA*x+C*+U*dT*xdC*dA*dG*xdC*dT*dG
*dT*+U*+U*xoC*oA,
(SEQ ID NO: 350)
oG*oU*+A*+G*dT*dT*dG*dA*xdC*dT*dG*dG
*x+C*+G*oA*oA,
(SEQ ID NO: 289)
oG*oG*x+C*x+C*xdC*dG*dG*dA*dT*xdC*dA
*xdC*+A*+G*oG*oA,
(SEQ ID NO: 351)
oA*A*+A*x+C*dT*dT*dG*xdC*dT*xdC*dA*d
G*x+C*+A*oG*oU,
(SEQ ID NO: 353)
oA*oG*+U*+U*dG*dA*xdC*dT*dG*dG*xdC*d
G*+A*+A*oG*oU,
(SEQ ID NO: 354)
xoC*xoC*x+C*+G*dG*dA*dT*xdC*dA*xdC*d
A*dG*+G*+A*xoC*oU,
(SEQ ID NO: 355)
x+C*x+C*oA*oU*dG*dA*xdC*dA*dA*dT*xdC
*dT*xoC*xoC*+G*x+C,
(SEQ ID NO: 356)
+A*+U*oG*oA*xdC*dA*dA*dT*xdC*dT*xdC*
xdC*oG*xoC*x+C*+A,
(SEQ ID NO: 358)
+U*x+C*oA*xoC*xdC*dT*xdC*dG*dG*xdC*x
dC*dT*xoC*oA*+G*x+C,
(SEQ ID NO: 359)
+A*x+C*xoC*xA*dG*dG*dT*dA*xdC*dA*dG*
dG*oU*oA*+G*+U,
(SEQ ID NO: 360)
x+C*+A*oG*oG*dT*dA*xdC*dA*dG*dG*dT*d
A*G*oU*+U*x+C,
(SEQ ID NO: 361)
x+C*+A*xoC*xoC*dA*dA*xdC*dA*xdC*dG*d
T*xdC*xoC*xoC*+U*x+C,
and
(SEQ ID NO: 362)
x+C*x+C*xA*oA*xdC*dA*xdC*dG*dT*xdC*x
dC*xdC*oU*xoC*+U*x+C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; and “*” indicates a phosphorothioate (PS) internucleoside linkage.

In some embodiments, the oligonucleotide is conjugated to an amine group at its 5′-end and comprises a structure selected from:

(SEQ ID NO: 276)
NH₂-(CH₂)₆-+G*x+C*oA*xoC*dG*dT*dG
*dT*dG*dG*xdC*dT*xoC*oA*+A*+G,
(SEQ ID NO: 348)
NH₂-(CH₂)₆-+A*x+C*xoC*oA*dA*xdC*d
A*xdC*dG*dT*xdC*xdC*xoC*oU*x+C*+U,
(SEQ ID NO: 354)
NH₂-(CH₂)₆-x+C*x+C*xoC*oG*dG*dA*d
T*xdC*dA*xdC*dA*dG*oG*oA*x+C*+U,
(SEQ ID NO: 350)
NH₂-(CH₂)₆-+G*+U*oA*oG*dT*dT*dG*d
A*xdC*dT*dG*dG*xoC*oG*+A*+A,
(SEQ ID NO: 345)
NH₂-(CH₂)₆-x+C*+A*oU*oG*dA*xdC*dA
*dA*dT*xdC*dT*xdC*xoC*G*x+C*x+C,
(SEQ ID NO: 286)
NH₂-(CH₂)₆-xoC*xoC*+A*+A*dA*xdC*d
T*dT*dG*xdC*dT*xdC*+A*+G*xoC*oA,
(SEQ ID NO: 352)
NH₂-(CH₂)₆-xoC*oU*+U*x+C*dA*dG*xd
C*dT*dG*dT*dT*dT*x+C*+A*oU*xoC,
(SEQ ID NO: 357)
NH₂-(CH₂)₆-x+C*+G*oU*xoC*dA*xdC*x
dC*dT*xdC*dG*dG*xdC*xoC*oU*x+C*+A,
(SEQ ID NO: 275)
NH₂-(CH₂)₆-x+C*+A*xoC*G*dT*dG*dT*
dG*dG*xdC*dT*xdC*oA*oA*+G*x+C,
(SEQ ID NO: 275)
NH₂-(CH₂)₆-xoC*oA*x+C*+G*dT*dG*dT
*dG*dG*xdC*dT*xdC*+A*+A*oG*xoC,
(SEQ ID NO: 276)
NH₂-(CH₂)₆-oG*xoC*+A*x+C*dG*dT*dG
*dT*dG*dG*xdC*dT*x+C*+A*oA*oG,
(SEQ ID NO: 342)
NH₂-(CH₂)₆-+A*x+C*oG*oU*dG*dT*dG*
dG*xdC*dT*xdC*dA*oA*oG*x+C*+A,
(SEQ ID NO: 342)
NH₂-(CH₂)₆-oA*xoC*+G*+U*dG*dT*dG*
dG*xdC*dT*xdC*dA*+A*+G*xoC*oA,
(SEQ ID NO: 278)
NH₂-(CH₂)₆-x+C*+A*oA*oA*xdC*dT*dT
*dG*xdC*dT*xdC*dA*oG*xoC*+A*+G,
(SEQ ID NO: 343)
NH₂-(CH₂)₆-+A*x+C*oU*oU*xdC*dA*dG
*xdC*dT*dG*dT*dT*oU*xoC*+A*+U,
(SEQ ID NO: 344)
NH₂-(CH₂)₆-+U*+A*oG*oU*dT*dG*dA*x
dC*dT*dG*dG*xdC*G*oA*+A*+G,
(SEQ ID NO: 281)
NH₂-(CH₂)₆-+G*x+C*xoC*xoC*dG*dG*d
A*dT*dC*dA*xdC*dA*oG*oG*+A*x+C,
(SEQ ID NO: 346)
NH₂-(CH₂)₆-+G*+U*xoC*oA*xdC*xdC*d
T*xdC*dG*dG*xdC*dC*oU*xoC*+A*+G,
(SEQ ID NO: 347)
NH₂-(CH₂)₆-x+C*x+C*xA*oG*dG*dT*dA
*xdC*dA*dG*dG*dT*oA*oG*+U*+U,
(SEQ ID NO: 286)
NH₂-(CH₂)₆-x+C*x+C*oA*oA*dA*xdC*d
T*dT*dG*xdC*dT*xdC*oA*oG*x+C*+A,
(SEQ ID NO: 349)
NH₂-(CH₂)₆-x+C*+A*xoC*oU*dT*xdC*d
A*dG*xdC*dT*dG*dT*oU*oU*x+C*+A,
(SEQ ID NO: 289)
NH₂-(CH₂)₆-+G*+G*xoC*xoC*xdC*dG*d
G*dA*dT*xdC*dA*xdC*oA*oG*+G*+A,
(SEQ ID NO: 351)
NH₂-(CH₂)₆-+A*+A*oA*xoC*dT*dT*dG*
xdC*dT*xdC*dA*dG*xoC*oA*+G*+U,
(SEQ ID NO: 352)
NH₂-(CH₂)₆-x+C*+U*oU*xoC*dA*dG*xd
C*dT*dG*dT*dT*dT*xoC*A*+U*x+C,
(SEQ ID NO: 353)
NH₂-(CH₂)₆-+A*+G*oU*oU*dG*dA*xdC*
dT*dG*dG*xdC*dG*oA*oA*+G*+U,
(SEQ ID NO: 278)
NH₂-(CH₂)₆-xoC*oA*+A*+A*xdC*dT*dT
*dG*xdC*dT*xdC*dA*+G*x+C*oA*oG,
(SEQ ID NO: 343)
NH₂-(CH₂)₆-oA*xoC*+U*+U*xdC*dA*dG
*xdC*dT*dG*dT*dT*+U*x+C*oA*oU,
(SEQ ID NO: 344)
NH₂-(CH₂)₆-oU*oA*+G*+U*dT*dG*dA*x
dC*dT*dG*dG*xdC*+G*+A*A*oG,
(SEQ ID NO: 281)
NH₂-(CH₂)₆-oG*xoC*x+C*x+C*dG*dG*d
A*dT*dC*dA*dC*dA*+G*+G*xA*xoC,
(SEQ ID NO: 349)
NH₂-(CH₂)₆-xoC*oA*x+C*+U*dT*xdC*d
A*dG*xdC*dT*dG*dT*+U*+U*xoC*oA,
(SEQ ID NO: 350)
NH₂-(CH₂)₆-oG*oU*+A*+G*dT*dT*dG*d
A*xdC*dT*dG*dG*x+C*+G*oA*oA,
(SEQ ID NO: 289)
NH₂-(CH₂)₆-oG*oG*x+C*x+C*xdC*dG*d
G*dA*dT*xdC*dA*xdC*+A*+G*oG*oA,
(SEQ ID NO: 351)
NH₂-(CH₂)₆-oA*oA*+A*x+C*dT*dT*dG*
xdC*dT*xdC*dA*dG*x+C*+A*oG*oU,
(SEQ ID NO: 353)
NH₂-(CH₂)₆-oA*oG*+U*+U*dG*dA*xdC*
dT*dG*dG*xdC*dG*+A*+A*oG*oU,
(SEQ ID NO: 354)
NH₂-(CH₂)₆-xoC*xoC*x+C*+G*dG*dA*d
T*xdC*dA*xdC*dA*dG*+G*+A*xoC*oU,
(SEQ ID NO: 355)
NH₂-(CH₂)₆-x+C*x+C*oA*oU*dG*dA*xd
C*dA*dA*dT*xdC*dT*xoC*xoC*+G*x+C,
(SEQ ID NO: 356)
NH₂-(CH₂)₆-+A*+U*oG*oA*xdC*dA*dA*
dT*xdC*dT*xdC*xdC*oG*xoC*x+C*+A,
(SEQ ID NO: 358)
NH₂-(CH₂)₆-+U*x+C*oA*xoC*xdC*dT*x
dC*dG*dG*xdC*xdC*dT*xoC*oA*+G*x+C,
(SEQ ID NO: 359)
NH₂-(CH₂)₆-+A*x+C*xoC*oA*dG*dG*dT
*dA*xdC*dA*dG*dG*oU*oA*+G*+U,
(SEQ ID NO: 360)
NH₂-(CH₂)₆-x+C*+A*oG*oG*dT*dA*xdC
*dA*dG*dG*dT*dA*oG*oU*+U*x+C,
(SEQ ID NO: 361)
NH₂-(CH₂)₆-x+C*+A*xoC*xoC*dA*dA*x
dC*dA*xdC*dG*dT*xdC*xoC*xoC*+U*x+C,
and
(SEQ ID NO: 362)
NH₂-(CH₂)₆-x+C*x+C*oA*oA*xdC*dA*x
dC*dG*dT*xdC*xdC*xdC*oU*xoC*+U*x+
C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; and “*” indicates a phosphorothioate (PS) internucleoside linkage,
and optionally wherein a phosphodiester linkage or other moiety is present between the 5′-NH₂—(CH₂)₆— and the oligonucleotide.

According to some aspects, methods of reducing DMPK expression in a muscle cell are provided herein. In some embodiments, a method comprises contacting the muscle cell with an effective amount of a complex disclosed herein to reduce DMPK expression in the muscle cell.

In some embodiments, reducing DMPK expression in the muscle cell comprises reducing the amount of DMPK RNA in the muscle cell, optionally wherein the DMPK RNA amount is reduced in the nucleus of the muscle cell, optionally wherein the DMPK RNA is a mutant DMPK mRNA.

In some embodiments, reducing DMPK expression in the muscle cell comprises reducing the amount of DMPK protein in the muscle cell.

According to some aspects, methods of treating myotonic dystrophy type 1 (DM1) are provided herein. In some embodiments, a method comprises administering to a subject in need thereof an effective amount of a complex disclosed herein.

In some embodiments, the administering results in a reduction of DMPK RNA in a muscle cell in the subject by at least 30%, optionally wherein the DMPK RNA is a DMPK mRNA.

In some embodiments, the administering results in a reduction of a DMPK RNA in the nucleus of a muscle cell in the subject, optionally wherein the DMPK RNA is a DMPK mRNA.

According to some aspects, oligonucleotides are provided herein. In some embodiments, an oligonucleotide comprises a structure selected from:

(SEQ ID NO: 302)
oC*oA*oU*oG*oG*dC*dA*dT*dA*dC*dA*
dC*dC*dT*dG*oG*oC*oC*oC*oG,
(SEQ ID NO: 303)
oC*oA*oC*oC*A*dA*dC*dA*xdC*dG*dT*
dC*dC*dC*dT*oC*oU*oC*oC*oU,
(SEQ ID NO: 304)
oU*oC*oA*oC*xC*dA*dA*dC*dA*xdC*dG
*dT*dC*dC*dC*oU*oC*oU*oC*oC,
(SEQ ID NO: 305)
oC*oC*oA*oU*oU*dC*dA*dC*dC*dA*dA*
dC*dA*xdC*dG*oU*oC*oC*oC*oU,
(SEQ ID NO: 306)
oU*oA*oC*xA*oG*dG*dT*dA*dG*dT*dT*
dC*dT*dC*dA*oU*oC*oC*oU*oG,
(SEQ ID NO: 307)
oG*oU*oA*C*xA*dG*dG*dT*dA*dG*dT*d
T*dC*dT*dC*A*oU*oC*oC*oU,
(SEQ ID NO: 308)
oA*oC*C*A*G*dG*dT*dA*dC*dA*dG*dG*
dT*dA*dG*oU*oU*oC*oU*oC,
(SEQ ID NO: 309)
oG*oA*oC*xC*xA*dG*dG*dT*dA*dC*dA*
dG*dG*dT*dA*oG*oU*oU*oC*oU,
(SEQ ID NO: 310)
oU*oG*xA*oC*oC*dA*dG*dG*dT*dA*dC*
dA*dG*dG*dT*oA*xoG*oU*oU*oC,
(SEQ ID NO: 311)
oC*oC*oC*oA*xA*dA*dC*dT*dT*dG*dC*
dT*dC*dA*dG*C*xA*oG*oU*oG,
(SEQ ID NO: 312)
oU*oG*xA*xC*xA*dA*dT*dC*dT*dC*xdC
*dG*dC*dC*dA*oG*oG*oU*oA*oG,
(SEQ ID NO: 313)
oA*oU*oG*oA*oC*dA*dA*dT*dC*dT*dC*
xdC*dG*dC*dC*oA*oG*oG*oU*oA,
(SEQ ID NO: 314)
oC*oA*oU*oG*oA*dC*dA*dA*dT*dC*dT*
dC*xdC*dG*dC*oC*oA*oG*oG*oU,
(SEQ ID NO: 315)
oC*oC*oA*oU*G*dA*dC*dA*dA*dT*dC*d
T*dC*xdC*dG*oC*xC*xA*oG*oG,
(SEQ ID NO: 316)
oG*oC*xC*xA*oU*dG*dA*dC*dA*dA*dT*
dC*dT*dC*xdC*G*oC*oC*oA*oG,
(SEQ ID NO: 246)
oG*oG*oC*xC*A*dT*dG*dA*dC*dA*dA*d
T*dC*dT*dC*oC*oG*oC*C*oA,
(SEQ ID NO: 317)
oU*oG*G*oC*oC*dA*dT*dG*dA*dC*dA*d
A*dT*dC*dT*oC*oC*oG*oC*oC,
(SEQ ID NO: 318)
oU*oG*oU*oG*oC*dA*xdC*dG*dT*dA*dG
*dC*dC*dA*dA*oG*oC*oC*oG*oG,
(SEQ ID NO: 319)
oC*oU*oG*oU*xG*dC*dA*xdC*G*dT*dA*
dG*dC*dC*dA*oA*oG*oC*oC*oG,
(SEQ ID NO: 320)
oC*xA*oC*xA*oG*xdC*dG*dG*dT*dC*dC
*dA*dG*dC*dA*G*oG*oA*U*oG,
(SEQ ID NO: 321)
oU*oG*oG*oC*oC*dA*dC*dA*dG*xdC*dG
*dG*dT*dC*dC*oA*oG*oC*oA*oG,
(SEQ ID NO: 322)
oA*oG*oC*oG*oC*dC*dC*dA*dC*dC*dA*
dG*dT*dC*dA*oC*A*oC*oU*oC,
(SEQ ID NO: 323)
oC*xA*oG*oC*oG*dC*dC*dC*dA*dC*dC*
dA*dG*dT*dC*oA*oC*xA*oC*oU,
(SEQ ID NO: 254)
oC*oC*oA*oG*C*dG*dC*dC*dC*dA*dC*d
C*dA*dG*dT*C*oA*oC*oA*oC,
(SEQ ID NO: 255)
oG*oC*oG*oA*xA*dT*dA*dC*dA*dC*dC*
dC*dA*dG*xdC*oG*oC*oC*oC*oA,
(SEQ ID NO: 256)
oG*oG*C*oG*oA*dA*dT*dA*dC*dA*dC*d
C*dC*dA*dG*oC*oG*oC*oC*oC,
(SEQ ID NO: 324)
oU*oU*oG*oU*oA*dG*dT*dG*dG*dA*xdC
*dG*dA*dT*dC*oU*oU*oG*oC*oC,
(SEQ ID NO: 325)
oC*oU*oU*oG*oU*dA*dG*dT*dG*dG*dA*
xdC*dG*dA*dT*oC*oU*oU*oG*oC,
(SEQ ID NO: 326)
oC*oC*oU*oU*xG*dT*dA*dG*dT*dG*dG*
dA*xdC*dG*dA*oU*oC*oU*oU*oG,
(SEQ ID NO: 327)
oC*oG*oG*oA*G*dA*dC*dC*dA*dT*dC*d
C*dC*dA*dG*oU*oC*oG*oA*oG,
(SEQ ID NO: 328)
oG*oA*oA*oU*oG*dT*dC*xdC*dG*dA*dC
*dA*dG*dT*dG*oU*oC*oU*oC*oC,
(SEQ ID NO: 329)
oC*G*oA*oA*oU*dG*dT*dC*xdC*dG*dA*
dC*dA*dG*dT*oG*oU*oC*oU*oC,
(SEQ ID NO: 330)
oG*oG*oG*oC*oC*dT*dG*dG*dG*dA*dC*
dC*dT*dC*dA*oC*oU*oG*oU*oC,
(SEQ ID NO: 331)
oU*oG*xC*xA*oC*dG*dT*dG*dT*dG*dG*
dC*dT*dC*dA*xA*oG*C*oA*oG,
(SEQ ID NO: 332)
oC*oC*xA*oC*oU*dT*dC*dA*dG*dC*dT*
dG*dT*dT*dT*oC*oA*oU*oC*oC,
(SEQ ID NO: 333)
oG*oC*oG*oU*oC*dA*dC*dC*dT*xdC*dG
*dG*dC*dC*dT*oC*oA*oG*oC*oC,
(SEQ ID NO: 334)
oA*oG*oC*oG*oU*dC*dA*dC*dC*dT*xdC
*dG*dG*dC*dC*oU*oC*oA*oG*oC,
(SEQ ID NO: 335)
oC*G*oU*oA*xG*dT*dT*dG*dA*dC*dT*d
G*dG*xdC*dG*xA*xA*oG*oU*oU,
(SEQ ID NO: 336)
oG*oG*oG*oC*oC*xdC*dG*dG*dA*dT*dC
*dA*dC*dA*dG*oG*oA*oC*oU*oG,
(SEQ ID NO: 337)
oU*oU*xG*oC*oC*dC*dA*dT*dC*dC*dA*
xdC*dG*dT*dC*oA*oG*oG*oG*oC,
(SEQ ID NO: 338)
oG*oG*xA*oC*oG*dG*dC*dC*xdC*dG*dG
*dC*dT*dT*dG*C*oU*oG*oC*oC,
(SEQ ID NO: 339)
oU*oG*G*xA*oA*dC*dA*xdC*dG*dG*dA*
xdC*dG*dG*dC*oC*oC*oG*oG*oC,
(SEQ ID NO: 340)
oC*oA*oU*oC*oC*dA*dA*dA*dA*xdC*dG
*dT*dG*dG*dA*oU*oU*oG*oG*G,
and
(SEQ ID NO: 341)
oG*oC*A*oU*oC*dC*dA*dA*dA*dA*xdC*
dG*dT*dG*dG*oA*oU*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

In some embodiments, the oligonucleotide is conjugated to an amine group at its 5′-end and comprises a structure selected from:

(SEQ ID NO: 302)
NH₂-(CH₂)₆-oC*oA*oU*oG*oG*dC*dA*d
T*dA*dC*dA*dC*dC*dT*dG*oG*oC*oC*o
C*oG,
(SEQ ID NO: 303)
NH₂-(CH₂)₆-oC*oA*oC*oC*oA*dA*dC*d
A*xdC*dG*dT*dC*dC*dC*dT*oC*oU*oC*
oC*oU,
(SEQ ID NO: 304)
NH₂-(CH₂)₆-oU*C*oA*oC*oC*dA*dA*dC
*dA*xdC*dG*dT*dC*dC*dC*oU*oC*oU*o
C*oC,
(SEQ ID NO: 305)
NH₂-(CH₂)₆-oC*oC*oA*oU*oU*dC*dA*d
C*dC*dA*dA*dC*dA*xdC*dG*oU*oC*oC*
oC*oU,
(SEQ ID NO: 306)
NH₂-(CH₂)₆-oU*oA*oC*oA*oG*dG*dT*d
A*dG*dT*dT*dC*dT*dC*dA*oU*oC*oC*o
U*oG,
(SEQ ID NO: 307)
NH₂-(CH₂)₆-oG*oU*oA*oC*xA*dG*dG*d
T*dA*dG*dT*dT*dC*dT*dC*oA*oU*oC*o
C*oU,
(SEQ ID NO: 308)
NH₂-(CH₂)₆-oA*oC*oC*oA*oG*dG*dT*d
A*dC*dA*dG*dG*dT*dA*dG*oU*oU*oC*o
U*oC,
(SEQ ID NO: 309)
NH₂-(CH₂)₆-oG*oA*C*oC*xA*dG*dG*dT
*dA*dC*dA*dG*dG*dT*dA*oG*oU*oU*oC
*oU,
(SEQ ID NO: 310)
NH₂-(CH₂)₆-oU*oG*oA*oC*oC*dA*dG*d
G*dT*dA*dC*dA*dG*dG*dT*oA*xoG*oU*
oU*oC,
(SEQ ID NO: 311)
NH₂-(CH₂)₆-oC*oC*C*oA*oA*dA*dC*dT
*dT*dG*dC*dT*dC*dA*dG*oC*xA*oG*oU
*oG,
(SEQ ID NO: 312)
NH₂-(CH₂)₆-oU*oG*oA*oC*oA*dA*dT*d
C*dT*dC*xdC*dG*dC*dC*dA*oG*oG*oU*
oA*oG,
(SEQ ID NO: 313)
NH₂-(CH₂)₆-oA*oU*oG*oA*oC*dA*dA*d
T*dC*dT*dC*xdC*dG*dC*dC*oA*oG*oG*
oU*oA,
(SEQ ID NO: 314)
NH₂-(CH₂)₆-oC*oA*oU*oG*oA*dC*dA*d
A*dT*dC*dT*dC*xdC*dG*dC*oC*oA*oG*
oG*oU,
(SEQ ID NO: 315)
NH₂-(CH₂)₆-oC*oC*oA*oU*G*dA*dC*dA
*dA*dT*dC*dT*dC*xdC*dG*oC*oC*xA*o
G*oG,
(SEQ ID NO: 316)
NH₂-(CH₂)₆-oG*oC*oC*oA*oU*dG*dA*d
C*dA*dA*dT*dC*dT*dC*xdC*oG*oC*oC*
oA*oG,
(SEQ ID NO: 246)
NH₂-(CH₂)₆-oG*oG*oC*C*oA*dT*dG*dA
*dC*dA*dA*dT*dC*dT*dC*oC*oG*oC*oC
*oA,
(SEQ ID NO: 317)
NH₂-(CH₂)₆-oU*G*oG*oC*oC*dA*dT*dG
*dA*dC*dA*dA*dT*dC*dT*oC*oC*xG*oC
*oC,
(SEQ ID NO: 318)
NH₂-(CH₂)₆-oU*oG*oU*oG*oC*dA*xdC*
dG*dT*dA*dG*dC*dC*dA*dA*oG*oC*oC*
oG*oG,
(SEQ ID NO: 319)
NH₂-(CH₂)₆-oC*oU*oG*oU*oG*dC*dA*x
dC*dG*dT*dA*dG*dC*dC*dA*oA*oG*oC*
oC*oG,
(SEQ ID NO: 320)
NH₂-(CH₂)₆-oC*oA*oC*oA*oG*xdC*dG*
dG*dT*dC*dC*dA*dG*dC*dA*oG*oG*oA*
oU*oG,
(SEQ ID NO: 321)
NH₂-(CH₂)₆-oU*oG*oG*oC*oC*dA*dC*d
A*dG*xdC*dG*dG*dT*dC*dC*oA*oG*oC*
oA*oG,
(SEQ ID NO: 322)
NH₂-(CH₂)₆-oA*oG*oC*oG*oC*dC*dC*d
A*dC*dC*dA*dG*dT*dC*dA*oC*xA*oC*o
U*oC,
(SEQ ID NO: 323)
NH₂-(CH₂)₆-oC*oA*oG*oC*G*dC*dC*dC
*dA*dC*dC*dA*dG*dT*dC*xA*oC*xA*oC
*oU,
(SEQ ID NO: 254)
NH₂-(CH₂)₆-oC*oC*oA*oG*oC*dG*dC*d
C*dC*dA*dC*dC*dA*dG*dT*oC*oA*oC*o
A*oC,
(SEQ ID NO: 255)
NH₂-(CH₂)₆-oG*oC*oG*oA*oA*dT*dA*d
C*dA*dC*dC*dC*dA*dG*xdC*oG*C*oC*o
C*oA,
(SEQ ID NO: 256)
NH₂-(CH₂)₆-oG*G*oC*oG*oA*dA*dT*dA
*dC*dA*dC*dC*dC*dA*dG*oC*oG*oC*oC
*oC,
(SEQ ID NO: 324)
NH₂-(CH₂)₆-oU*oU*oG*oU*oA*dG*dT*d
G*dG*dA*xdC*dG*dA*dT*dC*oU*oU*oG*
oC*oC,
(SEQ ID NO: 325)
NH₂-(CH₂)₆-oC*oU*oU*oG*oU*dA*dG*d
T*dG*dG*dA*xdC*dG*dA*dT*oC*oU*oU*
oG*oC,
(SEQ ID NO: 326)
NH₂-(CH₂)₆-oC*oC*oU*oU*oG*dT*dA*d
G*dT*dG*dG*dA*xdC*dG*dA*oU*oC*oU*
oU*oG,
(SEQ ID NO: 327)
NH₂-(CH₂)₆-oC*oG*oG*oA*oG*dA*dC*d
C*dA*dT*dC*dC*dC*dA*dG*oU*oC*oG*o
A*oG,
(SEQ ID NO: 328)
NH₂-(CH₂)₆-oG*oA*oA*oU*G*dT*dC*xd
C*dG*dA*dC*dA*dG*dT*dG*oU*oC*oU*o
C*oC,
(SEQ ID NO: 329)
NH₂-(CH₂)₆-oC*oG*oA*oA*oU*dG*dT*d
C*xdC*dG*dA*dC*dA*dG*dT*oG*oU*oC*
oU*oC,
(SEQ ID NO: 330)
NH₂-(CH₂)₆-oG*oG*oG*oC*oC*dT*dG*d
G*dG*dA*dC*dC*dT*dC*dA*oC*oU*oG*o
U*oC,
(SEQ ID NO: 331)
NH₂-(CH₂)₆-oU*oG*oC*oA*oC*dG*dT*d
G*dT*dG*dG*dC*dT*dC*dA*xA*G*oC*oA
*oG,
(SEQ ID NO: 332)
NH₂-(CH₂)₆-oC*oC*oA*oC*oU*dT*dC*d
A*dG*dC*dT*dG*dT*dT*dT*oC*oA*oU*o
C*oC,
(SEQ ID NO: 333)
NH₂-(CH₂)₆-oG*oC*oG*oU*oC*dA*dC*d
C*dT*xdC*dG*dG*dC*dC*dT*oC*oA*oG*
oC*oC,
(SEQ ID NO: 334)
NH₂-(CH₂)₆-oA*oG*oC*oG*oU*dC*dA*d
C*dC*dT*xdC*dG*dG*dC*dC*oU*oC*oA*
oG*oC,
(SEQ ID NO: 335)
NH₂-(CH₂)₆-oC*oG*oU*oA*G*dT*dT*dG
*dA*dC*dT*dG*dG*xdC*dG*xA*xA*oG*o
U*oU,
(SEQ ID NO: 336)
NH₂-(CH₂)₆-oG*oG*G*oC*oC*xdC*dG*d
G*dA*dT*dC*dA*dC*dA*dG*oG*oA*oC*o
U*oG,
(SEQ ID NO: 337)
NH₂-(CH₂)₆-oU*oU*G*oC*C*dC*dA*dT*
dC*dC*dA*xdC*dG*dT*dC*A*oG*oG*oG*
oC,
(SEQ ID NO: 338)
NH₂-(CH₂)₆-oG*oG*oA*oC*oG*dG*dC*d
C*xdC*dG*dG*dC*dT*dT*dG*oC*oU*oG*
oC*oC,
(SEQ ID NO: 339)
NH₂-(CH₂)₆-oU*oG*oG*oA*oA*dC*dA*x
dC*dG*dG*dA*xdC*dG*dG*dC*oC*xC*oG
*oG*oC,
(SEQ ID NO: 340)
NH₂-(CH₂)₆-oC*oA*oU*oC*oC*dA*dA*d
A*dA*xdC*dG*dT*dG*dG*dA*oU*oU*oG*
G*oG,
and
(SEQ ID NO: 341)
NH₂-(CH₂)₆-oG*oC*oA*oU*oC*dC*dA*d
A*dA*dA*xdC*dG*dT*dG*dG*A*oU*oU*o
G*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage,
and optionally wherein a phosphodiester linkage or other moiety is present between the 5′-NH₂—(CH₂)₆— and the oligonucleotide.

In some embodiments, an oligonucleotide comprises a structure selected from:

(SEQ ID NO: 276)
+G*x+C*oA*xoC*dG*dT*dG*dT*dG*dG*x
dC*dT*xoC*oA*+A*+G,
(SEQ ID NO: 348)
+A*x+C*xoC*oA*dA*xdC*dA*xdC*dG*dT
*xdC*xdC*xoC*oU*x+C*+U,
(SEQ ID NO: 354)
x+C*x+C*xoC*xG*dG*dA*dT*xdC*dA*xd
C*dA*dG*oG*oA*x+C*+U,
(SEQ ID NO: 350)
+G*+U*xA*xG*dT*dT*dG*dA*xdC*dT*dG
*dG*xoC*oG*+A*+A,
(SEQ ID NO: 345)
x+C*+A*oU*G*dA*xdC*dA*dA*dT*xdC*d
T*xdC*xoC*oG*x+C*x+C,
(SEQ ID NO: 286)
xoC*xoC*+A*+A*dA*xdC*dT*dT*dG*xdC
*dT*xdC*+A*+G*xoC*oA,
(SEQ ID NO: 352)
xoC*oU*+U*x+C*dA*dG*xdC*dT*dG*dT*
dT*dT*x+C*+A*oU*xoC,
(SEQ ID NO: 357)
x+C*+G*oU*xoC*dA*xdC*xdC*dT*xdC*d
G*dG*xdC*xoC*oU*x+C*+A,
(SEQ ID NO: 275)
x+C*+A*xoC*xG*dT*dG*dT*dG*dG*xdC*
dT*xdC*oA*oA*+G*x+C,
(SEQ ID NO: 275)
xoC*xA*x+C*+G*dT*dG*dT*dG*dG*xdC*
dT*xdC*+A*+A*oG*xoC,
(SEQ ID NO: 276)
oG*xoC*+A*x+C*dG*dT*dG*dT*G*dG*xd
C*dT*x+C*+A*A*oG,
(SEQ ID NO: 342)
+A*x+C*oG*oU*dG*dT*dG*dG*xdC*dT*x
dC*dA*oA*oG*x+C*+A,
(SEQ ID NO: 342)
oA*xoC*+G*+U*dG*dT*dG*dG*xdC*dT*x
dC*dA*+A*+G*xoC*oA,
(SEQ ID NO: 278)
x+C*+A*oA*xA*xdC*dT*dT*dG*xdC*dT*
xdC*dA*oG*xoC*+A*+G,
(SEQ ID NO: 343)
+A*x+C*oU*U*xdC*dA*dG*xdC*dT*dG*d
T*dT*oU*xoC*+A*+U,
(SEQ ID NO: 344)
+U*+A*oG*oU*dT*dG*dA*xdC*dT*dG*dG
*xdC*oG*oA*+A*+G,
(SEQ ID NO: 281)
+G*x+C*xoC*xoC*dG*dG*dA*dT*dC*dA*
xdC*dA*oG*oG*+A*x+C,
(SEQ ID NO: 346)
+G*+U*xoC*xA*xdC*xdC*dT*xdC*dG*dG
*xdC*dC*oU*xoC*+A*+G,
(SEQ ID NO: 347)
x+C*x+C*xA*G*dG*dT*dA*xdC*dA*dG*d
G*dT*A*oG*+U*+U,
(SEQ ID NO: 286)
x+C*x+C*xA*oA*dA*xdC*dT*dT*dG*xdC
*dT*xdC*oA*oG*x+C*+A,
(SEQ ID NO: 349)
x+C*+A*xoC*oU*dT*xdC*dA*dG*xdC*dT
*dG*dT*oU*oU*x+C*+A,
(SEQ ID NO: 289)
+G*+G*xoC*xoC*xdC*dG*dG*dA*dT*xdC
*dA*xdC*A*oG*+G*+A,
(SEQ ID NO: 351)
+A*+A*xA*xoC*dT*dT*dG*xdC*dT*xdC*
dA*dG*xoC*oA*+G*+U,
(SEQ ID NO: 352)
x+C*+U*oU*xoC*dA*dG*xdC*dT*dG*dT*
dT*dT*xoC*oA*+U*x+C,
(SEQ ID NO: 353)
+A*+G*oU*oU*dG*dA*xdC*dT*dG*dG*xd
C*dG*oA*oA*+G*+U,
(SEQ ID NO: 278)
xoC*oA*+A*+A*xdC*dT*dT*dG*xdC*dT*
xdC*dA*+G*x+C*xA*oG,
(SEQ ID NO: 343)
oA*xoC*+U*+U*xdC*dA*dG*xdC*dT*dG*
dT*dT*+U*x+C*oA*oU,
(SEQ ID NO: 344)
oU*oA*+G*+U*dT*dG*dA*xdC*dT*dG*dG
*xdC*+G*+A*xA*oG,
(SEQ ID NO: 281)
oG*xoC*x+C*x+C*dG*dG*dA*dT*dC*dA*
dC*dA*+G*+G*oA*xoC,
(SEQ ID NO: 349)
xoC*xA*x+C*+U*dT*xdC*dA*dG*xdC*dT
*dG*dT*+U*+U*xoC*oA,
(SEQ ID NO: 350)
oG*oU*+A*+G*dT*dT*dG*dA*xdC*dT*dG
*dG*x+C*+G*oA*oA,
(SEQ ID NO: 289)
oG*oG*x+C*x+C*xdC*dG*dG*dA*dT*xdC
*dA*xdC*+A*+G*oG*oA,
(SEQ ID NO: 351)
oA*oA*+A*x+C*dT*dT*dG*xdC*dT*xdC*
dA*dG*x+C*+A*oG*oU,
(SEQ ID NO: 353)
oA*oG*+U*+U*dG*dA*xdC*dT*dG*dG*xd
C*dG*+A*+A*oG*oU,
(SEQ ID NO: 354)
xoC*xoC*x+C*+G*dG*dA*dT*xdC*dA*xd
C*dA*dG*+G*+A*xoC*oU,
(SEQ ID NO: 355)
x+C*x+C*xA*oU*dG*dA*xdC*dA*dA*dT*
xdC*dT*xoC*xoC*+G*x+C,
(SEQ ID NO: 356)
+A*+U*oG*oA*xdC*dA*dA*dT*xdC*dT*x
dC*xdC*oG*xoC*x+C*+A,
(SEQ ID NO: 358)
+U*x+C*oA*xoC*xdC*dT*xdC*dG*dG*xd
C*xdC*dT*xoC*oA*+G*x+C,
(SEQ ID NO: 359)
+A*x+C*xoC*oA*dG*dG*dT*dA*xdC*dA*
dG*dG*oU*oA*+G*+U,
(SEQ ID NO: 360)
x+C*+A*oG*oG*dT*dA*xdC*dA*dG*dG*d
T*dA*oG*oU*+U*x+C,
(SEQ ID NO: 361)
x+C*+A*xoC*xoC*dA*dA*xdC*dA*xdC*d
G*dT*xdC*xoC*xoC*+U*x+C,
and
(SEQ ID NO: 362)
x+C*x+C*oA*oA*xdC*dA*xdC*dG*dT*xd
C*xdC*xdC*oU*xoC*+U*x+C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; “*” indicates a phosphorothioate (PS) internucleoside linkage.

In some embodiments, the oligonucleotide is conjugated to an amine group at its 5′-end and comprises a structure selected from:

(SEQ ID NO: 276)
NH₂-(CH₂)₆-+G*x+C*oA*xoC*dG*dT*dG
*dT*dG*dG*xdC*dT*xoC*oA*+A*+G,
(SEQ ID NO: 348)
NH₂-(CH₂)₆-+A*x+C*xoC*oA*dA*xdC*d
A*xdC*dG*dT*xdC*xdC*xoC*oU*x+C*+U,
(SEQ ID NO: 354)
NH₂-(CH₂)₆-x+C*x+C*xoC*oG*dG*dA*d
T*xdC*dA*xdC*dA*dG*oG*oA*x+C*+U,
(SEQ ID NO: 350)
NH₂-(CH₂)₆-+G*+U*oA*oG*dT*dT*dG*d
A*xdC*dT*dG*dG*xoC*oG*+A*+A,
(SEQ ID NO: 345)
NH₂-(CH₂)₆-x+C*+A*oU*oG*dA*xdC*dA
*dA*dT*xdC*dT*xdC*xoC*oG*x+C*x+C,
(SEQ ID NO: 286)
NH₂-(CH₂)₆-xoC*xoC*+A*+A*dA*xdC*d
T*dT*dG*xdC*dT*xdC*+A*+G*xoC*oA,
(SEQ ID NO: 352)
NH₂-(CH₂)₆-xoC*oU*+U*x+C*dA*dG*xd
C*dT*dG*dT*dT*dT*x+C*+A*oU*xoC,
(SEQ ID NO: 357)
NH₂-(CH₂)₆-x+C*+G*oU*xoC*dA*xdC*x
dC*dT*xdC*dG*dG*xdC*xoC*oU*x+C*+A,
(SEQ ID NO: 275)
NH₂-(CH₂)₆-x+C*+A*xoC*oG*dT*dG*dT
*dG*dG*xdC*dT*xdC*oA*oA*+G*x+C,
(SEQ ID NO: 275)
NH₂-(CH₂)₆-xoC*oA*x+C*+G*dT*dG*dT
*dG*dG*xdC*dT*xdC*+A*+A*oG*xoC,
(SEQ ID NO: 276)
NH₂-(CH₂)₆-oG*xoC*+A*x+C*dG*dT*dG
*dT*dG*dG*xdC*dT*x+C*+A*oA*oG,
(SEQ ID NO: 342)
NH₂-(CH₂)₆-+A*x+C*oG*oU*dG*dT*dG*
dG*xdC*dT*xdC*dA*oA*oG*x+C*+A,
(SEQ ID NO: 342)
NH₂-(CH₂)₆-oA*xoC*+G*+U*dG*dT*G*d
G*xdC*dT*xdC*dA*+A*+G*xoC*oA,
(SEQ ID NO: 278)
NH₂-(CH₂)₆-x+C*+A*oA*oA*xdC*dT*dT
*dG*xdC*dT*xdC*dA*oG*xoC*+A*+G,
(SEQ ID NO: 343)
NH₂-(CH₂)₆-+A*x+C*oU*oU*xdC*dA*dG
*xdC*dT*dG*dT*dT*oU*xoC*+A*+U,
(SEQ ID NO: 344)
NH₂-(CH₂)₆-+U*+A*G*oU*dT*dG*dA*xd
C*dT*dG*dG*xdC*oG*oA*+A*+G,
(SEQ ID NO: 281)
NH₂-(CH₂)₆-+G*x+C*xoC*xoC*dG*dG*d
A*dT*dC*dA*xdC*dA*oG*oG*+A*x+C,
(SEQ ID NO: 346)
NH₂-(CH₂)₆-+G*+U*xoC*oA*xdC*xdC*d
T*xdC*dG*dG*xdC*dC*oU*xoC*+A*+G,
(SEQ ID NO: 347)
NH₂-(CH₂)₆-x+C*x+C*oA*oG*dG*dT*dA
*xdC*dA*dG*dG*dT*oA*G*+U*+U,
(SEQ ID NO: 286)
NH₂-(CH₂)₆-x+C*x+C*A*A*dA*xdC*dT*
dT*dG*xdC*dT*xdC*oA*oG*x+C*+A,
(SEQ ID NO: 349)
NH₂-(CH₂)₆-x+C*+A*xoC*oU*dT*xdC*d
A*dG*xdC*dT*dG*dT*oU*oU*x+C*+A,
(SEQ ID NO: 289)
NH₂-(CH₂)₆-+G*+G*xoC*xoC*xdC*dG*d
G*dA*dT*xdC*dA*xdC*oA*oG*+G*+A,
(SEQ ID NO: 351)
NH₂-(CH₂)₆-+A*+A*oA*xoC*dT*dT*dG*
xdC*dT*xdC*dA*dG*xoC*oA*+G*+U,
(SEQ ID NO: 352)
NH₂-(CH₂)₆-x+C*+U*oU*xoC*dA*dG*xd
C*dT*dG*dT*dT*dT*xoC*oA*+U*x+C,
(SEQ ID NO: 353)
NH₂-(CH₂)₆-+A*+G*oU*oU*dG*dA*xdC*
dT*dG*dG*xdC*dG*oA*oA*+G*+U,
(SEQ ID NO: 278)
NH₂-(CH₂)₆-xoC*oA*+A*+A*xdC*dT*dT
*dG*xdC*dT*xdC*dA*+G*x+C*oA*oG,
(SEQ ID NO: 343)
NH₂-(CH₂)₆-oA*xoC*+U*+U*xdC*dA*dG
*xdC*dT*dG*dT*dT*+U*x+C*oA*oU,
(SEQ ID NO: 344)
NH₂-(CH₂)₆-oU*oA*+G*+U*dT*dG*dA*x
dC*dT*dG*dG*xdC*+G*+A*oA*oG,
(SEQ ID NO: 281)
NH₂-(CH₂)₆-oG*xoC*x+C*x+C*dG*dG*d
A*dT*dC*dA*dC*dA*+G*+G*oA*xoC,
(SEQ ID NO: 349)
NH₂-(CH₂)₆-xoC*oA*x+C*+U*dT*xdC*d
A*dG*xdC*dT*dG*dT*+U*+U*xoC*oA,
(SEQ ID NO: 350)
NH₂-(CH₂)₆-oG*oU*+A*+G*dT*dT*dG*d
A*xdC*dT*dG*dG*x+C*+G*oA*oA,
(SEQ ID NO: 289)
NH₂-(CH₂)₆-oG*oG*x+C*x+C*xdC*dG*d
G*dA*dT*xdC*dA*xdC*+A*+G*oG*oA,
(SEQ ID NO: 351)
NH₂-(CH₂)₆-oA*oA*+A*x+C*dT*dT*dG*
xdC*dT*xdC*dA*dG*x+C*+A*oG*oU,
(SEQ ID NO: 353)
NH₂-(CH₂)₆-oA*oG*+U*+U*dG*dA*xdC*
dT*dG*dG*xdC*dG*+A*+A*oG*oU,
(SEQ ID NO: 354)
NH₂-(CH₂)₆-xoC*xoC*x+C*+G*dG*dA*d
T*xdC*dA*xdC*dA*dG*+G*+A*xoC*oU,
(SEQ ID NO: 355)
NH₂-(CH₂)₆-x+C*x+C*oA*oU*dG*dA*xd
C*dA*dA*dT*xdC*dT*xoC*xoC*+G*x+C,
(SEQ ID NO: 356)
NH₂-(CH₂)₆-+A*+U*oG*oA*xdC*dA*dA*
dT*xdC*dT*xdC*xdC*oG*xoC*x+C*+A,
(SEQ ID NO: 358)
NH₂-(CH₂)₆-+U*x+C*oA*xoC*xdC*dT*x
dC*dG*dG*xdC*xdC*dT*xoC*oA*+G*x+C,
(SEQ ID NO: 359)
NH₂-(CH₂)₆-+A*x+C*xoC*oA*dG*dG*dT
*dA*xdC*dA*dG*dG*oU*oA*+G*+U,
(SEQ ID NO: 360)
NH₂-(CH₂)₆-x+C*+A*oG*oG*dT*dA*xdC
*dA*dG*dG*dT*dA*oG*oU*+U*x+C,
(SEQ ID NO: 361)
NH₂-(CH₂)₆-x+C*+A*xoC*xoC*dA*dA*x
dC*dA*xdC*dG*dT*xdC*xoC*xoC*+U*x+
C,
and
(SEQ ID NO: 362)
NH₂-(CH₂)₆-x+C*x+C*oA*oA*xdC*dA*x
dC*dG*dT*xdC*xdC*xdC*oU*xoC*+U*x+
C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; “*” indicates a phosphorothioate (PS) internucleoside linkage, and optionally wherein a phosphodiester linkage or other moiety is present between the 5′-NH₂—(CH₂)₆— and the oligonucleotide.

According to some aspects, compositions comprising an oligonucleotide are provided herein. In some embodiments, a composition comprises an oligonucleotide disclosed herein in sodium salt form.

### BRIEF DESCRIPTION OF THE DRAWINGS

FIGS. 1A-1H show that conjugates having an anti-TfR1 Fab conjugated to a DMPK-targeting oligonucleotide delivered oligonucleotide to various muscle tissues and reduced mouse Dmpk expression in a mouse model that expresses human TfR1. The DMPK-targeting oligonucleotide was conjugated to anti-TfR1 Fab 3M12-VH4/Vk3. FIG. 1A shows that the conjugate reduced mouse wild-type Dmpk in tibialis anterior by 79%. FIG. 1B shows that the conjugate reduced mouse wild-type Dmpk in gastrocnemius by 76%. FIG. 1C shows that the conjugate reduced mouse wild-type Dmpk in the heart by 70%. FIG. 1D shows that the conjugate reduced mouse wild-type Dmpk and in diaphragm by 88%. FIGS. 1E-1H show oligonucleotide distributions in tibialis anterior (FIG. 1E), gastrocnemius (FIG. 1F), heart (FIG. 1G), and diaphragm (FIG. 1H).

FIGS. 2A-2D show toxic human DMPK knockdown in heart (FIG. 2A), diaphragm (FIG. 2B), gastrocnemius (FIG. 2C) and tibialis anterior (FIG. 2D) muscle tissues of hTfR1/DMSXL mice after treatment with vehicle control or DMPK-targeting ASOs (ASO58, ASO47, ASO61, or ASO66) conjugated to anti-TfR1 Fab 3M12-VH4/Vκ3. (*, P≤0.05; **, P≤0.01; ***, P≤0.001; ****, P≤0.0001, as analyzed by one-way ANOVA).

### DETAILED DESCRIPTION OF INVENTION

Some aspects of the present disclosure provide oligonucleotides designed to target DMPK RNAs. In some embodiments, the disclosure provides oligonucleotides complementary with DMPK RNA that are useful for reducing levels of toxic DMPK having disease-associated repeat expansions, e.g., in a subject having or suspected of having myotonic dystrophy. In some embodiments, the oligonucleotides are designed to direct RNAse H mediated degradation of the target DMPK RNA. In some embodiments, the oligonucleotides are designed to direct RNAse H mediated degradation of the target DMPK RNA residing in the nucleus of cells, e.g., muscle cells (e.g., myotubes) or central nervous system (CNS) cells. In some embodiments, the oligonucleotides are designed to have desirable bioavailability and/or serum-stability properties. In some embodiments, the oligonucleotides are designed to have desirable binding affinity properties. In some embodiments, the oligonucleotides are designed to have desirable toxicity profiles. In some embodiments, the oligonucleotides are designed to have low-complement activation and/or cytokine induction properties.

In some aspects, the present disclosure provides complexes comprising muscle-targeting agents covalently linked to the DMPK-targeting oligonucleotides described herein for effective delivery of the oligonucleotides to muscle cells. In some embodiments, complexes are provided for targeting a DMPK allele that comprises an expanded disease-associated-repeat to treat subjects having DM1. In some embodiments, complexes provided herein may comprise oligonucleotides that inhibit expression of a DMPK allele comprising an expanded disease-associated-repeat. As another example, complexes may comprise oligonucleotides that interfere with the binding of a disease-associated DMPK mRNA to a muscleblind-like protein (e.g., MBNL1, 2, and/or (e.g., and) 3), thereby reducing a toxic effect of a disease-associated DMPK allele.

Further aspects of the disclosure, including a description of defined terms, are provided below.

### I. Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (7) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics Information System® www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

CDR-grafted antibody: The term “CDR-grafted antibody” refers to antibodies which comprise heavy and light chain variable region sequences from one species but in which the sequences of one or more of the CDR regions of VH and/or (e.g., and) VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleosides or two sets of nucleosides. In particular, complementary is a term that characterizes an extent of hydrogen bond pairing that brings about binding between two nucleosides or two sets of nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human transferrin receptor and non-human primate transferrin receptor) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Disease-associated-repeat: As used herein, the term “disease-associated-repeat” refers to a repeated nucleotide sequence at a genomic location for which the number of units of the repeated nucleotide sequence is correlated with and/or (e.g., and) directly or indirectly contributes to, or causes, genetic disease such as DM1. Each repeating unit of a disease associated repeat may be 2, 3, 4, 5 or more nucleotides in length. For example, in some embodiments, a disease associated repeat is a dinucleotide repeat. In some embodiments, a disease associated repeat is a trinucleotide repeat. In some embodiments, a disease associated repeat is a tetranucleotide repeat. In some embodiments, a disease associated repeat is a pentanucleotide repeat. In some embodiments, embodiments, the disease-associated-repeat comprises CAG repeats, CTG repeats, CUG repeats, CGG repeats, CCTG repeats, or a nucleotide complement of any thereof. In some embodiments, a disease-associated-repeat is in a non-coding portion of a gene. However, in some embodiments, a disease-associated-repeat is in a coding region of a gene. In some embodiments, a disease-associated-repeat is expanded from a normal state to a length that directly or indirectly contributes to, or causes, genetic disease. In some embodiments, a disease-associated-repeat is in RNA (e.g., an RNA transcript). In some embodiments, a disease-associated-repeat is in DNA (e.g., a chromosome, a plasmid). In some embodiments, a disease-associated-repeat is expanded in a chromosome of a germline cell. In some embodiments, a disease-associated-repeat is expanded in a chromosome of a somatic cell. In some embodiments, a disease-associated-repeat is expanded to a number of repeating units that is associated with congenital onset of disease. In some embodiments, a disease-associated-repeat is expanded to a number of repeating units that is associated with childhood onset of disease. In some embodiments, a disease-associated-repeat is expanded to a number of repeating units that is associated with adult onset of disease. In DM1, a trinucleotide repeat region of CTG units in the 3′ untranslated region (3′-UTR) of DMPK is disease-associated. A normal DMPK allele comprises about 5 to about 37 CTG repeat units, whereas in patients with DM1, the length of the CTG repeat region is significantly increased, up to hundreds or thousands of trinucleotide repeats.

DMPK: As used herein, the term “DMPK” refers to a gene that encodes myotonin-protein kinase (also known as myotonic dystrophy protein kinase or dystrophia myotonica protein kinase), a serine/threonine protein kinase. Substrates for this enzyme may include myogenin, the beta-subunit of the L-type calcium channels, and phospholemman. In some embodiments, DMPK may be a human (Gene ID: 1760), non-human primate (e.g., Gene ID: 456139, Gene ID: 715328), or rodent gene (e.g., Gene ID: 13400). In humans, a CTG repeat expansion in the 3′ non-coding, untranslated region of DMPK is associated with myotonic dystrophy type I (DM1). In addition, multiple human transcript variants (e.g., as annotated under GenBank RefSeq Accession Numbers: NM_001081563.2, NM_004409.4, NM_001081560.2, NM_001081562.2, NM_001288764.1, NM_001288765.1, and NM_001288766.1) have been characterized that encode different protein isoforms.

DMPK allele: As used herein, the term “DMPK allele” refers to any one of alternative forms (e.g., wild-type or mutant forms) of a DMPK gene. In some embodiments, a DMPK allele may encode for wild-type myotonin-protein kinase that retains its normal and typical functions. In some embodiments, a DMPK allele may comprise one or more disease-associated-repeat expansions. In some embodiments, normal subjects have two DMPK alleles comprising in the range of 5 to 37 repeat units. In some embodiments, the number of CTG repeat units in subjects having DM1 is in the range of about 50 to about 3,000 or more with higher numbers of repeats leading to an increased severity of disease. In some embodiments, mildly affected DM1 subjects have at least one DMPK allele having in the range of 50 to 150 repeat units. In some embodiments, subjects with classic DM1 have at least one DMPK allele having in the range of 100 to 1,000 or more repeat units. In some embodiments, subjects having DM1 with congenital onset may have at least one DMPK allele comprising more than 2,000 repeat units.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding non-human CDR sequences. In one embodiment, humanized anti-TfR1 antibodies and antigen binding portions are provided. Such antibodies may be generated by obtaining murine anti-TfR1 monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Isolated antibody: An “isolated antibody”, as used herein, is intended to refer to an antibody that is substantially free of other antibodies having different antigenic specificities (e.g., an isolated antibody that specifically binds transferrin receptor is substantially free of antibodies that specifically bind antigens other than transferrin receptor). An isolated antibody that specifically binds transferrin receptor complex may, however, have cross-reactivity to other antigens, such as transferrin receptor molecules from other species. Moreover, an isolated antibody may be substantially free of other cellular material and/or (e.g., and) chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Muscle-targeting agent: As used herein, the term, “muscle-targeting agent,” refers to a molecule that specifically binds to an antigen expressed on muscle cells. The antigen in or on muscle cells may be a membrane protein, for example an integral membrane protein or a peripheral membrane protein. Typically, a muscle-targeting agent specifically binds to an antigen on muscle cells that facilitates internalization of the muscle-targeting agent (and any associated molecular payload) into the muscle cells. In some embodiments, a muscle-targeting agent specifically binds to an internalizing, cell surface receptor on muscles and is capable of being internalized into muscle cells through receptor mediated internalization. In some embodiments, the muscle-targeting agent is a small molecule, a protein, a peptide, a nucleic acid (e.g., an aptamer), or an antibody. In some embodiments, the muscle-targeting agent is linked to a molecular payload.

Muscle-targeting antibody: As used herein, the term, “muscle-targeting antibody,” refers to a muscle-targeting agent that is an antibody that specifically binds to an antigen found in or on muscle cells. In some embodiments, a muscle-targeting antibody specifically binds to an antigen on muscle cells that facilitates internalization of the muscle-targeting antibody (and any associated molecular payment) into the muscle cells. In some embodiments, the muscle-targeting antibody specifically binds to an internalizing, cell surface receptor present on muscle cells. In some embodiments, the muscle-targeting antibody is an antibody that specifically binds to a transferrin receptor.

Myotonic dystrophy (DM): As used herein, the term “Myotonic dystrophy (DM)” refers to a genetic disease caused by mutations in the DMPK gene or CNBP (ZNF9) gene that is characterized by muscle loss, muscle weakening, and muscle function. Two types of the disease, myotonic dystrophy type 1 (DM1) and myotonic dystrophy type 2 (DM2), have been described. DM1 is associated with an expansion of a CTG trinucleotide repeat in the 3′ non-coding region of DMPK. DM2 is associated with an expansion of a CCTG tetranucleotide repeat in the first intron of ZNF9. In both DM1 and DM2, the nucleotide expansions lead to toxic RNA repeats capable of forming hairpin structures that bind critical intracellular proteins, e.g., muscleblind-like proteins, with high affinity. Myotonic dystrophy, the genetic basis for the disease, and related symptoms are described in the art (see, e.g. Thornton, C. A., “Myotonic Dystrophy” Neurol Clin. (2014), 32(3): 705-719.; and Konieczny et al. “Myotonic dystrophy: candidate small molecule therapeutics” Drug Discovery Today (2017), 22:11.) In some embodiments, subjects are born with a variation of DM1 called congenital myotonic dystrophy. Symptoms of congenital myotonic dystrophy are present from birth and include weakness of all muscles, breathing problems, clubfeet, developmental delays and intellectual disabilities. DM1 is associated with Online Mendelian Inheritance in Man (OMIM) Entry #160900. DM2 is associated with OMIM Entry #602668.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkages. In some embodiments, an oligonucleotide may comprise one or more phosphorothioate linkages, which may be in the Rp or Sp stereochemical conformation.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human transferrin receptor which can be generated using techniques well known in the art, such as, but not limited to, using human Ig phage libraries such as those disclosed in Jermutus et al., PCT publication No. WO 2005/007699 A2.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a disease-associated-repeat expansion, e.g., in a DMPK allele.

Transferrin receptor: As used herein, the term, “transferrin receptor” (also known as TFRC, CD71, p90, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-0-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

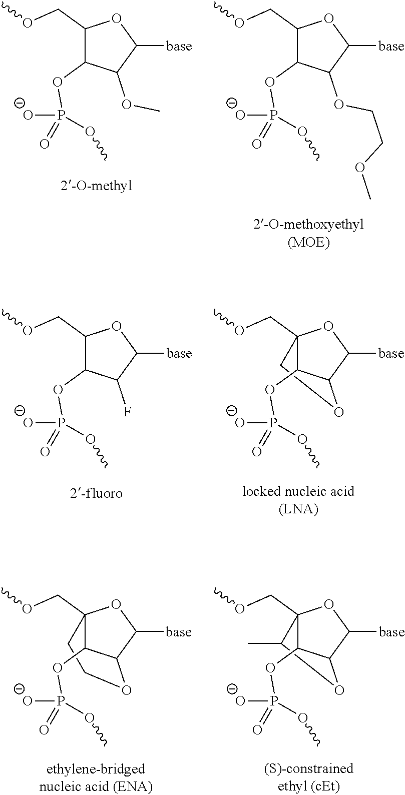

These examples are shown with phosphate groups, but any internucleoside linkages are contemplated between 2′-modified nucleosides.

### II. Complexes

Provided herein are complexes that comprise a targeting agent, e.g., an antibody, covalently linked to a molecular payload. In some embodiments, a complex comprises a muscle-targeting antibody covalently linked to an oligonucleotide. A complex may comprise an antibody that specifically binds a single antigenic site or that binds to at least two antigenic sites that may exist on the same or different antigens.

A complex may be used to modulate the activity or function of at least one gene, protein, and/or (e.g., and) nucleic acid. In some embodiments, the molecular payload present within a complex is responsible for the modulation of a gene, protein, and/or (e.g., and) nucleic acids. A molecular payload may be a small molecule, protein, nucleic acid, oligonucleotide, or any molecular entity capable of modulating the activity or function of a gene, protein, and/or (e.g., and) nucleic acid in a cell. In some embodiments, a molecular payload is an oligonucleotide that targets a disease-associated repeat in cells, e.g., muscle cells or CNS cells.

In some embodiments, a complex comprises a muscle-targeting agent, e.g., an anti-TfR1 antibody, covalently linked to a molecular payload, e.g., an antisense oligonucleotide that targets DMPK, such as a nucleic acid comprising a disease-associated repeat, e.g., a DMPK allele.

### A. Muscle-Targeting Agents

Some aspects of the disclosure provide muscle-targeting agents, e.g., for delivering a molecular payload to a muscle cell. In some embodiments, such muscle-targeting agents are capable of binding to a muscle cell, e.g., via specifically binding to an antigen on the muscle cell, and delivering an associated molecular payload to the muscle cell. In some embodiments, the molecular payload is bound (e.g., covalently bound) to the muscle targeting agent and is internalized into the muscle cell upon binding of the muscle targeting agent to an antigen on the muscle cell, e.g., via endocytosis. It should be appreciated that various types of muscle-targeting agents may be used in accordance with the disclosure, and that any muscle targets (e.g., muscle surface proteins) can be targeted by any type of muscle-targeting agent described herein. For example, the muscle-targeting agent may comprise, or consist of, a small molecule, a nucleic acid (e.g., DNA or RNA), a peptide (e.g., an antibody), a lipid (e.g., a microvesicle), or a sugar moiety (e.g., a polysaccharide). Exemplary muscle-targeting agents are described in further detail herein, however, it should be appreciated that the exemplary muscle-targeting agents provided herein are not meant to be limiting.

Some aspects of the disclosure provide muscle-targeting agents that specifically bind to an antigen on muscle, such as skeletal muscle, smooth muscle, or cardiac muscle. In some embodiments, any of the muscle-targeting agents provided herein bind to (e.g., specifically bind to) an antigen on a skeletal muscle cell, a smooth muscle cell, and/or (e.g., and) a cardiac muscle cell.

By interacting with muscle-specific cell surface recognition elements (e.g., cell membrane proteins), both tissue localization and selective uptake into muscle cells can be achieved. In some embodiments, molecules that are substrates for muscle uptake transporters are useful for delivering a molecular payload into muscle tissue. Binding to muscle surface recognition elements followed by endocytosis can allow even large molecules such as antibodies to enter muscle cells. As another example molecular payloads conjugated to transferrin or anti-TfR1 antibodies can be taken up by muscle cells via binding to transferrin receptor, which may then be endocytosed, e.g., via clathrin-mediated endocytosis.

The use of muscle-targeting agents may be useful for concentrating a molecular payload (e.g., oligonucleotide) in muscle while reducing toxicity associated with effects in other tissues. In some embodiments, the muscle-targeting agent concentrates a bound molecular payload in muscle cells as compared to another cell type within a subject. In some embodiments, the muscle-targeting agent concentrates a bound molecular payload in muscle cells (e.g., skeletal, smooth, or cardiac muscle cells) in an amount that is at least 1, 2, 3, 4, 5, 6, 7, 8, 9, 10, 15, 20, 30, 40, 50, 60, 70, 80, 90, or 100 times greater than an amount in non-muscle cells (e.g., liver, neuronal, blood, or fat cells). In some embodiments, a toxicity of the molecular payload in a subject is reduced by at least 1%, 2%, 3%, 4%, 5%, 10%, 15%, 20%, 25%, 30%, 35%, 40%, 45%, 50%, 55%, 60%, 65%, 70%, 75%, 80%, 90%, or 95% when it is delivered to the subject when bound to the muscle-targeting agent.

In some embodiments, to achieve muscle selectivity, a muscle recognition element (e.g., a muscle cell antigen) may be required. As one example, a muscle-targeting agent may be a small molecule that is a substrate for a muscle-specific uptake transporter. As another example, a muscle-targeting agent may be an antibody that enters a muscle cell via transporter-mediated endocytosis. As another example, a muscle targeting agent may be a ligand that binds to cell surface receptor on a muscle cell. It should be appreciated that while transporter-based approaches provide a direct path for cellular entry, receptor-based targeting may involve stimulated endocytosis to reach the desired site of action.

i. Muscle-Targeting Antibodies

In some embodiments, the muscle-targeting agent is an antibody. Generally, the high specificity of antibodies for their target antigen provides the potential for selectively targeting muscle cells (e.g., skeletal, smooth, and/or (e.g., and) cardiac muscle cells). This specificity may also limit off-target toxicity. Examples of antibodies that are capable of targeting a surface antigen of muscle cells have been reported and are within the scope of the disclosure. For example, antibodies that target the surface of muscle cells are described in Arahata K., et al. “Immunostaining of skeletal and cardiac muscle surface membrane with antibody against Duchenne muscular dystrophy peptide” Nature 1988; 333: 861-3; Song K. S., et al. “Expression of caveolin-3 in skeletal, cardiac, and smooth muscle cells. Caveolin-3 is a component of the sarcolemma and co-fractionates with dystrophin and dystrophin-associated glycoproteins” J Biol Chem 1996; 271: 15160-5; and Weisbart R. H. et al., “Cell type specific targeted intracellular delivery into muscle of a monoclonal antibody that binds myosin IIb” Mol Immunol. 2003 March, 39(13):78309; the entire contents of each of which are incorporated herein by reference.

a. Anti-Transferrin Receptor (TfR) Antibodies

Some aspects of the disclosure are based on the recognition that agents binding to transferrin receptor, e.g., anti-transferrin-receptor antibodies, are capable of targeting muscle cell. Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Accordingly, aspects of the disclosure provide binding proteins (e.g., antibodies) that bind to transferrin receptor. In some embodiments, binding proteins that bind to transferrin receptor are internalized, along with any bound molecular payload, into a muscle cell. As used herein, an antibody that binds to a transferrin receptor may be referred to interchangeably as an, transferrin receptor antibody, an anti-transferrin receptor antibody, or an anti-TfR1 antibody. Antibodies that bind, e.g. specifically bind, to a transferrin receptor may be internalized into the cell, e.g. through receptor-mediated endocytosis, upon binding to a transferrin receptor.

It should be appreciated that anti-TfR1 antibodies may be produced, synthesized, and/or (e.g., and) derivatized using several known methodologies, e.g. library design using phage display. Exemplary methodologies have been characterized in the art and are incorporated by reference (Diez, P. et al. “High-throughput phage-display screening in array format”, Enzyme and microbial technology, 2015, 79, 34-41.; Christoph M. H. and Stanley, J. R. “Antibody Phage Display: Technique and Applications” J Invest Dermatol. 2014, 134:2.; Engleman, Edgar (Ed.) “Human Hybridomas and Monoclonal Antibodies.” 1985, Springer.). In other embodiments, an anti-TfR1 antibody has been previously characterized or disclosed. Antibodies that specifically bind to transferrin receptor are known in the art (see, e.g. U.S. Pat. No. 4,364,934, filed Dec. 4, 1979, “Monoclonal antibody to a human early thymocyte antigen and methods for preparing same”; U.S. Pat. No. 8,409,573, filed Jun. 14, 2006, “Anti-CD71 monoclonal antibodies and uses thereof for treating malignant tumor cells”; U.S. Pat. No. 9,708,406, filed May 20, 2014, “Anti-transferrin receptor antibodies and methods of use”; U.S. Pat. No. 9,611,323, filed Dec. 19, 2014, “Low affinity blood brain barrier receptor antibodies and uses therefor”; WO 2015/098989, filed Dec. 24, 2014, “Novel anti-Transferrin receptor antibody that passes through blood-brain barrier”; Schneider C. et al. “Structural features of the cell surface receptor for transferrin that is recognized by the monoclonal antibody OKT9.” J Biol Chem. 1982, 257:14, 8516-8522.; Lee et al. “Targeting Rat Anti-Mouse Transferrin Receptor Monoclonal Antibodies through Blood-Brain Barrier in Mouse” 2000, J Pharmacol. Exp. Ther., 292: 1048-1052.).

In some embodiments, the anti-TfR1 antibody described herein binds to transferrin receptor with high specificity and affinity. In some embodiments, the anti-TfR1 antibody described herein specifically binds to any extracellular epitope of a transferrin receptor or an epitope that becomes exposed to an antibody. In some embodiments, anti-TfR1 antibodies provided herein bind specifically to transferrin receptor from human, non-human primates, mouse, rat, etc. In some embodiments, anti-TfR1 antibodies provided herein bind to human transferrin receptor. In some embodiments, the anti-TfR1 antibody described herein binds to an amino acid segment of a human or non-human primate transferrin receptor, as provided in SEQ ID NOs: 105-108. In some embodiments, the anti-TfR1 antibody described herein binds to an amino acid segment corresponding to amino acids 90-96 of a human transferrin receptor as set forth in SEQ ID NO: 105, which is not in the apical domain of the transferrin receptor.

In some embodiments, the anti-TfR1 antibodies described herein (e.g., Anti-TfR clone 8 in Table 2 below) bind an epitope in TfR1, wherein the epitope comprises residues in amino acids 214-241 and/or amino acids 354-381 of SEQ ID NO: 105. In some embodiments, the anti-TfR1 antibodies described herein bind an epitope comprising residues in amino acids 214-241 and amino acids 354-381 of SEQ ID NO: 105. In some embodiments, the anti-TfR1 antibodies described herein bind an epitope comprising one or more of residues Y222, T227, K231, H234, T367, S368, S370, T376, and S378 of human TfR1 as set forth in SEQ ID NO: 105. In some embodiments, the anti-TfR1 antibodies described herein bind an epitope comprising residues Y222, T227, K231, H234, T367, S368, S370, T376, and S378 of human TfR1 as set forth in SEQ ID NO: 105.

In some embodiments, the anti-TfR1 antibody described herein (e.g., 3M12 in Table 2 below and its variants) bind an epitope in TfR1, wherein the epitope comprises residues in amino acids 258-291 and/or amino acids 358-381 of SEQ ID NO: 105. In some embodiments, the anti-TfR1 antibodies (e.g., 3M12 in Table 2 below and its variants) described herein bind an epitope comprising residues in amino acids amino acids 258-291 and amino acids 358-381 of SEQ ID NO: 105. In some embodiments, the anti-TfR1 antibodies described herein (e.g., 3M12 in Table 2 below and its variants) bind an epitope comprising one or more of residues K261, S273, Y282, T362, S368, S370, and K371 of human TfR1 as set forth in SEQ ID NO: 105. In some embodiments, the anti-TfR1 antibodies described herein (e.g., 3M12 in Table 2 below and its variants) bind an epitope comprising residues K261, S273, Y282, T362, S368, S370, and K371 of human TfR1 as set forth in SEQ ID NO: 105.

An example human transferrin receptor amino acid sequence, corresponding to NCBI sequence NP_003225.2 (transferrin receptor protein 1 isoform 1, Homo sapiens) is as follows:

(SEQ ID NO: 105)
MMDQARSAFSNLFGGEPLSYTRFSLARQVDGDNSHVEMKLAVDEEE
NADNNTKANVTKPKRCSGSICYGTIAVIVFFLIGFMIGYLGYCKGV
EPKTECERLAGTESPVREEPGEDFPAARRLYWDDLKRKLSEKLDST
DFTGTIKLLNENSYVPREAGSQKDENLALYVENQFREFKLSKVWRD
QHFVKIQVKDSAQNSVIIVDKNGRLVYLVENPGGYVAYSKAATVTG
KLVHANFGTKKDFEDLYTPVNGSIVIVRAGKITFAEKVANAESLNA
IGVLIYMDQTKFPIVNAELSFFGHAHLGTGDPYTPGFPSFNHTQFP
PSRSSGLPNIPVQTISRAAAEKLFGNMEGDCPSDWKTDSTCRMVTS
ESKNVKLTVSNVLKEIKILNIFGVIKGFVEPDHYVVVGAQRDAWGP
GAAKSGVGTALLLKLAQMFSDMVLKDGFQPSRSIIFASWSAGDFGS
VGATEWLEGYLSSLHLKAFTYINLDKAVLGTSNFKVSASPLLYTLI
EKTMQNVKHPVTGQFLYQDSNWASKVEKLTLDNAAFPFLAYSGIPA
VSFCFCEDTDYPYLGTTMDTYKELIERIPELNKVARAAAEVAGQFV
IKLTHDVELNLDYERYNSQLLSFVRDLNQYRADIKEMGLSLQWLYS
ARGDFFRATSRLTTDFGNAEKTDRFVMKKLNDRVMRVEYHFLSPYV
SPKESPFRHVFWGSGSHTLPALLENLKLRKQNNGAFNETLFRNQLA
LATWTIQGAANALSGDVWDIDNEF.

An example non-human primate transferrin receptor amino acid sequence, corresponding to NCBI sequence NP_001244232.1(transferrin receptor protein 1, Macaca mulatta) is as follows:

(SEQ ID NO: 106)
MMDQARSAFSNLFGGEPLSYTRFSLARQVDGDNSHVEMKLGVDEEE
NTDNNTKPNGTKPKRCGGNICYGTIAVIIFFLIGFMIGYLGYCKGV
EPKTECERLAGTESPAREEPEEDFPAAPRLYWDDLKRKLSEKLDTT
DFTSTIKLLNENLYVPREAGSQKDENLALYIENQFREFKLSKVWRD
QHFVKIQVKDSAQNSVIIVDKNGGLVYLVENPGGYVAYSKAATVTG
KLVHANFGTKKDFEDLDSPVNGSIVIVRAGKITFAEKVANAESLNA
IGVLIYMDQTKFPIVKADLSFFGHAHLGTGDPYTPGFPSFNHTQFP
PSQSSGLPNIPVQTISRAAAEKLFGNMEGDCPSDWKTDSTCKMVTS
ENKSVKLTVSNVLKETKILNIFGVIKGFVEPDHYVVVGAQRDAWGP
GAAKSSVGTALLLKLAQMFSDMVLKDGFQPSRSIIFASWSAGDFGS
VGATEWLEGYLSSLHLKAFTYINLDKAVLGTSNFKVSASPLLYTLI
EKTMQDVKHPVTGRSLYQDSNWASKVEKLTLDNAAFPFLAYSGIPA
VSFCFCEDTDYPYLGTTMDTYKELVERIPELNKVARAAAEVAGQFV
IKLTHDTELNLDYERYNSQLLLFLRDLNQYRADVKEMGLSLQWLYS
ARGDFFRATSRLTTDFRNAEKRDKFVMKKLNDRVMRVEYYFLSPYV
SPKESPFRHVFWGSGSHTLSALLESLKLRRQNNSAFNETLFRNQLA
LATWTIQGAANALSGDVWDIDNEF

An example non-human primate transferrin receptor amino acid sequence, corresponding to NCBI sequence XP_005545315.1 (transferrin receptor protein 1, Macaca fascicularis) is as follows:

(SEQ ID NO: 107)
MMDQARSAFSNLFGGEPLSYTRFSLARQVDGDNSHVEMKLGVDEEE
NTDNNTKANGTKPKRCGGNICYGTIAVIIFFLIGFMIGYLGYCKGV
EPKTECERLAGTESPAREEPEEDFPAAPRLYWDDLKRKLSEKLDTT
DFTSTIKLLNENLYVPREAGSQKDENLALYIENQFREFKLSKVWRD
QHFVKIQVKDSAQNSVIIVDKNGGLVYLVENPGGYVAYSKAATVTG
KLVHANFGTKKDFEDLDSPVNGSIVIVRAGKITFAEKVANAESLNA
IGVLIYMDQTKFPIVKADLSFFGHAHLGTGDPYTPGFPSFNHTQFP
PSQSSGLPNIPVQTISRAAAEKLFGNMEGDCPSDWKTDSTCKMVTS
ENKSVKLTVSNVLKETKILNIFGVIKGFVEPDHYVVVGAQRDAWGP
GAAKSSVGTALLLKLAQMFSDMVLKDGFQPSRSIIFASWSAGDFGS
VGATEWLEGYLSSLHLKAFTYINLDKAVLGTSNFKVSASPLLYTLI
EKTMQDVKHPVTGRSLYQDSNWASKVEKLTLDNAAFPFLAYSGIPA
VSFCFCEDTDYPYLGTTMDTYKELVERIPELNKVARAAAEVAGQFV
IKLTHDTELNLDYERYNSQLLLFLRDLNQYRADVKEMGLSLQWLYS
ARGDFFRATSRLTTDFRNAEKRDKFVMKKLNDRVMRVEYYFLSPYV
SPKESPFRHVFWGSGSHTLSALLESLKLRRQNNSAFNETLFRNQLA
LATWTIQGAANALSGDVWDIDNEF.

An example mouse transferrin receptor amino acid sequence, corresponding to NCBI sequence NP_001344227.1 (transferrin receptor protein 1, Mus musculus) is as follows:

(SEQ ID NO: 108)
MMDQARSAFSNLFGGEPLSYTRFSLARQVDGDNSHVEMKLAADEEE
NADNNMKASVRKPKRFNGRLCFAAIALVIFFLIGFMSGYLGYCKRV
EQKEECVKLAETEETDKSETMETEDVPTSSRLYWADLKTLLSEKLN
SIEFADTIKQLSQNTYTPREAGSQKDESLAYYIENQFHEFKFSKVW
RDEHYVKIQVKSSIGQNMVTIVQSNGNLDPVESPEGYVAFSKPTEV
SGKLVHANFGTKKDFEELSYSVNGSLVIVRAGEITFAEKVANAQSF
NAIGVLIYMDKNKFPVVEADLALFGHAHLGTGDPYTPGFPSFNHTQ
FPPSQSSGLPNIPVQTISRAAAEKLFGKMEGSCPARWNIDSSCKLE
LSQNQNVKLIVKNVLKERRILNIFGVIKGYEEPDRYVVVGAQRDAL
GAGVAAKSSVGTGLLLKLAQVFSDMISKDGFRPSRSIIFASWTAGD
FGAVGATEWLEGYLSSLHLKAFTYINLDKVVLGTSNFKVSASPLLY
TLMGKIMQDVKHPVDGKSLYRDSNWISKVEKLSFDNAAYPFLAYSG
IPAVSFCFCEDADYPYLGTRLDTYEALTQKVPQLNQMVRTAAEVAG
QLIIKLTHDVELNLDYEMYNSKLLSFMKDLNQFKTDIRDMGLSLQW
LYSARGDYFRATSRLTTDFHNAEKTNRFVMREINDRIMKVEYHFLS
PYVSPRESPFRHIFWGSGSHTLSALVENLKLRQKNITAFNETLFRN
QLALATWTIQGVANALSGDIWNIDNEF

In some embodiments, an anti-TfR1 antibody binds to an amino acid segment of the receptor as follows: FVKIQVKDSAQNSVIIVDKNGRLVYLVENPGGYVAYSKAATVTGKLVHANFGTKKDFE DLYTPVNGSIVIVRAGKITFAEKVANAESLNAIGVLIYMDQTKFPIVNAELSFFGHAHLG TGDPYTPGFPSFNHTQFPPSRSSGLPNIPVQTISRAAAEKLFGNMEGDCPSDWKTDSTCR MVTSESKNVKLTVSNVLKE (SEQ ID NO: 109) and does not inhibit the binding interactions between transferrin receptors and transferrin and/or (e.g., and) human hemochromatosis protein (also known as HFE). In some embodiments, the anti-TfR1 antibody described herein does not bind an epitope in SEQ ID NO: 109.

Appropriate methodologies may be used to obtain and/or (e.g., and) produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256: 495-497). The antigen-of-interest may be used as the immunogen in any form or entity, e.g., recombinant or a naturally occurring form or entity. Hybridomas are screened using standard methods, e.g. ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, the anti-TfR1 antibody of the present disclosure comprises a VL domain and/or (e.g., and) a VH domain of any one of the anti-TfR1 antibodies selected from any one of Tables 2-7, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra.

In some embodiments, agents binding to transferrin receptor, e.g., anti-TfR1 antibodies, are capable of targeting muscle cell and/or (e.g., and) mediate the transportation of an agent across the blood brain barrier (e.g., to a CNS cell). Transferrin receptors are internalizing cell surface receptors that transport transferrin across the cellular membrane and participate in the regulation and homeostasis of intracellular iron levels. Some aspects of the disclosure provide transferrin receptor binding proteins, which are capable of binding to transferrin receptor. Antibodies that bind, e.g. specifically bind, to a transferrin receptor may be internalized into the cell, e.g. through receptor-mediated endocytosis, upon binding to a transferrin receptor.

Provided herein, in some aspects, are humanized antibodies that bind to transferrin receptor with high specificity and affinity. In some embodiments, the humanized anti-TfR1 antibody described herein specifically binds to any extracellular epitope of a transferrin receptor or an epitope that becomes exposed to an antibody. In some embodiments, the humanized anti-TfR1 antibodies provided herein bind specifically to transferrin receptor from human, non-human primates, mouse, rat, etc. In some embodiments, the humanized anti-TfR1 antibodies provided herein bind to human transferrin receptor. In some embodiments, the humanized anti-TfR1 antibody described herein binds to an amino acid segment of a human or non-human primate transferrin receptor, as provided in SEQ ID NOs: 105-108. In some embodiments, the humanized anti-TfR1 antibody described herein binds to an amino acid segment corresponding to amino acids 90-96 of a human transferrin receptor as set forth in SEQ ID NO: 105, which is not in the apical domain of the transferrin receptor. In some embodiments, the humanized anti-TfR1 antibodies described herein binds to TfR1 but does not bind to TfR2.

In some embodiments, an anti-TFR1 antibody specifically binds a TfR1 (e.g., a human or non-human primate TfR1) with binding affinity (e.g., as indicated by Kd) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, the anti-TfR1 antibodies described herein bind to TfR1 with a KD of sub-nanomolar range. In some embodiments, the anti-TfR1 antibodies described herein selectively bind to transferrin receptor 1 (TfR1) but do not bind to transferrin receptor 2 (TfR2). In some embodiments, the anti-TfR1 antibodies described herein bind to human TfR1 and cyno TfR1 (e.g., with a Kd of 10⁻⁷ M, 10⁻⁸ M, 10⁻⁹ M, 10⁻¹⁰ M, 10⁻¹¹ M, 10⁻¹² M, 10⁻¹³ M, or less), but do not bind to a mouse TfR1. The affinity and binding kinetics of the anti-TfR1 antibody can be tested using any suitable method including but not limited to biosensor technology (e.g., OCTET or BIACORE). In some embodiments, binding of any one of the anti-TfR1 antibodies described herein does not complete with or inhibit transferrin binding to the TfR1. In some embodiments, binding of any one of the anti-TfR1 antibodies described herein does not complete with or inhibit HFE-beta-2-microglobulin binding to the TfR1.

Non-limiting examples of anti-TfR1 antibodies are provided in Table 2.

TABLE 2
Examples of Anti-TfR1 Antibodies

| | No. | | | |
|---|---|---|---|---|
| Ab | system | IMGT | Kabat | Chothia |
| 3-A4 | CDR- | GFNIKDDY (SEQ ID NO: | DDYMY (SEQ ID NO: 7) | GFNIKDD (SEQ ID NO: 12) |
| | H1 | 1) | | |
| | CDR- | IDPENGDT (SEQ ID NO: | WIDPENGDTEYASKFQD | ENG (SEQ ID NO: 13) |
| | H2 | 2) | (SEQ ID NO: 8) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPENGDT | | |
| | | EYASKFQDKATVTADTSSNTAYLQLSSLTSEDTAVYYCTLWLRRGLDYWGQGTSVTVS | | |
| | | S (SEQ ID NO: 17) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-A4 | CDR- | GFNIKDDY (SEQ ID NO: | DDYMY (SEQ ID NO: 7) | GFNIKDD (SEQ ID NO: 12) |
| N54T* | H1 | 1) | | |
| | CDR- | IDPETGDT (SEQ ID NO: | WIDPETGDTEYASKFQD | ETG (SEQ ID NO: 21) |
| | H2 | 19) | (SEQ ID NO: 20) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS(SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPETGDT | | |
| | | EYASKFQDKATVTADTSSNTAYLQLSSLTSEDTAVYYCTLWLRRGLDYWGQGTSVTVS | | |
| | | S (SEQ ID NO: 22) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-A4 | CDR- | GFNIKDDY (SEQ ID NO: | DDYMY (SEQ ID NO: 7) | GFNIKDD (SEQ ID NO: 12) |
| N54S* | H1 | 1) | | |
| | CDR- | IDPESGDT (SEQ ID NO: | WIDPESGDTEYASKFQD | ESG (SEQ ID NO: 25) |
| | H2 | 23) | (SEQ ID NO: 24) | |
| | CDR- | TLWLRRGLDY (SEQ ID | WLRRGLDY (SEQ ID NO: 9) | LRRGLD (SEQ ID NO: 14) |
| | H3 | NO: 3) | | |
| | CDR- | KSLLHSNGYTY (SEQ ID | RSSKSLLHSNGYTYLF (SEQ | SKSLLHSNGYTY (SEQ ID |
| | L1 | NO: 4) | ID NO: 10) | NO: 15) |
| | CDR- | RMS (SEQ ID NO: 5) | RMSNLAS (SEQ ID NO: 11) | RMS (SEQ ID NO: 5) |
| | L2 | | | |
| | CDR- | MQHLEYPFT (SEQ ID | MQHLEYPFT (SEQ ID NO: 6) | HLEYPF (SEQ ID NO: 16) |
| | L3 | NO: 6) | | |
| | VH | EVQLQQSGAELVRPGASVKLSCTASGFNIKDDYMYWVKQRPEQGLEWIGWIDPESGDT | | |
| | | EYASKFQDKATVTADTSSNTAYLQLSSLTSEDTAVYYCTLWLRRGLDYWGQGTSVTVS | | |
| | | S (SEQ ID NO: 26) | | |
| | VL | DIVMTQAAPSVPVTPGESVSISCRSSKSLLHSNGYTYLFWFLQRPGQSPQLLIYRMSNLA | | |
| | | SGVPDRFSGSGSGTAFTLRISRVEAEDVGVYYCMQHLEYPFTFGGGTKLEIK (SEQ ID | | |
| | | NO: 18) | | |
| 3-M12 | CDR- | GYSITSGYY (SEQ ID | SGYYWN (SEQ ID NO: 33) | GYSITSGY (SEQ ID NO: |
| | H1 | NO: 27) | | 38) |
| | CDR- | ITFDGAN (SEQ ID NO: | YITFDGANNYNPSLKN (SEQ | FDG (SEQ ID NO: 39) |
| | H2 | 28) | ID NO: 34) | |
| | CDR- | TRSSYDYDVLDY (SEQ | SSYDYDVLDY (SEQ ID NO: | SYDYDVLD (SEQ ID NO: |
| | H3 | ID NO: 29) | 35) | 40) |
| | CDR- | QDISNF (SEQ ID NO: 30) | RASQDISNFLN (SEQ ID NO: | SQDISNF (SEQ ID NO: 41) |
| | L1 | | 36) | |
| | CDR- | YTS (SEQ ID NO: 31) | YTSRLHS (SEQ ID NO: 37) | YTS (SEQ ID NO: 31) |
| | L2 | | | |
| | CDR- | QQGHTLPYT (SEQ ID | QQGHTLPYT (SEQ ID NO: 32) | GHTLPY (SEQ ID NO: 42) |
| | L3 | NO: 32) | | |
| | VH | DVQLQESGPGLVKPSQSLSLTCSVTGYSITSGYYWNWIRQFPGNKLEWMGYITFDGAN | | |
| | | NYNPSLKNRISITRDTSKNQFFLKLTSVTTEDTATYYCTRSSYDYDVLDYWGQGTTLTV | | |
| | | SS (SEQ ID NO: 43) | | |
| | VL | DIQMTQTTSSLSASLGDRVTISCRASQDISNFLNWYQQRPDGTVKLLIYYTSRLHSGVPS | | |
| | | RFSGSGSGTDFSLTVSNLEQEDIATYFCQQGHTLPYTFGGGTKLEIK (SEQ ID NO: 44) | | |
| 5-H12 | CDR- | GYSFTDYC (SEQ ID NO: | DYCIN (SEQ ID NO: 51) | GYSFTDY (SEQ ID NO: 56) |
| | H1 | 45) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYCINWVNQRPGQGLEWIGWIYPGSGNTR | | |
| | | YSERFKGKATLTVDTSSNTAYMQLSSLTSEDSAVYFCAREDYYPYHGMDYWGQGTSV | | |
| | | TVSS (SEQ ID NO: 61) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| 5-H12 | CDR- | GYSFTDYY (SEQ ID | DYYIN (SEQ ID NO: 64) | GYSFTDY (SEQ ID NO: 56) |
| C33Y* | H1 | NO: 63) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYYINWVNQRPGQGLEWIGWIYPGSGNTR | | |
| | | YSERFKGKATLTVDTSSNTAYMQLSSLTSEDSAVYFCAREDYYPYHGMDYWGQGTSV | | |
| | | TVSS (SEQ ID NO: 65) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| 5-H12 | CDR- | GYSFTDYD (SEQ ID | DYDIN (SEQ ID NO: 67) | GYSFTDY (SEQ ID NO: 56) |
| C33D* | H1 | NO: 66) | | |
| | CDR- | IYPGSGNT (SEQ ID NO: | WIYPGSGNTRYSERFKG | GSG (SEQ ID NO: 57) |
| | H2 | 46) | (SEQ ID NO: 52) | |
| | CDR- | AREDYYPYHGMDY | EDYYPYHGMDY (SEQ ID | DYYPYHGMD (SEQ ID |
| | H3 | (SEQ ID NO: 47) | NO: 53) | NO: 58) |
| | CDR- | ESVDGYDNSF (SEQ ID | RASESVDGYDNSFMH (SEQ | SESVDGYDNSF (SEQ ID |
| | L1 | NO: 48) | ID NO: 54) | NO: 59) |
| | CDR- | RAS (SEQ ID NO: 49) | RASNLES (SEQ ID NO: 55) | RAS (SEQ ID NO: 49) |
| | L2 | | | |
| | CDR- | QQSSEDPWT (SEQ ID | QQSSEDPWT (SEQ ID NO: 50) | SSEDPW (SEQ ID NO: 60) |
| | L3 | NO: 50) | | |
| | VH | QIQLQQSGPELVRPGASVKISCKASGYSFTDYDINWVNQRPGQGLEWIGWIYPGSGNTRY | | |
| | | SERFKGKATLTVDTSSNTAYMQLSSLTSEDSAVYFCAREDYYPYHGMDYWGQGTSVTV | | |
| | | SS (SEQ ID NO: 68) | | |
| | VL | DIVLTQSPTSLAVSLGQRATISCRASESVDGYDNSFMHWYQQKPGQPPKLLIFRASNLES | | |
| | | GIPARFSGSGSRTDFTLTINPVEAADVATYYCQQSSEDPWTFGGGTKLEIK (SEQ ID NO: | | |
| | | 62) | | |
| Anti- | CDR- | GYSFTSYW (SEQ ID | SYWIG (SEQ ID NO: 144) | GYSFTSY (SEQ ID NO: |
| TfR | H1 | NO: 138) | | 149) |
| clone 8 | CDR- | IYPGDSDT (SEQ ID NO: | IIYPGDSDTRYSPSFQGQ | GDS (SEQ ID NO: 150) |
| | H2 | 139) | (SEQ ID NO: 145) | |
| | CDR- | ARFPYDSSGYYSFDY | FPYDSSGYYSFDY (SEQ ID | PYDSSGYYSFD (SEQ ID |
| | H3 | (SEQ ID NO: 140) | NO: 146) | NO: 151) |
| | CDR- | QSISSY (SEQ ID NO: | RASQSISSYLN (SEQ ID NO: | SQSISSY (SEQ ID NO: 152) |
| | L1 | 141) | 147) | |
| | CDR- | AAS (SEQ ID NO: 142) | AASSLQS (SEQ ID NO: 148) | AAS (SEQ ID NO: 142) |
| | L2 | | | |
| | CDR- | QQSYSTPLT (SEQ ID | QQSYSTPLT (SEQ ID NO: | SYSTPL (SEQ ID NO: 153) |
| | L3 | NO: 143) | 143) | |

*mutation positions are according to Kabat numbering of the respective VH sequences containing the mutations

In some embodiments, the anti-TfR1 antibody of the present disclosure is a humanized variant of any one of the anti-TfR1 antibodies provided in Table 2. In some embodiments, the anti-TfR1 antibody of the present disclosure comprises a CDR-H1, a CDR-H2, a CDR-H3, a CDR-L1, a CDR-L2, and a CDR-L3 that are the same as the CDR-H1, CDR-H2, and CDR-H3 in any one of the anti-TfR1 antibodies provided in Table 2, and comprises a humanized heavy chain variable region and/or (e.g., and) a humanized light chain variable region.

Examples of amino acid sequences of anti-TfR1 antibodies described herein are provided in Table 3.

TABLE 3
Variable Regions of Anti-TfR1 Antibodies

| Antibody | Variable Region Amino Acid Sequence** |
|---|---|
| 3A4 | V_{H}: |
| VH3 (N54T*)/ | EVQLVQSGSELKKPGASVKVSCTASGFNIKDDYM |
| Vκ4 | YWVRQPPGKGLEWIGWIDPETGDTEYASKFQDRV |
| | TVTADTSTNTAYMELSSLRSEDTAVYYCTLWLRR |
| | GLDYWGQGTLVTVSS (SEQ ID NO: 69) |
| | V_{L}: |
| | DIVMTQSPLSLPVTPGEPASISCRSSKSLLHSNG |
| | YTYLFWFQQRPGQSPRLLIYRMSNLASGVPDRFS |
| | GSGSGTDFTLKISRVEAEDVGVYYCMQHLEYPFT |
| | FGGGTKVEIK (SEQ ID NO: 70) |
| 3A4 | V_{H}: |
| VH3 (N54S*)/ | EVQLVQSGSELKKPGASVKVSCTASGFNIKDDYM |
| Vκ4 | YWVRQPPGKGLEWIGWIDPESGDTEYASKFQDRV |
| | TVTADTSTNTAYMELSSLRSEDTAVYYCTLWLRR |
| | GLDYWGQGTLVTVSS (SEQ ID NO: 71) |
| | V_{L}: |
| | DIVMTQSPLSLPVTPGEPASISCRSSKSLLHSNG |
| | YTYLFWFQQRPGQSPRLLIYRMSNLASGVPDRFS |
| | GSGSGTDFTLKISRVEAEDVGVYYCMQHLEYPFT |
| | FGGGTKVEIK (SEQ ID NO: 70) |
| 3A4 | VH: |
| VH3/Vκ4 | EVQLVQSGSELKKPGASVKVSCTASGFNIKDDYM |
| | YWVRQPPGKGLEWIGWIDPENGDTEYASKFQDRV |
| | TVTADTSTNTAYMELSSLRSEDTAVYYCTLWLRR |
| | GLDYWGQGTLVTVSS (SEQ ID NO: 72) |
| | VL: |
| | DIVMTQSPLSLPVTPGEPASISCRSSKSLLHSNG |
| | YTYLFWFQQRPGQSPRLLIYRMSNLASGVPDRFS |
| | GSGSGTDFTLKISRVEAEDVGVYYCMQHLEYPFT |
| | FGGGTKVEIK (SEQ ID NO: 70) |
| 3M12 | VH: |
| VH3/Vκ2 | QVQLQESGPGLVKPSQTLSLTCSVTGYSITSGYY |
| | WNWIRQPPGKGLEWMGYITFDGANNYNPSLKNRV |
| | SISRDTSKNQFSLKLSSVTAEDTATYYCTRSSYD |
| | YDVLDYWGQGTTVTVSS (SEQ ID NO: 73) |
| | VL: |
| | DIQMTQSPSSLSASVGDRVTITCRASQDISNFLN |
| | WYQQKPGQPVKLLIYYTSRLHSGVPSRFSGSGSG |
| | TDFTLTISSLQPEDFATYFCQQGHTLPYTFGQGT |
| | KLEIK (SEQ ID NO: 74) |
| 3M12 | VH: |
| VH3/Vκ3 | QVQLQESGPGLVKPSQTLSLTCSVTGYSITSGYY |
| | WNWIRQPPGKGLEWMGYITFDGANNYNPSLKNRV |
| | SISRDTSKNQFSLKLSSVTAEDTATYYCTRSSYD |
| | YDVLDYWGQGTTVTVSS (SEQ ID NO: 73) |
| | VL: |
| | DIQMTQSPSSLSASVGDRVTITCRASQDISNFLN |
| | WYQQKPGQPVKLLIYYTSRLHSGVPSRFSGSGSG |
| | TDFTLTISSLQPEDFATYYCQQGHTLPYTFGQGT |
| | KLEIK (SEQ ID NO: 75) |
| 3M12 | VH: |
| VH4/Vκ2 | QVQLQESGPGLVKPSQTLSLTCTVTGYSITSGYY |
| | WNWIRQPPGKGLEWIGYITFDGANNYNPSLKNRV |
| | SISRDTSKNQFSLKLSSVTAEDTATYYCTRSSYD |
| | YDVLDYWGQGTTVTVSS (SEQ ID NO: 76) |
| | VL: |
| | DIQMTQSPSSLSASVGDRVTITCRASQDISNFLN |
| | WYQQKPGQPVKLLIYYTSRLHSGVPSRFSGSGSG |
| | TDFTLTISSLQPEDFATYFCQQGHTLPYTFGQGT |
| | KLEIK (SEQ ID NO: 74) |
| 3M12 | VH: |
| VH4/Vκ3 | QVQLQESGPGLVKPSQTLSLTCTVTGYSITSGYY |
| | WNWIRQPPGKGLEWIGYITFDGANNYNPSLKNRV |
| | SISRDTSKNQFSLKLSSVTAEDTATYYCTRSSYD |
| | YDVLDYWGQGTTVTVSS (SEQ ID NO: 76) |
| | VL: |
| | DIQMTQSPSSLSASVGDRVTITCRASQDISNFLN |
| | WYQQKPGQPVKLLIYYTSRLHSGVPSRFSGSGSG |
| | TDFTLTISSLQPEDFATYYCQQGHTLPYTFGQGT |
| | KLEIK (SEQ ID NO: 75) |
| 5H12 | VH: |
| VH5 (C33Y*)/ | QVQLVQSGAEVKKPGASVKVSCKASGYSFTDYYI |
| Vκ3 | NWVRQAPGQGLEWMGWIYPGSGNTRYSERFKGRV |
| | TITRDTSASTAYMELSSLRSEDTAVYYCAREDYY |
| | PYHGMDYWGQGTLVTVSS (SEQ ID NO: 77) |
| | VL: |
| | DIVLTQSPDSLAVSLGERATINCRASESVDGYDN |
| | SFMHWYQQKPGQPPKLLIFRASNLESGVPDRESG |
| | SGSRTDFTLTISSLQAEDVAVYYCQQSSEDPWTF |
| | GQGTKLEIK (SEQ ID NO: 78) |
| 5H12 | VH: |
| VH5 (C33D*)/ | QVQLVQSGAEVKKPGASVKVSCKASGYSFTDYDI |
| Vκ4 | NWVRQAPGQGLEWMGWIYPGSGNTRYSERFKGRV |
| | TITRDTSASTAYMELSSLRSEDTAVYYCAREDYY |
| | PYHGMDYWGQGTLVTVSS (SEQ ID NO: 79) |
| | VL: |
| | DIVMTQSPDSLAVSLGERATINCRASESVDGYDN |
| | SFMHWYQQKPGQPPKLLIFRASNLESGVPDRFSG |
| | SGSGTDFTLTISSLQAEDVAVYYCQQSSEDPWTF |
| | GQGTKLEIK (SEQ ID NO: 80) |
| 5H12 | VH: |
| VH5 (C33Y*)/ | QVQLVQSGAEVKKPGASVKVSCKASGYSFTDYYI |
| Vκ4 | NWVRQAPGQGLEWMGWIYPGSGNTRYSERFKGRV |
| | TITRDTSASTAYMELSSLRSEDTAVYYCAREDYY |
| | PYHGMDYWGQGTLVTVSS (SEQ ID NO: 77) |
| | VL: |
| | DIVMTQSPDSLAVSLGERATINCRASESVDGYDN |
| | SFMHWYQQKPGQPPKLLIFRASNLESGVPDRFSG |
| | SGSGTDFTLTISSLQAEDVAVYYCQQSSEDPWTF |
| | GQGTKLEIK (SEQ ID NO: 80) |
| Anti-TfR | VH: |
| clone 8 | QVQLVQSGAEVKKPGESLKISCKGSGYSFTSYWI |
| | GWVRQMPGKGLEWMGIIYPGDSDTRYSPSFQGQV |
| | TISADKSISTAYLQWSSLKASDTAMYYCARFPYD |
| | SSGYYSFDYWGQGTLVTVSS (SEQ ID NO: |
| | 154) |
| | VL: |
| | DIQMTQSPSSLSASVGDRVTITCRASQSISSYLN |
| | WYQQKPGKAPKLLIYAASSLQSGVPSRFSGSGSG |
| | TDFTLTISSLQPEDFATYYCQQSYSTPLTFGGGT |
| | KVEIK (SEQ ID NO: 155) |

*mutation positions are according to Kabat numbering of the respective VH sequences containing the mutations
**CDRs according to the Kabat numbering system are bolded

In some embodiments, the anti-TfR1 antibody of the present disclosure comprises a VH comprising the CDR-H1, CDR-H2, and CDR-H3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprises one or more (e.g., 1, 2, 3, 4, 5, 6, 7, 8, 9, 10 or more) amino acid variations in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the CDR-H1, CDR-H2, and CDR-H3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VH provided in Table 3. Alternatively or in addition (e.g., in addition), the anti-TfR1 antibody of the present disclosure comprises a VL comprising the CDR-L1, CDR-L2, and CDR-L3 of any one of the anti-TfR1 antibodies provided in Table 3 and comprising an amino acid sequence that is at least 70% (e.g., at least 70%, at least 75%, at least 80%, at least 85%, at least 90%, at least 95%, at least 99%) identical in the framework regions as compared with the respective VL provided in Table 3. In some embodiments, the VH of the anti-TfR1 antibody is a humanized VH, and/or the VL of the anti-TfR1 antibody is a humanized VL.

In some embodiments, the anti-TfR1 antibody of the present disclosure comprises a VH comprising the amino acid sequence of SEQ ID NO: 69 and a VL comprising the amino acid sequence of SEQ ID NO: 70.

In some embodiments, the anti-TfR1 antibody of the present disclosure comprises a VH comprising the amino acid sequence of SEQ ID NO: 71 and a VL comprising the amino acid sequence of SEQ ID NO: 70.

In some embodiments, the anti-TfR1 antibody of the present disclosure comprises a VH comprising the amino acid sequence of SEQ ID NO: 72 and a VL comprising the amino acid sequence of SEQ ID NO: 70.

In some embodiments, the anti-TfR1 antibody of the present disclosure comprises a VH comprising the amino acid sequence of SEQ ID NO: 73 and a VL comprising the amino acid sequence of SEQ ID NO: 74.

In some embodiments, the anti-TfR1 antibody of the present disclosure comprises a VH comprising the amino acid sequence of SEQ ID NO: 73 and a VL comprising the amino acid sequence of SEQ ID NO: 75.

In some embodiments, the anti-TfR1 antibody of the present disclosure comprises a VH comprising the amino acid sequence of SEQ ID NO: 76 and a VL comprising the amino acid sequence of SEQ ID NO: 74.

In some embodiments, the anti-TfR1 antibody of the present disclosure comprises a VH comprising the amino acid sequence of SEQ ID NO: 76 and a VL comprising the amino acid sequence of SEQ ID NO: 75.

In some embodiments, the anti-TfR1 antibody of the present disclosure comprises a VH comprising the amino acid sequence of SEQ ID NO: 77 and a VL comprising the amino acid sequence of SEQ ID NO: 78.

In some embodiments, the anti-TfR1 antibody of the present disclosure comprises a VH comprising the amino acid sequence of SEQ ID NO: 79 and a VL comprising the amino acid sequence of SEQ ID NO: 80.

In some embodiments, the anti-TfR1 antibody of the present disclosure comprises a VH comprising the amino acid sequence of SEQ ID NO: 77 and a VL comprising the amino acid sequence of SEQ ID NO: 80.

In some embodiments, the anti-TfR1 antibody of the present disclosure comprises a VH comprising the amino acid sequence of SEQ ID NO: 154 and a VL comprising the amino acid sequence of SEQ ID NO: 155.

In some embodiments, the anti-TfR1 antibody described herein is a full-length IgG, which can include a heavy constant region and a light constant region from a human antibody. In some embodiments, the heavy chain of any of the anti-TfR1 antibodies as described herein may comprise a heavy chain constant region (CH) or a portion thereof (e.g., CH1, CH2, CH3, or a combination thereof). The heavy chain constant region can be of any suitable origin, e.g., human, mouse, rat, or rabbit. In one specific example, the heavy chain constant region is from a human IgG (a gamma heavy chain), e.g., IgG1, IgG2, or IgG4. An example of a human IgG1 constant region is given below:

(SEQ ID NO: 81)
ASTKGPSVFPLAPSSKSTSGGTAALGCLVKDYFPEPVTVSWNSGAL
TSGVHTFPAVLQSSGLYSLSSVVTVPSSSLGTQTYICNVNHKPSNT
KVDKKVEPKSCDKTHTCPPCPAPELLGGPSVFLFPPKPKDTLMISR
TPEVTCVVVDVSHEDPEVKFNWYVDGVEVHNAKTKPREEQYNSTYR
VVSVLTVLHQDWLNGKEYKCKVSNKALPAPIEKTISKAKGQPREPQ
VYTLPPSRDELTKNQVSLTCLVKGFYPSDIAVEWESNGQPENNYKT
TPPVLDSDGSFFLYSKLTVDKSRWQQGNVFSCSVMHEALHNHYTQK
SLSLSPGK

In some embodiments, the heavy chain of any of the anti-TfR1 antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcγ receptor binding (Bruhns, P., et al. (2009) and Xu, D. et al. (2000)). The mutant human IgG1 constant region is provided below (mutations bonded and underlined):

(SEQ ID NO: 82)
ASTKGPSVFPLAPSSKSTSGGTAALGCLVKDYFPEPVTVSWNSGAL
TSGVHTFPAVLQSSGLYSLSSVVTVPSSSLGTQTYICNVNHKPSNT
KVDKKVEPKSCDKTHTCPPCPAPEAAGGPSVFLFPPKPKDTLMISR
TPEVTCVVVDVSHEDPEVKFNWYVDGVEVHNAKTKPREEQYNSTYR
VVSVLTVLHQDWLNGKEYKCKVSNKALPAPIEKTISKAKGQPREPQ
VYTLPPSRDELTKNQVSLTCLVKGFYPSDIAVEWESNGQPENNYKT
TPPVLDSDGSFFLYSKLTVDKSRWQQGNVFSCSVMHEALHNHYTQK
SLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNA
LQSGNSQESVTEQDSKDSTYSLSSTLTLSKADYEKHKVYACEVTHQ
GLSSPVTKSENRGEC

Other antibody heavy and light chain constant regions are well known in the art, e.g., those provided in the IMGT database (www.imgt.org) or at www.vbase2.org/vbstat.php, both of which are incorporated by reference herein.

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 81 or SEQ ID NO: 82. In some embodiments, the anti-TfR1 antibody described herein comprises a heavy chain comprising any one of the VH as listed in Table 3 or any variants thereof and a heavy chain constant region as set forth in SEQ ID NO: 81. In some embodiments, the anti-TfR1 antibody described herein comprises heavy chain comprising any one of the VH as listed in Table 3 or any variants thereof and a heavy chain constant region as set forth in SEQ ID NO: 82.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of IgG heavy chain and light chain amino acid sequences of the anti-TfR1 antibodies described are provided in Table 4 below.

TABLE 4
Heavy chain and light chain sequences of examples of anti-TfR1 IgGs

| Antibody | IgG Heavy Chain/Light Chain Sequences** |
|---|---|
| 3A4 | Heavy Chain (with wild type human IgG1 constant region) |
| VH3 (N54T*)/ | EVQLVQSGSELKKPGASVKVSCTASGFNIKDDYMYWVRQPPGKGLEWIGWIDPE |
| Vκ4 | TGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 84) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with wild type human IgG1 constant region) |
| VH3 (N54S*)/ | EVQLVQSGSELKKPGASVKVSCTASGFNIKDDYMYWVRQPPGKGLEWIGWIDPE |
| Vκ4 | SGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 86) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with wild type human IgG1 constant region) |
| VH3/Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 87) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3M12 | Heavy Chain (with wild type human IgG1 constant region) |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region) |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK (SEQ ID NO: 88) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 3M12 | Heavy Chain (with wild type human IgG1 constant region) |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with wild type human IgG1 constant region) |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK (SEQ ID NO: 91) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 5H12 | Heavy Chain (with wild type human IgG1 constant region) |
| VH5 (C33Y*)/ | QVQLVQSGAEVKKPGASVKVSCKASGYSFTDYYINWVRQAPGQGLEWMGWIYP |
| Vκ3 | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 92) |
| | Light Chain (with kappa light chain constant region) |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with wild type human IgG1 constant region) |
| VH5 (C33D*)/ | QVQLVQSGAEVKKPGASVKVSCKASGYSFTDYDINWVRQAPGQGLEWMGWIYP |
| Vκ4 | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 94) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| 5H12 | Heavy Chain (with wild type human IgG1 constant region) |
| VH5 (C33Y*)/ | QVQLVQSGAEVKKPGASVKVSCKASGYSFTDYYINWVRQAPGQGLEWMGWIYP |
| Vκ4 | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 92) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| Anti-TfR | VH: |
| clone 8 | QVQLVQSGAEVKKPGESLKISCKGSGYSFTSYWIGWVRQMPGKGLEWMGIIYPG |
| | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGK (SEQ ID NO: 156) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 84, 86, 87, 88, 91, 92, 94, and 156. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 84, 86, 87, 88, 91, 92, 94, and 156. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 84, 86, 87, 88, 91, 92, 94, and 156. Alternatively or in addition (e.g., in addition), the anti-TfR1 antibody described herein comprises a light chain comprising the amino acid sequence of any one of SEQ ID NOs: 85, 89, 90, 93, 95 and 157.

In some embodiments, the anti-TfR1 antibody of the present disclosure comprises a heavy chain comprising the amino acid sequence of SEQ ID NO: 84 and a light chain comprising the amino acid sequence of SEQ ID NO: 85.

In some embodiments, the anti-TfR1 antibody of the present disclosure comprises a heavy chain comprising the amino acid sequence of SEQ ID NO: 86 and a light chain comprising the amino acid sequence of SEQ ID NO: 85.

In some embodiments, the anti-TfR1 antibody of the present disclosure comprises a heavy chain comprising the amino acid sequence of SEQ ID NO: 87 and a light chain comprising the amino acid sequence of SEQ ID NO: 85.

In some embodiments, the anti-TfR1 antibody of the present disclosure comprises a heavy chain comprising the amino acid sequence of SEQ ID NO: 88 and a light chain comprising the amino acid sequence of SEQ ID NO: 89.

In some embodiments, the anti-TfR1 antibody of the present disclosure comprises a heavy chain comprising the amino acid sequence of SEQ ID NO: 88 and a light chain comprising the amino acid sequence of SEQ ID NO: 90.

In some embodiments, the anti-TfR1 antibody of the present disclosure comprises a heavy chain comprising the amino acid sequence of SEQ ID NO: 91 and a light chain comprising the amino acid sequence of SEQ ID NO: 89.

In some embodiments, the anti-TfR1 antibody of the present disclosure comprises a heavy chain comprising the amino acid sequence of SEQ ID NO: 91 and a light chain comprising the amino acid sequence of SEQ ID NO: 90.

In some embodiments, the anti-TfR1 antibody of the present disclosure comprises a heavy chain comprising the amino acid sequence of SEQ ID NO: 92 and a light chain comprising the amino acid sequence of SEQ ID NO: 93.

In some embodiments, the anti-TfR1 antibody of the present disclosure comprises a heavy chain comprising the amino acid sequence of SEQ ID NO: 94 and a light chain comprising the amino acid sequence of SEQ ID NO: 95.

In some embodiments, the anti-TfR1 antibody of the present disclosure comprises a heavy chain comprising the amino acid sequence of SEQ ID NO: 92 and a light chain comprising the amino acid sequence of SEQ ID NO: 95.

In some embodiments, the anti-TfR1 antibody of the present disclosure comprises a heavy chain comprising the amino acid sequence of SEQ ID NO: 156 and a light chain comprising the amino acid sequence of SEQ ID NO: 157.

In some embodiments, the anti-TfR1 antibody is a Fab fragment, Fab′ fragment, or F(ab′)₂ fragment of an intact antibody (full-length antibody). Antigen binding fragment of an intact antibody (full-length antibody) can be prepared via routine methods (e.g., recombinantly or by digesting the heavy chain constant region of a full-length IgG using an enzyme such as papain). For example, F(ab′)₂ fragments can be produced by pepsin or papain digestion of an antibody molecule, and Fab fragments that can be generated by reducing the disulfide bridges of F(ab′)₂ fragments. In some embodiments, a heavy chain constant region in a Fab fragment of the anti-TfR1 antibody described herein comprises the amino acid sequence of:

(SEQ ID NO: 96)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHT

In some embodiments, the anti-TfR1 antibody described herein comprises a heavy chain comprising any one of the VH as listed in Table 3 or any variants thereof and a heavy chain constant region that is at least 80%, at least 85%, at least 90%, at least 95%, or at least 99% identical to SEQ ID NO: 96. In some embodiments, the anti-TfR1 antibody described herein comprises a heavy chain comprising any one of the VH as listed in Table 3 or any variants thereof and a heavy chain constant region that contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 96. In some embodiments, the anti-TfR1 antibody described herein comprises a heavy chain comprising any one of the VH as listed in Table 3 or any variants thereof and a heavy chain constant region as set forth in SEQ ID NO: 96.

In some embodiments, the anti-TfR1 antibody described herein comprises a light chain comprising any one of the VL as listed in Table 3 or any variants thereof and a light chain constant region that is at least 80%, at least 85%, at least 90%, at least 95%, or at least 99% identical to SEQ ID NO: 83. In some embodiments, the anti-TfR1 antibody described herein comprises a light chain comprising any one of the VL as listed in Table 3 or any variants thereof and a light chain constant region contains no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with SEQ ID NO: 83. In some embodiments, the anti-TfR1 antibody described herein comprises a light chain comprising any one of the VL as listed in Table 3 or any variants thereof and a light chain constant region set forth in SEQ ID NO: 83.

Examples of Fab heavy chain and light chain amino acid sequences of the anti-TfR1 antibodies described are provided in Table 5 below.

TABLE 5
Heavy chain and light chain sequences of examples of anti-TfR1 Fabs

| Antibody | Fab Heavy Chain/Light Chain Sequences** |
|---|---|
| 3A4 | Heavy Chain (with partial human IgG1 constant region) |
| VH3 (N54T*)/ | EVQLVQSGSELKKPGASVKVSCTASGFNIKDDYMYWVRQPPGKGLEWIGWIDPE |
| Vκ4 | TGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 97) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with partial human IgG1 constant region) |
| VH3 (N54S*)/ | EVQLVQSGSELKKPGASVKVSCTASGFNIKDDYMYWVRQPPGKGLEWIGWIDPE |
| Vκ4 | SGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 98) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3A4 | Heavy Chain (with partial human IgG1 constant region) |
| VH3/Vκ4 | EVQLVQSGSELKKPGASVKVSCTASGFNIKDDYMYWVRQPPGKGLEWIGWIDPE |
| | NGDTEYASKFQDRVTVTADTSTNTAYMELSSLRSEDTAVYYCTLWLRRGLDYW |
| | GQGTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKKVEPKS |
| | CDKTHT (SEQ ID NO: 99) |
| | Light Chain (with kappa light chain constant region) |
| | DIVMTQSPLSLPVTPGEPASISCRSSKSLLHSNGYTYLFWFQQRPGQSPRLLIYRMS |
| | NLASGVPDRFSGSGSGTDFTLKISRVEAEDVGVYYCMQHLEYPFTFGGGTKVEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 85) |
| 3M12 | Heavy Chain (with partial human IgG1 constant region) |
| VH3/Vκ2 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region) |
| VH3/Vκ3 | QVQLQESGPGLVKPSQTLSLTCSVTGYSITSGYYWNWIRQPPGKGLEWMGYITFD |
| | GANNYNPSLKNRVSISRDTSKNQFSLKLSSVTAEDTATYYCTRSSYDYDVLDYWG |
| | QGTTVTVSSASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVDKKVEPKSC |
| | DKTHT (SEQ ID NO: 100) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 3M12 | Heavy Chain (with partial human IgG1 constant region) |
| VH4/Vκ2 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYFCQQGHTLPYTFGQGTKLEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: 89) |
| 3M12 | Heavy Chain (with partial human IgG1 constant region) |
| VH4/Vκ3 | QVQLQESGPGLVKPSQTLSLTCTVTGYSITSGYYWNWIRQPPGKGLEWIGYITFDG |
| | ANNYNPSLKNRVSISRDTSKNQFSLKLSSVTAEDTATYYCTRSSYDYDVLDYWGQ |
| | GTTVTVSSASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDKKVEPKSCD |
| | KTHT (SEQ ID NO: 101) |
| | Light Chain (with kappa light chain constant region) |
| | DIQMTQSPSSLSASVGDRVTITCRASQDISNFLNWYQQKPGQPVKLLIYYTSRLHS |
| | GVPSRFSGSGSGTDFTLTISSLQPEDFATYYCQQGHTLPYTFGQGTKLEIKRTVAA |
| | PSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC (SEQ ID NO: |
| | 90) |
| 5H12 | Heavy Chain (with partial human IgG1 constant region) |
| VH5 (C33Y*)/ | QVQLVQSGAEVKKPGASVKVSCKASGYSFTDYYINWVRQAPGQGLEWMGWIYP |
| Vκ3 | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 102) |
| | Light Chain (with kappa light chain constant region) |
| | DIVLTQSPDSLAVSLGERATINCRASESVDGYDNSFMHWYQQKPGQPPKLLIFRAS |
| | NLESGVPDRFSGSGSRTDFTLTISSLQAEDVAVYYCQQSSEDPWTFGQGTKLEIKR |
| | TVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC (SEQ ID |
| | NO: 93) |
| 5H12 | Heavy Chain (with partial human IgG1 constant region) |
| VH5 (C33D*)/ | QVQLVQSGAEVKKPGASVKVSCKASGYSFTDYDINWVRQAPGQGLEWMGWIYP |
| Vκ4 | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 103) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| 5H12 | Heavy Chain (with partial human IgG1 constant region) |
| VH5 (C33Y*)/ | QVQLVQSGAEVKKPGASVKVSCKASGYSFTDYYINWVRQAPGQGLEWMGWIYP |
| Vκ4 | GSGNTRYSERFKGRVTITRDTSASTAYMELSSLRSEDTAVYYCAREDYYPYHGM |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 102) |
| | Light Chain (with kappa light chain constant region) |
| | DIVMTQSPDSLAVSLGERATINCRASESVDGYDNSFMHWYQQKPGQPPKLLIFRA |
| | SNLESGVPDRFSGSGSGTDFTLTISSLQAEDVAVYYCQQSSEDPWTFGQGTKLEIK |
| | RTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC (SEQ |
| | ID NO: 95) |
| Anti-TfR | VH: |
| clone 8 | QVQLVQSGAEVKKPGESLKISCKGSGYSFTSYWIGWVRQMPGKGLEWMGIIYPG |
| Version 1 | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHTCP (SEQ ID NO: 158) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |
| Anti-TfR | VH: |
| clone 8 | QVQLVQSGAEVKKPGESLKISCKGSGYSFTSYWIGWVRQMPGKGLEWMGIIYPG |
| Version 2 | DSDTRYSPSFQGQVTISADKSISTAYLQWSSLKASDTAMYYCARFPYDSSGYYSF |
| | DYWGQGTLVTVSSASTKGPSVFPLAPSSKSTSGGTAALGCLVKDYFPEPVTVSWN |
| | SGALTSGVHTFPAVLQSSGLYSLSSVVTVPSSSLGTQTYICNVNHKPSNTKVDKKV |
| | EPKSCDKTHT (SEQ ID NO: 159) |
| | VL: |
| | DIQMTQSPSSLSASVGDRVTITCRASQSISSYLNWYQQKPGKAPKLLIYAASSLQS |
| | GVPSRFSGSGSGTDFTLTISSLQPEDFATYYCQQSYSTPLTFGGGTKVEIKRTVAAP |
| | SVFIFPPSDEQLKSGTASVVCLLNNFYPREAKVQWKVDNALQSGNSQESVTEQDSK |
| | DSTYSLSSTLTLSKADYEKHKVYACEVTHQGLSSPVTKSFNRGEC (SEQ ID NO: |
| | 157) |

*mutation positions are according to Kabat numbering of the respective VH sequences containing the mutations
**CDRs according to the Kabat numbering system are bolded; VH/VL sequences underlined

In some embodiments, the anti-TfR1 antibody of the present disclosure comprises a heavy chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 97-103, 158 and 159. Alternatively or in addition (e.g., in addition), the anti-TfR1 antibody of the present disclosure comprises a light chain containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the light chain as set forth in any one of SEQ ID NOs: 85, 89, 90, 93, 95, and 157.

In some embodiments, the anti-TfR1 antibody described herein comprises a heavy chain comprising an amino acid sequence that is at least 75% (e.g., 75%, 80%, 85%, 90%, 95%, 98%, or 99%) identical to any one of SEQ ID NOs: 97-103, 158 and 159. Alternatively or in addition (e.g., in addition), the anti-TfR1 antibody described herein comprises a light chain comprising an amino acid sequence that is at least 75% (e.g., 75%, 80%, 85%, 90%, 95%, 98%, or 99%) identical to any one of SEQ ID NOs: 85, 89, 90, 93, 95, and 157. In some embodiments, the anti-TfR1 antibody described herein comprises a heavy chain comprising the amino acid sequence of any one of SEQ ID NOs: 97-103, 158 and 159. Alternatively or in addition (e.g., in addition), the anti-TfR1 antibody described herein comprises a light chain comprising the amino acid sequence of any one of SEQ ID NOs: 85, 89, 90, 93, 95, and 157.

In some embodiments, the anti-TfR1 antibody of the present disclosure comprises a heavy chain comprising the amino acid sequence of SEQ ID NO: 97 and a light chain comprising the amino acid sequence of SEQ ID NO: 85.

In some embodiments, the anti-TfR1 antibody of the present disclosure comprises a heavy chain comprising the amino acid sequence of SEQ ID NO: 98 and a light chain comprising the amino acid sequence of SEQ ID NO: 85.

In some embodiments, the anti-TfR1 antibody of the present disclosure comprises a heavy chain comprising the amino acid sequence of SEQ ID NO: 99 and a light chain comprising the amino acid sequence of SEQ ID NO: 85.

In some embodiments, the anti-TfR1 antibody of the present disclosure comprises a heavy chain comprising the amino acid sequence of SEQ ID NO: 100 and a light chain comprising the amino acid sequence of SEQ ID NO: 89.

In some embodiments, the anti-TfR1 antibody of the present disclosure comprises a heavy chain comprising the amino acid sequence of SEQ ID NO: 100 and a light chain comprising the amino acid sequence of SEQ ID NO: 90.

In some embodiments, the anti-TfR1 antibody of the present disclosure comprises a heavy chain comprising the amino acid sequence of SEQ ID NO: 101 and a light chain comprising the amino acid sequence of SEQ ID NO: 89.

In some embodiments, the anti-TfR1 antibody of the present disclosure comprises a heavy chain comprising the amino acid sequence of SEQ ID NO: 101 and a light chain comprising the amino acid sequence of SEQ ID NO: 90.

In some embodiments, the anti-TfR1 antibody of the present disclosure comprises a heavy chain comprising the amino acid sequence of SEQ ID NO: 102 and a light chain comprising the amino acid sequence of SEQ ID NO: 93.

In some embodiments, the anti-TfR1 antibody of the present disclosure comprises a heavy chain comprising the amino acid sequence of SEQ ID NO: 103 and a light chain comprising the amino acid sequence of SEQ ID NO: 95.

In some embodiments, the anti-TfR1 antibody of the present disclosure comprises a heavy chain comprising the amino acid sequence of SEQ ID NO: 102 and a light chain comprising the amino acid sequence of SEQ ID NO: 95.

In some embodiments, the anti-TfR1 antibody of the present disclosure comprises a heavy chain comprising the amino acid sequence of SEQ ID NO: 158 and a light chain comprising the amino acid sequence of SEQ ID NO: 157.

In some embodiments, the anti-TfR1 antibody of the present disclosure comprises a heavy chain comprising the amino acid sequence of SEQ ID NO: 159 and a light chain comprising the amino acid sequence of SEQ ID NO: 157.

### Other Known Anti-TfR1 Antibodies

Any other appropriate anti-TfR1 antibodies known in the art may be used as the muscle-targeting agent in the complexes disclosed herein. Examples of known anti-TfR1 antibodies, including associated references and binding epitopes, are listed in Table 6. In some embodiments, the anti-TfR1 antibody comprises the complementarity determining regions (CDR-H1, CDR-H2, CDR-H3, CDR-L1, CDR-L2, and CDR-L3) of any of the anti-TfR1 antibodies provided herein, e.g., anti-TfR1 antibodies listed in Table 6.

TABLE 6
List of anti-TfR1 antibody clones, including associated
references and binding epitope information.

| Antibody Clone | | |
|---|---|---|
| Name | Reference(s) | Epitope/Notes |
| OKT9 | U.S. Pat. No. 4,364,934, filed Dec. 4, 1979, | Apical domain of TfR1 |
| | entitled “MONOCLONAL ANTIBODY TO | (residues 305-366 of |
| | A HUMAN EARLY THYMOCYTE | human TfR1 sequence |
| | ANTIGEN AND METHODS FOR | XM_052730.3, available |
| | PREPARING SAME” | in GenBank) |
| | Schneider C. et al. “Structural features of the | |
| | cell surface receptor for transferrin that is | |
| | recognized by the monoclonal antibody | |
| | OKT9.” J Biol Chem. 1982, 257: 14, 8516- | |
| | 8522. | |
| (From JCR) | WO 2015/098989, filed Dec. 24, 2014, | Apical domain (residues |
| Clone M11 | “Novel anti-Transferrin receptor antibody | 230-244 and 326-347 of |
| Clone M23 | that passes through blood-brain barrier” | TfR1) and protease-like |
| Clone M27 | U.S. Pat. No. 9,994,641, filed | domain (residues 461- |
| Clone B84 | Dec. 24, 2014, “Novel anti-Transferrin | 473) |
| | receptor antibody that passes through | |
| | blood-brain barrier” | |
| (From | WO 2016/081643, filed May 26, 2016, | Apical domain and non- |
| Genentech) | entitled “ANTI-TRANSFERRIN | apical regions |
| 7A4, 8A2, 15D2, | RECEPTOR ANTIBODIES AND | |
| 10D11, 7B10, | METHODS OF USE” | |
| 15G11, 16G5, | U.S. Pat. No. 9,708,406, filed | |
| 13C3, 16G4, | May 20, 2014, “Anti-transferrin receptor | |
| 16F6, 7G7, 4C2, | antibodies and methods of use” | |
| 1B12, and 13D4 | | |
| (From Armagen) | Lee et al. “Targeting Rat Anti-Mouse | |
| 8D3 | Transferrin Receptor Monoclonal Antibodies | |
| | through Blood-Brain Barrier in Mouse” | |
| | 2000, J Pharmacol. Exp. Ther., 292: 1048- | |
| | 1052. | |
| | US Patent App. 2010/077498, filed | |
| | Sep. 11, 2008, entitled “COMPOSITIONS AND | |
| | METHODS FOR BLOOD-BRAIN | |
| | BARRIER DELIVERY IN THE MOUSE” | |
| OX26 | Haobam, B. et al. 2014. Rab17- | |
| | mediated recycling endosomes contribute to | |
| | autophagosome formation in response to | |
| | Group A Streptococcus invasion. Cellular | |
| | microbiology. 16: 1806-21. | |
| DF1513 | Ortiz-Zapater E et al. Trafficking of | |
| | the human transferrin receptor in plant cells: | |
| | effects of tyrphostin A23 and brefeldin A. | |
| | Plant J 48: 757-70 (2006). | |
| 1A1B2, 66IG10, | Commercially available anti- | Novus Biologicals |
| MEM-189, | transferrin receptor antibodies. | 8100 Southpark Way, A-8 |
| JF0956, 29806, | | Littleton CO 80120 |
| 1A1B2, | | |
| TFRC/1818, 1E6, | | |
| 66Ig10, | | |
| TFRC/1059, | | |
| Q1/71, 23D10, | | |
| 13E4, | | |
| TFRC/1149, ER- | | |
| MP21, YTA74.4, | | |
| BU54, 2B6, RI7217 | | |
| (From INSERM) | US Patent App. 2011/0311544A1, | Does not compete with |
| BA120g | filed Jun. 15, 2005, entitled “ANTI-CD71 | OKT9 |
| | MONOCLONAL ANTIBODIES AND | |
| | USES THEREOF FOR TREATING | |
| | MALIGNANT TUMOR CELLS” | |
| LUCA31 | U.S. Pat. No. 7,572,895, filed | “LUCA31 epitope” |
| | Jun. 7, 2004, entitled “TRANSFERRIN | |
| | RECEPTOR ANTIBODIES” | |
| (Salk Institute) | Trowbridge, I. S. et al. “Anti-transferrin | |
| B3/25 | receptor monoclonal antibody and toxin- | |
| T58/30 | antibody conjugates affect growth of | |
| | human tumour cells.” Nature, 1981, | |
| | volume 294, pages 171-173 | |
| R17 217.1.3, | Commercially available anti- | BioXcell |
| 5E9C11, | transferrin receptor antibodies. | 10 Technology Dr., Suite |
| OKT9 (BE0023 | | 2B |
| clone) | | West Lebanon, NH |
| | | 03784-1671 USA |
| BK19.9, B3/25, | Gatter, K. C. et al. “Transferrin receptors | |
| T56/14 and T58/1 | in human tissues: their distribution and | |
| | possible clinical relevance.” J Clin | |
| | Pathol. 1983 May; 36(5): 539-45. | |

Anti-TfR1 antibody
CDRH1 (SEQ ID NO: 372)
CDRH2 (SEQ ID NO: 373)
CDRH3 (SEQ ID NO: 374)
CDRL1 (SEQ ID NO: 375)
CDRL2 (SEQ ID NO: 376)
CDRL3 (SEQ ID NO: 377)
VH (SEQ ID NO: 378)
VL (SEQ ID NO: 379)

Additional Anti-TfR1 antibody SEQ ID NOs

| | VH/VL | CDR1 | CDR2 | CDR3 |
|---|---|---|---|---|
| VH1 | 387 | 380 | 381 | 374 |
| VH2 | 388 | 380 | 382 | 374 |
| VH3 | 389 | 380 | 383 | 374 |
| VH4 | 390 | 380 | 382 | 374 |
| VL1 | 391 | 375 | 376 | 115 |
| VL2 | 392 | 375 | 376 | 115 |
| VL3 | 393 | 375 | 384 | 377 |
| VL4 | 394 | 385 | 386 | 377 |

In some embodiments, anti-TfR1 antibodies of the present disclosure include one or more of the CDR-H (e.g., CDR-H1, CDR-H2, and CDR-H3) amino acid sequences from any one of the anti-TfR1 antibodies selected from Table 6. In some embodiments, anti-TfR1 antibodies include the CDR-L1, CDR-L2, and CDR-L3 as provided for any one of the anti-TfR1 antibodies selected from Table 6. In some embodiments, anti-TfR1 antibodies include the CDR-H1, CDR-H2, CDR-H3, CDR-L1, CDR-L2, and CDR-L3 as provided for any one of the anti-TfR1 antibodies selected from Table 6.

In some embodiments, anti-TfR1 antibodies of the disclosure include any antibody that includes a heavy chain variable domain and/or (e.g., and) a light chain variable domain of any anti-TfR1 antibody, such as any one of the anti-TfR1 antibodies selected from Table 6. In some embodiments, anti-TfR1 antibodies of the disclosure include any antibody that includes the heavy chain variable and light chain variable pairs of any anti-TfR1 antibody, such as any one of the anti-TfR1 antibodies selected from Table 6.

Aspects of the disclosure provide anti-TfR1 antibodies having a heavy chain variable (VH) and/or (e.g., and) a light chain variable (VL) domain amino acid sequence homologous to any of those described herein. In some embodiments, the anti-TfR1 antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any anti-TfR1 antibody, such as any one of the anti-TfR1 antibodies selected from Table 6. In some embodiments, the homologous heavy chain variable and/or (e.g., and) a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or (e.g., and) a light chain variable sequence excluding any of the CDR sequences provided herein. In some embodiments, any of the anti-TfR1 antibodies provided herein comprise a heavy chain variable sequence and a light chain variable sequence that comprises a framework sequence that is at least 75%, 80%, 85%, 90%, 95%, 98%, or 99% identical to the framework sequence of any anti-TfR1 antibody, such as any one of the anti-TfR1 antibodies selected from Table 6.

An example of a transferrin receptor antibody that may be used in accordance with the present disclosure is described in International Application Publication WO 2016/081643, incorporated herein by reference. The amino acid sequences of this antibody are provided in Table 7.

TABLE 7
Heavy chain and light chain CDRs of an example of a known anti-TfR1 antibody

| Sequence Type | Kabat | Chothia | Contact |
|---|---|---|---|
| CDR-H1 | SYWMH (SEQ ID | GYTFTSY (SEQ ID | TSYWMH (SEQ ID |
| | NO: 110) | NO: 116) | NO: 118) |
| CDR-H2 | EINPTNGRTNYIE | NPTNGR (SEQ ID | WIGEINPTNGRTN |
| | KFKS (SEQ ID | NO: 117) | (SEQ ID NO: 119) |
| | NO: 111) | | |
| CDR-H3 | GTRAYHY (SEQ | GTRAYHY (SEQ ID | ARGTRA |
| | ID NO: 112) | NO: 112) | (SEQ ID NO: 120) |
| CDR-L1 | RASDNLYSNLA | RASDNLYSNLA (SEQ | YSNLAWY (SEQ ID |
| | (SEQ ID NO: 113) | ID NO: 113) | NO: 121) |
| CDR-L2 | DATNLAD (SEQ | DATNLAD (SEQ ID | LLVYDATNLA (SEQ |
| | ID NO: 114) | NO: 114) | ID NO: 122) |
| CDR-L3 | QHFWGTPLT | QHFWGTPLT (SEQ ID | QHFWGTPL (SEQ |
| | (SEQ ID NO: 115) | NO: 115) | ID NO: 123) |

| | |
|---|---|
| Murine VH | QVQLQQPGAELVKPGASVKLSCKASGYTFTSYWMHWVKQRPGQGLEWIGEINP |
| | TNGRTNYIEKFKSKATLTVDKSSSTAYMQLSSLTSEDSAVYYCARGTRAYHYW |
| | GQGTSVTVSS (SEQ ID NO: 124) |
| Murine VL | DIQMTQSPASLSVSVGETVTITCRASDNLYSNLAWYQQKQGKSPQLLVYDATNL |
| | ADGVPSRFSGSGSGTQYSLKINSLQSEDFGTYYCQHFWGTPLTFGAGTKLELK |
| | (SEQ ID NO: 125) |
| Humanized VH | EVQLVQSGAEVKKPGASVKVSCKASGYTFTSYWMHWVRQAPGQRLEWIGEIN |
| | PTNGRTNYIEKFKSRATLTVDKSASTAYMELSSLRSEDTAVYYCARGTRAYHY |
| | WGQGTMVTVSS (SEQ ID NO: 128) |
| Humanized VL | DIQMTQSPSSLSASVGDRVTITCRASDNLYSNLAWYQQKPGKSPKLLVYDATNL |
| | ADGVPSRFSGSGSGTDYTLTISSLQPEDFATYYCQHFWGTPLTFGQGTKVEIK |
| | (SEQ ID NO: 129) |
| HC of chimeric | QVQLQQPGAELVKPGASVKLSCKASGYTFTSYWMHWVKQRPGQGLEWIGEINP |
| full-length IgG1 | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAV |
| | EWESNGQPENNYKTTPPVLDSDGSFFLYSKLTVDKSRWQQGNVFSCSVMHEAL |
| | HNHYTQKSLSLSPGK (SEQ ID NO: 132) |
| LC of chimeric | DIQMTQSPASLSVSVGETVTITCRASDNLYSNLAWYQQKQGKSPQLLVYDATNL |
| full-length IgG1 | ADGVPSRFSGSGSGTQYSLKINSLQSEDFGTYYCQHFWGTPLTFGAGTKLELKR |
| | TVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| | (SEQ ID NO: 133) |
| HC of fully human | EVQLVQSGAEVKKPGASVKVSCKASGYTFTSYWMHWVRQAPGQRLEWIGEIN |
| full-length IgG1 | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIA |
| | VEWESNGQPENNYKTTPPVLDSDGSFFLYSKLTVDKSRWQQGNVFSCSVMHEA |
| | LHNHYTQKSLSLSPGK (SEQ ID NO: 134) |
| LC of fully human | DIQMTQSPSSLSASVGDRVTITCRASDNLYSNLAWYQQKPGKSPKLLVYDATNL |
| full-length IgG1 | ADGVPSRFSGSGSGTDYTLTISSLQPEDFATYYCQHFWGTPLTFGQGTKVEIKRT |
| | VAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| | (SEQ ID NO: 135) |
| HC of chimeric | QVQLQQPGAELVKPGASVKLSCKASGYTFTSYWMHWVKQRPGQGLEWIGEINP |
| Fab | TNGRTNYIEKFKSKATLTVDKSSSTAYMQLSSLTSEDSAVYYCARGTRAYHYW |
| | GQGTSVTVSSASTKGPSVFPLAPSSKSTSGGTAALGCLVKDYFPEPVTVSWNSG |
| | ALTSGVHTFPAVLQSSGLYSLSSVVTVPSSSLGTQTYICNVNHKPSNTKVDKKVE |
| | PKSCDKTHTCP (SEQ ID NO: 136) |
| HC of fully human | EVQLVQSGAEVKKPGASVKVSCKASGYTFTSYWMHWVRQAPGQRLEWIGEIN |
| Fab | PTNGRTNYIEKFKSRATLTVDKSASTAYMELSSLRSEDTAVYYCARGTRAYHY |
| | WGQGTMVTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSNTKVDKKV |
| | EPKSCDKTHTCP (SEQ ID NO: 137) |

In some embodiments, the anti-TfR1 antibody of the present disclosure comprises a CDR-H1, a CDR-H2, and a CDR-H3 that are the same as the CDR-H1, CDR-H2, and CDR-H3 shown in Table 7. Alternatively or in addition (e.g., in addition), the anti-TfR1 antibody of the present disclosure comprises a CDR-L1, a CDR-L2, and a CDR-L3 that are the same as the CDR-L1, CDR-L2, and CDR-L3 shown in Table 7.

In some embodiments, the anti-TfR1 antibody of the present disclosure comprises a CDR-L3, which contains no more than 3 amino acid variations (e.g., no more than 3, 2, or 1 amino acid variation) as compared with the CDR-L3 as shown in Table 7. In some embodiments, the anti-TfR1 antibody of the present disclosure comprises a CDR-L3 containing one amino acid variation as compared with the CDR-L3 as shown in Table 7. In some embodiments, the anti-TfR1 antibody of the present disclosure comprises a CDR-L3 of QHFAGTPLT (SEQ ID NO: 126) (according to the Kabat and Chothia definition system) or QHFAGTPL (SEQ ID NO: 127) (according to the Contact definition system). In some embodiments, the anti-TfR1 antibody of the present disclosure comprises a CDR-H1, a CDR-H2, a CDR-H3, a CDR-L1 and a CDR-L2 that are the same as the CDR-H1, CDR-H2, and CDR-H3 shown in Table 7, and comprises a CDR-L3 of QHFAGTPLT (SEQ ID NO: 126) (according to the Kabat and Chothia definition system) or QHFAGTPL (SEQ ID NO: 127) (according to the Contact definition system).

In some embodiments, the anti-TfR1 antibody of the present disclosure comprises heavy chain CDRs that collectively are at least 80% (e.g., 80%, 85%, 90%, 95%, or 98%) identical to the heavy chain CDRs as shown in Table 7. Alternatively or in addition (e.g., in addition), the anti-TfR1 antibody of the present disclosure comprises light chain CDRs that collectively are at least 80% (e.g., 80%, 85%, 90%, 95%, or 98%) identical to the light chain CDRs as shown in Table 7.

In some embodiments, the anti-TfR1 antibody of the present disclosure comprises a VH comprising the amino acid sequence of SEQ ID NO: 124. Alternatively or in addition (e.g., in addition), the anti-TfR1 antibody of the present disclosure comprises a VL comprising the amino acid sequence of SEQ ID NO: 125.

In some embodiments, the anti-TfR1 antibody of the present disclosure comprises a VH comprising the amino acid sequence of SEQ ID NO: 128. Alternatively or in addition (e.g., in addition), the anti-TfR1 antibody of the present disclosure comprises a VL comprising the amino acid sequence of SEQ ID NO: 129.

In some embodiments, the anti-TfR1 antibody of the present disclosure comprises a VH containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 128. Alternatively or in addition (e.g., in addition), the anti-TfR1 antibody of the present disclosure comprises a VL containing no more than 15 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 129.

In some embodiments, the anti-TfR1 antibody of the present disclosure is a full-length IgG1 antibody, which can include a heavy constant region and a light constant region from a human antibody. In some embodiments, the heavy chain of any of the anti-TfR1 antibodies as described herein may comprises a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain), e.g., IgG1, IgG2, or IgG4. An example of human IgG1 constant region is given below:

(SEQ ID NO: 81)
ASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFF
LYSKLTVDKSRWQQGNVFSCSVMHEALHNHYTQKSLSLSPGK

In some embodiments, the light chain of any of the anti-TfR1 antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 83)
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC

In some embodiments, the anti-TfR1 antibody described herein is a chimeric antibody that comprises a heavy chain comprising the amino acid sequence of SEQ ID NO: 132. Alternatively or in addition (e.g., in addition), the anti-TfR1 antibody described herein comprises a light chain comprising the amino acid sequence of SEQ ID NO: 133.

In some embodiments, the anti-TfR1 antibody described herein is a fully human antibody that comprises a heavy chain comprising the amino acid sequence of SEQ ID NO: 134. Alternatively or in addition (e.g., in addition), the anti-TfR1 antibody described herein comprises a light chain comprising the amino acid sequence of SEQ ID NO: 135.

In some embodiments, the anti-TfR1 antibody is an antigen binding fragment (Fab) of an intact antibody (full-length antibody). In some embodiments, the anti-TfR1 Fab described herein comprises a heavy chain comprising the amino acid sequence of SEQ ID NO: 136. Alternatively or in addition (e.g., in addition), the anti-TfR1 Fab described herein comprises a light chain comprising the amino acid sequence of SEQ ID NO: 133. In some embodiments, the anti-TfR1 Fab described herein comprises a heavy chain comprising the amino acid sequence of SEQ ID NO: 137. Alternatively or in addition (e.g., in addition), the anti-TfR1 Fab described herein comprises a light chain comprising the amino acid sequence of SEQ ID NO: 135.

The anti-TfR1 antibodies described herein can be in any antibody form, including, but not limited to, intact (i.e., full-length) antibodies, antigen-binding fragments thereof (such as Fab, Fab′, F(ab′)2, Fv), single chain antibodies, bi-specific antibodies, or nanobodies. In some embodiments, the anti-TfR1 antibody described herein is an scFv. In some embodiments, the anti-TfR1 antibody described herein is an scFv-Fab (e.g., scFv fused to a portion of a constant region). In some embodiments, the anti-TfR1 antibody described herein is an scFv fused to a constant region (e.g., human IgG1 constant region as set forth in SEQ ID NO: 81).

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of an anti-TfR1 antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-TfR1 antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-TfR1 antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of an anti-TfR1 antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-TfR1 antibodies described herein may comprise a signal peptide in the heavy and/or (e.g., and) light chain sequence (e.g., a N-terminal signal peptide). In some embodiments, the anti-TfR1 antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MGWSCIILFLVATATGVHS (SEQ ID NO: 104).

In some embodiments, an antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

b. Other Muscle-Targeting Antibodies

In some embodiments, the muscle-targeting antibody is an antibody that specifically binds hemojuvelin, caveolin-3, Duchenne muscular dystrophy peptide, myosin IIb, or CD63. In some embodiments, the muscle-targeting antibody is an antibody that specifically binds a myogenic precursor protein. Exemplary myogenic precursor proteins include, without limitation, ABCG2, M-Cadherin/Cadherin-15, Caveolin-1, CD34, FoxK1, Integrin alpha 7, Integrin alpha 7 beta 1, MYF-5, MyoD, Myogenin, NCAM-1/CD56, Pax3, Pax7, and Pax9. In some embodiments, the muscle-targeting antibody is an antibody that specifically binds a skeletal muscle protein. Exemplary skeletal muscle proteins include, without limitation, alpha-Sarcoglycan, beta-Sarcoglycan, Calpain Inhibitors, Creatine Kinase MM/CKMM, eIF5A, Enolase 2/Neuron-specific Enolase, epsilon-Sarcoglycan, FABP3/H-FABP, GDF-8/Myostatin, GDF-11/GDF-8, Integrin alpha 7, Integrin alpha 7 beta 1, Integrin beta 1/CD29, MCAM/CD146, MyoD, Myogenin, Myosin Light Chain Kinase Inhibitors, NCAM-1/CD56, and Troponin I. In some embodiments, the muscle-targeting antibody is an antibody that specifically binds a smooth muscle protein. Exemplary smooth muscle proteins include, without limitation, alpha-Smooth Muscle Actin, VE-Cadherin, Caldesmon/CALD1, Calponin 1, Desmin, Histamine H2 R, Motilin R/GPR38, Transgelin/TAGLN, and Vimentin. However, it should be appreciated that antibodies to additional targets are within the scope of this disclosure and the exemplary lists of targets provided herein are not meant to be limiting.

c. Antibody Features/Alterations

In some embodiments, conservative mutations can be introduced into antibody sequences (e.g., CDRs or framework sequences) at positions where the residues are not likely to be involved in interacting with a target antigen (e.g., transferrin receptor), for example, as determined based on a crystal structure. In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to alter one or more functional properties of the antibody, such as serum half-life, complement fixation, Fc receptor binding and/or (e.g., and) antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or (e.g., and) CH3 domain (residues 341-447 of human IgG1) and/or (e.g., and) the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to decrease the half-life of the anti-transferrin receptor antibody in vivo. In some embodiments, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or (e.g., and) the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-transferrin receptor antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276: 6591-604).

In some embodiments, one or more amino in the constant region of a muscle-targeting antibody described herein can be replaced with a different amino acid residue such that the antibody has altered C1q binding and/or (e.g., and) reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or (e.g., and) to increase the affinity of the antibody for an Fcγ receptor. This approach is described further in International Publication No. WO 00/42072.

In some embodiments, the heavy and/or (e.g., and) light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind transferrin receptor, such that the variant, CDR-grafted, chimeric, humanized, or composite antibody has at least 50%, at least 60%, at least 70%, at least 80%, at least 90%, at least 95% or more binding to transferrin receptor relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

As provided herein, antibodies of this disclosure may optionally comprise constant regions or parts thereof. For example, a VL domain may be attached at its C-terminal end to a light chain constant domain like Cκ or Cλ. Similarly, a VH domain or portion thereof may be attached to all or part of a heavy chain like IgA, IgD, IgE, IgG, and IgM, and any isotype subclass. Antibodies may include suitable constant regions (see, for example, Kabat et al., Sequences of Proteins of Immunological Interest, No. 91-3242, National Institutes of Health Publications, Bethesda, Md. (1991)). Therefore, antibodies within the scope of this may disclosure include VH and VL domains, or an antigen binding portion thereof, combined with any suitable constant regions.

ii. Muscle-Targeting Peptides

Some aspects of the disclosure provide muscle-targeting peptides as muscle-targeting agents. Short peptide sequences (e.g., peptide sequences of 5-20 amino acids in length) that bind to specific cell types have been described. For example, cell-targeting peptides have been described in Vines e., et al., A. “Cell-penetrating and cell-targeting peptides in drug delivery” Biochim Biophys Acta 2008, 1786: 126-38; Jarver P., et al., “In vivo biodistribution and efficacy of peptide mediated delivery” Trends Pharmacol Sci 2010; 31: 528-35; Samoylova T. I., et al., “Elucidation of muscle-binding peptides by phage display screening” Muscle Nerve 1999; 22: 460-6; U.S. Pat. No. 6,329,501, issued on Dec. 11, 2001, entitled “METHODS AND COMPOSITIONS FOR TARGETING COMPOUNDS TO MUSCLE”; and Samoylov A. M., et al., “Recognition of cell-specific binding of phage display derived peptides using an acoustic wave sensor.” Biomol Eng 2002; 18: 269-72; the entire contents of each of which are incorporated herein by reference. By designing peptides to interact with specific cell surface antigens (e.g., receptors), selectivity for a desired tissue, e.g., muscle, can be achieved. Skeletal muscle-targeting has been investigated and a range of molecular payloads are able to be delivered. These approaches may have high selectivity for muscle tissue without many of the practical disadvantages of a large antibody or viral particle. Accordingly, in some embodiments, the muscle-targeting agent is a muscle-targeting peptide that is from 4 to 50 amino acids in length. In some embodiments, the muscle-targeting peptide is 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length. Muscle-targeting peptides can be generated using any of several methods, such as phage display.

In some embodiments, a muscle-targeting peptide may bind to an internalizing cell surface receptor that is overexpressed or relatively highly expressed in muscle cells, e.g. a transferrin receptor, compared with certain other cells. In some embodiments, a muscle-targeting peptide may target, e.g., bind to, a transferrin receptor. In some embodiments, a peptide that targets a transferrin receptor may comprise a segment of a naturally occurring ligand, e.g., transferrin. In some embodiments, a peptide that targets a transferrin receptor is as described in U.S. Pat. No. 6,743,893, filed Nov. 30, 2000, “RECEPTOR-MEDIATED UPTAKE OF PEPTIDES THAT BIND THE HUMAN TRANSFERRIN RECEPTOR”. In some embodiments, a peptide that targets a transferrin receptor is as described in Kawamoto, M. et al, “A novel transferrin receptor-targeted hybrid peptide disintegrates cancer cell membrane to induce rapid killing of cancer cells.” BMC Cancer. 2011 Aug. 18; 11:359. In some embodiments, a peptide that targets a transferrin receptor is as described in U.S. Pat. No. 8,399,653, filed May 20, 2011, “TRANSFERRIN/TRANSFERRIN RECEPTOR-MEDIATED SIRNA DELIVERY”.

As discussed above, examples of muscle targeting peptides have been reported. For example, muscle-specific peptides were identified using phage display library presenting surface heptapeptides. As one example a peptide having the amino acid sequence ASSLNIA (SEQ ID NO: 363) bound to C2C12 murine myotubes in vitro, and bound to mouse muscle tissue in vivo. Accordingly, in some embodiments, the muscle-targeting agent comprises the amino acid sequence ASSLNIA (SEQ ID NO: 363). This peptide displayed improved specificity for binding to heart and skeletal muscle tissue after intravenous injection in mice with reduced binding to liver, kidney, and brain. Additional muscle-specific peptides have been identified using phage display. For example, a 12 amino acid peptide was identified by phage display library for muscle targeting in the context of treatment for DMD. See, Yoshida D., et al., “Targeting of salicylate to skin and muscle following topical injections in rats.” Int J Pharm 2002; 231: 177-84; the entire contents of which are hereby incorporated by reference. Here, a 12 amino acid peptide having the sequence SKTFNTHPQSTP (SEQ ID NO: 364) was identified and this muscle-targeting peptide showed improved binding to C2C12 cells relative to the ASSLNIA (SEQ ID NO: 363) peptide.

An additional method for identifying peptides selective for muscle (e.g., skeletal muscle) over other cell types includes in vitro selection, which has been described in Ghosh D., et al., “Selection of muscle-binding peptides from context-specific peptide-presenting phage libraries for adenoviral vector targeting” J Virol 2005; 79: 13667-72; the entire contents of which are incorporated herein by reference. By pre-incubating a random 12-mer peptide phage display library with a mixture of non-muscle cell types, non-specific cell binders were selected out. Following rounds of selection the 12 amino acid peptide TARGEHKEEELI (SEQ ID NO: 365) appeared most frequently. Accordingly, in some embodiments, the muscle-targeting agent comprises the amino acid sequence TARGEHKEEELI (SEQ ID NO: 365).

A muscle-targeting agent may an amino acid-containing molecule or peptide. A muscle-targeting peptide may correspond to a sequence of a protein that preferentially binds to a protein receptor found in muscle cells. In some embodiments, a muscle-targeting peptide contains a high propensity of hydrophobic amino acids, e.g. valine, such that the peptide preferentially targets muscle cells. In some embodiments, a muscle-targeting peptide has not been previously characterized or disclosed. These peptides may be conceived of, produced, synthesized, and/or (e.g., and) derivatized using any of several methodologies, e.g. phage displayed peptide libraries, one-bead one-compound peptide libraries, or positional scanning synthetic peptide combinatorial libraries. Exemplary methodologies have been characterized in the art and are incorporated by reference (Gray, B. P. and Brown, K. C. “Combinatorial Peptide Libraries: Mining for Cell-Binding Peptides” Chem Rev. 2014, 114:2, 1020-1081.; Samoylova, T. I. and Smith, B. F. “Elucidation of muscle-binding peptides by phage display screening.” Muscle Nerve, 1999, 22:4. 460-6.). In some embodiments, a muscle-targeting peptide has been previously disclosed (see, e.g. Writer M. J. et al. “Targeted gene delivery to human airway epithelial cells with synthetic vectors incorporating novel targeting peptides selected by phage display.” J. Drug Targeting. 2004; 12:185; Cai, D. “BDNF-mediated enhancement of inflammation and injury in the aging heart.” Physiol Genomics. 2006, 24:3, 191-7.; Zhang, L. “Molecular profiling of heart endothelial cells.” Circulation, 2005, 112:11, 1601-11.; McGuire, M. J. et al. “In vitro selection of a peptide with high selectivity for cardiomyocytes in vivo.” J Mol Biol. 2004, 342:1, 171-82.). Exemplary muscle-targeting peptides comprise an amino acid sequence of the following group: CQAQGQLVC (SEQ ID NO: 366), CSERSMNFC (SEQ ID NO: 367), CPKTRRVPC (SEQ ID NO: 368), WLSEAGPVVTVRALRGTGSW (SEQ ID NO: 369), ASSLNIA (SEQ ID NO: 363), CMQHSMRVC (SEQ ID NO: 370), and DDTRHWG (SEQ ID NO: 371). In some embodiments, a muscle-targeting peptide may comprise about 2-25 amino acids, about 2-20 amino acids, about 2-15 amino acids, about 2-10 amino acids, or about 2-5 amino acids. Muscle-targeting peptides may comprise naturally-occurring amino acids, e.g. cysteine, alanine, or non-naturally-occurring or modified amino acids. Non-naturally occurring amino acids include 3-amino acids, homo-amino acids, proline derivatives, 3-substituted alanine derivatives, linear core amino acids, N-methyl amino acids, and others known in the art. In some embodiments, a muscle-targeting peptide may be linear; in other embodiments, a muscle-targeting peptide may be cyclic, e.g. bicyclic (see, e.g. Silvana, M. G. et al. Mol. Therapy, 2018, 26:1, 132-147.).

iii. Muscle-Targeting Receptor Ligands

A muscle-targeting agent may be a ligand, e.g. a ligand that binds to a receptor protein. A muscle-targeting ligand may be a protein, e.g. transferrin, which binds to an internalizing cell surface receptor expressed by a muscle cell. Accordingly, in some embodiments, the muscle-targeting agent is transferrin, or a derivative thereof that binds to a transferrin receptor. A muscle-targeting ligand may alternatively be a small molecule, e.g. a lipophilic small molecule that preferentially targets muscle cells relative to other cell types. Exemplary lipophilic small molecules that may target muscle cells include compounds comprising cholesterol, cholesteryl, stearic acid, palmitic acid, oleic acid, oleyl, linolene, linoleic acid, myristic acid, sterols, dihydrotestosterone, testosterone derivatives, glycerine, alkyl chains, trityl groups, and alkoxy acids.

iv. Muscle-Targeting Aptamers

A muscle-targeting agent may be an aptamer, e.g. an RNA aptamer, which preferentially targets muscle cells relative to other cell types. In some embodiments, a muscle-targeting aptamer has not been previously characterized or disclosed. These aptamers may be conceived of, produced, synthesized, and/or (e.g., and) derivatized using any of several methodologies, e.g. Systematic Evolution of Ligands by Exponential Enrichment. Exemplary methodologies have been characterized in the art and are incorporated by reference (Yan, A. C. and Levy, M. “Aptamers and aptamer targeted delivery” RNA biology, 2009, 6:3, 316-20.; Germer, K. et al. “RNA aptamers and their therapeutic and diagnostic applications.” Int. J. Biochem. Mol. Biol. 2013; 4: 27-40.). In some embodiments, a muscle-targeting aptamer has been previously disclosed (see, e.g. Phillippou, S. et al. “Selection and Identification of Skeletal-Muscle-Targeted RNA Aptamers.” Mol Ther Nucleic Acids. 2018, 10:199-214.; Thiel, W. H. et al. “Smooth Muscle Cell-targeted RNA Aptamer Inhibits Neointimal Formation.” Mol Ther. 2016, 24:4, 779-87.). Exemplary muscle-targeting aptamers include the A01B RNA aptamer and RNA Apt 14. In some embodiments, an aptamer is a nucleic acid-based aptamer, an oligonucleotide aptamer or a peptide aptamer. In some embodiments, an aptamer may be about 5-15 kDa, about 5-10 kDa, about 10-15 kDa, about 1-5 Da, about 1-3 kDa, or smaller.

v. Other Muscle-Targeting Agents

One strategy for targeting a muscle cell (e.g., a skeletal muscle cell) is to use a substrate of a muscle transporter protein, such as a transporter protein expressed on the sarcolemma. In some embodiments, the muscle-targeting agent is a substrate of an influx transporter that is specific to muscle tissue. In some embodiments, the influx transporter is specific to skeletal muscle tissue. Two main classes of transporters are expressed on the skeletal muscle sarcolemma, (1) the adenosine triphosphate (ATP) binding cassette (ABC) superfamily, which facilitate efflux from skeletal muscle tissue and (2) the solute carrier (SLC) superfamily, which can facilitate the influx of substrates into skeletal muscle. In some embodiments, the muscle-targeting agent is a substrate that binds to an ABC superfamily or an SLC superfamily of transporters. In some embodiments, the substrate that binds to the ABC or SLC superfamily of transporters is a naturally-occurring substrate. In some embodiments, the substrate that binds to the ABC or SLC superfamily of transporters is a non-naturally occurring substrate, for example, a synthetic derivative thereof that binds to the ABC or SLC superfamily of transporters.

In some embodiments, the muscle-targeting agent is any muscle targeting agent described herein (e.g., antibodies, nucleic acids, small molecules, peptides, aptamers, lipids, sugar moieties) that target SLC superfamily of transporters. In some embodiments, the muscle-targeting agent is a substrate of an SLC superfamily of transporters. SLC transporters are either equilibrative or use proton or sodium ion gradients created across the membrane to drive transport of substrates. Exemplary SLC transporters that have high skeletal muscle expression include, without limitation, the SATT transporter (ASCT1; SLC1A4), GLUT4 transporter (SLC2A4), GLUT7 transporter (GLUT7; SLC2A7), ATRC2 transporter (CAT-2; SLC7A2), LAT3 transporter (KIAA0245; SLC7A6), PHT1 transporter (PTR4; SLC15A4), OATP-J transporter (OATP5A1; SLC21A15), OCT3 transporter (EMT; SLC22A3), OCTN2 transporter (FLJ46769; SLC22A5), ENT transporters (ENT1; SLC29A1 and ENT2; SLC29A2), PAT2 transporter (SLC36A2), and SAT2 transporter (KIAA1382; SLC38A2). These transporters can facilitate the influx of substrates into skeletal muscle, providing opportunities for muscle targeting.

In some embodiments, the muscle-targeting agent is a substrate of an equilibrative nucleoside transporter 2 (ENT2) transporter. Relative to other transporters, ENT2 has one of the highest mRNA expressions in skeletal muscle. While human ENT2 (hENT2) is expressed in most body organs such as brain, heart, placenta, thymus, pancreas, prostate, and kidney, it is especially abundant in skeletal muscle. Human ENT2 facilitates the uptake of its substrates depending on their concentration gradient. ENT2 plays a role in maintaining nucleoside homeostasis by transporting a wide range of purine and pyrimidine nucleobases. The hENT2 transporter has a low affinity for all nucleosides (adenosine, guanosine, uridine, thymidine, and cytidine) except for inosine. Accordingly, in some embodiments, the muscle-targeting agent is an ENT2 substrate. Exemplary ENT2 substrates include, without limitation, inosine, 2′,3′-dideoxyinosine, and calofarabine. In some embodiments, any of the muscle-targeting agents provided herein are associated with a molecular payload (e.g., oligonucleotide payload). In some embodiments, the muscle-targeting agent is covalently linked to the molecular payload. In some embodiments, the muscle-targeting agent is non-covalently linked to the molecular payload.

In some embodiments, the muscle-targeting agent is a substrate of an organic cation/carnitine transporter (OCTN2), which is a sodium ion-dependent, high affinity carnitine transporter. In some embodiments, the muscle-targeting agent is carnitine, mildronate, acetylcarnitine, or any derivative thereof that binds to OCTN2. In some embodiments, the carnitine, mildronate, acetylcarnitine, or derivative thereof is covalently linked to the molecular payload (e.g., oligonucleotide payload).

A muscle-targeting agent may be a protein that is protein that exists in at least one soluble form that targets muscle cells. In some embodiments, a muscle-targeting protein may be hemojuvelin (also known as repulsive guidance molecule C or hemochromatosis type 2 protein), a protein involved in iron overload and homeostasis. In some embodiments, hemojuvelin may be full length or a fragment, or a mutant with at least 75%, at least 80%, at least 85%, at least 90%, at least 95%, at least 98% or at least 99% sequence identity to a functional hemojuvelin protein. In some embodiments, a hemojuvelin mutant may be a soluble fragment, may lack a N-terminal signaling, and/or (e.g., and) lack a C-terminal anchoring domain. In some embodiments, hemojuvelin may be annotated under GenBank RefSeq Accession Numbers NM_001316767.1, NM_145277.4, NM_202004.3, NM_213652.3, or NM_213653.3. It should be appreciated that a hemojuvelin may be of human, non-human primate, or rodent origin.

### B. Molecular Payloads

Some aspects of the disclosure provide molecular payloads, e.g., oligonucleotides designed to target DMPK RNAs to modulate the expression or the activity of DMPK. In some embodiments, modulating the expression or activity of DMPK comprises reducing levels of DMPK RNA and/or (e.g., and) protein. In some embodiments, the DMPK RNA is disease-associated, e.g., having a disease-associated repeat expansion or encoded from an allele having a disease-associated repeat expansion. In some embodiments, the DMPK RNA comprises a CUG repeat expansion, or the allele from which it is encoded comprises a CTG repeat expansion. In some embodiments, the disclosure provides oligonucleotides complementary with DMPK RNA that are useful for reducing levels of toxic DMPK having disease-associated repeat expansions, e.g., in a subject having or suspected of having myotonic dystrophy. In some embodiments, the oligonucleotides are designed to direct RNAse H mediated degradation of the target DMPK RNA. In some embodiments, the oligonucleotides are designed to direct RNAse H mediated degradation of the target DMPK RNA residing in the nucleus of cells, e.g., muscle cells (e.g., myotubes) or CNS cells (e.g., neurons). In some embodiments, the oligonucleotides are designed to have desirable bioavailability and/or serum-stability properties. In some embodiments, the oligonucleotides are designed to have desirable binding affinity properties. In some embodiments, the oligonucleotides are designed to have desirable toxicity profiles. In some embodiments, the oligonucleotides are designed to have low-complement activation and/or cytokine induction properties.

In some embodiments, the oligonucleotide is linked to, or otherwise associated with a muscle-targeting agent described herein. In some embodiments, such oligonucleotides are capable of targeting DMPK in a muscle cell, e.g., via specifically binding to a DMPK sequence in the muscle cell following delivery to the muscle cell by an associated muscle-targeting agent. It should be appreciated that various types of muscle-targeting agents may be used in accordance with the disclosure. In some embodiments, the oligonucleotide comprises a region of complementarity to a DMPK allele comprising a disease-associated-repeat expansion. Exemplary oligonucleotides targeting the DMPK RNA are described in further detail herein, however, it should be appreciated that the exemplary molecular payloads provided herein are not meant to be limiting.

i. Oligonucleotides

In some embodiments, the DMPK-targeting oligonucleotides described herein are designed to caused RNase H mediated degradation of DMPK mRNA. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

Examples of oligonucleotides useful for targeting DMPK are provided in US Patent Application Publication 20100016215A1, published on Jan. 1, 2010, entitled Compound And Method For Treating Myotonic Dystrophy; US Patent Application Publication 20130237585A1, published Jul. 19, 2010, Modulation Of Dystrophia Myotonica-Protein Kinase (DMPK) Expression; US Patent Application Publication 20150064181A1, published on Mar. 5, 2015, entitled “Antisense Conjugates For Decreasing Expression Of Dmpk”; US Patent Application Publication 20150238627A1, published on Aug. 27, 2015, entitled “Peptide-Linked Morpholino Antisense Oligonucleotides For Treatment Of Myotonic Dystrophy”; and US Patent Application Publication 20160304877A1, published on Oct. 20, 2016, entitled “Compounds And Methods For Modulation Of Dystrophia Myotonica-Protein Kinase (Dmpk) Expression,” the contents of each of which are incorporated herein in their entireties.

In some embodiments, oligonucleotides may have a region of complementarity to a sequence set forth as follows, which is an example human DMPK gene sequence (Gene ID 1760; NM 001081560.2):

(SEQ ID NO: 130)
AGGGGGGCTGGACCAAGGGGTGGGGAGAAGGGGAGGAGGCCTCGGCCGGC
CGCAGAGAGAAGTGGCCAGAGAGGCCCAGGGGACAGCCAGGGACAGGCAG
ACATGCAGCCAGGGCTCCAGGGCCTGGACAGGGGCTGCCAGGCCCTGTGA
CAGGAGGACCCCGAGCCCCCGGCCCGGGGAGGGGCCATGGTGCTGCCTGT
CCAACATGTCAGCCGAGGTGCGGCTGAGGCGGCTCCAGCAGCTGGTGTTG
GACCCGGGCTTCCTGGGGCTGGAGCCCCTGCTCGACCTTCTCCTGGGCGT
CCACCAGGAGCTGGGCGCCTCCGAACTGGCCCAGGACAAGTACGTGGCCG
ACTTCTTGCAGTGGGCGGAGCCCATCGTGGTGAGGCTTAAGGAGGTCCGA
CTGCAGAGGGACGACTTCGAGATTCTGAAGGTGATCGGACGCGGGGCGTT
CAGCGAGGTAGCGGTAGTGAAGATGAAGCAGACGGGCCAGGTGTATGCCA
TGAAGATCATGAACAAGTGGGACATGCTGAAGAGGGGCGAGGTGTCGTGC
TTCCGTGAGGAGAGGGACGTGTTGGTGAATGGGGACCGGCGGTGGATCAC
GCAGCTGCACTTCGCCTTCCAGGATGAGAACTACCTGTACCTGGTCATGG
AGTATTACGTGGGCGGGGACCTGCTGACACTGCTGAGCAAGTTTGGGGAG
CGGATTCCGGCCGAGATGGCGCGCTTCTACCTGGCGGAGATTGTCATGGC
CATAGACTCGGTGCACCGGCTTGGCTACGTGCACAGGGACATCAAACCCG
ACAACATCCTGCTGGACCGCTGTGGCCACATCCGCCTGGCCGACTTCGGC
TCTTGCCTCAAGCTGCGGGCAGATGGAACGGTGCGGTCGCTGGTGGCTGT
GGGCACCCCAGACTACCTGTCCCCCGAGATCCTGCAGGCTGTGGGCGGTG
GGCCTGGGACAGGCAGCTACGGGCCCGAGTGTGACTGGTGGGCGCTGGGT
GTATTCGCCTATGAAATGTTCTATGGGCAGACGCCCTTCTACGCGGATTC
CACGGCGGAGACCTATGGCAAGATCGTCCACTACAAGGAGCACCTCTCTC
TGCCGCTGGTGGACGAAGGGGTCCCTGAGGAGGCTCGAGACTTCATTCAG
CGGTTGCTGTGTCCCCCGGAGACACGGCTGGGCCGGGGTGGAGCAGGCGA
CTTCCGGACACATCCCTTCTTCTTTGGCCTCGACTGGGATGGTCTCCGGG
ACAGCGTGCCCCCCTTTACACCGGATTTCGAAGGTGCCACCGACACATGC
AACTTCGACTTGGTGGAGGACGGGCTCACTGCCATGGAGACACTGTCGGA
CATTCGGGAAGGTGCGCCGCTAGGGGTCCACCTGCCTTTTGTGGGCTACT
CCTACTCCTGCATGGCCCTCAGGGACAGTGAGGTCCCAGGCCCCACACCC
ATGGAACTGGAGGCCGAGCAGCTGCTTGAGCCACACGTGCAAGCGCCCAG
CCTGGAGCCCTCGGTGTCCCCACAGGATGAAACAGCTGAAGTGGCAGTTC
CAGCGGCTGTCCCTGCGGCAGAGGCTGAGGCCGAGGTGACGCTGCGGGAG
CTCCAGGAAGCCCTGGAGGAGGAGGTGCTCACCCGGCAGAGCCTGAGCCG
GGAGATGGAGGCCATCCGCACGGACAACCAGAACTTCGCCAGTCAACTAC
GCGAGGCAGAGGCTCGGAACCGGGACCTAGAGGCACACGTCCGGCAGTTG
CAGGAGCGGATGGAGTTGCTGCAGGCAGAGGGAGCCACAGCTGTCACGGG
GGTCCCCAGTCCCCGGGCCACGGATCCACCTTCCCATCTAGATGGCCCCC
CGGCCGTGGCTGTGGGCCAGTGCCCGCTGGTGGGGCCAGGCCCCATGCAC
CGCCGCCACCTGCTGCTCCCTGCCAGGGTCCCTAGGCCTGGCCTATCGGA
GGCGCTTTCCCTGCTCCTGTTCGCCGTTGTTCTGTCTCGTGCCGCCGCCC
TGGGCTGCATTGGGTTGGTGGCCCACGCCGGCCAACTCACCGCAGTCTGG
CGCCGCCCAGGAGCCGCCCGCGCTCCCTGAACCCTAGAACTGTCTTCGAC
TCCGGGGCCCCGTTGGAAGACTGAGTGCCCGGGGCACGGCACAGAAGCCG
CGCCCACCGCCTGCCAGTTCACAACCGCTCCGAGCGTGGGTCTCCGCCCA
GCTCCAGTCCTGTGATCCGGGCCCGCCCCCTAGCGGCCGGGGAGGGAGGG
GCCGGGTCCGCGGCCGGCGAACGGGGCTCGAAGGGTCCTTGTAGCCGGGA
ATGCTGCTGCTGCTGCTGCTGCTGCTGCTGCTGCTGCTGCTGCTGCTGCT
GCTGCTGCTGCTGGGGGGATCACAGACCATTTCTTTCTTTCGGCCAGGCT
GAGGCCCTGACGTGGATGGGCAAACTGCAGGCCTGGGAAGGCAGCAAGCC
GGGCCGTCCGTGTTCCATCCTCCACGCACCCCCACCTATCGTTGGTTCGC
AAAGTGCAAAGCTTTCTTGTGCATGACGCCCTGCTCTGGGGAGCGTCTGG
CGCGATCTCTGCCTGCTTACTCGGGAAATTTGCTTTTGCCAAACCCGCTT
TTTCGGGGATCCCGCGCCCCCCTCCTCACTTGCGCTGCTCTCGGAGCCCC
AGCCGGCTCCGCCCGCTTCGGCGGTTTGGATATTTATTGACCTCGTCCTC
CGACTCGCTGACAGGCTACAGGACCCCCAACAACCCCAATCCACGTTTTG
GATGCACTGAGACCCCGACATTCCTCGGTATTTATTGTCTGTCCCCACCT
AGGACCCCCACCCCCGACCCTCGCGAATAAAAGGCCCTCCATCTGCCCAA
AGCTCTGGA.

In some embodiments, oligonucleotides may have a region of complementarity to a sequence set forth as follows, which is an example mouse DMPK gene sequence (Gene ID 13400; NM_001190490.1). GAACTGGCCAGAGAGACCCAAGGGATAGTCAGGGACGGGCAGACATGCAGCTAGG GTTCTGGGGCCTGGACAGGGGCAGCCAGGCCCTGTGACGGGAAGACCCCGAGCTCC GGCCCGGGGAGGGGCCATGGTGTTGCCTGCCCAACATGTCAGCCGAAGTGCGGCTG AGGCAGCTCCAGCAGCTGGTGCTGGACCCAGGCTTCCTGGGACTGGAGCCCCTGCT CGACCTTCTCCTGGGCGTCCACCAGGAGCTGGGTGCCTCTCACCTAGCCCAGGACA AGTATGTGGCCGACTTCTTGCAGTGGGTGGAGCCCATTGCAGCAAGGCTTAAGGAG GTCCGACTGCAGAGGGATGATTTTGAGATTTTGAAGGTGATCGGGCGTGGGGCGTT CAGCGAGGTAGCGGTGGTGAAGATGAAACAGACGGGCCAAGTGTATGCCATGAAG ATTATGAATAAGTGGGACATGCTGAAGAGAGGCGAGGTGTCGTGCTTCCGGGAAGA AAGGGATGTATTAGTGAAAGGGGACCGGCGCTGGATCACACAGCTGCACTTTGCCT TCCAGGATGAGAACTACCTGTACCTGGTCATGGAATACTACGTGGGCGGGGACCTG CTAACGCTGCTGAGCAAGTTTGGGGAGCGGATCCCCGCCGAGATGGCTCGCTTCTA CCTGGCCGAGATTGTCATGGCCATAGACTCCGTGCACCGGCTGGGCTACGTGCACA GGGACATCAAACCAGATAACATTCTGCTGGACCGATGTGGGCACATTCGCCTGGCA GACTTCGGCTCCTGCCTCAAACTGCAGCCTGATGGAATGGTGAGGTCGCTGGTGGCT GTGGGCACCCCGGACTACCTGTCTCCTGAGATTCTGCAGGCCGTTGGTGGAGGGCCT GGGGCAGGCAGCTACGGGCCAGAGTGTGACTGGTGGGCACTGGGCGTGTTCGCCTA TGAGATGTTCTATGGGCAGACCCCCTTCTACGCGGACTCCACAGCCGAGACATATG CCAAGATTGTGCACTACAGGGAACACTTGTCGCTGCCGCTGGCAGACACAGTTGTC CCCGAGGAAGCTCAGGACCTCATTCGTGGGCTGCTGTGTCCTGCTGAGATAAGGCT AGGTCGAGGTGGGGCAGACTTCGAGGGTGCCACGGACACATGCAATTTCGATGTGG TGGAGGACCGGCTCACTGCCATGGTGAGCGGGGGCGGGGAGACGCTGTCAGACAT GCAGGAAGACATGCCCCTTGGGGTGCGCCTGCCCTTCGTGGGCTACTCCTACTGCTG CATGGCCTTCAGAGACAATCAGGTCCCGGACCCCACCCCTATGGAACTAGAGGCCC TGCAGTTGCCTGTGTCAGACTTGCAAGGGCTTGACTTGCAGCCCCCAGTGTCCCCAC CGGATCAAGTGGCTGAAGAGGCTGACCTAGTGGCTGTCCCTGCCCCTGTGGCTGAG GCAGAGACCACGGTAACGCTGCAGCAGCTCCAGGAAGCCCTGGAAGAAGAGGTTC TCACCCGGCAGAGCCTGAGCCGCGAGCTGGAGGCCATCCGGACCGCCAACCAGAAC TTCTCCAGCCAACTACAGGAGGCCGAGGTCCGAAACCGAGACCTGGAGGCGCATGT TCGGCAGCTACAGGAACGGATGGAGATGCTGCAGGCCCCAGGAGCCGCAGCCATC ACGGGGGTCCCCAGTCCCCGGGCCACGGATCCACCTTCCCATCTAGATGGCCCCCC GGCCGTGGCTGTGGGCCAGTGCCCGCTGGTGGGGCCAGGCCCCATGCACCGCCGTC ACCTGCTGCTCCCTGCCAGGATCCCTAGGCCTGGCCTATCCGAGGCGCGTTGCCTGC TCCTGTTCGCCGCTGCTCTGGCTGCTGCCGCCACACTGGGCTGCACTGGGTTGGTGG CCTATACCGGCGGTCTCACCCCAGTCTGGTGTTTCCCGGGAGCCACCTTCGCCCCCT GAACCCTAAGACTCCAAGCCATCTTTCATTTAGGCCTCCTAGGAAGGTCGAGCGAC CAGGGAGCGACCCAAAGCGTCTCTGTGCCCATCGCGCCCCCCCCCCCCCCCCACCG CTCCGCTCCACACTTCTGTGAGCCTGGGTCCCCACCCAGCTCCGCTCCTGTGATCCA GGCCTGCCACCTGGCGGCCGGGGAGGGAGGAACAGGGCTCGTGCCCAGCACCCCTG GTTCCTGCAGAGCTGGTAGCCACCGCTGCTGCAGCAGCTGGGCATTCGCCGACCTTG CTTTACTCAGCCCCGACGTGGATGGGCAAACTGCTCAGCTCATCCGATTTCACTTTT TCACTCTCCCAGCCATCAGTTACAAGCCATAAGCATGAGCCCCCTATTTCCAGGGAC ATCCCATTCCCATAGTGATGGATCAGCAAGACCTCTGCCAGCACACACGGAGTCTTT GGCTTCGGACAGCCTCACTCCTGGGGGTTGCTGCAACTCCTTCCCCGTGTACACGTC TGCACTCTAACAACGGAGCCACAGCTGCACTCCCCCCTCCCCCAAAGCAGTGTGGG TATTTATTGATCTTGTTATCTGACTCACTGACAGACTCCGGGACCCACGTTTTAGAT GCATTGAGACTCGACATTCCTCGGTATTTATTGTCTGTCCCCACCTACGACCTCCACT CCCGACCCTTGCGAATAAAATACTTCTGGTCTGCCCTAAA (SEQ ID NO: 131). In some embodiments, an oligonucleotide may have a region of complementarity to DMPK gene sequences of multiple species, e.g., selected from human, mouse and non-human species.

In some embodiments, the oligonucleotide may have region of complementarity to a mutant form of DMPK, for example, a mutant form as reported in Botta A. et al. “The CTG repeat expansion size correlates with the splicing defects observed in muscles from myotonic dystrophy type 1 patients.” J Med Genet. 2008 October; 45(10):639-46.; and Machuca-Tzili L. et al. “Clinical and molecular aspects of the myotonic dystrophies: a review.” Muscle Nerve. 2005 July; 32(1):1-18.; the contents of each of which are incorporated herein by reference in their entireties.

In some embodiments, an oligonucleotide provided herein is an antisense oligonucleotide targeting DMPK. In some embodiments, the oligonucleotide targeting is any one of the antisense oligonucleotides (e.g., a Gapmer) targeting DMPK as described in US Patent Application Publication US20160304877A1, published on Oct. 20, 2016, entitled “Compounds And Methods For Modulation Of Dystrophia Myotonica-Protein Kinase (DMPK) Expression,” incorporated herein by reference). In some embodiments, the DMPK targeting oligonucleotide targets a region of the DMPK gene sequence as set forth in Genbank accession No. NM_001081560.2 (SEQ ID NO: 130) or as set forth in Genbank accession No. NG_009784.1 (SEQ ID NO: 395).

In some embodiments, the DMPK targeting oligonucleotide comprises a nucleotide sequence comprising a region complementary to a target region that is at least 10 continuous nucleotides (e.g., at least 10, at least 12, at least 14, at least 16, at least 18, at least 20 or more continuous nucleotides) in SEQ ID NO: 130.

In some embodiments, the DMPK targeting oligonucleotide comprise a gapmer motif. “Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region can be referred to as a “gap segment” and the external regions can be referred to as “wing segments.” In some embodiments, the DMPK targeting oligonucleotide comprises one or more modified nucleosides, and/or (e.g., and) one or more modified internucleoside linkages. In some embodiments, the internucleoside linkage is a phosphorothioate linkage. In some embodiments, the oligonucleotide comprises a full phosphorothioate backbone. In some embodiments, the oligonucleotide is a DNA gapmer with cET ends (e.g., 3-10-3; cET-DNA-cET). In some embodiments, the DMPK targeting oligonucleotide comprises one or more 6′-(S)—CH₃ biocyclic nucleosides, one or more β-D-2′-deoxyribonucleotides, and/or (e.g., and) one or more 5-methylcytosine nucleosides.

a. Oligonucleotide Size/Sequence

Oligonucleotides may be of a variety of different lengths, e.g., depending on the format. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, the oligonucleotide is 8 to 50 nucleotides in length, 8 to 40 nucleotides in length, 8 to 30 nucleotides in length, 10 to 15 nucleotides in length, 10 to 20 nucleotides in length, 15 to 25 nucleotides in length, 21 to 23 nucleotides in lengths, 15 to 20 nucleotides in length, 20 to 25 nucleotides in length, etc.

In some embodiments, a nucleic acid sequence of an oligonucleotide for purposes of the present disclosure is “complementary” to a target nucleic acid when it is specifically hybridizable to the target nucleic acid. In some embodiments, an oligonucleotide hybridizing to a target nucleic acid (e.g., an mRNA or pre-mRNA molecule) results in modulation of activity or expression of the target (e.g., decreased mRNA translation, altered pre-mRNA splicing, exon skipping, target mRNA degradation, etc.). In some embodiments, a nucleic acid sequence of an oligonucleotide has a sufficient degree of complementarity to its target nucleic acid such that it does not hybridize non-target sequences under conditions in which avoidance of non-specific binding is desired, e.g., under physiological conditions. Thus, in some embodiments, an oligonucleotide may be at least 80%, at least 85%, at least 90%, at least 91%, at least 92%, at least 93%, at least 94%, at least 95%, at least 96%, at least 97%, at least 98%, at least 99% or 100% complementary to the consecutive nucleotides of a target nucleic acid. In some embodiments a complementary nucleotide sequence need not be 100% complementary to that of its target to be specifically hybridizable or specific for a target nucleic acid. In certain embodiments, oligonucleotides comprise one or more mismatched nucleobases relative to the target nucleic acid. In certain embodiments, activity relating to the target is reduced by such mismatch, but activity relating to a non-target is reduced by a greater amount (i.e., selectivity for the target nucleic acid is increased and off-target effects are decreased).

In some embodiments, an oligonucleotide comprises region of complementarity to a target nucleic acid that is in the range of 8 to 15, 8 to 30, 8 to 40, or 10 to 50, or 5 to 50, 15 to 20, 20 to 25, or 5 to 40 nucleotides in length. In some embodiments, a region of complementarity of an oligonucleotide to a target nucleic acid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the region of complementarity is complementary with at least 8 consecutive nucleotides of a target nucleic acid. In some embodiments, an oligonucleotide may contain 1, 2 or 3 base mismatches compared to the portion of the consecutive nucleotides of target nucleic acid. In some embodiments the oligonucleotide may have up to 3 mismatches over 15 bases, or up to 2 mismatches over 10 bases.

In some embodiments, an oligonucleotide comprises at least 10, 11, 12, 13, 14, 15, 16, 17, 18, 19, or 20 consecutive nucleotides of a sequence comprising any one of SEQ ID NOs: 231-362. In some embodiments, an oligonucleotide comprises a sequence comprising any one of SEQ ID NOs: 231-362. In some embodiments, an oligonucleotide comprises a sequence that shares at least 70%, 75%, 80%, 85%, 90%, 95%, or 97% sequence identity with at least 12 or at least 15 consecutive nucleotides of any one of SEQ ID NOs: 231-362.

In some embodiments, an oligonucleotide comprises a region of complementarity to nucleotide sequence set forth in any one of SEQ ID NOs: 160-230. In some embodiments, an oligonucleotide comprises at least 10, 11, 12, 13, 14, 15, 16, 17, 18, 19, or 20 nucleotides (e.g., consecutive nucleotides) that are complementary to a nucleotide sequence set forth in any one of SEQ ID NOs: 160-230. In some embodiments, an oligonucleotide comprises a sequence that is at least 70%, 75%, 80%, 85%, 90%, 95%, 97%; 99%, or 100% complementary with at least 12 or at least 15 consecutive nucleotides of any one of SEQ ID NOs: 160-230.

In some embodiments, the oligonucleotide is complementary (e.g., at least 85% at least 90%, at least 95%, or 100%) to a target sequence of any one of the oligonucleotides provided herein (e.g., the oligonucleotides listed in Table 8, Table 9, and Table 10). In some embodiments, such target sequence is 100% complementary to the oligonucleotide listed in Table 8, Table 9, or Table 10.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein (e.g., the oligonucleotides listed in Table 8, Table 9, and Table 10) may independently and optionally be uracil bases (U's), and/or any one or more of the U's may independently and optionally be T's.

b. Oligonucleotide Modifications:

The oligonucleotides described herein may be modified, e.g., comprise a modified sugar moiety, a modified internucleoside linkage, a modified nucleotide or nucleoside and/or (e.g., and) combinations thereof. In addition, in some embodiments, oligonucleotides may exhibit one or more of the following properties: do not mediate alternative splicing; are not immune stimulatory; are nuclease resistant; have improved cell uptake compared to unmodified oligonucleotides; are not toxic to cells or mammals; have improved endosomal exit internally in a cell; minimizes TLR stimulation; or avoid pattern recognition receptors. Any of the modified chemistries or formats of oligonucleotides described herein can be combined with each other. For example, one, two, three, four, five, or more different types of modifications can be included within the same oligonucleotide.

In some embodiments, certain nucleotide or nucleoside modifications may be used that make an oligonucleotide into which they are incorporated more resistant to nuclease digestion than the native oligodeoxynucleotide or oligoribonucleotide molecules; these modified oligonucleotides survive intact for a longer time than unmodified oligonucleotides. Specific examples of modified oligonucleotides include those comprising modified backbones, for example, modified internucleoside linkages such as phosphorothioates, phosphotriesters, methyl phosphonates, short chain alkyl or cycloalkyl intersugar linkages or short chain heteroatomic or heterocyclic intersugar linkages. Accordingly, oligonucleotides of the disclosure can be stabilized against nucleolytic degradation such as by the incorporation of a modification, e.g., a nucleotide or nucleoside modification.

In some embodiments, an oligonucleotide may be of up to 50 or up to 100 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides or nucleosides of the oligonucleotide are modified nucleotides/nucleosides. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides or nucleosides of the oligonucleotide are modified nucleotides/nucleosides. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides or nucleosides of the oligonucleotide are modified nucleotides/nucleosides. Optionally, the oligonucleotides may have every nucleotide or nucleoside except 1, 2, 3, 4, 5, 6, 7, 8, 9, or 10 nucleotides/nucleosides modified. Oligonucleotide modifications are described further herein.

c. Modified Nucleosides

In some embodiments, the oligonucleotide described herein comprises at least one nucleoside modified at the 2′ position of the sugar. In some embodiments, an oligonucleotide comprises at least one 2′-modified nucleoside. In some embodiments, all of the nucleosides in the oligonucleotide are 2′-modified nucleosides.

In some embodiments, the oligonucleotide described herein comprises one or more non-bicyclic 2′-modified nucleosides, e.g., 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) modified nucleoside.

In some embodiments, the oligonucleotide described herein comprises one or more 2′-4′ bicyclic nucleosides in which the ribose ring comprises a bridge moiety connecting two atoms in the ring, e.g., connecting the 2′-O atom to the 4′-C atom via a methylene (LNA) bridge, an ethylene (ENA) bridge, or a (S)-constrained ethyl (cEt) bridge. Examples of LNAs are described in International Patent Application Publication WO/2008/043753, published on Apr. 17, 2008, and entitled “RNA Antagonist Compounds For The Modulation Of PCSK9”, the contents of which are incorporated herein by reference in its entirety. Examples of ENAs are provided in International Patent Publication No. WO 2005/042777, published on May 12, 2005, and entitled “APP/ENA Antisense”; Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties. Examples of cEt are provided in U.S. Pat. Nos. 7,101,993; 7,399,845 and 7,569,686, each of which is herein incorporated by reference in its entirety.

In some embodiments, the oligonucleotide comprises a modified nucleoside disclosed in one of the following United States Patent or Patent Application Publications: U.S. Pat. No. 7,399,845, issued on Jul. 15, 2008, and entitled “6-Modified Bicyclic Nucleic Acid Analogs”; U.S. Pat. No. 7,741,457, issued on Jun. 22, 2010, and entitled “6-Modified Bicyclic Nucleic Acid Analogs”; U.S. Pat. No. 8,022,193, issued on Sep. 20, 2011, and entitled “6-Modified Bicyclic Nucleic Acid Analogs”; U.S. Pat. No. 7,569,686, issued on Aug. 4, 2009, and entitled “Compounds And Methods For Synthesis Of Bicyclic Nucleic Acid Analogs”; U.S. Pat. No. 7,335,765, issued on Feb. 26, 2008, and entitled “Novel Nucleoside And Oligonucleotide Analogues”; U.S. Pat. No. 7,314,923, issued on Jan. 1, 2008, and entitled “Novel Nucleoside And Oligonucleotide Analogues”; U.S. Pat. No. 7,816,333, issued on Oct. 19, 2010, and entitled “Oligonucleotide Analogues And Methods Utilizing The Same” and US Publication Number 2011/0009471 now U.S. Pat. No. 8,957,201, issued on Feb. 17, 2015, and entitled “Oligonucleotide Analogues And Methods Utilizing The Same”, the entire contents of each of which are incorporated herein by reference for all purposes.

In some embodiments, the oligonucleotide comprises at least one modified nucleoside that results in an increase in Tm of the oligonucleotide in a range of 1° C., 2° C., 3° C., 4° C., or 5° C. compared with an oligonucleotide that does not have the at least one modified nucleoside. The oligonucleotide may have a plurality of modified nucleosides that result in a total increase in Tm of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the modified nucleoside.

The oligonucleotide may comprise a mix of nucleosides of different kinds. For example, an oligonucleotide may comprise a mix of 2′-deoxyribonucleosides or ribonucleosides and 2′-fluoro modified nucleosides. An oligonucleotide may comprise a mix of deoxyribonucleosides or ribonucleosides and 2′-O-Me modified nucleosides. An oligonucleotide may comprise a mix of 2′-fluoro modified nucleosides and 2′-O-methyl modified nucleosides. An oligonucleotide may comprise a mix of bridged nucleosides and 2′-fluoro or 2′-O-methyl modified nucleosides. An oligonucleotide may comprise a mix of non-bicyclic 2′-modified nucleosides (e.g., 2′-O-MOE) and 2′-4′ bicyclic nucleosides (e.g., LNA, ENA, cEt). An oligonucleotide may comprise a mix of 2′-fluoro modified nucleosides and 2′-O-Me modified nucleosides. An oligonucleotide may comprise a mix of 2′-4′ bicyclic nucleosides and 2′-MOE, 2′-fluoro, or 2′-O-Me modified nucleosides. An oligonucleotide may comprise a mix of non-bicyclic 2′-modified nucleosides (e.g., 2′-MOE, 2′-fluoro, or 2′-O-Me) and 2′-4′ bicyclic nucleosides (e.g., LNA, ENA, cEt).

The oligonucleotide may comprise alternating nucleosides of different kinds. For example, an oligonucleotide may comprise alternating 2′-deoxyribonucleosides or ribonucleosides and 2′-fluoro modified nucleosides. An oligonucleotide may comprise alternating deoxyribonucleosides or ribonucleosides and 2′-O-Me modified nucleosides. An oligonucleotide may comprise alternating 2′-fluoro modified nucleosides and 2′-O-Me modified nucleosides. An oligonucleotide may comprise alternating bridged nucleosides and 2′-fluoro or 2′-O-methyl modified nucleosides. An oligonucleotide may comprise alternating non-bicyclic 2′-modified nucleosides (e.g., 2′-O-MOE) and 2′-4′ bicyclic nucleosides (e.g., LNA, ENA, cEt). An oligonucleotide may comprise alternating 2′-4′ bicyclic nucleosides and 2′-MOE, 2′-fluoro, or 2′-O-Me modified nucleosides. An oligonucleotide may comprise alternating non-bicyclic 2′-modified nucleosides (e.g., 2′-MOE, 2′-fluoro, or 2′-O-Me) and 2′-4′ bicyclic nucleosides (e.g., LNA, ENA, cEt).

In some embodiments, an oligonucleotide described herein comprises a 5′-vinylphosphonate modification, one or more abasic residues, and/or one or more inverted abasic residues.

d. Internucleoside Linkages/Backbones

In some embodiments, oligonucleotide may contain a phosphorothioate or other modified internucleoside linkage. In some embodiments, the oligonucleotide comprises phosphorothioate internucleoside linkages. In some embodiments, the oligonucleotide comprises phosphorothioate internucleoside linkages between at least two nucleosides. In some embodiments, the oligonucleotide comprises phosphorothioate internucleoside linkages between all nucleosides. For example, in some embodiments, oligonucleotides comprise modified internucleoside linkages at the first, second, and/or (e.g., and) third internucleoside linkage at the 5′ or 3′ end of the nucleotide sequence.

Phosphorus-containing linkages that may be used include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

In some embodiments, oligonucleotides may have heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497).

e. Stereospecific Oligonucleotides

In some embodiments, internucleotidic phosphorus atoms of oligonucleotides are chiral, and the properties of the oligonucleotides by adjusted based on the configuration of the chiral phosphorus atoms. In some embodiments, appropriate methods may be used to synthesize P-chiral oligonucleotide analogs in a stereocontrolled manner (e.g., as described in Oka N, Wada T, Stereocontrolled synthesis of oligonucleotide analogs containing chiral internucleotidic phosphorus atoms. Chem Soc Rev. 2011 December; 40(12):5829-43.) In some embodiments, phosphorothioate containing oligonucleotides comprise nucleoside units that are joined together by either substantially all Sp or substantially all Rp phosphorothioate intersugar linkages are provided. In some embodiments, such phosphorothioate oligonucleotides having substantially chirally pure intersugar linkages are prepared by enzymatic or chemical synthesis, as described, for example, in U.S. Pat. No. 5,587,261, issued on Dec. 12, 1996, the contents of which are incorporated herein by reference in their entirety. In some embodiments, chirally controlled oligonucleotides provide selective cleavage patterns of a target nucleic acid. For example, in some embodiments, a chirally controlled oligonucleotide provides single site cleavage within a complementary sequence of a nucleic acid, as described, for example, in US Patent Application Publication 20170037399 A1, published on Feb. 2, 2017, entitled “CHIRAL DESIGN”, the contents of which are incorporated herein by reference in their entirety.

h. Gapmers

In some embodiments, the oligonucleotide described herein is a gapmer. A gapmer oligonucleotide generally has the formula 5′-X-Y-Z-3′, with X and Z as flanking regions around a gap region Y. In some embodiments, flanking region X of formula 5′-X-Y-Z-3′ is also referred to as X region, flanking sequence X, 5′ wing region X, or 5′ wing segment. In some embodiments, flanking region Z of formula 5′-X-Y-Z-3′ is also referred to as Z region, flanking sequence Z, 3′ wing region Z, or 3′ wing segment. In some embodiments, gap region Y of formula 5′-X-Y-Z-3′ is also referred to as Y region, Y segment, or gap-segment Y. In some embodiments, each nucleoside in the gap region Y is a 2′-deoxyribonucleoside, and neither the 5′ wing region X or the 3′ wing region Z contains any 2′-deoxyribonucleosides. In some embodiments, a gapmer oligonucleotide comprises a region of complementarity to at least 15 consecutive nucleosides (e.g., at least 15, at least 16, at least 17, at least 18, at least 19, or 20 consecutive nucleosides) of a target sequence provided in Table 8 (e.g., any one of SEQ ID NOs: 160-230) and/or comprises at least 15 consecutive nucleosides (e.g., at least 15, at least 16, at least 17, at least 18, at least 19, or 20 consecutive nucleosides) of the nucleotide sequence of an antisense sequence in Table 8, 9 or 10, or ASO structure provided in Table 9 or 10 (e.g., any one of SEQ ID NOs: 231-362), wherein each thymine base (T) may independently and optionally be replaced with a uracil base (U), and each U may independently and optionally be replaced with a T.

In some embodiments, the Y region is a contiguous stretch of nucleotides, e.g., a region of 6 or more DNA nucleotides, which are capable of recruiting an RNAse, such as RNAse H. In some embodiments, the gapmer binds to the target nucleic acid, at which point an RNAse is recruited and can then cleave the target nucleic acid. In some embodiments, the Y region is flanked both 5′ and 3′ by regions X and Z comprising high-affinity modified nucleosides, e.g., one to six high-affinity modified nucleosides. Examples of high affinity modified nucleosides include, but are not limited to, 2′-modified nucleosides (e.g., 2′-MOE, 2′O-Me, 2′-F) or 2′-4′ bicyclic nucleosides (e.g., LNA, cEt, ENA). In some embodiments, the flanking sequences X and Z may be of 1-20 nucleotides, 1-8 nucleotides, or 1-5 nucleotides in length. The flanking sequences X and Z may be of similar length or of dissimilar lengths. In some embodiments, the gap-segment Y may be a nucleotide sequence of 5-20 nucleotides, 5-15 nucleotides, 5-12 nucleotides, or 6-10 nucleotides in length.

In some embodiments, the gap region of the gapmer oligonucleotides may contain modified nucleosides known to be acceptable for efficient RNase H action in addition to DNA nucleosides, such as C4′-substituted nucleosides, acyclic nucleosides, and arabino-configured nucleosides. In some embodiments, the gap region comprises one or more unmodified internucleosides. In some embodiments, one or both flanking regions each independently comprise one or more phosphorothioate internucleoside linkages (e.g., phosphorothioate internucleoside linkages or other linkages) between at least two, at least three, at least four, at least five or more nucleotides. In some embodiments, the gap region and two flanking regions each independently comprise modified internucleoside linkages (e.g., phosphorothioate internucleoside linkages or other linkages) between at least two, at least three, at least four, at least five or more nucleotides.

A gapmer may be produced using appropriate methods. Representative U.S. patents, U.S. patent publications, and PCT publications that teach the preparation of gapmers include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; 5,700,922; 5,898,031; 7,015,315; 7,101,993; 7,399,845; 7,432,250; 7,569,686; 7,683,036; 7,750,131; 8,580,756; 9,045,754; 9,428,534; 9,695,418; 10,017,764; 10,260,069; 9,428,534; 8,580,756; U.S. patent publication Nos. US20050074801, US20090221685; US20090286969, US20100197762, and US20110112170; PCT publication Nos. WO2004069991; WO2005023825; WO2008049085 and WO2009090182; and EP Patent No. EP2,149,605, each of which is herein incorporated by reference in its entirety.

In some embodiments, the gapmer is 10-40 nucleosides in length. For example, the gapmer may be 10-40, 10-35, 10-30, 10-25, 10-20, 10-15, 15-40, 15-35, 15-30, 15-25, 15-20, 20-40, 20-35, 20-30, 20-25, 25-40, 25-35, 25-30, 30-40, 30-35, or 35-40 nucleosides in length. In some embodiments, the gapmer is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleosides in length.

In some embodiments, the gap region Y in the gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosine.

In some embodiments, the 5′ wing region of the gapmer (X in the 5′-X-Y-Z-3′ formula) and the 3′ wing region of the gapmer (Z in the 5′-X-Y-Z-3′ formula) are independently 1-20 nucleosides long. For example, the 5′ wing region of the gapmer (X in the 5′-X-Y-Z-3′ formula) and the 3′ wing region of the gapmer (Z in the 5′-X-Y-Z-3′ formula) may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the 5′ wing region of the gapmer (X in the 5′-X-Y-Z-3′ formula) and the 3′ wing region of the gapmer (Z in the 5′-X-Y-Z-3′ formula) are independently 1, 2, 3, 4, 5, 6, 7, 8, 9, 10, 11, 12, 13, 14, 15, 16, 17, 18, 19, or 20 nucleosides long. In some embodiments, the 5′ wing region of the gapmer (X in the 5′-X-Y-Z-3′ formula) and the 3′ wing region of the gapmer (Z in the 5′-X-Y-Z-3′ formula) are of the same length. In some embodiments, the 5′ wing region of the gapmer (X in the 5′-X-Y-Z-3′ formula) and the 3′ wing region of the gapmer (Z in the 5′-X-Y-Z-3′ formula) are of different lengths. In some embodiments, the 5′ wing region of the gapmer (X in the 5′-X-Y-Z-3′ formula) is longer than the 3′ wing region of the gapmer (Z in the 5′-X-Y-Z-3′ formula). In some embodiments, the 5′ wing region of the gapmer (X in the 5′-X-Y-Z-3′ formula) is shorter than the 3′ wing region of the gapmer (Z in the 5′-X-Y-Z-3′ formula).

In some embodiments, the gapmer comprises a 5′-X-Y-Z-3′ of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6- 11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12-3, 4-12-7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions in the 5′-X-Y-Z-3′ gapmer.

In some embodiments, one or more nucleosides in the 5′ wing region of the gapmer (X in the 5′-X-Y-Z-3′ formula) or the 3′ wing region of the gapmer (Z in the 5′-X-Y-Z-3′ formula) are modified nucleosides (e.g., high-affinity modified nucleosides). In some embodiments, the modified nucleoside (e.g., high-affinity modified nucleosides) is a 2′-modified nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA)).

In some embodiments, one or more nucleosides in the 5′ wing region of the gapmer (X in the 5′-X-Y-Z-3′ formula) are high-affinity modified nucleosides. In some embodiments, each nucleoside in the 5′ wing region of the gapmer (X in the 5′-X-Y-Z-3′ formula) is a high-affinity modified nucleoside. In some embodiments, one or more nucleosides in the 3′ wing region of the gapmer (Z in the 5′-X-Y-Z-3′ formula) are high-affinity modified nucleosides. In some embodiments, each nucleoside in the 3′ wing region of the gapmer (Z in the 5′-X-Y-Z-3′ formula) is a high-affinity modified nucleoside. In some embodiments, one or more nucleosides in the 5′ wing region of the gapmer (X in the 5′-X-Y-Z-3′ formula) are high-affinity modified nucleosides and one or more nucleosides in the 3′ wing region of the gapmer (Z in the 5′-X-Y-Z-3′ formula) are high-affinity modified nucleosides. In some embodiments, each nucleoside in the 5′ wing region of the gapmer (X in the 5′-X-Y-Z-3′ formula) is a high-affinity modified nucleoside and each nucleoside in the 3′wing region of the gapmer (Z in the 5′-X-Y-Z-3′ formula) is high-affinity modified nucleoside.

In some embodiments, the 5′ wing region of the gapmer (X in the 5′-X-Y-Z-3′ formula) comprises the same high affinity nucleosides as the 3′ wing region of the gapmer (Z in the 5′-X-Y-Z-3′ formula). For example, the 5′ wing region of the gapmer (X in the 5′-X-Y-Z-3′ formula) and the 3′ wing region of the gapmer (Z in the 5′-X-Y-Z-3′ formula) may comprise one or more non-bicyclic 2′-modified nucleosides (e.g., 2′-MOE or 2′-O-Me). In another example, the 5′ wing region of the gapmer (X in the 5′-X-Y-Z-3′ formula) and the 3′ wing region of the gapmer (Z in the 5′-X-Y-Z-3′ formula) may comprise one or more 2′-4′ bicyclic nucleosides (e.g., LNA or cEt). In some embodiments, each nucleoside in the 5′ wing region of the gapmer (X in the 5′-X-Y-Z-3′ formula) and the 3′ wing region of the gapmer (Z in the 5′-X-Y-Z-3′ formula) is a non-bicyclic 2′-modified nucleoside (e.g., 2′-MOE or 2′-O-Me). In some embodiments, each nucleoside in the 5′ wing region of the gapmer (X in the 5′-X-Y-Z-3′ formula) and the 3′ wing region of the gapmer (Z in the 5′-X-Y-Z-3′ formula) is a 2′-4′ bicyclic nucleoside (e.g., LNA or cEt).

In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a non-bicyclic 2′-modified nucleosides (e.g., 2′-MOE or 2′-O-Me) and each nucleoside in Y is a 2′-deoxyribonucleoside. In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X and Z is a 2′-4′ bicyclic nucleosides (e.g., LNA or cEt) and each nucleoside in Y is a 2′-deoxyribonucleoside. In some embodiments, the 5′ wing region of the gapmer (X in the 5′-X-Y-Z-3′ formula) comprises different high affinity nucleosides as the 3′ wing region of the gapmer (Z in the 5′-X-Y-Z-3′ formula). For example, the 5′ wing region of the gapmer (X in the 5′-X-Y-Z-3′ formula) may comprise one or more non-bicyclic 2′-modified nucleosides (e.g., 2′-MOE or 2′-O-Me) and the 3′ wing region of the gapmer (Z in the 5′-X-Y-Z-3′ formula) may comprise one or more 2′-4′ bicyclic nucleosides (e.g., LNA or cEt). In another example, the 3′ wing region of the gapmer (Z in the 5′-X-Y-Z-3′ formula) may comprise one or more non-bicyclic 2′-modified nucleosides (e.g., 2′-MOE or 2′-O-Me) and the 5′ wing region of the gapmer (X in the 5′-X-Y-Z-3′ formula) may comprise one or more 2′-4′ bicyclic nucleosides (e.g., LNA or cEt).

In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a non-bicyclic 2′-modified nucleoside (e.g., 2′-MOE or 2′-O-Me), each nucleoside in Z is a 2′-4′ bicyclic nucleoside (e.g., LNA or cEt), and each nucleoside in Y is a 2′-deoxyribonucleoside. In some embodiments, the gapmer comprises a 5′-X-Y-Z-3′ configuration, wherein X and Z are independently 1-7 (e.g., 1, 2, 3, 4, 5, 6, or 7) nucleosides in length and Y is 6-10 (e.g., 6, 7, 8, 9, or 10) nucleosides in length, wherein each nucleoside in X is a 2′-4′ bicyclic nucleoside (e.g., LNA or cEt), each nucleoside in Z is a non-bicyclic 2′-modified nucleoside (e.g., 2′-MOE or 2′-O-Me) and each nucleoside in Y is a 2′-deoxyribonucleoside.

In some embodiments, the 5′ wing region of the gapmer (X in the 5′-X-Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, the 3′ wing region of the gapmer (Z in the 5′-X-Y-Z-3′ formula) comprises one or more non-bicyclic 2′-modified nucleosides (e.g., 2′-MOE or 2′-O-Me) and one or more 2′-4′ bicyclic nucleosides (e.g., LNA or cEt). In some embodiments, both the 5′ wing region of the gapmer (X in the 5′-X-Y-Z-3′ formula) and the 3′ wing region of the gapmer (Z in the 5′-X-Y-Z-3′ formula) comprise one or more non-bicyclic 2′-modified nucleosides (e.g., 2′-MOE or 2′-O-Me) and one or more 2′-4′ bicyclic nucleosides (e.g., LNA or cEt).

In some embodiments, the gapmer comprises a 5′-X-Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X-Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, the gapmer comprises a 5′-X-Y-Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

Non-limiting examples of gapmers configurations with a mix of non-bicyclic 2′-modified nucleoside (e.g., 2′-MOE or 2′-O-Me) and 2′-4′ bicyclic nucleosides (e.g., LNA or cEt) in the 5′wing region of the gapmer (X in the 5′-X-Y-Z-3′ formula) and/or the 3′wing region of the gapmer (Z in the 5′-X-Y-Z-3′ formula) include: BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAA; KKK-(D)n-KKKAA; LLL-(D)n-LLLAA; BBB-(D)n-BBBEE; KKK-(D)n-KKKEE; LLL-(D)n-LLLEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BBB-(D)n-BBBAAA; KKK-(D)n-KKKAAA; LLL-(D)n-LLLAAA; BBB-(D)n-BBBEEE; KKK-(D)n-KKKEEE; LLL-(D)n-LLLEEE; BABA-(D)n-ABAB; KAKA-(D)n-AKAK; LALA-(D)n-ALAL; BEBE-(D)n-EBEB; KEKE-(D)n-EKEK; LELE-(D)n-ELEL; BABA-(D)n-ABAB; KAKA-(D)n-AKAK; LALA-(D)n-ALAL; BEBE-(D)n-EBEB; KEKE-(D)n-EKEK; LELE-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; ABAB-(D)n-ABAB; AKAK-(D)n-AKAK; ALAL-(D)n-ALAL; EBEB-(D)n-EBEB; EKEK-(D)n-EKEK; ELEL-(D)n-ELEL; AABB-(D)n-BBAA; BBAA-(D)n-AABB; AAKK-(D)n-KKAA; AALL-(D)n-LLAA; EEBB-(D)n-BBEE; EEKK-(D)n-KKEE; EELL-(D)n-LLEE; AABB-(D)n-BBAA; AAKK-(D)n-KKAA; AALL-(D)n-LLAA; EEBB-(D)n-BBEE; EEKK-(D)n-KKEE; EELL-(D)n-LLEE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; BBB-(D)n-BBA; KKK-(D)n-KKA; LLL-(D)n-LLA; BBB-(D)n-BBE; KKK-(D)n-KKE; LLL-(D)n-LLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBA; AKKK-(D)n-KKKA; ALLL-(D)n-LLLA; EBBB-(D)n-BBBE; EKKK-(D)n-KKKE; ELLL-(D)n-LLLE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; ABBB-(D)n-BBBAA; AKKK-(D)n-KKKAA; ALLL-(D)n-LLLAA; EBBB-(D)n-BBBEE; EKKK-(D)n-KKKEE; ELLL-(D)n-LLLEE; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBB; AAKKK-(D)n-KKK; AALLL-(D)n-LLL; EEBBB-(D)n-BBB; EEKKK-(D)n-KKK; EELLL-(D)n-LLL; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; AABBB-(D)n-BBBA; AAKKK-(D)n-KKKA; AALLL-(D)n-LLLA; EEBBB-(D)n-BBBE; EEKKK-(D)n-KKKE; EELLL-(D)n-LLLE; ABBAABB-(D)n-BB; AKKAAKK-(D)n-KK; ALLAALLL-(D)n-LL; EBBEEBB-(D)n-BB; EKKEEKK-(D)n-KK; ELLEELL-(D)n-LL; ABBAABB-(D)n-BB; AKKAAKK-(D)n-KK; ALLAALL-(D)n-LL; EBBEEBB-(D)n-BB; EKKEEKK-(D)n-KK; ELLEELL-(D)n-LL; ABBABB-(D)n-BBB; AKKAKK-(D)n-KKK; ALLALLL-(D)n-LLL; EBBEBB-(D)n-BBB; EKKEKK-(D)n-KKK; ELLELL-(D)n-LLL; ABBABB-(D)n-BBB; AKKAKK-(D)n-KKK; ALLALL-(D)n-LLL; EBBEBB-(D)n-BBB; EKKEKK-(D)n-KKK; ELLELL-(D)n-LLL; EEEK-(D)n-EEEEEEEE; EEK-(D)n-EEEEEEEEE; EK-(D)n-EEEEEEEEEE; EK-(D)n-EEEKK; K-(D)n-EEEKEKE; K-(D)n-EEEKEKEE; K-(D)n-EEKEK; EK-(D)n-EEEEKEKE; EK-(D)n-EEEKEK; EEK-(D)n-KEEKE; EK-(D)n-EEKEK; EK-(D)n-KEEK; EEK-(D)n-EEEKEK; EK-(D)n-KEEEKEE; EK-(D)n-EEKEKE; EK-(D)n-EEEKEKE; and EK-(D)n-EEEEKEK; wherein “A” represents a 2′-modified nucleoside; “B” represents a 2′-4′ bicyclic nucleoside; “K” represents a constrained ethyl nucleoside (cEt); “L” represents an LNA nucleoside; and “E” represents a 2′-MOE modified ribonucleoside; “D” represents a 2′-deoxyribonucleoside; “n” represents the length of the gap segment (Y in the 5′-X-Y-Z-3′ configuration) and is an integer between 1-20.

In some embodiments, any one of the gapmers described herein comprises one or more modified nucleoside linkages (e.g., a phosphorothioate linkage) in each of the X, Y, and Z regions. In some embodiments, each internucleoside linkage in the any one of the gapmers described herein is a phosphorothioate linkage. In some embodiments, each of the X, Y, and Z regions independently comprises a mix of phosphorothioate linkages and phosphodiester linkages. In some embodiments, each internucleoside linkage in the gap region Y is a phosphorothioate linkage, the 5′ wing region X comprises a mix of phosphorothioate linkages and phosphodiester linkages, and the 3′ wing region Z comprises a mix of phosphorothioate linkages and phosphodiester linkages.

Non-limiting examples of DMPK-targeting oligonucleotides are provided in Table 8, Table 9, and Table 10.

TABLE 8
Examples of DMPK-targeting
oligonucleotides (ASOs)

| SEQ | | SEQ | |
|---|---|---|---|
| ID | Target Sequence^{†} | ID | Antisense Sequence^{†} |
| NO: | (5′ to 3′) | NO: | (5′ to 3′) |
| 160 | CGGGCCAGGTGTATGCCATG | 231 | CATGGCATACACCTGGCCCG |
| 161 | AGGAGAGGGACGTGTTGGTG | 232 | CACCAACACGTCCCTCTCCT |
| 162 | GGAGAGGGACGTGTTGGTGA | 233 | TCACCAACACGTCCCTCTCC |
| 163 | AGGGACGTGTTGGTGAATGG | 234 | CCATTCACCAACACGTCCCT |
| 164 | CAGGATGAGAACTACCTGTA | 235 | TACAGGTAGTTCTCATCCTG |
| 165 | AGGATGAGAACTACCTGTAC | 236 | GTACAGGTAGTTCTCATCCT |
| 166 | GAGAACTACCTGTACCTGGT | 237 | ACCAGGTACAGGTAGTTCTC |
| 167 | AGAACTACCTGTACCTGGTC | 238 | GACCAGGTACAGGTAGTTCT |
| 168 | GAACTACCTGTACCTGGTCA | 239 | TGACCAGGTACAGGTAGTTC |
| 169 | CACTGCTGAGCAAGTTTGGG | 240 | CCCAAACTTGCTCAGCAGTG |
| 170 | CTACCTGGCGGAGATTGTCA | 241 | TGACAATCTCCGCCAGGTAG |
| 171 | TACCTGGCGGAGATTGTCAT | 242 | ATGACAATCTCCGCCAGGTA |
| 172 | ACCTGGCGGAGATTGTCATG | 243 | CATGACAATCTCCGCCAGGT |
| 173 | CCTGGCGGAGATTGTCATGG | 244 | CCATGACAATCTCCGCCAGG |
| 174 | CTGGCGGAGATTGTCATGGC | 245 | GCCATGACAATCTCCGCCAG |
| 175 | TGGCGGAGATTGTCATGGCC | 246 | GGCCATGACAATCTCCGCCA |
| 176 | GGCGGAGATTGTCATGGCCA | 247 | TGGCCATGACAATCTCCGCC |
| 177 | CCGGCTTGGCTACGTGCACA | 248 | TGTGCACGTAGCCAAGCCGG |
| 178 | CGGCTTGGCTACGTGCACAG | 249 | CTGTGCACGTAGCCAAGCCG |
| 179 | CATCCTGCTGGACCGCTGTG | 250 | CACAGCGGTCCAGCAGGATG |
| 180 | CTGCTGGACCGCTGTGGCCA | 251 | TGGCCACAGCGGTCCAGCAG |
| 181 | GAGTGTGACTGGTGGGCGCT | 252 | AGCGCCCACCAGTCACACTC |
| 182 | AGTGTGACTGGTGGGCGCTG | 253 | CAGCGCCCACCAGTCACACT |
| 183 | GTGTGACTGGTGGGCGCTGG | 254 | CCAGCGCCCACCAGTCACAC |
| 184 | TGGGCGCTGGGTGTATTCGC | 255 | GCGAATACACCCAGCGCCCA |
| 185 | GGGCGCTGGGTGTATTCGCC | 256 | GGCGAATACACCCAGCGCCC |
| 186 | GGCAAGATCGTCCACTACAA | 257 | TTGTAGTGGACGATCTTGCC |
| 187 | GCAAGATCGTCCACTACAAG | 258 | CTTGTAGTGGACGATCTTGC |
| 188 | CAAGATCGTCCACTACAAGG | 259 | CCTTGTAGTGGACGATCTTG |
| 189 | CTCGACTGGGATGGTCTCCG | 260 | CGGAGACCATCCCAGTCGAG |
| 190 | GGAGACACTGTCGGACATTC | 261 | GAATGTCCGACAGTGTCTCC |
| 191 | GAGACACTGTCGGACATTCG | 262 | CGAATGTCCGACAGTGTCTC |
| 192 | GACAGTGAGGTCCCAGGCCC | 263 | GGGCCTGGGACCTCACTGTC |
| 193 | CTGCTTGAGCCACACGTGCA | 264 | TGCACGTGTGGCTCAAGCAG |
| 194 | GGATGAAACAGCTGAAGTGG | 265 | CCACTTCAGCTGTTTCATCC |
| 195 | GGCTGAGGCCGAGGTGACGC | 266 | GCGTCACCTCGGCCTCAGCC |
| 196 | GCTGAGGCCGAGGTGACGCT | 267 | AGCGTCACCTCGGCCTCAGC |
| 197 | AACTTCGCCAGTCAACTACG | 268 | CGTAGTTGACTGGCGAAGTT |
| 198 | CAGTCCTGTGATCCGGGCCC | 269 | GGGCCCGGATCACAGGACTG |
| 199 | GCCCTGACGTGGATGGGCAA | 270 | TTGCCCATCCACGTCAGGGC |
| 200 | GGCAGCAAGCCGGGCCGTCC | 271 | GGACGGCCCGGCTTGCTGCC |
| 201 | GCCGGGCCGTCCGTGTTCCA | 272 | TGGAACACGGACGGCCCGGC |
| 202 | CCCAATCCACGTTTTGGATG | 273 | CATCCAAAACGTGGATTGGG |
| 203 | CCAATCCACGTTTTGGATGC | 274 | GCATCCAAAACGTGGATTGG |
| 204 | GCTTGAGCCACACGTG | 275 | CACGTGTGGCTCAAGC |
| 205 | CTTGAGCCACACGTGC | 276 | GCACGTGTGGCTCAAG |
| 206 | TGCTTGAGCCACACGT | 277 | ACGTGTGGCTCAAGCA |
| 207 | CTGCTGAGCAAGTTTG | 278 | CAAACTTGCTCAGCAG |
| 208 | ATGAAACAGCTGAAGT | 279 | ACTTCAGCTGTTTCAT |
| 209 | CTTCGCCAGTCAACTA | 280 | TAGTTGACTGGCGAAG |
| 210 | GTCCTGTGATCCGGGC | 281 | GCCCGGATCACAGGAC |
| 211 | GGCGGAGATTGTCATG | 282 | CATGACAATCTCCGCC |
| 212 | CTGAGGCCGAGGTGAC | 283 | GTCACCTCGGCCTCAG |
| 213 | AACTACCTGTACCTGG | 284 | CCAGGTACAGGTAGTT |
| 214 | AGAGGGACGTGTTGGT | 285 | ACCAACACGTCCCTCT |
| 215 | TGCTGAGCAAGTTTGG | 286 | CCAAACTTGCTCAGCA |
| 216 | TGAAACAGCTGAAGTG | 287 | CACTTCAGCTGTTTCA |
| 217 | TTCGCCAGTCAACTAC | 288 | GTAGTTGACTGGCGAA |
| 218 | TCCTGTGATCCGGGCC | 289 | GGCCCGGATCACAGGA |
| 219 | ACTGCTGAGCAAGTTT | 290 | AAACTTGCTCAGCAGT |
| 220 | GATGAAACAGCTGAAG | 291 | CTTCAGCTGTTTCATC |
| 221 | ACTTCGCCAGTCAACT | 292 | AGTTGACTGGCGAAGT |
| 222 | AGTCCTGTGATCCGGG | 293 | CCCGGATCACAGGACT |
| 223 | GCGGAGATTGTCATGG | 294 | CCATGACAATCTCCGC |
| 224 | TGGCGGAGATTGTCAT | 295 | ATGACAATCTCCGCCA |
| 225 | TGAGGCCGAGGTGACG | 296 | CGTCACCTCGGCCTCA |
| 226 | GCTGAGGCCGAGGTGA | 297 | TCACCTCGGCCTCAGC |
| 227 | ACTACCTGTACCTGGT | 298 | ACCAGGTACAGGTAGT |
| 228 | GAACTACCTGTACCTG | 299 | CAGGTACAGGTAGTTC |
| 229 | GAGGGACGTGTTGGTG | 300 | CACCAACACGTCCCTC |
| 230 | GAGAGGGACGTGTTGG | 301 | CCAACACGTCCCTCTC |

^{†}Each thymine base (T) in any one of the oligonucleotides and/or target sequences provided in Table 8 may independently and optionally be replaced with a uracil base (U). Target sequences listed in Table 8 contain Ts, but binding of a DMPK-targeting oligonucleotide to RNA and/or DNA is contemplated.

TABLE 9
Examples of DMPK-targeting oligonucleotides (ASOs)

| ASO | SEQ ID | Antisense Sequence^{†} | ASO Structure^{†} |
|---|---|---|---|
| Name | NO: | (5′ to 3′) | (5′ to 3′) |
| ASO1 | 302 | CAUGGCATACACCTGGCCCG | oC*oA*oU*oG*oG*dC*dA*dT*dA*dC*dA* |
| | | | dC*dC*dT*dG*oG*oC*oC*oC*oG |
| ASO2 | 303 | CACCAACACGTCCCTCUCCU | oC*oA*oC*oC*oA*dA*dC*dA*xdC*dG*dT |
| | | | *dC*dC*dC*dT*oC*oU*oC*oC*oU |
| ASO3 | 304 | UCACCAACACGTCCCUCUCC | oU*oC*A*oC*oC*dA*dA*dC*dA*xdC*dG* |
| | | | dT*dC*dC*dC*oU*oC*oU*oC*oC |
| ASO4 | 305 | CCAUUCACCAACACGUCCCU | oC*oC*oA*oU*oU*dC*dA*dC*dC*dA*dA* |
| | | | dC*dA*xdC*dG*oU*oC*oC*oC*oU |
| ASO5 | 306 | UACAGGTAGTTCTCAUCCUG | QU*oA*oC*oA*G*dG*dT*dA*dG*dT*dT*d |
| | | | C*dT*dC*dA*oU*oC*oC*oU*oG |
| ASO6 | 307 | GUACAGGTAGTTCTCAUCCU | oG*oU*oA*oC*oA*dG*dG*dT*dA*dG*dT* |
| | | | dT*dC*dT*dC*oA*oU*oC*oC*oU |
| ASO7 | 308 | ACCAGGTACAGGTAGUUCUC | oA*oC*oC*oA*oG*dG*dT*dA*dC*dA*dG* |
| | | | dG*dT*dA*dG*oU*oU*oC*oU*oC |
| ASO8 | 309 | GACCAGGTACAGGTAGUUCU | oG*oA*oC*oC*oA*dG*dG*dT*dA*dC*dA* |
| | | | dG*dG*dT*dA*oG*oU*oU*oC*oU |
| ASO9 | 310 | UGACCAGGTACAGGTAGUUC | oU*oG*oA*oC*oC*dA*dG*dG*dT*dA*dC* |
| | | | dA*dG*dG*dT*oA*xoG*oU*oU*oC |
| ASO10 | 311 | CCCAAACTTGCTCAGCAGUG | oC*oC*oC*oA*oA*dA*dC*dT*dT*dG*dC* |
| | | | dT*dC*dA*dG*oC*oA*oG*oU*oG |
| ASO11 | 312 | UGACAATCTCCGCCAGGUAG | oU*oG*oA*oC*oA*dA*dT*dC*dT*dC*xdC |
| | | | *dG*dC*dC*dA*oG*oG*oU*oA*oG |
| ASO12 | 313 | AUGACAATCTCCGCCAGGUA | oA*oU*oG*oA*oC*dA*dA*dT*dC*dT*dC* |
| | | | xdC*dG*dC*dC*oA*oG*G*oU*oA |
| ASO13 | 314 | CAUGACAATCTCCGCCAGGU | oC*oA*oU*oG*oA*dC*dA*dA*dT*dC*dT* |
| | | | dC*xdC*dG*dC*oC*oA*oG*oG*oU |
| ASO14 | 315 | CCAUGACAATCTCCGCCAGG | oC*oC*oA*oU*oG*dA*dC*dA*dA*dT*dC* |
| | | | dT*dC*xdC*dG*oC*oC*oA*oG*oG |
| ASO15 | 316 | GCCAUGACAATCTCCGCCAG | oG*oC*oC*oA*oU*dG*dA*dC*dA*dA*dT* |
| | | | dC*dT*dC*xdC*oG*oC*oC*oA*oG |
| ASO16 | 246 | GGCCATGACAATCTCCGCCA | oG*oG*oC*oC*oA*dT*dG*dA*dC*dA*dA* |
| | | | dT*dC*dT*dC*oC*oG*oC*oC*oA |
| ASO17 | 317 | UGGCCATGACAATCTCCGCC | oU*oG*oG*oC*oC*dA*dT*dG*dA*dC*dA* |
| | | | dA*dT*dC*dT*oC*oC*oG*oC*oC |
| ASO18 | 318 | UGUGCACGTAGCCAAGCCGG | oU*oG*oU*oG*oC*dA*xdC*dG*dT*dA*dG |
| | | | *dC*dC*dA*dA*oG*oC*oC*oG*oG |
| ASO19 | 319 | CUGUGCACGTAGCCAAGCCG | oC*oU*oG*oU*oG*dC*dA*xdC*dG*dT*dA |
| | | | *dG*dC*dC*dA*oA*oG*oC*oC*oG |
| ASO20 | 320 | CACAGCGGTCCAGCAGGAUG | oC*oA*oC*oA*oG*xdC*dG*dG*dT*dC*dC |
| | | | *dA*dG*dC*dA*oG*oG*A*oU*oG |
| ASO21 | 321 | UGGCCACAGCGGTCCAGCAG | oU*oG*oG*oC*oC*dA*dC*dA*dG*xdC*dG |
| | | | *dG*dT*dC*dC*oA*oG*oC*oA*oG |
| ASO22 | 322 | AGCGCCCACCAGTCACACUC | oA*oG*oC*oG*oC*dC*dC*dA*dC*dC*dA* |
| | | | dG*dT*dC*oA*oC*oA*oC*oU*oC |
| ASO23 | 323 | CAGCGCCCACCAGTCACACU | oC*oA*oG*oC*oG*dC*dC*dC*dA*dC*dC* |
| | | | dA*dG*dT*dC*oA*oC*oA*oC*oU |
| ASO24 | 254 | CCAGCGCCCACCAGTCACAC | oC*oC*oA*oG*oC*dG*dC*dC*dC*dA*dC* |
| | | | dC*dA*dG*dT*oC*oA*oC*oA*oC |
| ASO25 | 255 | GCGAATACACCCAGCGCCCA | oG*oC*oG*oA*oA*dT*dA*dC*dA*dC*dC* |
| | | | dc*dA*dG*xdC*oG*oC*oC*oC*oA |
| ASO26 | 256 | GGCGAATACACCCAGCGCCC | oG*oG*oC*oG*oA*dA*dT*dA*dC*dA*dC* |
| | | | dc*dC*dA*dG*oC*oG*oC*oC*oC |
| ASO27 | 324 | UUGUAGTGGACGATCUUGCC | oU*oU*oG*oU*oA*dG*dT*dG*dG*dA*xdC |
| | | | *dG*dA*dT*dC*oU*oU*oG*oC*oC |
| ASO28 | 325 | CUUGUAGTGGACGATCUUGC | oC*oU*oU*oG*oU*dA*dG*dT*dG*dG*dA* |
| | | | xdC*dG*dA*dT*oC*oU*oU*G*oC |
| ASO29 | 326 | CCUUGTAGTGGACGAUCUUG | oC*oC*oU*oU*oG*dT*dA*dG*dT*dG*dG* |
| | | | dA*xdC*dG*dA*oU*oC*oU*oU*oG |
| ASO30 | 327 | CGGAGACCATCCCAGUCGAG | oC*oG*oG*oA*oG*dA*dC*dC*dA*dT*dC* |
| | | | dC*dC*dA*dG*oU*oC*oG*oA*oG |
| ASO31 | 328 | GAAUGTCCGACAGTGUCUCC | oG*oA*oA*oU*oG*dT*dC*xdC*dG*dA*dC |
| | | | *dA*dG*dT*dG*oU*oC*oU*oC*oC |
| ASO32 | 329 | CGAAUGTCCGACAGTGUCUC | oC*oG*oA*oA*oU*dG*dT*dC*xdC*dG*dA |
| | | | *dC*dA*dG*dT*oG*oU*oC*oU*oC |
| ASO33 | 330 | GGGCCTGGGACCTCACUGUC | oG*oG*oG*oC*oC*dT*dG*dG*dG*dA*dC* |
| | | | dC*dT*dC*dA*oC*oU*oG*oU*oC |
| ASO34 | 331 | UGCACGTGTGGCTCAAGCAG | oU*oG*oC*oA*oC*dG*dT*dG*dT*dG*dG* |
| | | | dC*dT*dC*dA*oA*oG*oC*oA*oG |
| ASO35 | 332 | CCACUTCAGCTGTTTCAUCC | oC*oC*oA*oC*oU*dT*dC*dA*dG*dC*dT* |
| | | | dG*dT*dT*dT*oC*oA*oU*oC*oC |
| ASO36 | 333 | GCGUCACCTCGGCCTCAGCC | oG*oC*oG*oU*oC*dA*dC*dC*dT*xdC*dG |
| | | | *dG*dC*dC*dT*oC*oA*oG*oC*oC |
| ASO37 | 334 | AGCGUCACCTCGGCCUCAGC | OA*oG*oC*oG*oU*dC*dA*dC*dC*dT*xdC |
| | | | *dG*dG*dC*dC*oU*oC*oA*oG*oC |
| ASO38 | 335 | CGUAGTTGACTGGCGAAGUU | oC*oG*oU*oA*oG*dT*dT*dG*dA*dC*dT* |
| | | | dG*dG*xdC*dG*oA*oA*oG*oU*oU |
| ASO39 | 336 | GGGCCCGGATCACAGGACUG | oG*oG*oG*oC*oC*xdC*dG*dG*dA*dT*dC |
| | | | *dA*dC*dA*dG*oG*oA*oC*oU*oG |
| ASO40 | 337 | UUGCCCATCCACGTCAGGGC | oU*oU*oG*oC*oC*dC*dA*dT*dC*dC*dA* |
| | | | xdC*dG*dT*dC*oA*oG*oG*oG*oC |
| ASO41 | 338 | GGACGGCCCGGCTTGCUGCC | oG*oG*oA*oC*oG*dG*dC*dC*xdC*dG*dG |
| | | | *dC*dT*dT*dG*oC*oU*oG*oC*oC |
| ASO42 | 339 | UGGAACACGGACGGCCCGGC | oU*oG*oG*A*A*dC*dA*xdC*dG*dG*dA*x |
| | | | dC*dG*dG*dC*oC*oC*oG*oG*oC |
| ASO43 | 340 | CAUCCAAAACGTGGAUUGGG | oC*oA*oU*oC*oC*dA*dA*dA*dA*xdC*dG |
| | | | *dT*dG*dG*dA*oU*oU*oG*oG*oG |
| ASO44 | 341 | GCAUCCAAAACGTGGAUUGG | oG*oC*oA*oU*oC*dC*dA*dA*dA*dA*xdC |
| | | | *dG*dT*dG*dG*oA*oU*oU*oG*oG |

^{†}Each thymine base (T) in any one of the oligonucleotides provided in Table 9 may independently and optionally be replaced with a uracil base (U),and each U may independently and optionally be replaced with a T. Each U and each cytidine base (C) may alternatively, or in addition (e.g., in addition) be independently and optionally methylated.
“xdC” is 5-methyl-deoxycytidine;
“dN” is 2′-deoxyribonucleoside;
“oN” is 2′-MOE modified ribonucleoside;
“oC” is 5-methyl-2′-MOE-cytidine;
“oU” is 5-methyl-2′-MOE-uridine;
“xoG” is 7-methyl-2′-MOE-guanosine;
“*” indicates a phosphorothioate (PS) internucleoside linkage.
Each ASO listed in Table 9 has a fully PS backbone and a gapmer configuration 5′-X-Y-Z-3′ of EEEEE-(D)₁₀-EEEEE, where “E” specifies a 2′-MOE modified ribonucleoside;
“D” specifies a 2′-deoxyribonucleoside,and the subscript number indicates the number of 2′-deoxyribonucleosides in Y. Each ASO can optionally be modified with NH₂-(CH₂)₆ at its 5′ end,and the linkage between the NH₂-(CH₂)₆ and the 5′ terminal nucleoside is optionally a phosphodiester linkage.

TABLE 10
Examples of DMPK-targeting oligonucleotides (ASOs)

| | SEQ | Antisense | |
|---|---|---|---|
| ASO | ID | Sequence^{†} | ASO Structure^{†} |
| Name | NO: | (5′ to 3′) | (5′ to 3′) |
| ASO45 | 275 | CACGTGTGGCTCAAGC | x+C*+A*xoC*oG*dT*dG*dT*dG*dG*xdC*dT*xdC*oA |
| | | | *oA*+G*x+C |
| ASO46 | 275 | CACGTGTGGCTCAAGC | xoC*oA*x+C*+G*dT*dG*dT*dG*dG*xdC*dT*xdC*+A |
| | | | *+A*oG*xoC |
| ASO47 | 276 | GCACGTGTGGCTCAAG | +G*x+C*oA*xoC*dG*dT*dG*dT*dG*dG*xdC*dT*xoC |
| | | | *oA*+A*+G |
| ASO48 | 276 | GCACGTGTGGCTCAAG | oG*xoC*+A*x+C*dG*dT*dG*dT*dG*dG*xdC*dT*x+C |
| | | | *+A*oA*oG |
| ASO49 | 342 | ACGUGTGGCTCAAGCA | +A*x+C*oG*oU*dG*dT*dG*dG*xdC*dT*xdC*dA*oA* |
| | | | oG*x+C*+A |
| ASO50 | 342 | ACGUGTGGCTCAAGCA | oA*xoC*+G*+U*dG*dT*dG*dG*xdC*dT*xdC*dA*+A* |
| | | | +G*xoC*oA |
| ASO51 | 278 | CAAACTTGCTCAGCAG | x+C*+A*oA*oA*xdC*dT*dT*dG*xdC*dT*xdC*dA*oG |
| | | | *xoC*+A*+G |
| ASO52 | 343 | ACUUCAGCTGTTUCAU | +A*x+C*oU*oU*xdC*dA*dG*xdC*dT*dG*dT*dT*oU* |
| | | | xoC*+A*+U |
| ASO53 | 344 | UAGUTGACTGGCGAAG | +U*+A*oG*oU*dT*dG*dA*xdC*dT*dG*dG*xdC*oG*o |
| | | | A*+A*+G |
| ASO54 | 281 | GCCCGGATCACAGGAC | +G*x+C*xoC*xoC*dG*dG*dA*dT*dC*dA*xdC*dA*oG |
| | | | *oG*+A*x+C |
| ASO55 | 345 | CAUGACAATCTCCGCC | x+C*+A*oU*oG*dA*xdC*dA*dA*dT*xdC*dT*xdC*xo |
| | | | C*oG*x+C*x+C |
| ASO56 | 346 | GUCACCTCGGCCUCAG | +G*+U*xoC*oA*xdC*xdC*dT*xdC*dG*dG*xdC*dC*o |
| | | | U*xoC*+A*+G |
| ASO57 | 347 | CCAGGTACAGGTAGUU | x+C*x+C*oA*oG*dG*dT*dA*xdC*dA*dG*dG*dT*oA* |
| | | | oG*+U*+U |
| ASO58 | 348 | ACCAACACGTCCCUCU | +A*x+C*xoC*oA*dA*xdC*dA*xdC*dG*dT*xdC*xdC* |
| | | | xoC*oU*x+C*+U |
| ASO59 | 286 | CCAAACTTGCTCAGCA | x+C*x+C*oA*A*dA*xdC*dT*dT*dG*xdC*dT*xdC*oA |
| | | | *oG*x+C*+A |
| ASO60 | 349 | CACUTCAGCTGTUUCA | x+C*+A*xoC*oU*dT*xdC*dA*dG*xdC*dT*dG*dT*oU |
| | | | *oU*x+C*+A |
| ASO61 | 350 | GUAGTTGACTGGCGAA | +G*+U*oA*oG*dT*dT*dG*dA*xdC*dT*dG*dG*xoC*o |
| | | | G*+A*+A |
| ASO62 | 289 | GGCCCGGATCACAGGA | +G*+G*xoC*xoC*xdC*dG*dG*dA*dT*xdC*dA*xdC*o |
| | | | A*oG*+G*+A |
| ASO63 | 351 | AAACTTGCTCAGCAGU | +A*+A*oA*xoC*dT*dT*dG*xdC*dT*xdC*dA*dG*xoC |
| | | | *oA*+G*+U |
| ASO64 | 352 | CUUCAGCTGTTTCAUC | x+C*+U*oU*xoC*dA*dG*xdC*dT*dG*dT*dT*dT*xoC |
| | | | *oA*+U*x+C |
| ASO65 | 353 | AGUUGACTGGCGAAGU | +A*+G*oU*oU*dG*dA*xdC*dT*dG*dG*xdC*dG*oA*o |
| | | | A*+G*+U |
| ASO66 | 354 | CCCGGATCACAGGACU | x+C*x+C*xoC*oG*dG*dA*dT*xdC*dA*xdC*dA*dG*o |
| | | | G*oA*x+C*+U |
| ASO67 | 278 | CAAACTTGCTCAGCAG | xoC*oA*+A*+A*xdC*dT*dT*dG*xdC*dT*xdC*dA*+G |
| | | | *x+C*oA*oG |
| ASO68 | 343 | ACUUCAGCTGTTUCAU | oA*xoC*+U*+U*xdC*dA*dG*xdC*dT*dG*dT*dT*+U* |
| | | | x+C*oA*oU |
| ASO69 | 344 | UAGUTGACTGGCGAAG | oU*oA*+G*+U*dT*dG*dA*xdC*dT*dG*dG*xdC*+G*+ |
| | | | A*oA*oG |
| ASO70 | 281 | GCCCGGATCACAGGAC | oG*xoC*x+C*x+C*dG*dG*dA*dT*dC*dA*dC*dA*+G* |
| | | | +G*oA*xoC |
| ASO71 | 286 | CCAAACTTGCTCAGCA | xoC*xoC*+A*+A*dA*xdC*dT*dT*dG*xdC*dT*xdC*+ |
| | | | A*+G*xoC*oA |
| ASO72 | 349 | CACUTCAGCTGTUUCA | xoC*oA*x+C*+U*dT*xdC*dA*dG*xdC*dT*dG*dT*+U |
| | | | *+U*xoC*oA |
| ASO73 | 350 | GUAGTTGACTGGCGAA | oG*oU*+A*+G*dT*dT*dG*dA*xdC*dT*dG*dG*x+C*+ |
| | | | G*oA*oA |
| ASO74 | 289 | GGCCCGGATCACAGGA | oG*oG*x+C*x+C*xdC*dG*dG*dA*dT*xdC*dA*xdC*+ |
| | | | A*+G*oG*oA |
| ASO75 | 351 | AAACTTGCTCAGCAGU | oA*oA*+A*x+C*dT*dT*dG*xdC*dT*xdC*dA*dG*x+C |
| | | | *+A*oG*oU |
| ASO76 | 352 | CUUCAGCTGTTTCAUC | xoC*oU*+U*x+C*dA*dG*xdC*dT*dG*dT*dT*dT*x+C |
| | | | *+A*oU*xoC |
| ASO77 | 353 | AGUUGACTGGCGAAGU | oA*oG*+U*+U*dG*dA*xdC*dT*dG*dG*xdC*dG*+A*+ |
| | | | A*oG*oU |
| ASO78 | 354 | CCCGGATCACAGGACU | xoC*xoC*x+C*+G*dG*dA*dT*xdC*dA*xdC*dA*dG*+ |
| | | | G*+A*xoC*oU |
| ASO79 | 355 | CCAUGACAATCTCCGC | x+C*x+C*oA*oU*dG*dA*xdC*dA*dA*dT*xdC*dT*xo |
| | | | C*xoC*+G*x+C |
| ASO80 | 356 | AUGACAATCTCCGCCA | +A*+U*oG*oA*xdC*dA*dA*dT*xdC*dT*xdC*xdC*oG |
| | | | *xoC*x+C*+A |
| ASO81 | 357 | CGUCACCTCGGCCUCA | x+C*+G*oU*xoC*dA*xdC*xdC*dT*xdC*dG*dG*xdC* |
| | | | xoC*oU*x+C*+A |
| ASO82 | 358 | UCACCTCGGCCTCAGC | +U*x+C*oA*xoC*xdC*dT*xdC*dG*dG*xdC*xdC*dT* |
| | | | xoC*oA*+G*x+C |
| ASO83 | 359 | ACCAGGTACAGGUAGU | +A*x+C*xoC*oA*dG*dG*dT*dA*xdC*dA*dG*dG*oU* |
| | | | OA*+G*+U |
| ASO84 | 360 | CAGGTACAGGTAGUUC | x+C*+A*oG*oG*dT*dA*xdC*dA*dG*dG*dT*dA*oG*o |
| | | | U*+U*x+C |
| ASO85 | 361 | CACCAACACGTCCCUC | x+C*+A*xoC*xoC*dA*dA*xdC*dA*xdC*dG*dT*xdC* |
| | | | xoC*xoC*+U*x+C |
| ASO86 | 362 | CCAACACGTCCCUCUC | x+C*x+C*oA*oA*xdC*dA*xdC*dG*dT*xdC*xdC*xdC |
| | | | *oU*xoC*+U*x+C |

^{†}Each thymine base (T) in any one of the oligonucleotides provided in Table 10 may independently and optionally be replaced with a uracil base (U),and each U may independently and optionally be replaced with a T.
Each U and each cytidine base (C) may alternatively, or in addition (e.g., in addition) be independently and optionally methylated.
“xdC” is 5-methyl-deoxycytidine;
“dN” is 2′-deoxyribonucleoside;
“oN” is 2′-MOE modified ribonucleoside;
“xoC” is 5-methyl-2′-MOE-cytidine;
“x+C” is 5-methyl LNA cytidine;
“+N” is an LNA nucleoside;
“oU” is 5-methyl-2′-MOE-uridine;
“+U” is 5-methyl LNA uridine;
“*”indicates a phosphorothioate (PS) internucleoside linkage.
Each ASO listed in Table 10 has a fully PS backbone and a gapmer configuration 5′-X-Y-Z-3′ of LLEE-(D)₈-EELL or EELL-(D)₈-LLEE, where “E” specifies a 2′-MOE modified ribonucleoside;
“L” is LNA;
“D” specifies a 2′-deoxyribonucleoside,and the subscript number indicates the number of 2′-deoxyribonucleosides in Y.
Each ASO can optionally be modified with NH₂-(CH₂)₆ at its 5′ end,and the linkage between the NH₂-(CH₂)₆ and the 5′ terminal nucleoside is optionally a phosphodiester linkage.

In some embodiments, a DMPK-targeting oligonucleotide described herein is 15-25 nucleosides (e.g., 15-20, 15, 16, 17, 18, 19, 20, 21, 22, 23, 24, or 25 nucleosides) in length and comprises a region of complementarity to at least 15 consecutive nucleosides (e.g., at least 15, at least 16, at least 17, at least 18, at least 19, or 20 consecutive nucleosides) of any one of SEQ ID NOs: 160-230. In some embodiments, the DMPK-targeting oligonucleotide comprises a 5′-X-Y-Z-3′ configuration, wherein X comprises 3-7 (e.g., 3-5, 3, 4, 5, 6, or 7) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-15 (e.g., 6-10, 6, 7, 8, 9, 10, 11, 12, 13, 14, or 15) linked 2′-deoxyribonucleosides, wherein each cytosine in Y is optionally and independently a 5-methyl-cytosine; and Z comprises 3-7 (e.g., 3-5, 3, 4, 5, 6, or 7) linked nucleosides, wherein at least one of the nucleosides in Z is a 2′-modified nucleoside (e.g., 2′-MOE modified nucleoside, 2′-O-Me modified nucleoside, LNA, cEt, or ENA).

In some embodiments, a DMPK-targeting oligonucleotide described herein comprises at least 15 consecutive nucleosides (e.g., at least 15, at least 16, at least 17, at least 18, at least 19, or 20 consecutive nucleosides) of the nucleotide sequence of any one of SEQ ID NOs: 231-362, wherein each thymine base (T) may independently and optionally be replaced with a uracil base (U), and each U may independently and optionally be replaced with a T. In some embodiments, the DMPK-targeting oligonucleotide comprises a 5′-X-Y-Z-3′ configuration, wherein X comprises 3-7 (e.g., 3-5, 3, 4, 5, 6, or 7) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-15 (e.g., 6-10, 6, 7, 8, 9, 10, 11, 12, 13, 14, or 15) linked 2′-deoxyribonucleosides, wherein each cytosine in Y is optionally and independently a 5-methyl-cytosine; and Z comprises 3-7 (e.g., 3-5, 3, 4, 5, 6, or 7) linked nucleosides, wherein at least one of the nucleosides in Z is a 2′-modified nucleoside (e.g., 2′-MOE modified nucleoside, 2′-O-Me modified nucleoside, LNA, cEt, or ENA).

In some embodiments, a DMPK-targeting oligonucleotide described herein comprises the nucleotide sequence of any one of SEQ ID NOs: 231-362, wherein each thymine base (T) may independently and optionally be replaced with a uracil base (U), and each U may independently and optionally be replaced with a T. In some embodiments, the DMPK-targeting oligonucleotide comprises a 5′-X-Y-Z-3′ configuration, wherein X comprises 3-7 (e.g., 3-5, 3, 4, 5, 6, or 7) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-15 (e.g., 6-10, 6, 7, 8, 9, 10, 11, 12, 13, 14, or 15) linked 2′-deoxyribonuclsides, wherein each cytosine in Y is optionally and independently a 5-methyl-cytosine; and Z comprises 3-7 (e.g., 3-5, 3, 4, 5, 6, or 7) linked nucleosides, wherein at least one of the nucleosides in Z is a 2′-modified nucleoside (e.g., 2′-MOE modified nucleoside, 2′-O-Me modified nucleoside, LNA, cEt, or ENA).

In some embodiments, a DMPK-targeting oligonucleotide described herein is 15-25 nucleosides (e.g., 15-20, 15, 16, 17, 18, 19, 20, 21, 22, 23, 24, or 25 nucleosides) in length, comprises a region of complementarity to at least 15 consecutive nucleosides (e.g., at least 15, at least 16, at least 17, at least 18, at least 19, or 20 consecutive nucleosides) of any one of SEQ ID NOs: 160-230, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, each nucleoside in X is a 2′-modified nucleoside and/or (e.g., and) each nucleoside in Z is a 2′-modified nucleoside. In some embodiments, the 2′-modified nucleoside is a 2′-4′ bicyclic nucleoside (e.g., LNA, cEt, or ENA) or a non-bicyclic 2′-modified nucleoside (e.g., 2′-MOE modified nucleoside or 2′-O-Me modified nucleoside).

In some embodiments, a DMPK-targeting oligonucleotide described herein is 15-25 nucleosides (e.g., 15-20, 15, 16, 17, 18, 19, 20, 21, 22, 23, 24, or 25 nucleosides) in length, comprises a region of complementarity to at least 15 consecutive nucleosides (e.g., at least 15, at least 16, at least 17, at least 18, at least 19, or 20 consecutive nucleosides) of any one of SEQ ID NOs: 160-230, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, each nucleoside in X is a non-bicyclic 2′-modified nucleoside (e.g., 2′-MOE modified nucleoside) and/or (e.g., and) each nucleoside in Z is a non-bicyclic 2′-modified nucleoside (e.g., 2′-MOE modified nucleoside). In some embodiments, each nucleoside in X is a 2′-4′ bicyclic nucleoside (e.g., LNA, cEt, or ENA) and/or (e.g., and) each nucleoside in Z is a 2′-4′ bicyclic nucleoside (e.g., LNA, cEt, or ENA).

In some embodiments, a DMPK-targeting oligonucleotide described herein is 15-25 nucleosides (e.g., 15-20, 15, 16, 17, 18, 19, 20, 21, 22, 23, 24, or 25 nucleosides) in length, comprises a region of complementarity to at least 15 consecutive nucleosides (e.g., at least 15, at least 16, at least 17, at least 18, at least 19, or 20 consecutive nucleosides) of any one of SEQ ID NOs: 160-230, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, X comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside), and/or (e.g., and) Z comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside).

In some embodiments, a DMPK-targeting oligonucleotide described herein comprises at least 15 consecutive nucleosides (e.g., at least 15, at least 16, at least 17, at least 18, at least 19, or 20 consecutive nucleosides) of the nucleotide sequence of any one of SEQ ID NOs: 231-362, wherein each thymine base (T) may independently and optionally be replaced with a uracil base (U), and each U may independently and optionally be replaced with a T, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, each nucleoside in X is a 2′-modified nucleoside and/or (e.g., and) each nucleoside in Z is a 2′-modified nucleoside. In some embodiments, the 2′-modified nucleoside is a 2′-4′ bicyclic nucleoside (e.g., LNA, cEt, or ENA) or a non-bicyclic 2′-modified nucleoside (e.g., 2′-MOE modified nucleoside or 2′-O-Me modified nucleoside).

In some embodiments, a DMPK-targeting oligonucleotide described herein comprises at least 15 consecutive nucleosides (e.g., at least 15, at least 16, at least 17, at least 18, at least 19, or 20 consecutive nucleosides) of the nucleotide sequence of any one of SEQ ID NOs: 231-362, wherein each thymine base (T) may independently and optionally be replaced with a uracil base (U), and each U may independently and optionally be replaced with a T, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, each nucleoside in X is a non-bicyclic 2′-modified nucleoside (e.g., 2′-MOE modified nucleoside) and/or (e.g., and) each nucleoside in Z is a non-bicyclic 2′-modified nucleoside (e.g., 2′-MOE modified nucleoside). In some embodiments, each nucleoside in X is a 2′-4′ bicyclic nucleoside (e.g., LNA, cEt, or ENA) and/or (e.g., and) each nucleoside in Z is a 2′-4′ bicyclic nucleoside (e.g., LNA, cEt, or ENA).

In some embodiments, a DMPK-targeting oligonucleotide described herein comprises at least 15 consecutive nucleosides (e.g., at least 15, at least 16, at least 17, at least 18, at least 19, or 20 consecutive nucleosides) of the nucleotide sequence of any one of SEQ ID NOs: 231-362, wherein each thymine base (T) may independently and optionally be replaced with a uracil base (U), and each U may independently and optionally be replaced with a T, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, X comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside), and/or (e.g., and) Z comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside).

In some embodiments, a DMPK-targeting oligonucleotide described herein comprises the nucleotide sequence of any one of SEQ ID NOs: 231-362, wherein each thymine base (T) may independently and optionally be replaced with a uracil base (U), and each U may independently and optionally be replaced with a T, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, each nucleoside in X is a 2′-modified nucleoside and/or (e.g., and) each nucleoside in Z is a 2′-modified nucleoside. In some embodiments, the 2′-modified nucleoside is a 2′-4′ bicyclic nucleoside (e.g., LNA, cEt, or ENA) or a non-bicyclic 2′-modified nucleoside (e.g., 2′-MOE modified nucleoside or 2′-O-Me modified nucleoside).

In some embodiments, a DMPK-targeting oligonucleotide described herein comprises the nucleotide sequence of any one of SEQ ID NOs: 231-362, wherein each thymine base (T) may independently and optionally be replaced with a uracil base (U), and each U may independently and optionally be replaced with a T, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, each nucleoside in X is a non-bicyclic 2′-modified nucleoside (e.g., 2′-MOE modified nucleoside) and/or (e.g., and) each nucleoside in Z is a non-bicyclic 2′-modified nucleoside (e.g., 2′-MOE modified nucleoside). In some embodiments, each nucleoside in X is a 2′-4′ bicyclic nucleoside (e.g., LNA, cEt, or ENA) and/or (e.g., and) each nucleoside in Z is a 2′-4′ bicyclic nucleoside (e.g., LNA, cEt, or ENA).

In some embodiments, a DMPK-targeting oligonucleotide described herein comprises the nucleotide sequence of any one of SEQ ID NOs: 231-362, wherein each thymine base (T) may independently and optionally be replaced with a uracil base (U), and each U may independently and optionally be replaced with a T, and comprises a 5′-X-Y-Z-3′ configuration, wherein at least one of the nucleosides in X is a 2′-modified nucleoside (e.g., 2′-MOE modified nucleoside, 2′-O-Me modified nucleoside, LNA, cEt, or ENA); wherein each cytosine in Y is optionally and independently a 5-methyl-cytosine; and wherein at least one of the nucleosides in Z is a 2′-modified nucleoside (e.g., 2′-MOE modified nucleoside, 2′-O-Me modified nucleoside, LNA, cEt, or ENA). In some embodiments, X comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside), and/or (e.g., and) Z comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside).

In some embodiments, the DMPK-targeting oligonucleotide comprises one or more phosphorothioate internucleoside linkages. In some embodiments, each internucleoside linkage in the DMPK-targeting oligonucleotide is a phosphorothioate internucleoside linkage. In some embodiments, the DMPK-targeting oligonucleotide comprises one or more phosphodiester internucleoside linkages, optionally wherein the phosphodiester internucleoside linkages are in X and/or Z. In some embodiments, the DMPK-targeting oligonucleotide comprises one or more phosphorothioate internucleoside linkages and one or more phosphodiester internucleoside linkages. In some embodiments, the DMPK-targeting oligonucleotide comprises 1 phosphodiester internucleoside linkage (PO), 2 PO, 3 PO, 4 PO, 5 PO, 6 PO, 7 PO, 8 PO, 9 PO, 10 PO, 11 PO, 12 PO, 13 PO, 14 PO, 15 PO, 16 PO, 17 PO, 18 PO, 19 PO, 20 PO, 21 PO, 22 PO, 23 PO, 24 PO, 25 PO, 26 PO, 27 PO, 28 PO, or 29 PO, and the remaining internucleoside linkages are phosphorothioate internucleoside linkages (PS). For example, a 20-nucleotide DMPK-targeting oligonucleotide may comprise 1 PO and 18 PS, 2 PO and 17 PS, 3 PO and 16 PS, 4 PO and 15 PS, 5 PO and 14 PS, 6 PO and 13 PS, 7 PO and 12 PS, 8 PO and 11 PS, 9 PO and 10 PS, 10 PO and 9 PS, 11 PO and 8 PS, 12 PO and 7 PS, 13 PO and 6 PS, 14 PO and 5 PS, 15 PO and 4 PS, 16 PO and 3 PS, 17 PO and 2 PS, or 18 PO and 1 PS. In some embodiments, each internucleoside linkage in the gap region Y is a phosphorothioate internucleoside linkage, X comprises one or more phosphorothioate internucleoside linkages and one or more phosphodiester internucleoside linkages, and Z comprises one or more phosphorothioate internucleoside linkages and one or more phosphodiester internucleoside linkages. In some embodiments, each internucleoside linkage in the gap region Y is a phosphorothioate internucleoside linkage, each internucleoside linkage in X is a phosphorothioate internucleoside linkage, and Z comprises one or more phosphorothioate internucleoside linkages and one or more phosphodiester internucleoside linkages. In some embodiments, each internucleoside linkage in the gap region Y is a phosphorothioate internucleoside linkage, X comprises one or more phosphorothioate internucleoside linkages and one or more phosphodiester internucleoside linkages, and each internucleoside linkage in Z is a phosphorothioate internucleoside linkage. For example, a DMPK-targeting oligonucleotide may comprise wing regions X and Z having mixed phosphodiester/phosphorothioate backbones and a gap region Y having a fully phosphorothioate backbone, or may comprise one wing region (i.e., X or Z) having a mixed phosphodiester/phosphorothioate backbone, the other wing region having a fully phosphorothioate backbone and a gap region Y having a fully phosphorothioate backbone. In some embodiments, gap region Y comprises one or more phosphorothioate internucleoside linkages and one or more phosphodiester internucleoside linkages and wing regions X and Y each independently either have a fully phosphorothioate backbone or comprise one or more phosphorothioate internucleoside linkages and one or more phosphodiester internucleoside linkages. For example, a DMPK-targeting oligonucleotide may comprise wing regions X and Z having mixed phosphodiester/phosphorothioate backbones and a gap region Y having a mixed phosphodiester/phosphorothioate backbone.

In some embodiments, an antisense oligonucleotide is provided of the formula:

(L)_{X1}(E)_{X2}(L)_{X3}(D)_{X4}(L)_{X5}(E)_{X6}(L)_{X7}:
wherein each (L) is a 2′-4′ bicyclic nucleoside,
wherein each (E) is a non-bicyclic 2′-modified nucleoside,
wherein each (D) is 2′-deoxyribonucleoside,
wherein X1 is independently an integer from 0 to 5 representing the number of instances of the corresponding L,
wherein X2 is independently an integer from 0 to 5 representing the number of instances of the corresponding E,
wherein X3 is independently an integer from 0 to 5 representing the number of instances of the corresponding L,
wherein X4 is independently an integer from 5 to 12 representing the number of instances of D,
wherein X5 is independently an integer from 0 to 5 representing the number of instances of the corresponding L,
wherein X6 is independently an integer from 0 to 5 representing the number of instances of the corresponding E,
wherein X7 is independently an integer from 0 to 5 representing the number of instances of the corresponding L, and
wherein at least one of X1, X2, and X3 is in the range of 1 to 5 and at least one of X5, X6, and X7 is in the range of 1 to 5.

In some embodiments, X1, X3, X5, and X7 are each 0 and X2 and X6 are independently 1, 2, 3, 4, or 5.

In some embodiments, X1, X2, X5, and X6 are each 0 and X3 and X7 are independently 1, 2, 3, 4, or 5.

In some embodiments, X3 and X5 are each 0 and X1, X2, X6 and X7 are independently 1, 2, 3, 4, or 5.

In some embodiments, X1 and X7 are each 0 and X2, X3, X5 and X6 are independently 1, 2, 3, 4, or 5.

In some embodiments, X4 is 5, 6, 7, 8, 9, or 10.

In some embodiments, the 2′-4′ bicyclic nucleoside is selected from LNA, cEt, and ENA nucleosides. In some embodiments, the non-bicyclic 2′-modified nucleoside is a 2′-MOE modified nucleoside or a 2′-OMe modified nucleoside.

In some embodiments, the nucleosides of the oligonucleotides are joined together by phosphorothioate internucleoside linkages, phosphodiester internucleoside linkages or a combination thereof. In some embodiments, the oligonucleotide comprises only phosphorothioate internucleoside linkages joining each nucleoside. In some embodiments, the oligonucleotide comprises at least one phosphorothioate internucleoside linkage. In some embodiments, the oligonucleotide comprises a mix of phosphorothioate and phosphodiester internucleoside linkages. In some embodiments, the oligonucleotide comprises only phosphorothioate internucleoside linkages joining each pair of 2′-deoxyribonucleosides and a mix of phosphorothioate and phosphodiester internucleoside linkages joining the remaining nucleosides.

In some embodiments, the oligonucleotide comprises a 5′-X-Y-Z-3′configuration of:

| X | Y | Z |
|---|---|---|
| EEEEE | (D)₁₀ | EEEEE, |
| EEF | (D)₁₀ | EEE, |
| EEEEE | (D)₁₀ | EEEE, |
| EEEEE | (D)₁₀ | EE, |
| LLL | (D)₁₀ | LLL, |
| EELL | (D)₈ | LLEE, |
| LLEE | (D)₈ | EELL, or |
| LLEEE | (D)₁₀ | EEELL, |

wherein “E” is a 2′-MOE modified ribonucleoside; “L” is LNA; “D” is 2′-deoxyribonucleoside; and “10” or “8” is the number of the 2′-deoxyribonucleoside in Y, and wherein the oligonucleotide comprises phosphorothioate internucleoside linkages, phosphodiester internucleoside linkages or a combination thereof.

In some embodiments, in any one of the DMPK-targeting oligonucleotide described herein, each cytidine (e.g., a 2′-modified cytidine) in X and/or Z is optionally and independently a 5-methyl-cytidine, and/or each uridine (e.g., a 2′-modified uridine) in X and/or Z is optionally and independently a 5-methyl-uridine.

In some embodiments, the DMPK-targeting oligonucleotide is selected from the ASOs listed in Table 8, Table 9, and Table 10. In some embodiments, the DMPK-targeting oligonucleotide is complementary to a target sequence listed in Table 8.

In some embodiments, the DMPK-targeting oligonucleotide is complementary to any one of SEQ ID NOs: 205, 211, 214, 217, 222, 215, 220, and 225. In some embodiments, the DMPK-targeting oligonucleotide is complementary to any one of SEQ ID NOs: 205, 214, 215, and 220. In some embodiments, the DMPK-targeting oligonucleotide is complementary to any one of SEQ ID NOs: 211, 217, 222, and 225. In some embodiments, the DMPK-targeting oligonucleotide is complementary to any one of SEQ ID NOs: 205, 214, 217, and 222. In some embodiments, the DMPK-targeting oligonucleotide is complementary to any one of SEQ ID NOs: 211, 215, 220, and 225.

In some embodiments, the DMPK-targeting oligonucleotide comprises a nucleobase sequence of any one of SEQ ID NOs: 276, 282, 285, 286, 288, 291, 293, 296, 345, 348, 350, 352, 354, and 357. In some embodiments, the DMPK-targeting oligonucleotide comprises a nucleobase sequence of any one of SEQ ID NOs: 276, 285, 286, 291, 348, and 352. In some embodiments, the DMPK-targeting oligonucleotide comprises a nucleobase sequence of any one of SEQ ID NOs: 282, 288, 293, 296, 345, 350, 354, and 357. In some embodiments, the DMPK-targeting oligonucleotide comprises a nucleobase sequence of any one of SEQ ID NOs: 276, 285, 288, 293, 348, 350, and 354. In some embodiments, the DMPK-targeting oligonucleotide comprises a nucleobase sequence of any one of SEQ ID NOs: 282, 286, 291, 296, 345, 352, and 357. In some embodiments, each thymine base (T) of the DMPK-targeting oligonucleotide may independently and optionally be replaced with a uracil base (U), and each U may independently and optionally be replaced with a T.

In some embodiments, the DMPK-targeting oligonucleotide comprises a structure selected from:

(SEQ ID NO: 276)
+G*x+C*oA*xoC*dG*dT*dG*dT*dG*dG*xdC*dT*xoC*oA*+A*+
G,
(SEQ ID NO: 345)
x+C*+A*oU*oG*dA*xdC*dA*dA*dT*xdC*dT*xdC*xoC*oG*x+C
*x+C,
(SEQ ID NO: 348)
+A*x+C*xoC*oA*dA*xdC*dA*xdC*dG*dT*xdC*xdC*xoC*oU*x
+C*+U,
(SEQ ID NO: 350)
+G*+U*oA*oG*dT*dT*dG*dA*xdC*dT*dG*dG*xoC*oG*+A*+A,
(SEQ ID NO: 354)
x+C*x+C*xoC*oG*dG*dA*dT*xdC*dA*xdC*dA*dG*oG*oA*x+C
*+U,
(SEQ ID NO: 286)
xoC*xoC*+A*+A*dA*xdC*dT*dT*dG*xdC*dT*xdC*+A*+G*xoC
*oA,
(SEQ ID NO: 352)
xoC*oU*+U*x+C*dA*dG*xdC*dT*dG*dT*dT*dT*x+C*+A*oU*x
oC,
and
(SEQ ID NO: 357)
x+C*+G*oU*xoC*dA*xdC*xdC*dT*xdC*dG*dG*xdC*xoC*oU*x
+C*+A,

wherein “+N” is an LNA nucleoside; “x+C” is 5-methyl LNA cytidine; “xdC” is 5-methyl-deoxycytidine; “dN” is 2′-deoxyribonucleoside; “oN” is 2′-MOE modified ribonucleoside; “xoC” is 5-methyl-2′-MOE-cytidine; “oU” is 5-methyl-2′-MOE-uridine; and “*” indicates a phosphorothioate (PS) internucleoside linkage.

In some embodiments, the DMPK-targeting oligonucleotide comprises a structure selected from:

(SEQ ID NO: 276)
+G*x+C*oA*xoC*dG*dT*dG*dT*dG*dG*xdC*dT*xoC*oA*+A*+
G,
(SEQ ID NO: 348)
+A*x+C*xoC*oA*dA*xdC*dA*xdC*dG*dT*xdC*xdC*xoC*oU*x
+C*+U,
(SEQ ID NO: 286)
xoC*xoC*+A*+A*dA*xdC*dT*dT*dG*xdC*dT*xdC*+A*+G*xoC
*oA,
and
(SEQ ID NO: 352)
xoC*oU*+U*x+C*dA*dG*xdC*dT*dG*dT*dT*dT*x+C*+A*oU*x
oC,

wherein “+N” is an LNA nucleoside; “x+C” is 5-methyl LNA cytidine; “xdC” is 5-methyl-deoxycytidine; “dN” is 2′-deoxyribonucleoside; “oN” is 2′-MOE modified ribonucleoside; “xoC” is 5-methyl-2′-MOE-cytidine; “oU” is 5-methyl-2′-MOE-uridine; and “*” indicates a phosphorothioate (PS) internucleoside linkage.

In some embodiments, the DMPK-targeting oligonucleotide comprises a structure selected from:

(SEQ ID NO: 345)
x+C*+A*oU*oG*dA*xdC*dA*dA*dT*xdC*dT*xdC*xoC*oG*x+C
*x+C,
(SEQ ID NO: 350)
+G*+U*A*oG*dT*dT*dG*dA*xdC*dT*dG*dG*xoC*oG*+A*+A,
(SEQ ID NO: 354)
x+C*x+C*xoC*oG*dG*dA*dT*xdC*dA*xdC*dA*dG*oG*oA*x+C
*+U,
and
(SEQ ID NO: 357)
x+C*+G*oU*xoC*dA*xdC*xdC*dT*xdC*dG*dG*xdC*xoC*oU*x
+C*+A,

wherein “+N” is an LNA nucleoside; “x+C” is 5-methyl LNA cytidine; “xdC” is 5-methyl-deoxycytidine; “dN” is 2′-deoxyribonucleoside; “oN” is 2′-MOE modified ribonucleoside; “xoC” is 5-methyl-2′-MOE-cytidine; “oU” is 5-methyl-2′-MOE-uridine; and “*” indicates a phosphorothioate (PS) internucleoside linkage.

In some embodiments, the DMPK-targeting oligonucleotide comprises a structure selected from:

(SEQ ID NO: 276)
+G*x+C*oA*xoC*dG*dT*dG*dT*dG*dG*xdC*dT*xoC*oA*+A*+
G,
(SEQ ID NO: 348)
+A*x+C*xoC*oA*dA*xdC*dA*xdC*dG*dT*xdC*xdC*xoC*oU*x
+C*+U,
(SEQ ID NO: 350)
+G*+U*oA*oG*dT*dT*dG*dA*xdC*dT*dG*dG*xoC*oG*+A*+A,
and
(SEQ ID NO: 354)
x+C*x+C*xoC*oG*dG*dA*dT*xdC*dA*xdC*dA*dG*oG*oA*x+C
*+U,

wherein “+N” is an LNA nucleoside; “x+C” is 5-methyl LNA cytidine; “xdC” is 5-methyl-deoxycytidine; “dN” is 2′-deoxyribonucleoside; “oN” is 2′-MOE modified ribonucleoside; “xoC” is 5-methyl-2′-MOE-cytidine; “oU” is 5-methyl-2′-MOE-uridine; and “*” indicates a phosphorothioate (PS) internucleoside linkage.

In some embodiments, the DMPK-targeting oligonucleotide comprises a structure selected from:

(SEQ ID NO: 345)
x+C*+A*oU*oG*dA*xdC*dA*dA*dT*xdC*dT*xdC*xoC*oG*x+C
*x+C,
(SEQ ID NO: 286)
xoC*xoC*+A*+A*dA*xdC*dT*dT*dG*xdC*dT*xdC*+A*+G*xoC
*oA,
(SEQ ID NO: 352)
xoC*oU*+U*x+C*dA*dG*xdC*dT*dG*dT*dT*dT*x+C*+A*oU*x
oC,
and
(SEQ ID NO: 357)
x+C*+G*oU*xoC*dA*xdC*xdC*dT*xdC*dG*dG*xdC*xoC*oU*x
+C*+A,

wherein “+N” is an LNA nucleoside; “x+C” is 5-methyl LNA cytidine; “xdC” is 5-methyl-deoxycytidine; “dN” is 2′-deoxyribonucleoside; “oN” is 2′-MOE modified ribonucleoside; “xoC” is 5-methyl-2′-MOE-cytidine; “oU” is 5-methyl-2′-MOE-uridine; and “*” indicates a phosphorothioate (PS) internucleoside linkage.

In some embodiments, any one of the DMPK-targeting oligonucleotides can be in salt form, e.g., as sodium, potassium, or magnesium salts.

In some embodiments, the 5′ or 3′ nucleoside (e.g., terminal nucleoside) of any one of the oligonucleotides described herein (e.g., the oligonucleotides listed in Table 8, Table 9, and Table 10) is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of any of the oligonucleotides described herein (e.g., the oligonucleotides listed in Table 8, Table 9, and Table 10) is conjugated to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of any one of the oligonucleotides described herein (e.g., the oligonucleotides listed in Table 8, Table 9, and Table 10) is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ— and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via the amine group.

### C. Linkers

Complexes described herein generally comprise a linker that covalently links any one of the anti-TfR1 antibodies described herein to a molecular payload. A linker comprises at least one covalent bond. In some embodiments, a linker may be a single bond, e.g., a disulfide bond or disulfide bridge, that covalently links an anti-TfR1 antibody to a molecular payload. However, in some embodiments, a linker may covalently link any one of the anti-TfR1 antibodies described herein to a molecular payload through multiple covalent bonds. In some embodiments, a linker may be a cleavable linker. However, in some embodiments, a linker may be a non-cleavable linker. A linker is typically stable in vitro and in vivo, and may be stable in certain cellular environments. Additionally, typically a linker does not negatively impact the functional properties of either the anti-TfR1 antibody or the molecular payload. Examples and methods of synthesis of linkers are known in the art (see, e.g. Kline, T. et al. “Methods to Make Homogenous Antibody Drug Conjugates.” Pharmaceutical Research, 2015, 32:11, 3480-3493.; Jain, N. et al. “Current ADC Linker Chemistry” Pharm Res. 2015, 32:11, 3526-3540.; McCombs, J. R. and Owen, S. C. “Antibody Drug Conjugates: Design and Selection of Linker, Payload and Conjugation Chemistry” AAPS J. 2015, 17:2, 339-351.).

A linker typically will contain two different reactive species that allow for attachment to both the anti-TfR1 antibody and a molecular payload. In some embodiments, the two different reactive species may be a nucleophile and/or an electrophile. In some embodiments, a linker contains two different electrophiles or nucleophiles that are specific for two different nucleophiles or electrophiles. In some embodiments, a linker is covalently linked to an anti-TfR1 antibody via conjugation to a lysine residue or a cysteine residue of the anti-TfR1 antibody. In some embodiments, a linker is covalently linked to a cysteine residue of an anti-TfR1 antibody via a maleimide-containing linker, wherein optionally the maleimide-containing linker comprises a maleimidocaproyl or maleimidomethyl cyclohexane-1-carboxylate group. In some embodiments, a linker is covalently linked to a cysteine residue of an anti-TfR1 antibody or thiol functionalized molecular payload via a 3-arylpropionitrile functional group. In some embodiments, a linker is covalently linked to a lysine residue of an anti-TfR1 antibody. In some embodiments, a linker is covalently linked to an anti-TfR1 antibody and/or (e.g., and) a molecular payload, independently, via an amide bond, a carbamate bond, a hydrazide, a triazole, a thioether, and/or a disulfide bond.

i. Cleavable Linkers

A cleavable linker may be a protease-sensitive linker, a pH-sensitive linker, or a glutathione-sensitive linker. These linkers are typically cleavable only intracellularly and are preferably stable in extracellular environments, e.g., extracellular to a muscle cell or a CNS cell.

Protease-sensitive linkers are cleavable by protease enzymatic activity. These linkers typically comprise peptide sequences and may be 2-10 amino acids, about 2-5 amino acids, about 5-10 amino acids, about 10 amino acids, about 5 amino acids, about 3 amino acids, or about 2 amino acids in length. In some embodiments, a peptide sequence may comprise naturally-occurring amino acids, e.g. cysteine, alanine, or non-naturally-occurring or modified amino acids. Non-naturally occurring amino acids include j-amino acids, homo-amino acids, proline derivatives, 3-substituted alanine derivatives, linear core amino acids, N-methyl amino acids, and others known in the art. In some embodiments, a protease-sensitive linker comprises a valine-citrulline or alanine-citrulline sequence. In some embodiments, a protease-sensitive linker can be cleaved by a lysosomal protease, e.g. cathepsin B, and/or (e.g., and) an endosomal protease.

A pH-sensitive linker is a covalent linkage that readily degrades in high or low pH environments. In some embodiments, a pH-sensitive linker may be cleaved at a pH in a range of 4 to 6. In some embodiments, a pH-sensitive linker comprises a hydrazone or cyclic acetal. In some embodiments, a pH-sensitive linker is cleaved within an endosome or a lysosome.

In some embodiments, a glutathione-sensitive linker comprises a disulfide moiety. In some embodiments, a glutathione-sensitive linker is cleaved by a disulfide exchange reaction with a glutathione species inside a cell. In some embodiments, the disulfide moiety further comprises at least one amino acid, e.g., a cysteine residue.

In some embodiments, a linker comprises a valine-citrulline sequence (e.g., as described in U.S. Pat. No. 6,214,345, incorporated herein by reference). In some embodiments, before conjugation, a linker comprises a structure of:

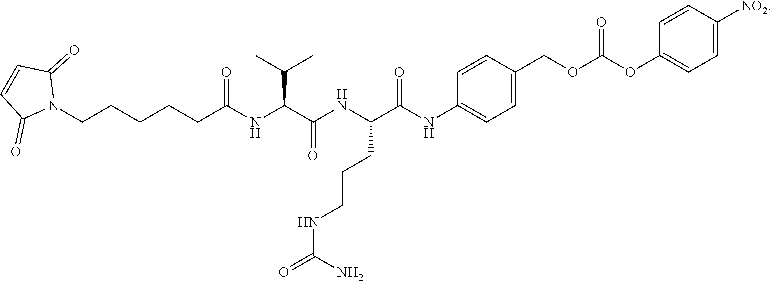 [SMILES: CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1]

In some embodiments, after conjugation, a linker comprises a structure of:

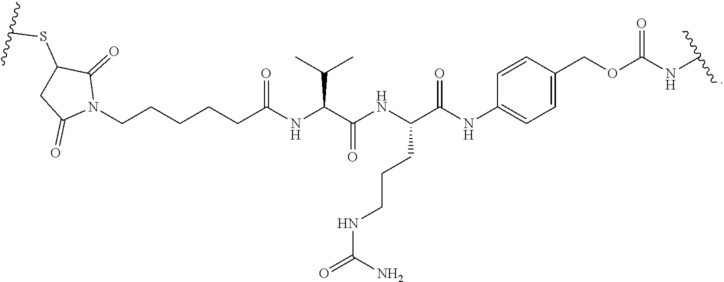 [SMILES: *NC(=O)OCc1ccc(NC(=O)[C@H](CCCNC(N)=O)NC(=O)[C@@H](NC(=O)CCCCCN2C(=O)CC(S*)C2=O)C(C)C)cc1]

In some embodiments, before conjugation, a linker comprises a structure of:

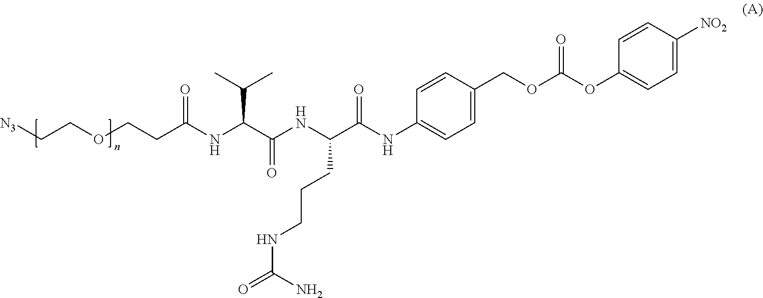

wherein n is any number from 0-10. In some embodiments, n is 3.

In some embodiments, a linker comprises a structure of:

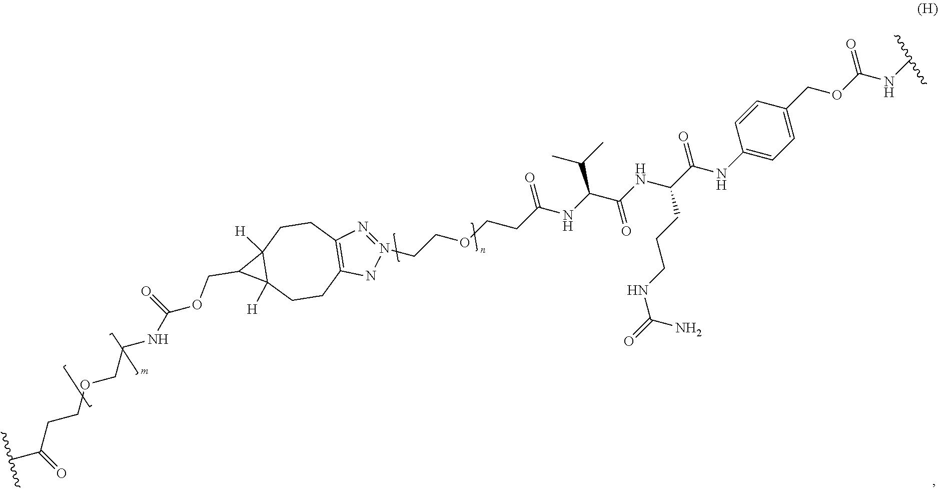

wherein n is any number from 0-10, wherein m is any number from 0-10. In some embodiments, n is 3 and/or (e.g., and) m is 4.

In some embodiments, a linker comprises a structure of:

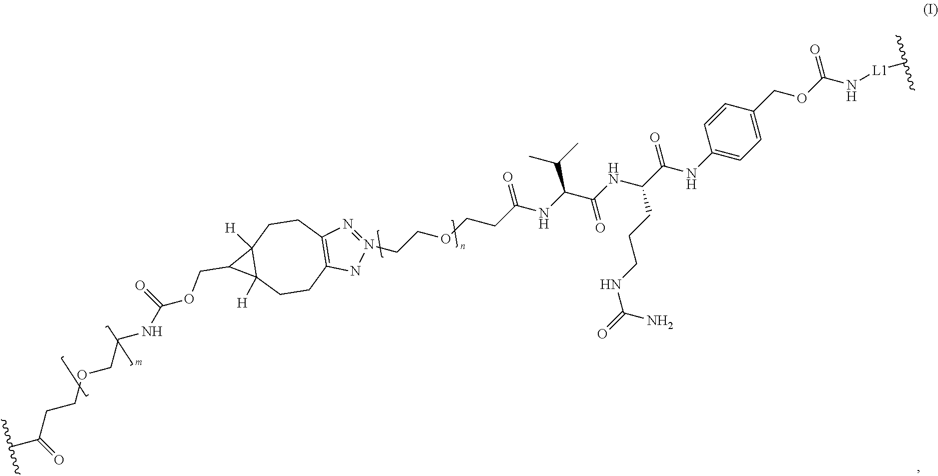

wherein n is any number from 0-10, wherein m is any number from 0-10. In some embodiments, n is 3 and/or (e.g., and) m is 4.
ii. Non-Cleavable Linkers

In some embodiments, non-cleavable linkers may be used. Generally, a non-cleavable linker cannot be readily degraded in a cellular or physiological environment. In some embodiments, a non-cleavable linker comprises an optionally substituted alkyl group, wherein the substitutions may include halogens, hydroxyl groups, oxygen species, and other common substitutions. In some embodiments, a linker may comprise an optionally substituted alkyl, an optionally substituted alkylene, an optionally substituted arylene, a heteroarylene, a peptide sequence comprising at least one non-natural amino acid, a truncated glycan, a sugar or sugars that cannot be enzymatically degraded, an azide, an alkyne-azide, a peptide sequence comprising a LPXT sequence, a thioether, a biotin, a biphenyl, repeating units of polyethylene glycol or equivalent compounds, acid esters, acid amides, sulfamides, and/or an alkoxy-amine linker. In some embodiments, sortase-mediated ligation can be utilized to covalently link an anti-TfR1 antibody comprising a LPXT sequence to a molecular payload comprising a (G)ₙ sequence (see, e.g. Proft T. Sortase-mediated protein ligation: an emerging biotechnology tool for protein modification and immobilization. Biotechnol Lett. 2010, 32(1):1-10.).

In some embodiments, a linker may comprise a substituted alkylene, an optionally substituted alkenylene, an optionally substituted alkynylene, an optionally substituted cycloalkylene, an optionally substituted cycloalkenylene, an optionally substituted arylene, an optionally substituted heteroarylene further comprising at least one heteroatom selected from N, O, and S; an optionally substituted heterocyclylene further comprising at least one heteroatom selected from N, O, and S, an imino, an optionally substituted nitrogen species, an optionally substituted oxygen species 0, an optionally substituted sulfur species, or a poly(alkylene oxide), e.g. polyethylene oxide or polypropylene oxide. In some embodiments, a linker may be a non-cleavable N-gamma-maleimidobutyryl-oxysuccinimide ester (GMBS) linker.

iii. Linker Conjugation

In some embodiments, a linker is covalently linked to an anti-TfR1 antibody and/or (e.g., and) molecular payload via a phosphate, thioether, ether, carbon-carbon, carbamate, or amide bond. In some embodiments, a linker is covalently linked to an oligonucleotide through a phosphate or phosphorothioate group, e.g. a terminal phosphate of an oligonucleotide backbone. In some embodiments, a linker is covalently linked to an anti-TfR1 antibody, through a lysine or cysteine residue present on the anti-TfR1 antibody.

In some embodiments, a linker, or a portion thereof is covalently linked to an anti-TfR1 antibody and/or (e.g., and) molecular payload by a cycloaddition reaction between an azide and an alkyne to form a triazole, wherein the azide or the alkyne may be located on the anti-TfR1 antibody, molecular payload, or the linker. In some embodiments, an alkyne may be a cyclic alkyne, e.g., a cyclooctyne. In some embodiments, an alkyne may be bicyclononyne (also known as bicyclo[6.1.0]nonyne or BCN) or substituted bicyclononyne. In some embodiments, a cyclooctyne is as described in International Patent Application Publication WO2011136645, published on Nov. 3, 2011, entitled, “Fused Cyclooctyne Compounds And Their Use In Metal-free Click Reactions”. In some embodiments, an azide may be a sugar or carbohydrate molecule that comprises an azide. In some embodiments, an azide may be 6-azido-6-deoxygalactose or 6-azido-N-acetylgalactosamine. In some embodiments, a sugar or carbohydrate molecule that comprises an azide is as described in International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”. In some embodiments, a cycloaddition reaction between an azide and an alkyne to form a triazole, wherein the azide or the alkyne may be located on the anti-TfR1 antibody, molecular payload, or the linker is as described in International Patent Application Publication WO2014065661, published on May 1, 2014, entitled, “Modified antibody, antibody-conjugate and process for the preparation thereof”; or International Patent Application Publication WO2016170186, published on Oct. 27, 2016, entitled, “Process For The Modification Of A Glycoprotein Using A Glycosyltransferase That Is Or Is Derived From A β(1,4)-N-Acetylgalactosaminyltransferase”.

In some embodiments, a linker comprises a spacer, e.g., a polyethylene glycol spacer or an acyl/carbomoyl sulfamide spacer, e.g., a HydraSpace™ spacer. In some embodiments, a spacer is as described in Verkade, J. M. M. et al., “A Polar Sulfamide Spacer Significantly Enhances the Manufacturability, Stability, and Therapeutic Index of Antibody-Drug Conjugates”, Antibodies, 2018, 7, 12.

In some embodiments, a linker is covalently linked to an anti-TfR1 antibody and/or (e.g., and) molecular payload by the Diels-Alder reaction between a dienophile and a diene/hetero-diene, wherein the dienophile or the diene/hetero-diene may be located on the anti-TfR1 antibody, molecular payload, or the linker. In some embodiments a linker is covalently linked to an anti-TfR1 antibody and/or (e.g., and) molecular payload by other pericyclic reactions such as an ene reaction. In some embodiments, a linker is covalently linked to an anti-TfR1 antibody and/or (e.g., and) molecular payload by an amide, thioamide, or sulfonamide bond reaction. In some embodiments, a linker is covalently linked to an anti-TfR1 antibody and/or (e.g., and) molecular payload by a condensation reaction to form an oxime, hydrazone, or semicarbazide group existing between the linker and the anti-TfR1 antibody and/or (e.g., and) molecular payload.

In some embodiments, a linker is covalently linked to an anti-TfR1 antibody and/or (e.g., and) molecular payload by a conjugate addition reactions between a nucleophile, e.g. an amine or a hydroxyl group, and an electrophile, e.g. a carboxylic acid, carbonate, or an aldehyde. In some embodiments, a nucleophile may exist on a linker and an electrophile may exist on an anti-TfR1 antibody or molecular payload prior to a reaction between a linker and an anti-TfR1 antibody or molecular payload. In some embodiments, an electrophile may exist on a linker and a nucleophile may exist on an anti-TfR1 antibody or molecular payload prior to a reaction between a linker and an anti-TfR1 antibody or molecular payload. In some embodiments, an electrophile may be an azide, pentafluorophenyl, a silicon centers, a carbonyl, a carboxylic acid, an anhydride, an isocyanate, a thioisocyanate, a succinimidyl ester, a sulfosuccinimidyl ester, a maleimide, an alkyl halide, an alkyl pseudohalide, an epoxide, an episulfide, an aziridine, an aryl, an activated phosphorus center, and/or an activated sulfur center. In some embodiments, a nucleophile may be an optionally substituted alkene, an optionally substituted alkyne, an optionally substituted aryl, an optionally substituted heterocyclyl, a hydroxyl group, an amino group, an alkylamino group, an anilido group, and/or a thiol group.

In some embodiments, a linker comprises a valine-citrulline sequence covalently linked to a reactive chemical moiety (e.g., an azide moiety or a BCN moiety for click chemistry). In some embodiments, a linker comprising a valine-citrulline sequence covalently linked to a reactive chemical moiety (e.g., an azide moiety for click chemistry) comprises a structure of:

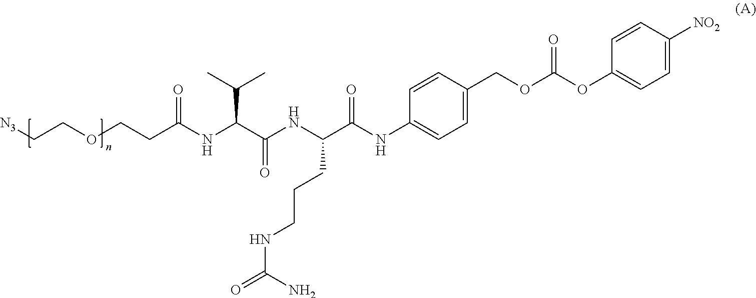

wherein n is any number from 0-10. In some embodiments, n is 3.

In some embodiments, a linker comprising the structure of Formula (A) is covalently linked (e.g., optionally via additional chemical moieties) to a molecular payload (e.g., an oligonucleotide). In some embodiments, a linker comprising the structure of Formula (A) is covalently linked to an oligonucleotide, e.g., through a nucleophilic substitution with amine-L1-oligonucleotides forming a carbamate bond, yielding a compound comprising a structure of:

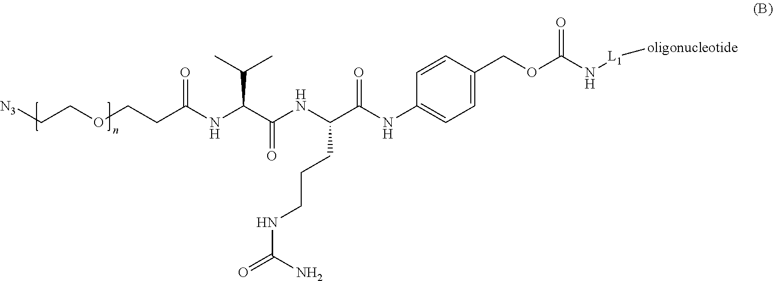

wherein n is any number from 0-10. In some embodiments, n is 3.

In some embodiments, the compound of Formula (B) is further covalently linked via a triazole to additional moieties, wherein the triazole is formed by a click reaction between the azide of Formula (A) or Formula (B) and an alkyne provided on a bicyclononyne. In some embodiments, a compound comprising a bicyclononyne comprises a structure of:

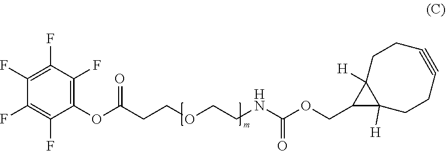

wherein m is any number from 0-10. In some embodiments, m is 4.

In some embodiments, the azide of the compound of structure (B) forms a triazole via a click reaction with the alkyne of the compound of structure (C), forming a compound comprising a structure of:

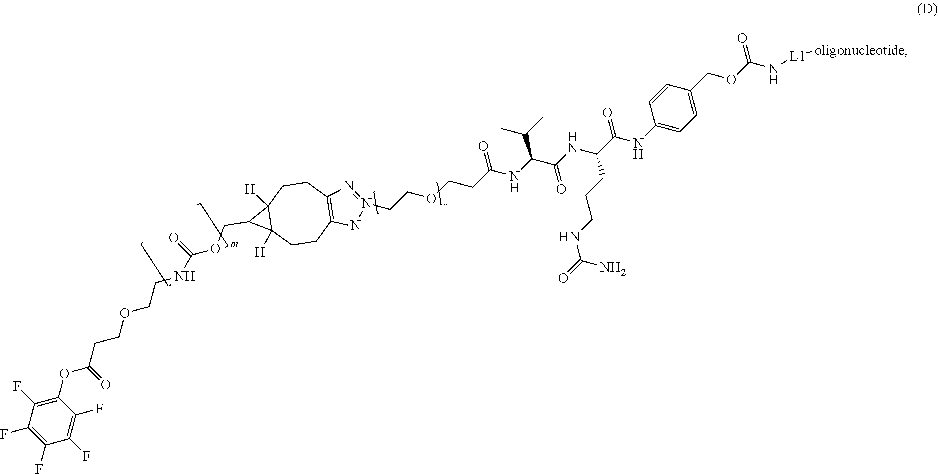

wherein n is any number from 0-10, and wherein m is any number from 0-10. In some embodiments, n is 3 and m is 4.

In some embodiments, the compound of structure (D) is further covalently linked to a lysine of the anti-TfR1 antibody, forming a complex comprising a structure of:

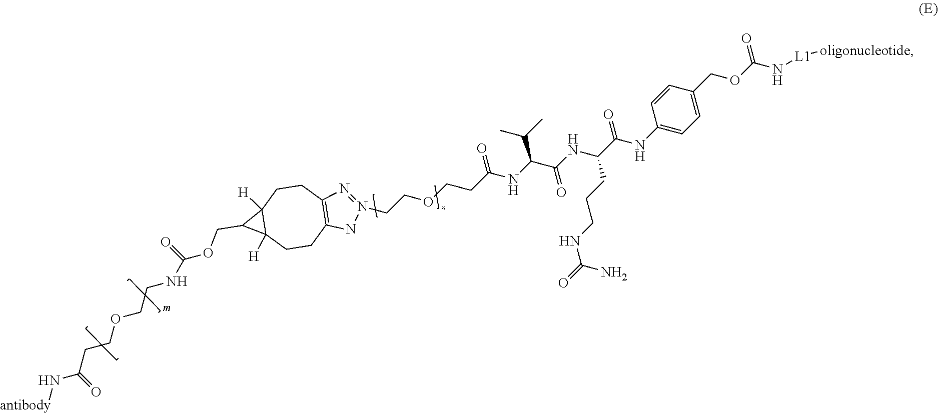

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the compound of Formula (C) is further covalently linked to a lysine of the anti-TfR1 antibody, forming a compound comprising a structure of:

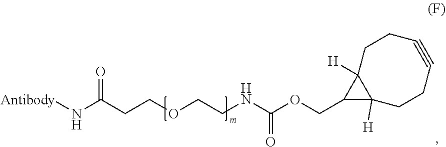

wherein m is 0-15 (e.g., 4). It should be understood that the amide shown adjacent the anti-TfR1 antibody in Formula (F) results from a reaction with an amine of the anti-TfR1 antibody, such as a lysine epsilon amine.

In some embodiments, the azide of the compound of structure (B) forms a triazole via a click reaction with the alkyne of the compound of structure (F), forming a complex comprising a structure of:

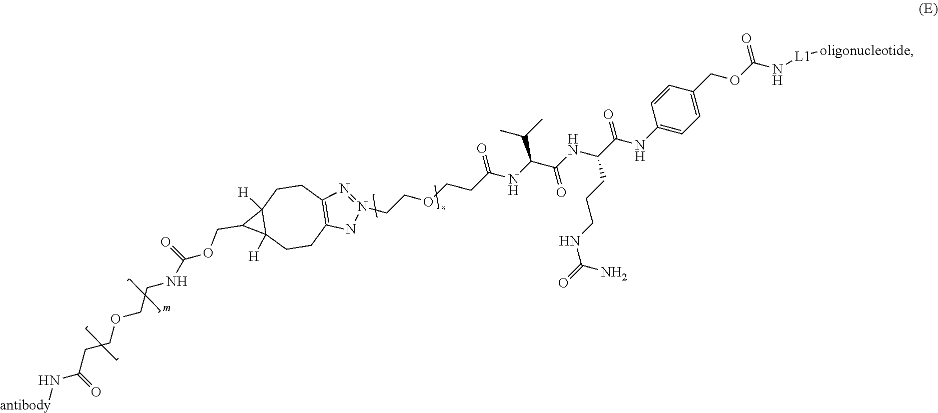

wherein n is any number from 0-10, wherein m is any number from 0-10. In some embodiments, n is 3 and/or (e.g., and) m is 4. It should be understood that the amide shown adjacent the anti-TfR1 antibody in Formula (E) results from a reaction with an amine of the anti-TfR1 antibody, such as a lysine epsilon amine.

In some embodiments, the azide of the compound of structure (A) forms a triazole via a click reaction with the alkyne of the compound of structure (F), forming a compound comprising a structure of:

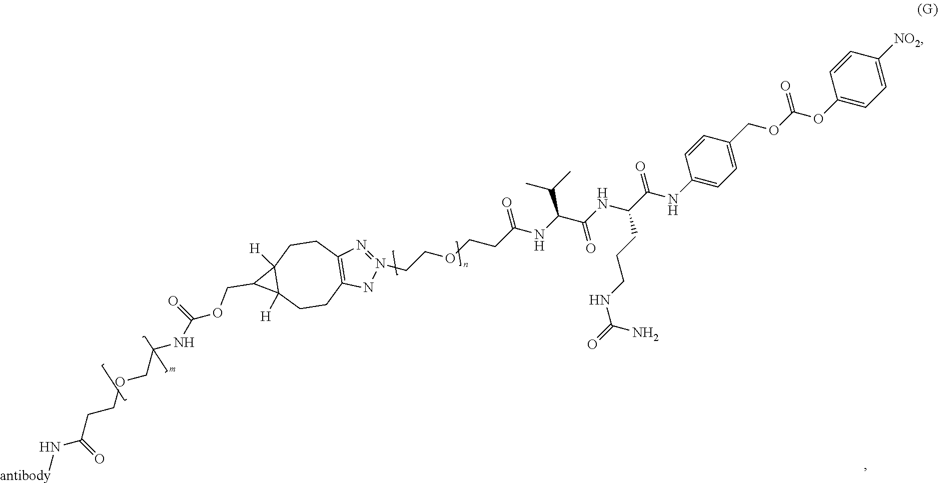

wherein n is any number from 0-10, wherein m is any number from 0-10. In some embodiments, n is 3 and/or (e.g., and) m is 4. In some embodiments, an oligonucleotide is covalently linked to a compound comprising a structure of formula (G), thereby forming a complex comprising a structure of formula (E). It should be understood that the amide shown adjacent the anti-TfR1 antibody in Formula (G) results from a reaction with an amine of the anti-TfR1 antibody, such as a lysine epsilon amine.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

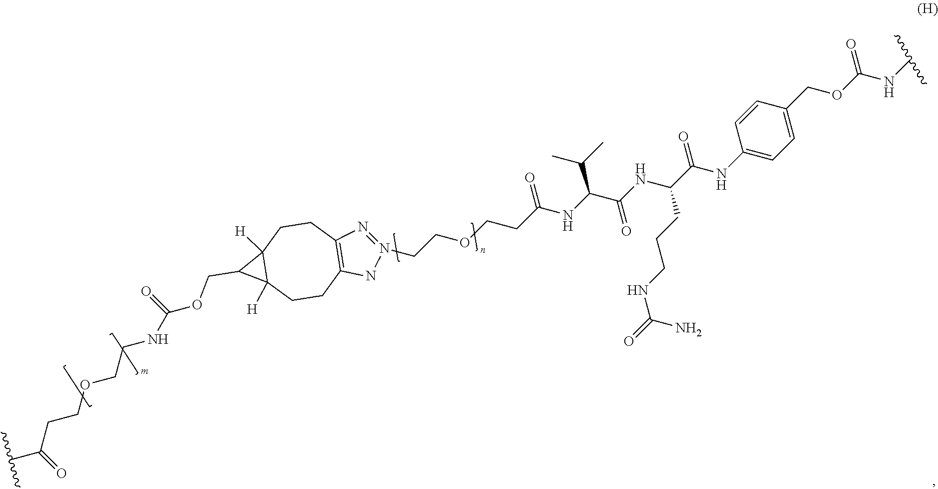

wherein n is any number from 0-10, wherein m is any number from 0-10. In some embodiments, n is 3 and/or (e.g., and) m is 4.

In some embodiments, in any one of the complexes described herein, the anti-TfR1 antibody is covalently linked via a lysine of the anti-TfR1 antibody to a molecular payload (e.g., an oligonucleotide) via a linker comprising a structure of:

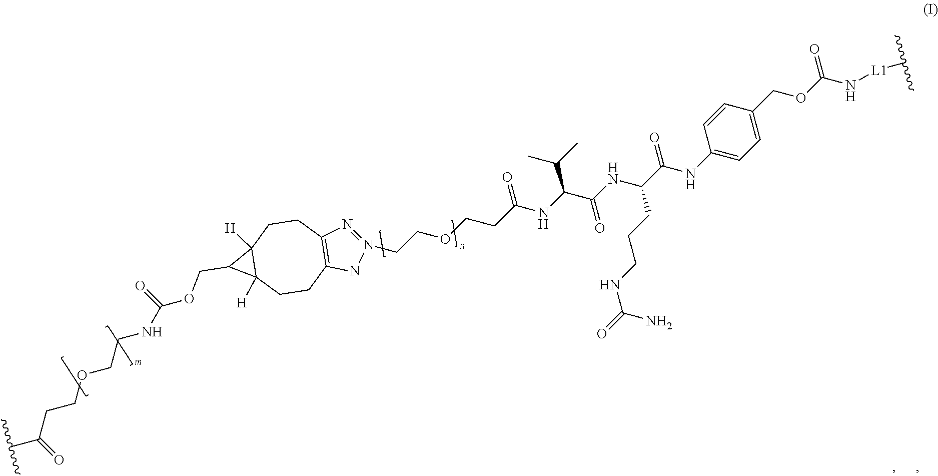

wherein n is any number from 0-10, wherein m is any number from 0-10. In some embodiments, n is 3 and/or (e.g., and) m is 4.

In some embodiments, in formulae (B), (D), (E), and (I), L1 is a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof, wherein each R^{A} is independently hydrogen or substituted or unsubstituted alkyl. In some embodiments, L1 is

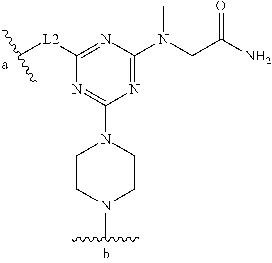

wherein L2 is

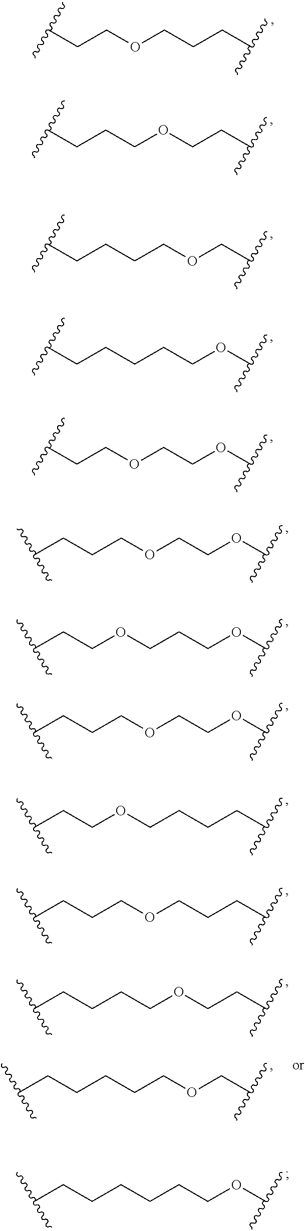

wherein a labels the site directly linked to the carbamate moiety of formulae (B), (D), (E), and (I); and b labels the site covalently linked (directly or via additional chemical moieties) to the oligonucleotide.

In some embodiments, L1 is:

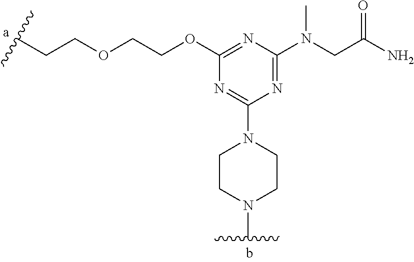 [SMILES: *CCOCCOc1nc(N(C)CC(N)=O)nc(N2CCN(*)CC2)n1]

wherein a labels the site directly linked to the carbamate moiety of formulae (B), (D), (E), and (I); and b labels the site covalently linked (directly or via additional chemical moieties) to the oligonucleotide.

In some embodiments, L1 is

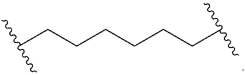 [SMILES: *CCCCCC*]

In some embodiments, L1 is linked to a 5′ phosphate of the oligonucleotide. In some embodiments, L1 is linked to a 5′ phosphate of the oligonucleotide. In some embodiments, the linkage of L1 to a 5′ phosphate of the oligonucleotide forms a phosphodiester bond between L1 and the oligonucleotide.

In some embodiments, L1 is optional (e.g., need not be present).

In some embodiments, any one of the complexes described herein has a structure of:

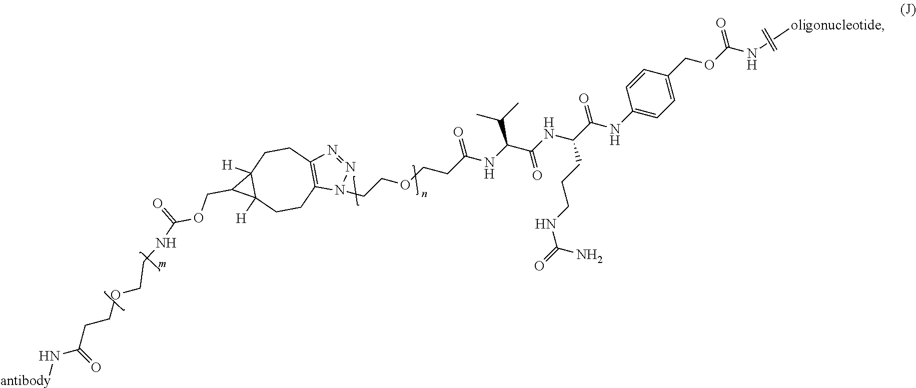

wherein n is 0-15 (e.g., 3) and m is 0-15 (e.g., 4). It should be understood that the amide shown adjacent the anti-TfR1 antibody in Formula (J) results from a reaction with an amine of the anti-TfR1 antibody, such as a lysine epsilon amine.

In some embodiments, any one of the complexes described herein has a structure of:

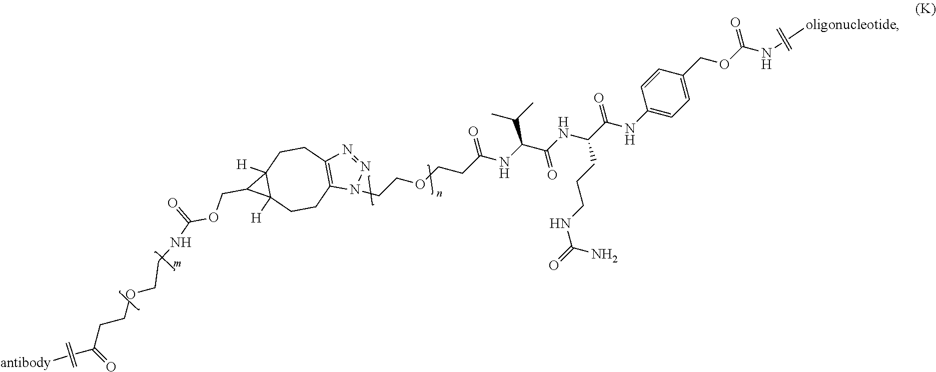

wherein n is 0-15 (e.g., 3) and m is 0-15 (e.g., 4).

In some embodiments, the oligonucleotide is modified to comprise an amine group at the 5′ end, the 3′ end, or internally (e.g., as an amine functionalized nucleobase), prior to linking to a compound, e.g., a compound of formula (A) or formula (G).

Although linker conjugation is described in the context of anti-TfR1 antibodies and oligonucleotide molecular payloads, it should be understood that use of such linker conjugation on other muscle-targeting agents, such as other muscle-targeting antibodies, and/or on other molecular payloads is contemplated.

### D. Examples of Antibody-Molecular Payload Complexes

Further provided herein are non-limiting examples of complexes comprising any one the anti-TfR1 antibodies described herein covalently linked to any of the molecular payloads (e.g., an oligonucleotide) described herein. In some embodiments, the anti-TfR1 antibody (e.g., any one of the anti-TfR1 antibodies provided in Tables 2-7) is covalently linked to a molecular payload (e.g., an oligonucleotide such as the oligonucleotides provided in Table 8, Table 9, and Table 10) via a linker. Any of the linkers described herein may be used. In some embodiments, if the molecular payload is an oligonucleotide, the linker is linked to the 5′ end of the oligonucleotide, the 3′ end of the oligonucleotide, or to an internal site of the oligonucleotide. In some embodiments, the linker is linked to the anti-TfR1 antibody via a thiol-reactive linkage (e.g., via a cysteine in the anti-TfR1 antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMPK-targeting oligonucleotide (e.g., a DMPK-targeting oligonucleotide listed in Table 8, Table 9, or Table 10).

An example of a structure of a complex comprising an anti-TfR1 antibody covalently linked to a molecular payload via a linker is provided below:

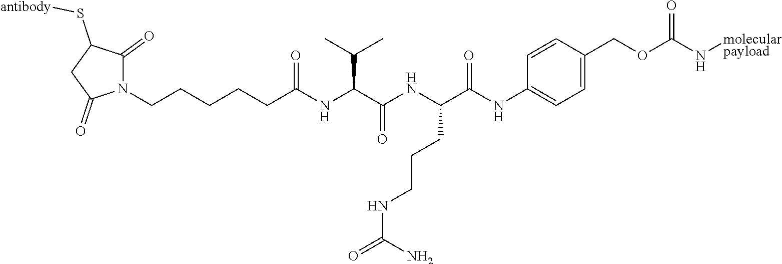

wherein the linker is linked to the antibody via a thiol-reactive linkage (e.g., via a cysteine in the antibody). In some embodiments, the molecular payload is a DMPK-targeting oligonucleotide (e.g., a DMPK-targeting oligonucleotide listed in Table 8, Table 9, or Table 10).

Another example of a structure of a complex comprising an anti-TfR1 antibody covalently linked to a molecular payload via a linker is provided below:

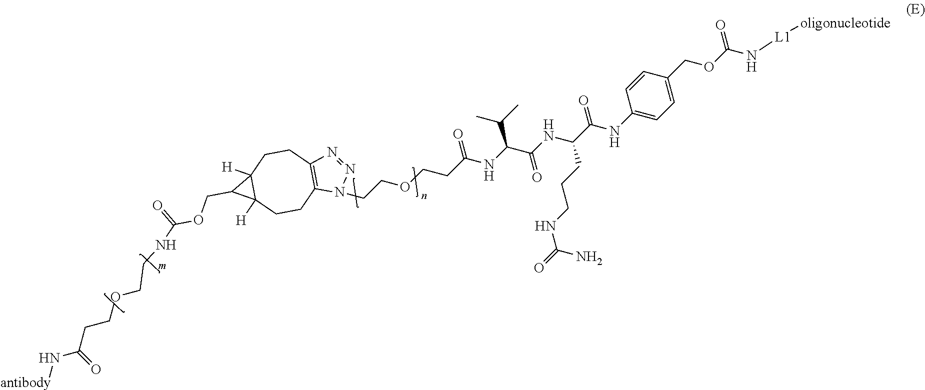

wherein n is a number between 0-10, wherein m is a number between 0-10, wherein the linker is linked to the antibody via an amine group (e.g., on a lysine residue), and/or (e.g., and) wherein the linker is linked to the oligonucleotide (e.g., at the 5′ end, 3′ end, or internally). In some embodiments, the linker is linked to the antibody via a lysine, the linker is linked to the oligonucleotide at the 5′ end, n is 3, and m is 4. In some embodiments, the molecular payload is a DMPK-targeting oligonucleotide (e.g., a DMPK-targeting oligonucleotide listed in Table 8, Table 9, or Table 10).

In some embodiments, L1 is

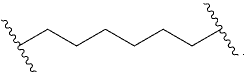 [SMILES: *CCCCCC*]

It should be understood that the amide shown adjacent the anti-TfR1 antibody in Formula (E) results from a reaction with an amine of the anti-TfR1 antibody, such as a lysine epsilon amine.

It should be appreciated that antibodies can be linked to molecular payloads with different stoichiometries, a property that may be referred to as a drug to antibody ratios (DAR) with the “drug” being the molecular payload. In some embodiments, one molecular payload is linked to an antibody (DAR=1). In some embodiments, two molecular payloads are linked to an antibody (DAR=2). In some embodiments, three molecular payloads are linked to an antibody (DAR=3). In some embodiments, four molecular payloads are linked to an antibody (DAR=4). In some embodiments, a mixture of different complexes, each having a different DAR, is provided. In some embodiments, an average DAR of complexes in such a mixture may be in a range of 1 to 3, 1 to 4, 1 to 5 or more. DAR may be increased by conjugating molecular payloads to different sites on an antibody and/or (e.g., and) by conjugating multimers to one or more sites on antibody. For example, a DAR of 2 may be achieved by conjugating a single molecular payload to two different sites on an antibody or by conjugating a dimer molecular payload to a single site of an antibody.

In some embodiments, the complex described herein comprises an anti-TfR1 antibody described herein (e.g., the antibodies provided in Tables 2-7) covalently linked to a molecular payload. In some embodiments, the complex described herein comprises an anti-TfR1 antibody described herein (e.g., the antibodies provided in Tables 2-7) covalently linked to molecular payload via a linker (e.g., a linker comprising a valine-citrulline sequence). In some embodiments, the linker (e.g., a linker comprising a valine-citrulline sequence) is linked to the antibody (e.g., an anti-TfR1 antibody described herein) via a thiol-reactive linkage (e.g., via a cysteine in the antibody). In some embodiments, the linker (e.g., a linker comprising a valine-citrulline sequence) is linked to the antibody (e.g., an anti-TfR1 antibody described herein) via an amine group (e.g., via a lysine in the antibody). In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a CDR-H1, a CDR-H2, a CDR-H3, a CDR-L1, a CDR-L2, and a CDR-L3 of any one of the antibodies listed in Table 2. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a VH comprising the amino acid sequence of SEQ ID NO: 69, SEQ ID NO: 71, or SEQ ID NO: 72, and a VL comprising the amino acid sequence of SEQ ID NO: 70. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 74. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a VH comprising the amino acid sequence of SEQ ID NO: 73 or SEQ ID NO: 76, and a VL comprising the amino acid sequence of SEQ ID NO: 75. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a VH comprising the amino acid sequence of SEQ ID NO: 77, and a VL comprising the amino acid sequence of SEQ ID NO: 78. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a VH comprising the amino acid sequence of SEQ ID NO: 77 or SEQ ID NO: 79, and a VL comprising the amino acid sequence of SEQ ID NO: 80. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a VH comprising the amino acid sequence of SEQ ID NO: 154, and a VL comprising the amino acid sequence of SEQ ID NO: 155. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 84, SEQ ID NO: 86 or SEQ ID NO: 87 and a light chain comprising the amino acid sequence of SEQ ID NO: 85. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 89. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 88 or SEQ ID NO: 91, and a light chain comprising the amino acid sequence of SEQ ID NO: 90. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92 or SEQ ID NO: 94, and a light chain comprising the amino acid sequence of SEQ ID NO: 95. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 92, and a light chain comprising the amino acid sequence of SEQ ID NO: 93. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 156, and a light chain comprising the amino acid sequence of SEQ ID NO: 157. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 97, SEQ ID NO: 98, or SEQ ID NO: 99 and a light chain comprising the amino acid sequence of SEQ ID NO: 85. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 89. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 100 or SEQ ID NO: 101 and a light chain comprising the amino acid sequence of SEQ ID NO: 90. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 and a light chain comprising the amino acid sequence of SEQ ID NO: 93. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 102 or SEQ ID NO: 103 and a light chain comprising the amino acid sequence of SEQ ID NO: 95. In some embodiments, the molecular payload is a DMPK-targeting oligonucleotide (e.g., a DMPK-targeting oligonucleotide listed in Table 8, Table 9, or Table 10).

In some embodiments, the complex described herein comprises an anti-TfR1 antibody covalently linked to a molecular payload, wherein the anti-TfR1 antibody comprises a heavy chain comprising the amino acid sequence of SEQ ID NO: 158 or SEQ ID NO: 159 and a light chain comprising the amino acid sequence of SEQ ID NO: 157. In some embodiments, the molecular payload is a DMPK-targeting oligonucleotide (e.g., a DMPK-targeting oligonucleotide listed in Table 8, Table 9, or Table 10).

In any of the example complexes described herein, in some embodiments, the anti-TfR1 antibody is covalently linked to the molecular payload via a linker comprising a structure of:

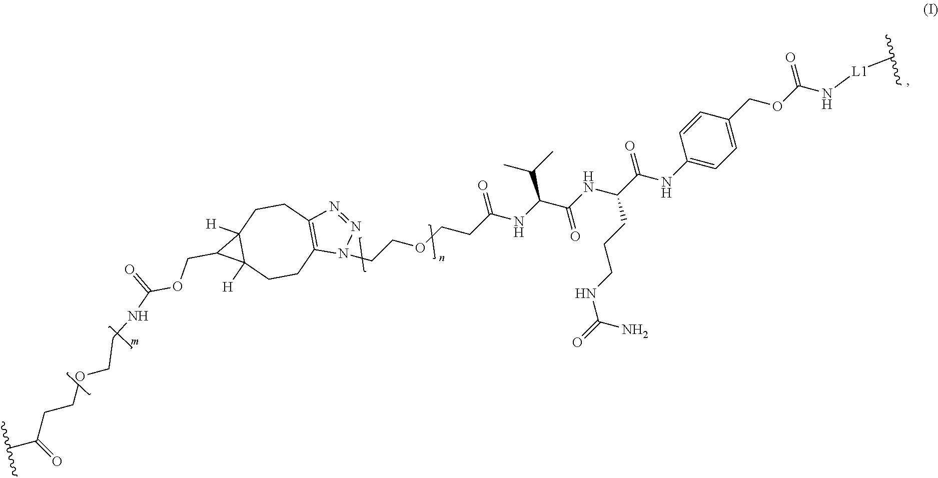

wherein n is 3, m is 4, and L1 is

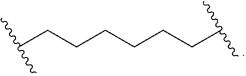 [SMILES: *CCCCCC*]

In some embodiments, the complex described herein comprises an anti-TfR1 antibody covalently linked to the 5′ end of a DMPK-targeting oligonucleotide (e.g., a DMPK-targeting oligonucleotide listed in Table 8, Table 9, or Table 10) via a lysine in the anti-TfR1 antibody, wherein the anti-TfR1 antibody comprises a CDR-H1, a CDR-H2, a CDR-H3, a CDR-L1, a CDR-L2, and a CDR-L3 of any one of the antibodies listed in Table 2, wherein the complex has a structure of:

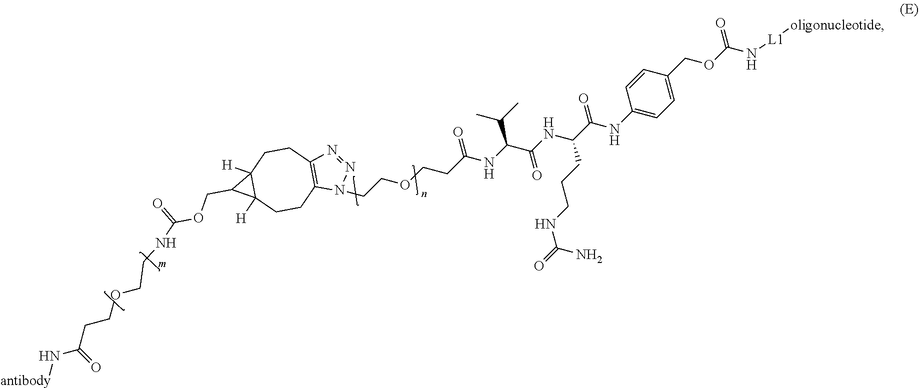

wherein n is 3 and m is 4, and wherein L1 is

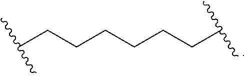 [SMILES: *CCCCCC*]

It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMPK-targeting oligonucleotide (e.g., a DMPK-targeting oligonucleotide listed in Table 8, Table 9, or Table 10) via a lysine in the anti-TfR1 antibody, wherein the anti-TfR1 antibody comprises a VH and VL of any one of the antibodies listed in Table 3, wherein the complex has a structure of:

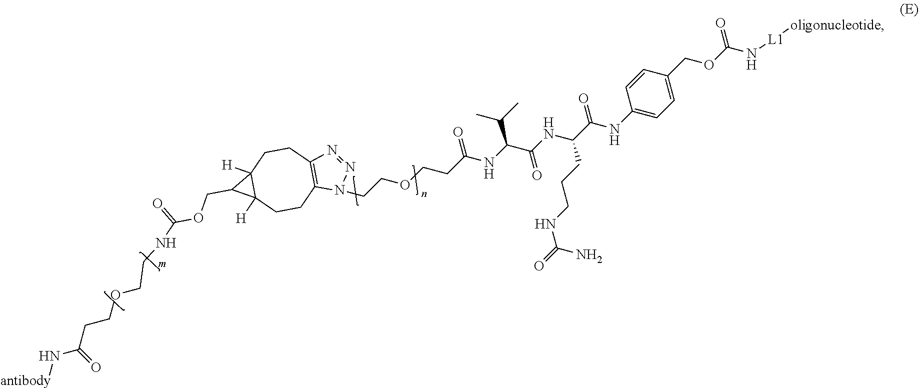

wherein n is 3 and m is 4, and wherein L1 is

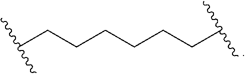 [SMILES: *CCCCCC*]

It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 antibody covalently linked to the 5′ end of a DMPK-targeting oligonucleotide (e.g., a DMPK-targeting oligonucleotide listed in Table 8, Table 9, or Table 10) via a lysine in the anti-TfR1 antibody, wherein the anti-TfR1 antibody comprises a heavy chain and light chain of any one of the antibodies listed in Table 4, wherein the complex has a structure of:

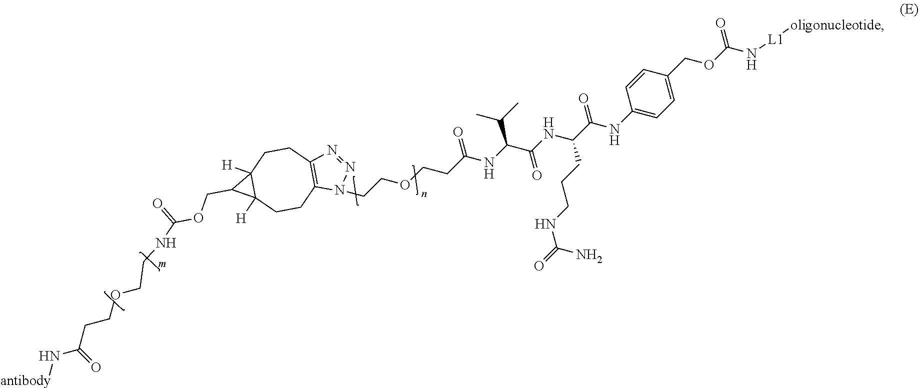

wherein n is 3 and m is 4, and wherein L1 is

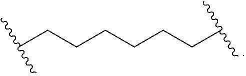 [SMILES: *CCCCCC*]

It should be understood that the amide shown adjacent the anti-TfR1 antibody in Formula (E) results from a reaction with an amine of the anti-TfR1 antibody, such as a lysine epsilon amine.

In some embodiments, the complex described herein comprises an anti-TfR1 Fab covalently linked to the 5′ end of a DMPK-targeting oligonucleotide (e.g., a DMPK-targeting oligonucleotide listed in Table 8, Table 9, or Table 10) via a lysine in the anti-TfR1 antibody, wherein the anti-TfR1 Fab comprises a heavy chain and light chain of any one of the antibodies listed in Table 5, wherein the complex has a structure of:

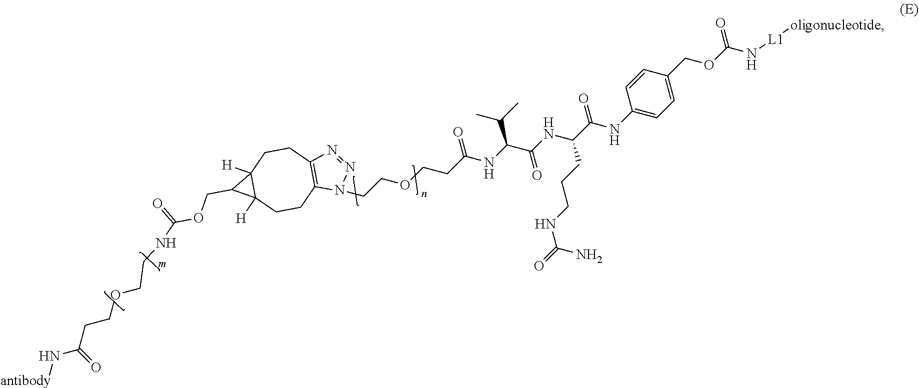

wherein n is 3 and m is 4, and wherein L1 is

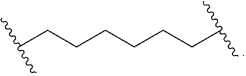 [SMILES: *CCCCCC*]

It should be understood that the amide shown adjacent the anti-TfR1 antibody in Formula (E) results from a reaction with an amine of the anti-TfR1 antibody, such as a lysine epsilon amine.

In some embodiments, L1 is linked to a 5′ phosphate of the oligonucleotide. In some embodiments, L1 is linked to a 5′ phosphate of the oligonucleotide. In some embodiments, the linkage of L1 to a 5′ phosphate of the oligonucleotide forms a phosphodiester bond between L1 and the oligonucleotide.

In some embodiments, L1 is optional (e.g., need not be present).

In some embodiments, the DMPK-targeting oligonucleotide of a complex described herein comprises a structure selected from: +G*x+C*oA*xoC*dG*dT*dG*dT*dG*dG*xdC*dT*xoC*oA*+A*+G (SEQ ID NO: 276), x+C*+A*oU*oG*dA*xdC*dA*dA*dT*xdC*dT*xdC*xoC*oG*x+C*x+C (SEQ ID NO: 345), +A*x+C*xoC*oA*dA*xdC*dA*xdC*dG*dT*xdC*xdC*xoC*oU*x+C*+U (SEQ ID NO: 348), +G*+U*oA*oG*dT*dT*dG*dA*xdC*dT*dG*dG*xoC*oG*+A*+A (SEQ ID NO: 350), x+C*x+C*xoC*oG*dG*dA*dT*xdC*dA*xdC*dA*dG*oG*oA*x+C*+U (SEQ ID NO: 354), xoC*xoC*+A*+A*dA*xdC*dT*dT*dG*xdC*dT*xdC*+A*+G*xoC*oA (SEQ ID NO: 286), xoC*oU*+U*x+C*dA*dG*xdC*dT*dG*dT*dT*dT*x+C*+A*oU*xoC (SEQ ID NO: 352), and x+C*+G*oU*xoC*dA*xdC*xdC*dT*xdC*dG*dG*xdC*xoC*oU*x+C*+A (SEQ ID NO: 357), wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

### III. Formulations

Complexes provided herein may be formulated in any suitable manner. Generally, complexes provided herein are formulated in a manner suitable for pharmaceutical use. For example, complexes can be delivered to a subject using a formulation that minimizes degradation, facilitates delivery and/or (e.g., and) uptake, or provides another beneficial property to the complexes in the formulation. In some embodiments, provided herein are compositions comprising complexes and pharmaceutically acceptable carriers. Such compositions can be suitably formulated such that when administered to a subject, either into the immediate environment of a target cell or systemically, a sufficient amount of the complexes enter target cells (e.g., muscle cells or CNS cells). In some embodiments, complexes are formulated in buffer solutions such as phosphate-buffered saline solutions, liposomes, micellar structures, and capsids.

It should be appreciated that, in some embodiments, compositions may include separately one or more components of complexes provided herein (e.g., muscle-targeting agents, linkers, molecular payloads, or precursor molecules of any one of them).

In some embodiments, complexes are formulated in water or in an aqueous solution (e.g., water with pH adjustments). In some embodiments, complexes are formulated in basic buffered aqueous solutions (e.g., PBS). In some embodiments, formulations as disclosed herein comprise an excipient. In some embodiments, an excipient confers to a composition improved stability, improved absorption, improved solubility and/or (e.g., and) therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil).

In some embodiments, a complex or component thereof (e.g., oligonucleotide or antibody) is lyophilized for extending its shelf-life and then made into a solution before use (e.g., administration to a subject). Accordingly, an excipient in a composition comprising a complex, or component thereof, described herein may be a lyoprotectant (e.g., mannitol, lactose, polyethylene glycol, or polyvinyl pyrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, polyol (for example, glycerol, propylene glycol, and liquid polyethylene glycol, and the like), and suitable mixtures thereof. In some embodiments, formulations include isotonic agents, for example, sugars, polyalcohols such as mannitol, sorbitol, and sodium chloride in the composition. Sterile injectable solutions can be prepared by incorporating the complexes in a required amount in a selected solvent with one or a combination of ingredients enumerated above, as required, followed by filtered sterilization.

In some embodiments, a composition may contain at least about 0.1% of the complex, or component thereof, or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### IV. Methods of Use/Treatment

Complexes comprising a muscle-targeting agent covalently linked to a molecular payload as described herein are effective in treating myotonic dystrophy. In some embodiments, complexes are effective in treating myotonic dystrophy type 1 (DM1). In some embodiments, DM1 is associated with an expansion of a CTG/CUG trinucleotide repeat in the 3′ non-coding region of DMPK. In some embodiments, the nucleotide expansions lead to toxic RNA repeats capable of forming hairpin structures that bind critical intracellular proteins, e.g., muscleblind-like proteins, with high affinity.

In some embodiments, a subject may be a human subject, a non-human primate subject, a rodent subject, or any suitable mammalian subject. In some embodiments, a subject may have myotonic dystrophy. In some embodiments, a subject has a DMPK allele, which may optionally contain a disease-associated repeat. In some embodiments, a subject may have a DMPK allele with an expanded disease-associated-repeat that comprises about 2-10 repeat units, about 2-50 repeat units, about 2-100 repeat units, about 50-1,000 repeat units, about 50-500 repeat units, about 50-250 repeat units, about 50-100 repeat units, about 500-10,000 repeat units, about 500-5,000 repeat units, about 500-2,500 repeat units, about 500-1,000 repeat units, or about 1,000-10,000 repeat units. In some embodiments, a subject is suffering from symptoms of DM1, e.g., muscle atrophy or muscle loss. In some embodiments, a subject is not suffering from symptoms of DM1. In some embodiments, subjects have congenital myotonic dystrophy.

An aspect of the disclosure includes methods involving administering to a subject an effective amount of a complex as described herein. In some embodiments, an effective amount of a pharmaceutical composition that comprises a complex comprising a muscle-targeting agent covalently linked to a molecular payload can be administered to a subject in need of treatment. In some embodiments, a pharmaceutical composition comprising a complex as described herein may be administered by a suitable route, which may include intravenous administration, e.g., as a bolus or by continuous infusion over a period of time. In some embodiments, intravenous administration may be performed by intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a nebulized or lyophilized form may be reconstituted with an aqueous or liquid solution.

Compositions for intravenous administration may contain various carriers such as vegetable oils, dimethylacetamide, dimethylformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In some embodiments, a pharmaceutical composition that comprises a complex comprising a muscle-targeting agent covalently linked to a molecular payload is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a muscle-targeting agent covalently linked to a molecular payload is administered at an effective concentration that confers therapeutic effect on a subject. Effective amounts vary, as recognized by those skilled in the art, depending on the severity of the disease, unique characteristics of the subject being treated, e.g., age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g., the half-life of the complex in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with DM1, e.g., muscle atrophy or muscle weakness, through measures of a subject's self-reported outcomes, e.g., mobility, self-care, usual activities, pain/discomfort, and anxiety/depression, or by quality-of-life indicators, e.g., lifespan.

In some embodiments, a pharmaceutical composition that comprises a complex comprising a muscle-targeting agent covalently linked to a molecular payload described herein is administered to a subject at an effective concentration sufficient to inhibit activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### Additional Embodiments

1. A complex comprising an anti-transferrin receptor 1 (TfR1) antibody covalently linked to an oligonucleotide configured for reducing expression or activity of DMPK, wherein the anti-TfR1 antibody comprises a heavy chain complementarity determining region 1 (CDR-H1), a heavy chain complementarity determining region 2 (CDR-H2), a heavy chain complementarity determining region 3 (CDR-H3), a light chain complementarity determining region 1 (CDR-L1), a light chain complementarity determining region 2 (CDR-L2), a light chain complementarity determining region 3 (CDR-L3) of any of the anti-TfR1 antibodies listed in Tables 2-7,
and wherein the oligonucleotide comprises a 5′-X-Y-Z-3′ configuration, wherein
X comprises 3-7 linked nucleosides, wherein at least one of the nucleosides in X is a 2′-modified nucleoside;
Y comprises 6-15 linked 2′-deoxyribonucleosides, wherein each cytosine in Y is optionally and independently a 5-methyl-cytosine; and
Z comprises 3-7 linked nucleosides, wherein at least one of the nucleosides in Z is a 2′-modified nucleoside.

2. The complex of embodiment 1, wherein
X comprises 3-5 linked nucleosides, wherein at least one of the nucleosides in X is a 2′-modified nucleoside;
Y comprises 6-10 linked 2′-deoxyribonucleosides, wherein each cytosine in Y is optionally and independently a 5-methyl-cytosine; and
Z comprises 3-5 linked nucleosides, wherein at least one of the nucleosides in Z is a 2′-modified nucleoside.

3. The complex of embodiment 1 or embodiment 2, wherein the anti-TfR1 antibody comprises a heavy chain variable region (VH) and a light chain variable region (VL) of any of the anti-TfR1 antibodies listed in Table 3.

4. The complex of any one of embodiments 1 to 3, wherein the anti-TfR1 antibody comprises a heavy chain variable region (VH) comprising an amino acid sequence at least 95% identical to SEQ ID NO: 76 and/or a light chain variable region (VL) comprising an amino acid sequence at least 95% identical to SEQ ID NO: 75,
optionally wherein the anti-TfR1 antibody comprises a VH comprising the amino acid sequence of SEQ ID NO: 76 and a VL comprising the amino acid sequence of SEQ ID NO: 75.

5. The complex of any one of embodiments 1 to 3, wherein the anti-TfR1 antibody is a Fab, optionally wherein the Fab comprises a heavy chain and a light chain of any of the anti-TfR1 Fabs listed in Table 5.

6. The complex of embodiment 5, wherein the Fab comprises a heavy chain comprising an amino acid sequence at least 85% identical to SEQ ID NO: 101 and/or a light chain comprising an amino acid sequence at least 85% identical to SEQ ID NO: 90, optionally wherein the Fab comprises a heavy chain comprising the amino acid sequence of SEQ ID NO: 101 and a light chain comprising the amino acid sequence of SEQ ID NO: 90.

7. The complex of any one of embodiments 1 to 6, wherein the antibody and the oligonucleotide are covalently linked via a linker.

8. The complex of embodiment 7, wherein the linker is a cleavable linker.

9. The complex of embodiment 7 or embodiment 8, wherein the linker comprises a valine-citrulline sequence.

10. The complex of any one of embodiments 1 to 9, wherein the oligonucleotide is 15 to 25 nucleosides in length and comprises a region of complementarity to at least 15 consecutive nucleosides of any one of SEQ ID NOs: 160-230, optionally wherein the oligonucleotide is 15 to 20 nucleosides in length.

11. The complex of any one of embodiments 1 to 10, wherein the oligonucleotide comprises at least 15 consecutive nucleosides of any one of SEQ ID NOs: 231-362, wherein each thymine base (T) may independently and optionally be replaced with a uracil base (U), and each U may independently and optionally be replaced with a T.

12. The complex of any one of embodiments 1 to 11, wherein each nucleoside in X is a 2′-modified nucleoside and/or each nucleoside in Z is a 2′-modified nucleoside, optionally wherein each 2′-modified nucleoside is independently a 2′-4′ bicyclic nucleoside or a non-bicyclic 2′-modified nucleoside.

13. The complex of any one of embodiments 1 to 12, wherein each nucleoside in X is a non-bicyclic 2′-modified nucleoside and/or each nucleoside in Z is a non-bicyclic 2′-modified nucleoside, optionally wherein the non-bicyclic 2′-modified nucleoside is a 2′-MOE modified nucleoside.

14. The complex of any one of embodiments 1 to 12, wherein the oligonucleotide comprises a 5′-X-Y-Z-3′ configuration of:

| X | Y | Z |
|---|---|---|
| EEEEE | (D)₁₀ | EEEEE, |
| EEE | (D)₁₀ | EEE, |
| EEEEE | (D)₁₀ | EEEE, |
| EEEEE | (D)₁₀ | EE, |
| LLL | (D)₁₀ | LLL, |
| EELL | (D)₈ | LLEE, |
| LLEE | (D)₈ | EELL, or |
| LLEEE | (D)₁₀ | EEELL, |

wherein “E” is a 2′-MOE modified ribonucleoside; “L” is LNA; “D” is 2′-deoxyribonucleoside; and “10” or “8” is the number of the 2′-deoxyribonucleosides in Y.

15. The complex of any one of embodiments 1 to 14, wherein the oligonucleotide comprises one or more phosphorothioate internucleoside linkages.

16. The complex of any one of embodiments 1 to 15, wherein each internucleoside linkage in the oligonucleotide is a phosphorothioate internucleoside linkage.

17. The complex of any one of embodiments 1 to 15, wherein the oligonucleotide comprises one or more phosphodiester internucleoside linkages, optionally wherein the one or more phosphodiester internucleoside linkages are in X and or Z.

18. The complex of any one of embodiments 1 to 17, wherein the oligonucleotide comprises a structure selected from:

(SEQ ID NO: 302)
oC*oA*oU*oG*G*dC*dA*dT*dA*dC*dA*dC*dC*dT*dG*oG*oC*
oC*oC*oG,
(SEQ ID NO: 303)
oC*oA*oC*oC*oA*dA*dC*dA*xdC*dG*dT*dC*dC*dC*dT*oC*o
U*oC*oC*oU,
(SEQ ID NO: 304)
oU*oC*oA*oC*oC*dA*dA*dC*dA*xdC*dG*dT*dC*dC*dC*oU*o
C*oU*oC*oC,
(SEQ ID NO: 305)
oC*oC*oA*oU*oU*dC*dA*dC*dC*dA*dA*dC*dA*xdC*dG*oU*C
*oC*oC*oU,
(SEQ ID NO: 306)
oU*oA*oC*oA*oG*dG*dT*dA*dG*dT*dT*dC*dT*dC*dA*oU*C*
oC*oU*oG,
(SEQ ID NO: 307)
oG*oU*oA*oC*oA*dG*dG*dT*dA*dG*dT*dT*dC*dT*dC*oA*oU
*oC*oC*oU,
(SEQ ID NO: 308)
oA*oC*oC*oA*oG*G*dT*dA*dC*dA*dG*dG*dT*dA*dG*oU*oU*
oC*oU*oC,
(SEQ ID NO: 309)
oG*oA*oC*oC*oA*dG*dG*dT*dA*dC*dA*dG*dG*dT*dA*G*oU*
oU*oC*oU,
(SEQ ID NO: 310)
oU*oG*oA*oC*oC*dA*dG*dG*dT*dA*dC*dA*dG*dG*dT*oA*xo
G*oU*oU*oC,
(SEQ ID NO: 311)
oC*oC*oC*A*oA*dA*dC*dT*dT*dG*dC*dT*dC*dA*dG*oC*oA*
oG*oU*oG,
(SEQ ID NO: 312)
oU*oG*oA*C*oA*dA*dT*dC*dT*dC*xdC*dG*dC*dC*dA*oG*oG
*oU*oA*oG,
(SEQ ID NO: 313)
oA*oU*oG*oA*oC*dA*dA*dT*dC*dT*dC*xdC*dG*dC*dC*oA*o
G*oG*oU*oA,
(SEQ ID NO: 314)
oC*oA*oU*oG*oA*dC*dA*dA*dT*dC*dT*dC*xdC*dG*dC*oC*o
A*oG*oG*oU,
(SEQ ID NO: 315)
oC*oC*oA*oU*G*dA*dC*dA*dA*dT*dC*dT*dC*xdC*dG*oC*oC
*oA*oG*oG,
(SEQ ID NO: 316)
oG*oC*oC*oA*oU*G*dA*dC*dA*dA*dT*dC*dT*dC*xdC*oG*oC
*oC*oA*oG,
(SEQ ID NO: 246)
oG*oG*oC*oC*oA*dT*dG*dA*dC*dA*dA*dT*dC*dT*dC*oC*oG
*oC*oC*oA,
(SEQ ID NO: 317)
oU*oG*oG*C*oC*dA*dT*dG*dA*dC*dA*dA*dT*dC*dT*oC*oC*
oG*oC*oC,
(SEQ ID NO: 318)
oU*oG*oU*oG*oC*dA*xdC*dG*dT*dA*dG*dC*dC*dA*dA*oG*o
C*oC*oG*oG,
(SEQ ID NO: 319)
oC*oU*oG*oU*oG*dC*dA*xdC*dG*dT*dA*dG*dC*dC*dA*oA*o
G*oC*oC*oG,
(SEQ ID NO: 320)
oC*oA*oC*oA*oG*xdC*dG*dG*dT*dC*dC*dA*dG*dC*dA*oG*o
G*oA*oU*oG,
(SEQ ID NO: 321)
oU*oG*oG*C*oC*dA*dC*dA*dG*xdC*dG*dG*dT*dC*dC*oA*oG
*oC*oA*oG,
(SEQ ID NO: 322)
OA*oG*oC*oG*oC*dC*dC*dA*dC*dC*dA*dG*dT*dC*dA*oC*oA
*oC*oU*oC,
(SEQ ID NO: 323)
oC*oA*oG*oC*oG*dC*dC*dC*dA*dC*dC*dA*dG*dT*dC*oA*oC
*oA*oC*oU,
(SEQ ID NO: 254)
oC*C*oA*oG*oC*dG*dC*dC*dC*dA*dC*dC*dA*dG*dT*oC*oA*
oC*oA*oC,
(SEQ ID NO: 255)
oG*oC*oG*oA*A*dT*dA*dC*dA*dC*dC*dC*dA*dG*xdC*oG*oC
*oC*oC*oA,
(SEQ ID NO: 256)
oG*oG*oC*oG*oA*dA*dT*dA*dC*dA*dC*dC*dC*dA*dG*oC*oG
*oC*oC*oC,
(SEQ ID NO: 324)
oU*oU*G*oU*oA*dG*dT*dG*dG*dA*xdC*dG*dA*dT*dC*oU*oU
*oG*oC*oC,
(SEQ ID NO: 325)
oC*oU*oU*oG*oU*dA*dG*dT*dG*dG*dA*xdC*dG*dA*dT*oC*o
U*oU*oG*oC,
(SEQ ID NO: 326)
oC*oC*oU*oU*oG*dT*dA*dG*dT*dG*dG*dA*xdC*dG*dA*oU*o
C*oU*oU*oG,
(SEQ ID NO: 327)
oC*oG*oG*oA*oG*dA*dC*dC*dA*dT*dC*dC*dC*dA*dG*oU*oC
*oG*oA*oG,
(SEQ ID NO: 328)
oG*oA*oA*oU*oG*dT*dC*xdC*dG*dA*dC*dA*dG*dT*dG*U*oC
*oU*oC*oC,
(SEQ ID NO: 329)
oC*oG*oA*A*oU*G*dT*dC*xdC*dG*dA*dC*dA*dG*dT*oG*oU*
oC*oU*oC,
(SEQ ID NO: 330)
oG*oG*oG*oC*oC*dT*dG*dG*dG*dA*dC*dC*dT*dC*dA*oC*oU
*oG*oU*oC,
(SEQ ID NO: 331)
oU*oG*oC*oA*oC*dG*dT*dG*dT*G*dG*dC*dT*dC*dA*oA*oG*
oC*oA*oG,
(SEQ ID NO: 332)
oC*C*oA*oC*oU*dT*dC*dA*dG*dC*dT*dG*dT*dT*dT*oC*oA*
oU*oC*oC,
(SEQ ID NO: 333)
oG*oC*oG*oU*oC*dA*dC*dC*dT*xdC*dG*dG*dC*dC*dT*C*oA
*oG*oC*oC,
(SEQ ID NO: 334)
oA*oG*oC*oG*oU*dC*dA*dC*dC*dT*xdC*dG*dG*dC*dC*oU*C
*oA*oG*oC,
(SEQ ID NO: 335)
oC*oG*oU*oA*oG*dT*dT*dG*dA*dC*dT*dG*dG*xdC*dG*oA*o
A*oG*oU*oU,
(SEQ ID NO: 336)
oG*oG*oG*oC*oC*xdC*G*dG*dA*dT*dC*dA*dC*dA*dG*oG*oA
*oC*oU*oG,
(SEQ ID NO: 337)
oU*oU*G*oC*oC*dC*dA*dT*dC*dC*dA*xdC*dG*dT*dC*oA*oG
*oG*oG*oC,
(SEQ ID NO: 338)
oG*oG*oA*oC*G*dG*dC*dC*xdC*dG*dG*dC*dT*dT*dG*oC*oU
*oG*oC*oC,
(SEQ ID NO: 339)
oU*oG*oG*oA*oA*dC*dA*xdC*dG*dG*dA*xdC*dG*dG*dC*oC*
oC*oG*oG*oC,
(SEQ ID NO: 340)
oC*oA*oU*C*oC*dA*dA*dA*dA*xdC*dG*dT*G*dG*dA*oU*oU*
oG*oG*oG,
and
(SEQ ID NO: 341)
oG*oC*oA*oU*oC*dC*dA*dA*dA*dA*xdC*dG*dT*dG*dG*oA*o
U*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

19. The complex of embodiment 18, wherein the oligonucleotide is conjugated to an amine group at its 5′-end and comprises a structure selected from:

(SEQ ID NO: 302)
NH₂-(CH₂)₆-oC*oA*oU*oG*oG*dC*dA*dT*dA*dC*dA*dC*dC*
dT*dG*oG*oC*oC*oC*oG,
(SEQ ID NO: 303)
NH₂-(CH₂)₆-oC*oA*oC*oC*oA*dA*dC*dA*xdC*dG*dT*dC*dC
*dC*dT*oC*oU*oC*oC*oU,
(SEQ ID NO: 304)
NH₂-(CH₂)₆-oU*oC*oA*oC*oC*dA*dA*dC*dA*xdC*dG*dT*dC
*dC*dC*oU*oC*oU*oC*oC,
(SEQ ID NO: 305)
NH₂-(CH₂)₆-oC*oC*oA*oU*oU*dC*dA*dC*dC*dA*dA*dC*dA*
xdC*dG*oU*oC*oC*oC*oU,
(SEQ ID NO: 306)
NH₂-(CH₂)₆-oU*oA*oC*oA*oG*dG*dT*dA*dG*dT*dT*dC*dT*
dC*dA*oU*oC*oC*oU*oG,
(SEQ ID NO: 307)
NH₂-(CH₂)₆-oG*oU*oA*oC*oA*dG*dG*dT*dA*dG*dT*dT*dC*
dT*dC*oA*oU*oC*oC*oU,
(SEQ ID NO: 308)
NH₂-(CH₂)₆-oA*oC*C*oA*G*dG*dT*dA*dC*dA*dG*dG*dT*dA
*dG*oU*oU*oC*oU*oC,
(SEQ ID NO: 309)
NH₂-(CH₂)₆-oG*oA*oC*oC*oA*dG*dG*dT*dA*dC*dA*dG*dG*
dT*dA*oG*oU*oU*C*oU,
(SEQ ID NO: 310)
NH₂-(CH₂)₆-oU*oG*oA*oC*oC*dA*dG*dG*dT*dA*dC*dA*dG*
dG*dT*oA*xoG*oU*oU*oC,
(SEQ ID NO: 311)
NH₂-(CH₂)₆-oC*oC*oC*oA*A*dA*dC*dT*dT*dG*dC*dT*dC*d
A*dG*oC*oA*G*oU*oG,
(SEQ ID NO: 312)
NH₂-(CH₂)₆-oU*oG*oA*oC*A*dA*dT*dC*dT*dC*xdC*dG*dC*
dC*dA*oG*oG*oU*oA*oG,
(SEQ ID NO: 313)
NH₂-(CH₂)₆-oA*oU*oG*oA*oC*dA*dA*dT*dC*dT*dC*xdC*dG
*dC*dC*oA*oG*oG*oU*oA,
(SEQ ID NO: 314)
NH₂-(CH₂)₆-oC*oA*oU*G*oA*dC*dA*dA*dT*dC*dT*dC*xdC*
dG*dC*oC*oA*oG*oG*oU,
(SEQ ID NO: 315)
NH₂-(CH₂)₆-oC*oC*oA*oU*G*dA*dC*dA*dA*dT*dC*dT*dC*x
dC*dG*oC*oC*oA*oG*oG,
(SEQ ID NO: 316)
NH₂-(CH₂)₆-oG*oC*oC*oA*oU*dG*dA*dC*dA*dA*dT*dC*dT*
dC*xdC*oG*oC*C*oA*oG,
(SEQ ID NO: 246)
NH₂-(CH₂)₆-oG*oG*C*C*oA*dT*dG*dA*dC*dA*dA*dT*dC*dT
*dC*oC*oG*oC*oC*oA,
(SEQ ID NO: 317)
NH₂-(CH₂)₆-oU*oG*oG*oC*oC*dA*dT*dG*dA*dC*dA*dA*dT*
dC*dT*oC*oC*G*oC*oC,
(SEQ ID NO: 318)
NH₂-(CH₂)₆-oU*oG*oU*oG*oC*dA*xdC*dG*dT*dA*dG*dC*dC
*dA*dA*oG*oC*oC*oG*oG,
(SEQ ID NO: 319)
NH₂-(CH₂)₆-oC*oU*oG*oU*oG*dC*dA*xdC*dG*dT*dA*dG*dC
*dC*dA*oA*oG*oC*C*oG,
(SEQ ID NO: 320)
NH₂-(CH₂)₆-oC*oA*oC*oA*oG*xdC*dG*dG*dT*dC*dC*dA*dG
*dC*dA*oG*oG*oA*oU*oG,
(SEQ ID NO: 321)
NH₂-(CH₂)₆-oU*oG*oG*oC*oC*dA*dC*dA*dG*xdC*dG*dG*dT
*dC*dC*oA*oG*oC*oA*oG,
(SEQ ID NO: 322)
NH₂-(CH₂)₆-oA*oG*oC*G*oC*dC*dC*dA*dC*dC*dA*dG*dT*d
C*dA*oC*oA*oC*oU*oC,
(SEQ ID NO: 323)
NH₂-(CH₂)₆-oC*oA*oG*oC*oG*dC*dC*dC*dA*dC*dC*dA*dG*
dT*dC*oA*oC*A*oC*oU,
(SEQ ID NO: 254)
NH₂-(CH₂)₆-oC*oC*oA*oG*C*dG*dC*dC*dC*dA*dC*dC*dA*d
G*dT*oC*oA*oC*oA*oC,
(SEQ ID NO: 255)
NH₂-(CH₂)₆-oG*oC*oG*oA*oA*dT*dA*dC*dA*dC*dC*dC*dA*
dG*xdC*oG*oC*oC*oC*oA,
(SEQ ID NO: 256)
NH₂-(CH₂)₆-oG*G*oC*oG*oA*dA*dT*dA*dC*dA*dC*dC*dC*d
A*dG*C*oG*oC*oC*oC,
(SEQ ID NO: 324)
NH₂-(CH₂)₆-oU*oU*oG*oU*oA*dG*dT*dG*dG*dA*xdC*dG*dA
*dT*dC*oU*oU*oG*oC*oC,
(SEQ ID NO: 325)
NH₂-(CH₂)₆-oC*oU*oU*oG*oU*dA*dG*dT*dG*dG*dA*xdC*dG
*dA*dT*oC*oU*oU*oG*oC,
(SEQ ID NO: 326)
NH₂-(CH₂)₆-oC*oC*oU*oU*oG*dT*dA*dG*dT*dG*dG*dA*xdC
*dG*dA*oU*oC*oU*oU*oG,
(SEQ ID NO: 327)
NH₂-(CH₂)₆-oC*oG*oG*oA*G*dA*dC*dC*dA*dT*dC*dC*dC*d
A*dG*oU*oC*oG*oA*oG,
(SEQ ID NO: 328)
NH₂-(CH₂)₆-oG*oA*oA*oU*G*dT*dC*xdC*dG*dA*dC*dA*dG*
dT*dG*oU*oC*oU*oC*oC,
(SEQ ID NO: 329)
NH₂-(CH₂)₆-oC*oG*oA*oA*oU*dG*dT*dC*xdC*dG*dA*dC*dA
*dG*dT*oG*oU*C*oU*oC,
(SEQ ID NO: 330)
NH₂-(CH₂)₆-oG*oG*oG*oC*oC*dT*dG*dG*dG*dA*dC*dC*dT*
dC*dA*oC*oU*oG*oU*oC,
(SEQ ID NO: 331)
NH₂-(CH₂)₆-oU*oG*oC*oA*oC*dG*dT*dG*dT*dG*dG*dC*dT*
dC*dA*oA*oG*C*oA*oG,
(SEQ ID NO: 332)
NH₂-(CH₂)₆-oC*oC*A*oC*oU*dT*dC*dA*dG*dC*dT*dG*dT*d
T*dT*oC*oA*oU*oC*oC,
(SEQ ID NO: 333)
NH₂-(CH₂)₆-oG*C*oG*oU*oC*dA*dC*dC*dT*xdC*dG*dG*dC*
dC*dT*oC*oA*oG*C*C,
(SEQ ID NO: 334)
NH₂-(CH₂)₆-oA*oG*oC*oG*oU*dC*dA*dC*dC*dT*xdC*dG*dG
*dC*dC*oU*oC*A*oG*oC,
(SEQ ID NO: 335)
NH₂-(CH₂)₆-oC*oG*oU*oA*G*dT*dT*dG*dA*dC*dT*dG*dG*x
dC*dG*oA*oA*oG*oU*oU,
(SEQ ID NO: 336)
NH₂-(CH₂)₆-oG*oG*oG*oC*oC*xdC*dG*dG*dA*dT*dC*dA*dC
*dA*dG*oG*oA*oC*oU*oG,
(SEQ ID NO: 337)
NH₂-(CH₂)₆-oU*oU*oG*oC*C*dC*dA*dT*dC*dC*dA*xdC*dG*
dT*dC*A*oG*oG*oG*oC,
(SEQ ID NO: 338)
NH₂-(CH₂)₆-oG*oG*oA*oC*oG*dG*dC*dC*xdC*dG*dG*dC*dT
*dT*dG*oC*oU*oG*oC*oC,
(SEQ ID NO: 339)
NH₂-(CH₂)₆-oU*oG*oG*oA*oA*dC*dA*xdC*dG*dG*dA*xdC*d
G*dG*dC*oC*oC*oG*oG*oC,
(SEQ ID NO: 340)
NH₂-(CH₂)₆-oC*oA*oU*oC*oC*dA*dA*dA*dA*xdC*dG*dT*dG
*dG*dA*oU*oU*oG*oG*oG,
and
(SEQ ID NO: 341)
NH₂-(CH₂)₆-oG*oC*oA*oU*oC*dC*dA*dA*dA*dA*xdC*dG*dT
*dG*dG*oA*oU*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage,
and optionally wherein a phosphodiester linkage or other moiety is present between the 5′-NH₂—(CH₂)₆— and the oligonucleotide.

20. The complex of any one of embodiments 1 to 17, wherein the oligonucleotide comprises a structure selected from:

(SEQ ID NO: 275)
x+C*+A*xoC*oG*dT*dG*dT*dG*dG*xdC*dT*xdC*oA*oA*+G*x
+C,
(SEQ ID NO: 275)
xoC*oA*x+C*+G*dT*dG*dT*dG*dG*xdC*dT*xdC*+A*+A*oG*x
oC,
(SEQ ID NO: 276)
+G*x+C*oA*xoC*dG*dT*dG*dT*dG*dG*xdC*dT*xoC*oA*+A*+
G,
(SEQ ID NO: 276)
oG*xoC*+A*x+C*dG*dT*dG*dT*dG*dG*xdC*dT*x+C*+A*oA*o
G,
(SEQ ID NO: 342)
+A*x+C*oG*oU*dG*dT*dG*dG*xdC*dT*xdC*dA*oA*oG*x+C*+
A,
(SEQ ID NO: 342)
oA*xoC*+G*+U*dG*dT*dG*dG*xdC*dT*xdC*dA*+A*+G*xoC*o
A,
(SEQ ID NO: 278)
x+C*+A*oA*oA*xdC*dT*dT*dG*xdC*dT*xdC*dA*oG*xoC*+A*
+G,
(SEQ ID NO: 343)
+A*x+C*oU*oU*xdC*dA*dG*xdC*dT*dG*dT*dT*oU*xoC*+A*+
U,
(SEQ ID NO: 344)
+U*+A*oG*oU*dT*dG*dA*xdC*dT*dG*dG*xdC*oG*oA*+A*+G,
(SEQ ID NO: 281)
+G*x+C*xoC*xoC*dG*dG*dA*dT*dC*dA*xdC*dA*oG*oG*+A*x
+C,
(SEQ ID NO: 345)
x+C*+A*oU*oG*dA*xdC*dA*dA*dT*xdC*dT*xdC*xoC*G*x+C*
x+C,
(SEQ ID NO: 346)
+G*+U*xoC*oA*xdC*xdC*dT*xdC*dG*dG*xdC*dC*oU*xoC*+A
*+G,
(SEQ ID NO: 347)
x+C*o+C*oA*oG*dG*dT*dA*xdC*dA*dG*dG*dT*oA*oG*+U*+
U,
(SEQ ID NO: 348)
+A*x+C*xoC*oA*dA*xdC*dA*xdC*dG*dT*xdC*xdC*xoC*oU*x
+C*+U,
(SEQ ID NO: 286)
x+C*o+C*oA*oA*dA*xdC*dT*dT*dG*xdC*dT*xdC*oA*oG*x+C
*+A,
(SEQ ID NO: 349)
x+C*+A*xoC*oU*dT*xdC*dA*dG*xdC*dT*dG*dT*oU*oU*x+C*
+A,
(SEQ ID NO: 350)
+G*+U*oA*oG*dT*dT*dG*dA*xdC*dT*dG*dG*xoC*oG*+A*+A,
(SEQ ID NO: 289)
+G*+G*xoC*xoC*xdC*dG*dG*dA*dT*xdC*dA*xdC*oA*oG*+G*
+A,
(SEQ ID NO: 351)
+A*+A*oA*xoC*dT*dT*dG*xdC*dT*xdC*dA*dG*xoC*oA*+G*+
U,
(SEQ ID NO: 352)
x+C*+U*oU*xoC*dA*dG*xdC*dT*dG*dT*dT*dT*xoC*oA*+U*x
+C,
(SEQ ID NO: 353)
+A*+G*oU*oU*dG*dA*xdC*dT*dG*dG*xdC*dG*oA*oA*+G*+U,
(SEQ ID NO: 354)
x+C*x+C*xoC*oG*dG*dA*dT*xdC*dA*xdC*dA*dG*oG*oA*x+C
*+U,
(SEQ ID NO: 278)
xoC*oA*+A*+A*xdC*dT*dT*dG*xdC*dT*xdC*dA*+G*x+C*oA*
oG,
(SEQ ID NO: 343)
oA*xoC*+U*+U*xdC*dA*dG*xdC*dT*dG*dT*dT*+U*x+C*oA*o
U,
(SEQ ID NO: 344)
oU*oA*+G*+U*dT*dG*dA*xdC*dT*dG*dG*xdC*+G*+A*oA*oG,
(SEQ ID NO: 281)
oG*xoC*x+C*o+C*dG*dG*dA*dT*dC*dA*dC*dA*+G*+G*oA*xo
C,
(SEQ ID NO: 286)
xoC*xoC*+A*+A*dA*xdC*dT*dT*dG*xdC*dT*xdC*+A*+G*xoC
*oA,
(SEQ ID NO: 349)
xoC*oA*x+C*+U*dT*xdC*dA*dG*xdC*dT*dG*dT*+U*+U*xoC*
oA,
(SEQ ID NO: 350)
oG*oU*+A*+G*dT*dT*dG*dA*xdC*dT*dG*dG*x+C*+G*oA*oA,
(SEQ ID NO: 289)
oG*oG*x+C*x+C*xdC*dG*dG*dA*dT*xdC*dA*xdC*+A*+G*oG*
oA,
(SEQ ID NO: 351)
oA*oA*+A*o+C*dT*dT*dG*xdC*dT*xdC*dA*dG*x+C*+A*oG*o
U,
(SEQ ID NO: 352)
xoC*oU*+U*x+C*dA*dG*xdC*dT*dG*dT*dT*dT*x+C*+A*oU*x
oC,
(SEQ ID NO: 353)
oA*oG*+U*+U*dG*dA*xdC*dT*dG*dG*xdC*dG*+A*+A*oG*oU,
(SEQ ID NO: 354)
xoC*xoC*x+C*+G*dG*dA*dT*xdC*dA*xdC*dA*dG*+G*+A*xoC
*oU,
(SEQ ID NO: 355)
x+C*x+C*oA*oU*dG*dA*xdC*dA*dA*dT*xdC*dT*xoC*xoC*+G
*x+C,
(SEQ ID NO: 356)
+A*+U*oG*oA*xdC*dA*dA*dT*xdC*dT*xdC*xdC*oG*xoC*x+C
*+A,
(SEQ ID NO: 357)
x+C*+G*oU*xoC*dA*xdC*xdC*dT*xdC*dG*dG*xdC*xoC*oU*x
+C*+A,
(SEQ ID NO: 358)
+U*x+C*oA*xoC*xdC*dT*xdC*dG*dG*xdC*xdC*dT*xoC*oA*+
G*x+C,
(SEQ ID NO: 359)
+A*x+C*xoC*oA*dG*dG*dT*dA*xdC*dA*dG*dG*oU*A*+G*+U,
(SEQ ID NO: 360)
x+C*+A*oG*oG*dT*dA*xdC*dA*dG*dG*dT*dA*oG*oU*+U*x+
C,
(SEQ ID NO: 361)
x+C*+A*xoC*xoC*dA*dA*xdC*dA*xdC*dG*dT*xdC*xoC*xoC*
+U*x+C,
and
(SEQ ID NO: 362)
x+C*o+C*oA*oA*xdC*dA*xdC*dG*dT*xdC*xdC*xdC*oU*xoC*
+U*x+C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; and “*” indicates a phosphorothioate (PS) internucleoside linkage.

21. The complex of embodiment 20, wherein the oligonucleotide is conjugated to an amine group at its 5′-end and comprises a structure selected from:

(SEQ ID NO: 275)
NH₂-(CH₂)₆-X+C*+A*xoC*oG*dT*dG*dT*dG*dG*xdC*dT*xdC
*oA*oA*+G*x+C,
(SEQ ID NO: 275)
NH₂-(CH₂)₆-XoC*oA*x+C*+G*dT*dG*dT*dG*dG*xdC*dT*xdC
*+A*+A*oG*xoC,
(SEQ ID NO: 276)
NH₂-(CH₂)₆-+G*x+C*oA*xoC*dG*dT*dG*dT*dG*dG*xdC*dT*
xoC*oA*+A*+G,
(SEQ ID NO: 276)
NH₂-(CH₂)₆-oG*xoC*+A*x+C*dG*dT*dG*dT*dG*dG*xdC*dT*
x+C*+A*oA*oG,
(SEQ ID NO: 342)
NH₂-(CH₂)₆-+A*x+C*oG*oU*dG*dT*dG*dG*xdC*dT*xdC*dA*
oA*oG*x+C*+A,
(SEQ ID NO: 342)
NH₂-(CH₂)₆-oA*xoC*+G*+U*dG*dT*dG*dG*xdC*dT*xdC*dA*
+A*+G*xoC*oA,
(SEQ ID NO: 278)
NH₂-(CH₂)₆-X+C*+A*oA*oA*xdC*dT*dT*dG*xdC*dT*xdC*dA
*oG*xoC*+A*+G,
(SEQ ID NO: 343)
NH₂-(CH₂)₆-+A*x+C*oU*oU*xdC*dA*dG*xdC*dT*dG*dT*dT*
oU*xoC*+A*+U,
(SEQ ID NO: 344)
NH₂-(CH₂)₆-+U*+A*oG*oU*dT*dG*dA*xdC*dT*dG*dG*xdC*o
G*oA*+A*+G,
(SEQ ID NO: 281)
NH₂-(CH₂)₆-+G*x+C*xoC*xoC*dG*dG*dA*dT*dC*dA*xdC*dA
*oG*oG*+A*x+C,
(SEQ ID NO: 345)
NH₂-(CH₂)₆-x+C*+A*oU*oG*dA*xdC*dA*dA*dT*xdC*dT*xdC
*xoC*oG*x+C*x+C,
(SEQ ID NO: 346)
NH₂-(CH₂)₆-+G*+U*xoC*oA*xdC*xdC*dT*xdC*dG*dG*xdC*d
C*oU*xoC*+A*+G,
(SEQ ID NO: 347)
NH₂-(CH₂)₆-X+C*x+C*oA*oG*dG*dT*dA*xdC*dA*dG*dG*dT*
oA*oG*+U*+U,
(SEQ ID NO: 348)
NH₂-(CH₂)₆-+A*x+C*xoC*oA*dA*xdC*dA*xdC*dG*dT*xdC*x
dC*xoC*oU*x+C*+U,
(SEQ ID NO: 286)
NH₂-(CH₂)₆-X+C*x+C**A*oA*dA*xdC*dT*dT*dG*xdC*dT*xd
C*oA*oG*x+C*+A,
(SEQ ID NO: 349)
NH₂-(CH₂)₆-X+C*+A*xoC*oU*dT*xdC*dA*dG*xdC*dT*dG*dT
*oU*oU*x+C*+A,
(SEQ ID NO: 350)
NH₂-(CH₂)₆-+G*+U*oA*G*dT*dT*dG*dA*xdC*dT*dG*dG*xoC
*oG*+A*+A,
(SEQ ID NO: 289)
NH₂-(CH₂)₆-+G*+G*xoC*xoC*xdC*dG*dG*dA*dT*xdC*dA*xd
C*oA*oG*+G*+A,
(SEQ ID NO: 351)
NH₂-(CH₂)₆-+A*+A*oA*xoC*dT*dT*dG*xdC*dT*xdC*dA*dG*
xoC*oA*+G*+U,
(SEQ ID NO: 352)
NH₂-(CH₂)₆-X+C*+U*oU*xoC*dA*dG*xdC*dT*dG*dT*dT*dT*
xoC*oA*+U*x+C,
(SEQ ID NO: 353)
NH₂-(CH₂)₆-+A*+G*oU*oU*dG*dA*xdC*dT*dG*dG*xdC*dG*o
A*oA*+G*+U,
(SEQ ID NO: 354)
NH₂-(CH₂)₆-x+C*x+C*xoC*oG*dG*dA*dT*xdC*dA*xdC*dA*d
G*oG*oA*x+C*+U,
(SEQ ID NO: 278)
NH₂-(CH₂)₆-XoC*oA*+A*+A*xdC*T*dT*dG*xdC*dT*xdC*dA*
+G*x+C*oA*oG,
(SEQ ID NO: 343)
NH₂-(CH₂)₆-oA*xoC*+U*+U*xdC*dA*dG*xdC*dT*dG*dT*dT*
+U*x+C*oA*oU,
(SEQ ID NO: 344)
NH₂-(CH₂)₆-oU*oA*+G*+U*dT*dG*dA*xdC*dT*dG*dG*xdC*+
G*+A*oA*oG,
(SEQ ID NO: 281)
NH₂-(CH₂)₆-oG*xoC*x+C*x+C*dG*dG*dA*dT*dC*dA*dC*dA*
+G*+G*oA*xoC,
(SEQ ID NO: 286)
NH₂-(CH₂)₆-XoC*X0C*+A*+A*dA*xdC*dT*dT*dG*xdC*dT*xd
C*+A*+G*xoC*oA,
(SEQ ID NO: 349)
NH₂-(CH₂)₆-XoC*oA*x+C*+U*dT*xdC*dA*dG*xdC*dT*dG*dT
*+U*+U*xoC*oA,
(SEQ ID NO: 350)
NH₂-(CH₂)₆-oG*oU*+A*+G*dT*dT*dG*dA*xdC*dT*dG*dG*x+
C*+G*oA*oA,
(SEQ ID NO: 289)
NH₂-(CH₂)₆-oG*oG*x+C*x+C*xdC*dG*dG*dA*dT*xdC*dA*xd
C*+A*+G*oG*oA,
(SEQ ID NO: 351)
NH₂-(CH₂)₆-oA*oA*+A*x+C*dT*dT*dG*xdC*dT*xdC*dA*dG*
x+C*+A*oG*oU,
(SEQ ID NO: 352)
NH₂-(CH₂)₆-XoC*oU*+U*x+C*dA*dG*xdC*dT*dG*dT*dT*dT*
x+C*+A*oU*xoC,
(SEQ ID NO: 353)
NH₂-(CH₂)₆-oA*oG*+U*+U*dG*dA*xdC*dT*dG*dG*xdC*dG*+
A*+A*oG*oU,
(SEQ ID NO: 354)
NH₂-(CH₂)₆-XoC*xoC*x+C*+G*dG*dA*dT*xdC*dA*xdC*dA*d
G*+G*+A*xoC*oU,
(SEQ ID NO: 355)
NH₂-(CH₂)₆-X+C*x+C*oA*oU*dG*dA*xdC*dA*dA*dT*xdC*dT
*xoC*xoC*+G*x+C,
(SEQ ID NO: 356)
NH₂-(CH₂)₆-+A*+U*oG*A*xdC*dA*dA*dT*xdC*dT*xdC*xdC*
oG*xoC*x+C*+A,
(SEQ ID NO: 357)
NH₂-(CH₂)₆-X+C*+G*oU*xoC*dA*xdC*xdC*dT*xdC*dG*dG*x
dC*xoC*oU*x+C*+A,
(SEQ ID NO: 358)
NH₂-(CH₂)₆-+U*x+C*oA*xoC*xdC*dT*xdC*dG*dG*xdC*xdC*
dT*xoC*oA*+G*x+C,
(SEQ ID NO: 359)
NH₂-(CH₂)₆-+A*x+C*xoC*oA*dG*dG*dT*dA*xdC*dA*dG*dG*
U*oA*+G*+U,
(SEQ ID NO: 360)
NH₂-(CH₂)₆-X+C*+A*oG*oG*dT*dA*xdC*dA*dG*dG*dT*dA*o
G*oU*+U*x+C,
(SEQ ID NO: 361)
NH₂-(CH₂)₆-X+C*+A*xoC*xoC*dA*dA*xdC*dA*xdC*dG*dT*x
dC*xoC*xoC*+U*x+C,
and
(SEQ ID NO: 362)
NH₂-(CH₂)₆-X+C*x+C*oA*oA*xdC*dA*xdC*dG*dT*xdC*xdC*
xdC*oU*xoC*+U*x+C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; and “*” indicates a phosphorothioate (PS) internucleoside linkage,
and optionally wherein a phosphodiester linkage or other moiety is present between the 5′-NH₂—(CH₂)₆— and the oligonucleotide.

22. A method of reducing DMPK expression in a muscle cell, the method comprising contacting the muscle cell with an effective amount of the complex of any one of embodiments 1 to 21 to reduce DMPK expression in the muscle cell.

23. The method of embodiment 22, wherein reducing DMPK expression in the muscle cell comprises reducing the amount of DMPK RNA in the muscle cell, optionally wherein the DMPK RNA amount is reduced in the nucleus of the muscle cell.

24. The method of embodiment 22 or embodiment 23, wherein reducing DMPK expression in the muscle cell comprises reducing the amount of DMPK protein in the muscle cell.

25. A method of treating myotonic dystrophy type 1 (DM1), the method comprising administering to a subject in need thereof an effective amount of the complex of any one of embodiments 1 to 21, wherein the subject has a mutant DMPK allele comprising disease-associated CTG repeats.

26. The method of embodiment 25, wherein the administering results in a reduction of DMPK mRNA in a muscle cell in the subject by at least 30%.

27. The method of embodiment 25 or embodiment 26, wherein the administering results in a reduction of a DMPK mRNA in the nucleus of a muscle cell in the subject.

28. An oligonucleotide comprising a structure selected from:

(SEQ ID NO: 302)
oC*oA*oU*oG*G*dC*dA*dT*dA*dC*dA*dC*dC*dT*dG*oG*C*o
C*oC*oG,
(SEQ ID NO: 303)
oC*A*oC*oC*A*dA*dC*dA*xdC*dG*dT*dC*dC*dC*dT*oC*oU*
oC*oC*oU,
(SEQ ID NO: 304)
oU*oC*oA*oC*oC*dA*dA*dC*dA*xdC*dG*dT*dC*dC*dC*oU*o
C*oU*oC*oC,
(SEQ ID NO: 305)
oC*oC*oA*oU*oU*dC*dA*dC*dC*dA*dA*dC*dA*xdC*dG*oU*o
C*oC*C*oU,
(SEQ ID NO: 306)
oU*oA*oC*oA*oG*G*dT*dA*dG*dT*dT*dC*dT*dC*dA*oU*oC*
oC*oU*oG,
(SEQ ID NO: 307)
oG*oU*oA*oC*oA*dG*dG*dT*dA*dG*dT*dT*dC*dT*dC*oA*oU
*oC*oC*oU,
(SEQ ID NO: 308)
oA*oC*oC*oA*G*dG*dT*dA*dC*dA*dG*dG*dT*dA*dG*oU*oU*
oC*oU*oC,
(SEQ ID NO: 309)
oG*oA*oC*oC*oA*dG*dG*dT*dA*dC*dA*dG*dG*dT*dA*G*oU*
oU*oC*oU,
(SEQ ID NO: 310)
oU*oG*oA*oC*oC*dA*dG*dG*dT*dA*dC*dA*dG*dG*dT*oA*xo
G*oU*oU*oC,
(SEQ ID NO: 311)
oC*oC*C*oA*A*dA*dC*dT*T*dG*dC*dT*dC*dA*dG*oC*oA*oG
*oU*oG,
(SEQ ID NO: 312)
oU*oG*oA*oC*oA*dA*dT*dC*dT*dC*xdC*dG*dC*dC*dA*oG*o
G*oU*oA*oG,
(SEQ ID NO: 313)
oA*oU*G*oA*oC*A*dA*dT*dC*dT*dC*xdC*dG*dC*dC*oA*oG*
oG*oU*oA,
(SEQ ID NO: 314)
oC*oA*oU*oG*oA*dC*dA*dA*dT*dC*dT*dC*xdC*dG*dC*oC*o
A*oG*oG*oU,
(SEQ ID NO: 315)
oC*C*oA*oU*G*dA*dC*dA*dA*dT*dC*dT*dC*xdC*dG*C*oC*o
A*oG*oG,
(SEQ ID NO: 316)
oG*oC*oC*oA*oU*dG*dA*dC*dA*dA*dT*dC*dT*dC*xdC*oG*o
C*oC*oA*oG,
(SEQ ID NO: 246)
oG*oG*oC*C*A*dT*dG*dA*dC*dA*dA*dT*dC*dT*dC*oC*oG*o
C*oC*oA,
(SEQ ID NO: 317)
oU*G*oG*oC*C*dA*T*dG*dA*dC*dA*dA*dT*dC*dT*oC*oC*oG
*oC*oC,
(SEQ ID NO: 318)
oU*oG*oU*oG*oC*dA*xdC*dG*dT*dA*dG*dC*dC*dA*dA*oG*o
C*oC*oG*oG,
(SEQ ID NO: 319)
oC*oU*oG*oU*oG*dC*dA*xdC*dG*dT*dA*dG*dC*dC*dA*oA*o
G*oC*oC*oG,
(SEQ ID NO: 320)
oC*oA*oC*oA*oG*xdC*G*dG*dT*dC*dC*dA*dG*dC*dA*oG*oG
*oA*oU*oG,
(SEQ ID NO: 321)
oU*oG*oG*oC*oC*dA*dC*dA*dG*xdC*dG*dG*dT*dC*dC*oA*o
G*oC*oA*oG,
(SEQ ID NO: 322)
oA*oG*oC*oG*oC*dC*dC*dA*dC*dC*dA*dG*dT*dC*dA*oC*oA
*oC*oU*oC,
(SEQ ID NO: 323)
oC*oA*oG*oC*G*dC*dC*dC*dA*dC*dC*dA*dG*dT*dC*oA*oC*
oA*oC*oU,
(SEQ ID NO: 254)
oC*oC*A*oG*oC*dG*dC*dC*dC*dA*dC*dC*dA*dG*dT*oC*oA*
oC*oA*oC,
(SEQ ID NO: 255)
oG*oC*oG*oA*A*T*dA*dC*dA*dC*dC*dC*dA*dG*xdC*oG*oC*
oC*oC*oA,
(SEQ ID NO: 256)
oG*oG*oC*oG*oA*dA*dT*dA*dC*dA*dC*dC*dC*dA*dG*oC*oG
*oC*oC*oC,
(SEQ ID NO: 324)
oU*oU*oG*oU*oA*dG*dT*dG*dG*dA*xdC*dG*dA*dT*dC*oU*o
U*G*oC*oC,
(SEQ ID NO: 325)
oC*oU*oU*oG*oU*dA*dG*dT*dG*dG*dA*xdC*dG*dA*dT*oC*o
U*oU*oG*oC,
(SEQ ID NO: 326)
oC*oC*oU*oU*G*dT*dA*dG*dT*dG*dG*dA*xdC*dG*dA*oU*oC
*oU*oU*oG,
(SEQ ID NO: 327)
oC*G*oG*oA*G*dA*dC*dC*dA*dT*dC*dC*dC*dA*dG*oU*oC*o
G*oA*oG,
(SEQ ID NO: 328)
oG*oA*oA*oU*G*dT*dC*xdC*dG*dA*dC*dA*dG*dT*dG*oU*oC
*oU*oC*oC,
(SEQ ID NO: 329)
oC*oG*oA*oA*oU*dG*dT*dC*xdC*dG*dA*dC*dA*dG*dT*oG*o
U*oC*oU*oC,
(SEQ ID NO: 330)
oG*oG*oG*oC*oC*dT*dG*dG*dG*dA*dC*dC*dT*dC*dA*oC*oU
*oG*oU*oC,
(SEQ ID NO: 331)
oU*oG*oC*oA*oC*dG*dT*dG*dT*dG*dG*dC*dT*dC*dA*oA*oG
*oC*oA*oG,
(SEQ ID NO: 332)
oC*oC*oA*oC*oU*dT*dC*dA*dG*dC*dT*dG*dT*dT*dT*oC*oA
*oU*oC*oC,
(SEQ ID NO: 333)
oG*oC*oG*oU*C*dA*dC*dC*dT*xdC*dG*dG*dC*dC*dT*oC*oA
*oG*oC*oC,
(SEQ ID NO: 334)
oA*oG*oC*oG*oU*dC*dA*dC*dC*dT*xdC*dG*dG*dC*dC*oU*o
C*oA*G*oC,
(SEQ ID NO: 335)
oC*oG*oU*oA*oG*dT*dT*dG*dA*dC*dT*dG*dG*xdC*dG*oA*o
A*oG*oU*oU,
(SEQ ID NO: 336)
oG*oG*oG*oC*oC*xdC*dG*dG*dA*dT*dC*dA*dC*dA*dG*oG*A
*oC*oU*oG,
(SEQ ID NO: 337)
oU*oU*oG*oC*oC*dC*dA*dT*dC*dC*dA*xdC*dG*dT*dC*A*oG
*oG*oG*oC,
(SEQ ID NO: 338)
oG*oG*oA*oC*oG*dG*dC*dC*xdC*dG*dG*dC*dT*dT*dG*oC*o
U*oG*oC*oC,
(SEQ ID NO: 339)
oU*G*oG*oA*oA*dC*dA*xdC*dG*dG*dA*xdC*dG*dG*dC*oC*o
C*oG*G*oC,
(SEQ ID NO: 340)
oC*oA*oU*oC*oC*dA*dA*dA*dA*xdC*dG*dT*dG*dG*dA*oU*o
U*oG*oG*oG,
and
(SEQ ID NO: 341)
oG*oC*oA*oU*oC*dC*dA*dA*dA*dA*xdC*dG*dT*dG*dG*oA*o
U*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

29. The oligonucleotide of embodiment 28, wherein the oligonucleotide is conjugated to an amine group at its 5′-end and comprises a structure selected from:

(SEQ ID NO: 302)
NH₂-(CH₂)₆-oC*oA*oU*oG*oG*dC*dA*dT*dA*dC*dA*dC*dC*dT*dG*oG*oC*oC*oC*oG,
(SEQ ID NO: 303)
NH₂-(CH₂)₆-oC*oA*oC*oC*oA*dA*dC*dA*xdC*dG*dT*dC*dC*dC*dT*oC*oU*oC*C*oU,
(SEQ ID NO: 304)
NH₂-(CH₂)₆-oU*oC*oA*oC*oC*dA*dA*dC*dA*xdC*dG*dT*dC*dC*dC*oU*oC*oU*oC*oC,
(SEQ ID NO: 305)
NH₂-(CH₂)₆-oC*oC*oA*oU*oU*dC*dA*dC*dC*dA*dA*dC*dA*xdC*dG*oU*oC*C*oC*oU,
(SEQ ID NO: 306)
NH₂-(CH₂)₆-oU*oA*oC*oA*oG*dG*dT*dA*dG*dT*dT*dC*dT*dC*dA*oU*oC*oC*oU*oG,
(SEQ ID NO: 307)
NH₂-(CH₂)₆-oG*oU*oA*oC*oA*dG*dG*dT*dA*dG*dT*dT*dC*dT*dC*oA*oU*oC*oC*oU,
(SEQ ID NO: 308)
NH₂-(CH₂)₆-oA*oC*oC*oA*oG*dG*dT*dA*dC*dA*dG*dG*dT*dA*dG*oU*oU*oC*oU*oC,
(SEQ ID NO: 309)
NH₂-(CH₂)₆-oG*oA*oC*oC*oA*dG*dG*dT*dA*dC*dA*dG*dG*dT*dA*oG*oU*oU*oC*oU,
(SEQ ID NO: 310)
NH₂-(CH₂)₆-oU*oG*oA*C*oC*dA*dG*dG*dT*dA*dC*dA*dG*dG*dT*oA*xoG*oU*oU*oC,
(SEQ ID NO: 311)
NH₂-(CH₂)₆-oC*oC*oC*oA*oA*dA*dC*dT*dT*dG*dC*dT*dC*dA*dG*oC*oA*oG*oU*oG,
(SEQ ID NO: 312)
NH₂-(CH₂)₆-oU*oG*oA*oC*oA*dA*dT*dC*dT*dC*xdC*dG*dC*dC*dA*oG*oG*oU*oA*oG,
(SEQ ID NO: 313)
NH₂-(CH₂)₆-oA*oU*oG*oA*oC*dA*dA*dT*dC*dT*dC*xdC*dG*dC*dC*oA*oG*oG*oU*oA,
(SEQ ID NO: 314)
NH₂-(CH₂)₆-oC*oA*oU*oG*oA*dC*dA*dA*dT*dC*dT*dC*xdC*dG*dC*oC*oA*oG*oG*oU,
(SEQ ID NO: 315)
NH₂-(CH₂)₆-oC*oC*oA*oU*oG*dA*dC*dA*dA*dT*dC*dT*dC*xdC*dG*oC*oC*oA*oG*oG,
(SEQ ID NO: 316)
NH₂-(CH₂)₆-oG*oC*oC*oA*oU*dG*dA*dC*dA*dA*dT*dC*dT*dC*xdC*oG*oC*oC*oA*oG,
(SEQ ID NO: 246)
NH₂-(CH₂)₆-oG*oG*oC*oC*oA*dT*dG*dA*dC*dA*dA*dT*dC*dT*dC*oC*oG*oC*oC*oA,
(SEQ ID NO: 317)
NH₂-(CH₂)₆-oU*oG*oG*oC*oC*dA*dT*dG*dA*dC*dA*dA*dT*dC*dT*oC*oC*oG*oC*oC,
(SEQ ID NO: 318)
NH₂-(CH₂)₆-oU*oG*oU*oG*oC*dA*xdC*dG*dT*dA*dG*dC*dC*dA*dA*oG*oC*oC*oG*oG,
(SEQ ID NO: 319)
NH₂-(CH₂)₆-oC*oU*oG*oU*oG*dC*dA*xdC*dG*dT*dA*dG*dC*dC*dA*oA*oG*oC*oC*oG,
(SEQ ID NO: 320)
NH₂-(CH₂)₆-oC*oA*oC*oA*oG*xdC*dG*dG*dT*dC*dC*dA*dG*dC*dA*oG*oG*oA*oU*oG,
(SEQ ID NO: 321)
NH₂-(CH₂)₆-oU*oG*oG*oC*oC*dA*dC*dA*dG*xdC*dG*dG*dT*dC*dC*oA*oG*oC*oA*oG,
(SEQ ID NO: 322)
NH₂-(CH₂)₆-oA*oG*oC*oG*oC*dC*dC*dA*dC*dC*dA*dG*dT*dC*dA*oC*oA*oC*oU*oC,
(SEQ ID NO: 323)
NH₂-(CH₂)₆-oC*oA*oG*oC*oG*dC*dC*dC*dA*dC*dC*dA*dG*dT*dC*oA*oC*oA*oC*oU,
(SEQ ID NO: 254)
NH₂-(CH₂)₆-oC*oC*oA*oG*oC*dG*dC*dC*dC*dA*dC*dC*dA*dG*dT*oC*oA*oC*oA*oC,
(SEQ ID NO: 255)
NH₂-(CH₂)₆-oG*oC*oG*oA*oA*dT*dA*dC*dA*dC*dC*dC*dA*dG*xdC*oG*oC*oC*oC*oA,
(SEQ ID NO: 256)
NH₂-(CH₂)₆-oG*oG*oC*oG*oA*dA*dT*dA*dC*dA*dC*dC*dC*dA*dG*oC*oG*oC*oC*oC,
(SEQ ID NO: 324)
NH₂-(CH₂)₆-oU*oU*oG*oU*oA*dG*dT*dG*dG*dA*xdC*dG*dA*dT*dC*oU*oU*oG*oC*oC,
(SEQ ID NO: 325)
NH₂-(CH₂)₆-oC*oU*oU*oG*oU*dA*dG*dT*dG*dG*dA*xdC*dG*dA*dT*oC*oU*oU*oG*oC,
(SEQ ID NO: 326)
NH₂-(CH₂)₆-oC*oC*oU*oU*oG*dT*dA*dG*dT*dG*dG*dA*xdC*dG*dA*oU*oC*oU*oU*oG,
(SEQ ID NO: 327)
NH₂-(CH₂)₆-oC*oG*oG*oA*oG*dA*dC*dC*dA*dT*dC*dC*dC*dA*dG*oU*oC*oG*oA*oG,
(SEQ ID NO: 328)
NH₂-(CH₂)₆-oG*oA*oA*oU*oG*dT*dC*xdC*dG*dA*dC*dA*dG*dT*dG*oU*oC*oU*oC*oC,
(SEQ ID NO: 329)
NH₂-(CH₂)₆-oC*oG*oA*oA*oU*dG*dT*dC*xdC*dG*dA*dC*dA*dG*dT*oG*oU*oC*oU*oC,
(SEQ ID NO: 330)
NH₂-(CH₂)₆-oG*oG*oG*oC*oC*dT*dG*dG*dG*dA*dC*dC*dT*dC*dA*oC*oU*oG*oU*oC,
(SEQ ID NO: 331)
NH₂-(CH₂)₆-oU*oG*oC*oA*oC*dG*dT*dG*dT*dG*dG*dC*dT*dC*dA*oA*oG*oC*oA*oG,
(SEQ ID NO: 332)
NH₂-(CH₂)₆-oC*oC*oA*oC*oU*dT*dC*dA*dG*dC*dT*dG*dT*dT*dT*oC*oA*oU*oC*oC,
(SEQ ID NO: 333)
NH₂-(CH₂)₆-oG*oC*oG*oU*oC*dA*dC*dC*dT*xdC*dG*dG*dC*dC*dT*oC*oA*oG*oC*oC,
(SEQ ID NO: 334)
NH₂-(CH₂)₆-oA*oG*oC*oG*oU*dC*dA*dC*dC*dT*xdC*dG*dG*dC*dC*oU*oC*oA*oG*oC,
(SEQ ID NO: 335)
NH₂-(CH₂)₆-oC*oG*oU*oA*oG*dT*dT*dG*dA*dC*dT*dG*dG*xdC*dG*oA*oA*oG*oU*oU,
(SEQ ID NO: 336)
NH₂-(CH₂)₆-oG*oG*oG*oC*C*xdC*dG*dG*dA*dT*dC*dA*dC*dA*dG*oG*oA*oC*oU*oG,
(SEQ ID NO: 337)
NH₂-(CH₂)₆-oU*oU*oG*oC*oC*dC*dA*dT*dC*dC*dA*xdC*dG*dT*dC*oA*oG*oG*oG*oC,
(SEQ ID NO: 338)
NH₂-(CH₂)₆-oG*oG*oA*oC*oG*dG*dC*dC*xdC*dG*dG*dC*dT*dT*dG*oC*oU*oG*oC*oC,
(SEQ ID NO: 339)
NH₂-(CH₂)₆-oU*oG*oG*oA*oA*dC*dA*xdC*dG*dG*dA*xdC*dG*dG*dC*oC*oC*oG*oG*oC,
(SEQ ID NO: 340)
NH₂-(CH₂)₆-oC*oA*oU*oC*oC*dA*dA*dA*dA*xdC*dG*dT*dG*dG*dA*oU*oU*oG*oG*oG,
and
(SEQ ID NO: 341)
NH₂-(CH₂)₆-oG*oC*oA*oU*oC*dC*dA*dA*dA*dA*xdC*dG*dT*dG*dG*oA*oU*oU*oG*oG,

wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage,
and optionally wherein a phosphodiester linkage or other moiety is present between the 5′-NH₂—(CH₂)₆— and the oligonucleotide.

30. An oligonucleotide comprising a structure selected from:

(SEQ ID NO: 275)
x+C*+A*xoC*oG*dT*dG*dT*dG*dG*xdC*dT*xdC*oA*oA*+G*x+C,
(SEQ ID NO: 275)
xoC*oA*x+C*+G*dT*dG*dT*dG*dG*xdC*dT*xdC*+A*+A*oG*xoC,
(SEQ ID NO: 276)
+G*x+C*oA*xoC*dG*dT*dG*dT*dG*dG*xdC*dT*xoC*oA*+A*+G,
(SEQ ID NO: 276)
oG*xoC*+A*x+C*dG*dT*dG*dT*dG*dG*xdC*dT*x+C*+A*oA*oG,
(SEQ ID NO: 342)
+A*x+C*oG*oU*G*dT*dG*dG*xdC*dT*xdC*dA*oA*oG*x+C*+A,
(SEQ ID NO: 342)
oA*xoC*+G*+U*dG*dT*dG*dG*xdC*dT*xdC*dA*+A*+G*xoC*oA,
(SEQ ID NO: 278)
x+C*+A*oA*oA*xdC*dT*dT*dG*xdC*dT*xdC*dA*oG*xoC*+A*+G,
(SEQ ID NO: 343)
+A*x+C*oU*oU*xdC*dA*dG*xdC*dT*dG*dT*dT*oU*xoC*+A*+U,
(SEQ ID NO: 344)
+U*+A*oG*oU*dT*dG*dA*xdC*dT*dG*dG*xdC*oG*oA*+A*+G,
(SEQ ID NO: 281)
+G*x+C*xoC*xoC*dG*dG*dA*dT*dC*dA*xdC*dA*oG*oG*+A*x+C,
(SEQ ID NO: 345)
x+C*+A*oU*oG*dA*xdC*dA*dA*dT*xdC*dT*xdC*xoC*oG*x+C*x+C,
(SEQ ID NO: 346)
+G*+U*xoC*oA*xdC*xdC*dT*xdC*dG*dG*xdC*dC*oU*xoC*+A*+G,
(SEQ ID NO: 347)
x+C*x+C*oA*oG*dG*dT*dA*xdC*dA*dG*dG*dT*oA*oG*+U*+U,
(SEQ ID NO: 348)
+A*x+C*xoC*oA*dA*xdC*dA*xdC*dG*dT*xdC*xdC*xoC*oU*x+C*+U,
(SEQ ID NO: 286)
x+C*x+C*oA*oA*dA*xdC*dT*dT*dG*xdC*dT*xdC*oA*oG*x+C*+A,
(SEQ ID NO: 349)
x+C*+A*xoC*oU*dT*xdC*dA*dG*xdC*dT*dG*dT*oU*oU*x+C*+A,
(SEQ ID NO: 350)
+G*+U*oA*oG*dT*dT*dG*dA*xdC*dT*dG*dG*xoC*oG*+A*+A,
(SEQ ID NO: 289)
+G*+G*xoC*xoC*xdC*dG*dG*dA*dT*xdC*dA*xdC*oA*oG*+G*+A,
(SEQ ID NO: 351)
+A*+A*A*xoC*T*dT*dG*xdC*dT*xdC*dA*dG*xoC*A*+G*+U,
(SEQ ID NO: 352)
x+C*+U*oU*xoC*dA*dG*xdC*dT*dG*dT*dT*dT*xoC*oA*+U*x+C,
(SEQ ID NO: 353)
+A*+G*oU*oU*dG*dA*xdC*dT*dG*dG*xdC*dG*oA*oA*+G*+U,
(SEQ ID NO: 354)
x+C*x+C*xoC*oG*dG*dA*dT*xdC*dA*xdC*dA*dG*oG*oA*x+C*+U,
(SEQ ID NO: 278)
XoC*oA*+A*+A*xdC*dT*dT*dG*xdC*dT*xdC*dA*+G*x+C*oA*oG,
(SEQ ID NO: 343)
oA*xoC*+U*+U*xdC*dA*dG*xdC*dT*dG*dT*dT*+U*x+C*oA*oU,
(SEQ ID NO: 344)
oU*oA*+G*+U*dT*dG*dA*xdC*dT*dG*dG*xdC*+G*+A*oA*oG,
(SEQ ID NO: 281)
oG*xoC*x+C*x+C*dG*dG*dA*dT*dC*dA*dC*dA*+G*+G*oA*xoC,
(SEQ ID NO: 286)
xoC*xoC*+A*+A*dA*xdC*dT*dT*dG*xdC*dT*xdC*+A*+G*xoC*oA,
(SEQ ID NO: 349)
xoC*oA*x+C*+U*dT*xdC*dA*dG*xdC*dT*dG*dT*+U*+U*xoC*oA,
(SEQ ID NO: 350)
oG*oU*+A*+G*dT*dT*dG*dA*xdC*dT*dG*dG*x+C*+G*oA*oA,
(SEQ ID NO: 289)
oG*oG*x+C*x+C*xdC*dG*dG*dA*dT*xdC*dA*xdC*+A*+G*oG*oA,
(SEQ ID NO: 351)
oA*oA*+A*x+C*dT*dT*dG*xdC*dT*xdC*dA*dG*x+C*+A*oG*oU,
(SEQ ID NO: 352)
xoC*oU*+U*x+C*dA*dG*xdC*dT*dG*dT*dT*dT*x+C*+A*oU*xoC,
(SEQ ID NO: 353)
oA*oG*+U*+U*dG*dA*xdC*dT*dG*dG*xdC*dG*+A*+A*oG*oU,
(SEQ ID NO: 354)
xoC*xoC*x+C*+G*dG*dA*dT*xdC*dA*xdC*dA*dG*+G*+A*xoC*oU,
(SEQ ID NO: 355)
x+C*x+C*oA*oU*dG*dA*xdC*dA*dA*dT*xdC*dT*xoC*xoC*+G*x+C,
(SEQ ID NO: 356)
+A*+U*oG*oA*xdC*dA*dA*dT*xdC*dT*xdC*xdC*oG*xoC*x+C*+A,
(SEQ ID NO: 357)
x+C*+G*oU*xoC*dA*xdC*xdC*dT*xdC*dG*dG*xdC*xoC*oU*x+C*+A,
(SEQ ID NO: 358)
+U*x+C*oA*xoC*xdC*dT*xdC*dG*dG*xdC*xdC*dT*xoC*oA*+G*x+C,
(SEQ ID NO: 359)
+A*x+C*xoC*oA*dG*dG*dT*dA*xdC*dA*dG*dG*oU*oA*+G*+U,
(SEQ ID NO: 360)
x+C*+A*oG*G*dT*dA*xdC*dA*dG*dG*dT*dA*oG*oU*+U*x+C,
(SEQ ID NO: 361)
x+C*+A*xoC*xoC*dA*dA*xdC*dA*xdC*dG*dT*xdC*xoC*xoC*+U*x+C,
and
(SEQ ID NO: 362)
x+C*x+C*oA*oA*xdC*dA*xdC*dG*dT*xdC*xdC*xdC*oU*xoC*+U*x+C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; “*” indicates a phosphorothioate (PS) internucleoside linkage.

31. The oligonucleotide of embodiment 30, wherein the oligonucleotide is conjugated to an amine group at its 5′-end and comprises a structure selected from:

(SEQ ID NO: 275)
NH₂-(CH₂)₆-x+C*+A*xoC*G*dT*dG*dT*dG*dG*xdC*dT*xdC*oA*oA*+G*x+C,
(SEQ ID NO: 275)
NH₂-(CH₂)₆-xoC*oA*x+C*+G*dT*dG*dT*dG*dG*xdC*dT*xdC*+A*+A*oG*xoC,
(SEQ ID NO: 276)
NH₂-(CH₂)₆-+G*x+C*oA*xoC*dG*dT*dG*dT*dG*dG*xdC*dT*xoC*oA*+A*+G,
(SEQ ID NO: 276)
NH₂-(CH₂)₆-oG*xoC*+A*x+C*dG*dT*dG*dT*dG*dG*xdC*dT*x+C*+A*oA*oG,
(SEQ ID NO: 342)
NH₂-(CH₂)₆-+A*x+C*oG*oU*dG*dT*dG*dG*xdC*dT*xdC*dA*oA*oG*x+C*+A,
(SEQ ID NO: 342)
NH₂-(CH₂)₆-oA*xoC*+G*+U*dG*dT*dG*dG*xdC*dT*xdC*dA*+A*+G*xoC*oA,
(SEQ ID NO: 278)
NH₂-(CH₂)₆-x+C*+A*oA*oA*xdC*dT*dT*dG*xdC*dT*xdC*dA*oG*xoC*+A*+G,
(SEQ ID NO: 343)
NH₂-(CH₂)₆-+A*x+C*oU*oU*xdC*dA*dG*xdC*dT*dG*dT*dT*oU*xoC*+A*+U,
(SEQ ID NO: 344)
NH₂-(CH₂)₆-+U*+A*oG*oU*dT*dG*dA*xdC*dT*dG*dG*xdC*oG*oA*+A*+G,
(SEQ ID NO: 281)
NH₂-(CH₂)₆-+G*x+C*xoC*xoC*dG*dG*dA*dT*dC*dA*xdC*dA*oG*oG*+A*x+C,
(SEQ ID NO: 345)
NH₂-(CH₂)₆-x+C*+A*oU*G*dA*xdC*dA*dA*dT*xdC*dT*xdC*xoC*oG*x+C*x+C,
(SEQ ID NO: 346)
NH₂-(CH₂)₆-+G*+U*xoC*oA*xdC*xdC*dT*xdC*dG*dG*xdC*dC*oU*xoC*+A*+G,
(SEQ ID NO: 347)
NH₂-(CH₂)₆-x+C*x+C*oA*oG*dG*dT*dA*xdC*dA*dG*dG*dT*oA*oG*+U*+U,
(SEQ ID NO: 348)
NH₂-(CH₂)₆-+A*x+C*xoC*oA*dA*xdC*dA*xdC*dG*dT*xdC*xdC*xoC*oU*x+C*+U,
(SEQ ID NO: 286)
NH₂-(CH₂)₆-x+C*x+C*oA*oA*dA*xdC*dT*dT*dG*xdC*dT*xdC*oA*oG*x+C*+A,
(SEQ ID NO: 349)
NH₂-(CH₂)₆-x+C*+A*xoC*oU*dT*xdC*dA*dG*xdC*dT*dG*dT*oU*oU*x+C*+A,
(SEQ ID NO: 350)
NH₂-(CH₂)₆-+G*+U*oA*G*dT*dT*dG*dA*xdC*dT*dG*dG*xoC*oG*+A*+A,
(SEQ ID NO: 289)
NH₂-(CH₂)₆-+G*+G*xoC*xoC*xdC*dG*dG*dA*dT*xdC*dA*xdC*oA*oG*+G*+A,
(SEQ ID NO: 351)
NH₂-(CH₂)₆-+A*+A*oA*xoC*dT*dT*dG*xdC*dT*xdC*dA*dG*xoC*oA*+G*+U,
(SEQ ID NO: 352)
NH₂-(CH₂)₆-x+C*+U*oU*xoC*dA*dG*xdC*dT*dG*dT*dT*dT*xoC*oA*+U*x+C,
(SEQ ID NO: 353)
NH₂-(CH₂)₆-+A*+G*oU*oU*dG*dA*xdC*dT*dG*dG*xdC*dG*oA*oA*+G*+U,
(SEQ ID NO: 354)
NH₂-(CH₂)₆-x+C*x+C*xoC*oG*dG*dA*dT*xdC*dA*xdC*dA*dG*oG*oA*x+C*+U,
(SEQ ID NO: 278)
NH₂-(CH₂)₆-xoC*oA*+A*+A*xdC*dT*dT*dG*xdC*dT*xdC*dA*+G*x+C*oA*oG,
(SEQ ID NO: 343)
NH₂-(CH₂)₆-oA*xoC*+U*+U*xdC*dA*dG*xdC*dT*dG*dT*dT*+U*x+C*oA*oU,
(SEQ ID NO: 344)
NH₂-(CH₂)₆-oU*oA*+G*+U*dT*dG*dA*xdC*dT*dG*dG*xdC*+G*+A*oA*oG,
(SEQ ID NO: 281)
NH₂-(CH₂)₆-oG*xoC*x+C*x+C*dG*dG*dA*dT*dC*dA*dC*dA*+G*+G*oA*xoC,
(SEQ ID NO: 286)
NH₂-(CH₂)₆-xoC*xoC*+A*+A*dA*xdC*dT*dT*dG*xdC*dT*xdC*+A*+G*xoC*oA,
(SEQ ID NO: 349)
NH₂-(CH₂)₆-xoC*oA*x+C*+U*dT*xdC*dA*dG*xdC*dT*dG*dT*+U*+U*xoC*oA,
(SEQ ID NO: 350)
NH₂-(CH₂)₆-oG*oU*+A*+G*dT*T*dG*dA*xdC*dT*dG*dG*x+C*+G*oA*oA,
(SEQ ID NO: 289)
NH₂-(CH₂)₆-oG*oG*x+C*x+C*xdC*dG*dG*dA*dT*xdC*dA*xdC*+A*+G*oG*oA,
(SEQ ID NO: 351)
NH₂-(CH₂)₆-oA*oA*+A*x+C*dT*dT*dG*xdC*dT*xdC*dA*dG*x+C*+A*oG*oU,
(SEQ ID NO: 352)
NH₂-(CH₂)₆-xoC*oU*+U*x+C*dA*dG*xdC*dT*dG*dT*dT*dT*x+C*+A*oU*xoC,
(SEQ ID NO: 353)
NH₂-(CH₂)₆-oA*oG*+U*+U*dG*dA*xdC*dT*dG*dG*xdC*dG*+A*+A*oG*oU,
(SEQ ID NO: 354)
NH₂-(CH₂)₆-xoC*xoC*x+C*+G*dG*dA*dT*xdC*dA*xdC*dA*dG*+G*+A*xoC*oU,
(SEQ ID NO: 355)
NH₂-(CH₂)₆-x+C*x+C*oA*oU*dG*dA*xdC*dA*dA*dT*xdC*dT*xoC*xoC*+G*x+C,
(SEQ ID NO: 356)
NH₂-(CH₂)₆-+A*+U*oG*oA*xdC*dA*dA*dT*xdC*dT*xdC*xdC*oG*xoC*x+C*+A,
(SEQ ID NO: 357)
NH₂-(CH₂)₆-x+C*+G*oU*xoC*dA*xdC*xdC*dT*xdC*dG*dG*xdC*xoC*oU*x+C*+A,
(SEQ ID NO: 358)
NH₂-(CH₂)₆-+U*x+C*oA*xoC*xdC*dT*xdC*dG*dG*xdC*xdC*dT*xoC*oA*+G*x+C,
(SEQ ID NO: 359)
NH₂-(CH₂)₆-+A*x+C*xoC*A*dG*dG*dT*dA*xdC*dA*dG*dG*oU*oA*+G*+U,
(SEQ ID NO: 360)
NH₂-(CH₂)₆-x+C*+A*oG*oG*dT*dA*xdC*dA*dG*dG*dT*dA*oG*oU*+U*x+C,
(SEQ ID NO: 361)
NH₂-(CH₂)₆-x+C*+A*xoC*xoC*dA*dA*xdC*dA*xdC*dG*dT*xdC*xoC*xoC*+U*x+C,
and
(SEQ ID NO: 362)
NH₂-(CH₂)₆-x+C*x+C*oA*oA*xdC*dA*xdC*dG*dT*xdC*xdC*xdC*oU*xoC*+U*x+C,

wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; “*” indicates a phosphorothioate (PS) internucleoside linkage,
and optionally wherein a phosphodiester linkage or other moiety is present between the 5′-NH₂—(CH₂)₆— and the oligonucleotide.

32. A composition comprising the oligonucleotide of any one of embodiments 28 to 31 in sodium salt form.

### EXAMPLES

### Example 1. In Vitro Activity of DMPK-Targeting Oligonucleotides (ASOs)

Gapmer antisense oligonucleotides (ASOs) for targeting DMPK were generated. Each individual oligonucleotide was evaluated for its ability to target DMPK in cells at two doses: 500 pM (low dose) and 50 nM (high dose).

Briefly, DM1 C15 immortalized myoblasts were cultured in T-75 flasks until near confluency (˜80% confluent). Myoblasts were then detached with trypsin and seeded into 96-well microplates at a density of 50,000 cells/well. Cells were allowed to recover overnight before the growth media was washed out and replaced with a no-serum media to induce differentiation into myotubes. Differentiation proceeded for seven days prior to treatment with DMPK-targeting oligonucleotides.

On day seven following induction of differentiation, DM1 C15 myotubes were transfected with an individual oligonucleotide using 0.3 μL of Lipofectamine MessengerMax per well. All oligonucleotides were tested at both 500 pM and 50 nM final concentrations in biological triplicates. After treatment with oligonucleotides, cells were incubated for 72 hours prior to being harvested for total RNA. cDNA was synthesized from the total RNA extracts and qPCR was performed to determine expression levels of DMPK in technical quadruplicate. All qPCR data were analyzed using a traditional 2^{-ΔΔCT} method and were normalized to a plate-based negative control that comprised cells treated with vehicle control (0.3 μL/well Lipofectamine MessengerMax without any oligonucleotide). Results from these experiments are shown in Table 11. ‘Transcript remaining’ for each antisense oligonucleotide in Table 11 refers to the expression level of DMPK in cells treated with the ASO relative to the expression in negative control vehicle-treated cells (wherein the expression level of the negative control was normalized to equal 1.00).

The majority of tested DMPK-targeting gapmer ASOs demonstrated a reduction in DMPK expression in differentiated myotubes at both the low and high dose concentrations tested. These data demonstrate that the ASOs shown in Table 9 are capable of targeting DMPK in cells, suggesting that muscle-targeting complexes comprising antisense oligonucleotides (e.g., a DMPK-targeting oligonucleotide provided herein) would be capable of targeting DMPK in muscle tissues in vivo.

TABLE 11
DMPK knockdown in CL5 cells.

Potency in CL5 Cells

| | 50 nM | 500 pM |
|---|---|---|
| ASO^{†} | Transcript remaining | Transcript remaining |
| ASO1 | 0.26 | 0.68 |
| ASO2 | 0.18 | 0.62 |
| ASO3 | 0.16 | 0.53 |
| ASO4 | 0.13 | 0.61 |
| ASO5 | 0.27 | 0.8 |
| ASO6 | 0.34 | 0.68 |
| ASO7 | 0.23 | 0.84 |
| ASO8 | 0.29 | 0.57 |
| ASO9 | 0.36 | 0.78 |
| ASO10 | 0.38 | 0.63 |
| ASO11 | 0.33 | 0.61 |
| ASO12 | 0.4 | 0.58 |
| ASO13 | 0.29 | 0.71 |
| ASO14 | 0.24 | 0.65 |
| ASO15 | 0.19 | 0.69 |
| ASO16 | 0.25 | 0.58 |
| ASO17 | 0.27 | 0.7 |
| ASO18 | 0.25 | 0.75 |
| ASO19 | 0.3 | 0.74 |
| ASO20 | 0.4 | 0.93 |
| ASO21 | 0.31 | 0.7 |
| ASO22 | 0.36 | 0.7 |
| ASO23 | 0.35 | 0.63 |
| ASO24 | 0.29 | 0.64 |
| ASO25 | 0.3 | 0.93 |
| ASO26 | 0.31 | 0.8 |
| ASO27 | 0.19 | 0.83 |
| ASO28 | 0.11 | 0.76 |
| ASO29 | 0.34 | 0.83 |
| ASO30 | 0.2 | 0.86 |
| ASO31 | 0.37 | 0.7 |
| ASO32 | 0.36 | 0.6 |
| ASO33 | 0.23 | 0.63 |
| ASO34 | 0.36 | 0.89 |
| ASO35 | 0.36 | 0.78 |
| ASO36 | 0.19 | 0.8 |
| ASO37 | 0.35 | 0.78 |
| ASO38 | 0.38 | 0.75 |
| ASO39 | 0.38 | 0.42 |
| ASO40 | 0.33 | 0.78 |
| ASO41 | 0.31 | 0.42 |
| ASO42 | 0.39 | 1.02 |
| ASO43 | 0.22 | 0.81 |
| ASO44 | 0.27 | 0.97 |

^{†}ASOs have the structures as shown in Table 9.

### Example 2. In Vivo Activity of Conjugates Containing Anti-TfR1 Fab Conjugated to DMPK-Targeting Oligonucleotide in Mice Expressing Human TfR1

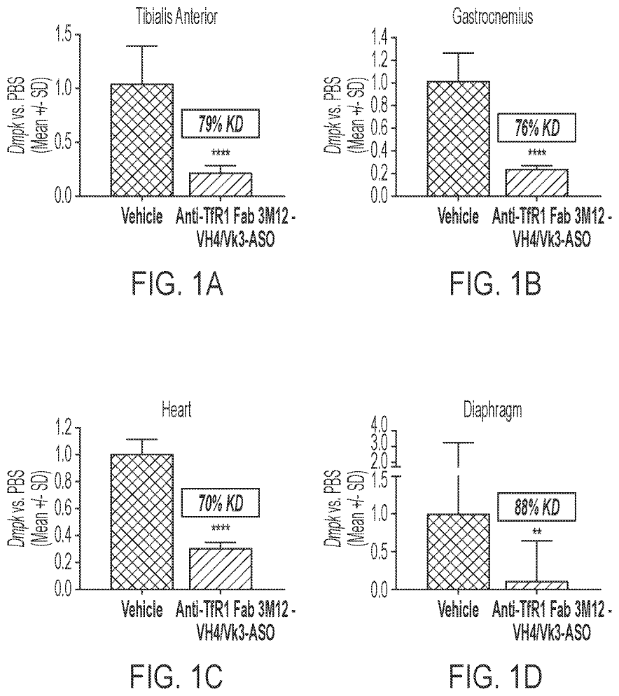

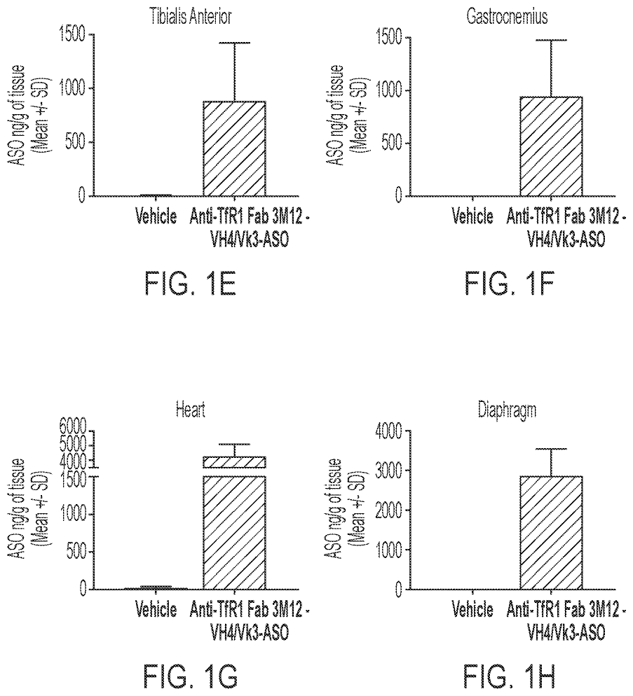

Conjugates containing anti-TfR1 Fab 3M12-VH4/Vκ3 conjugated to a DMPK-targeting oligonucleotide were tested in a mouse model that expresses human TfR1. The anti-TfR1 Fab 3M12-VH4/Vκ3 was covalently linked to a DMPK-targeting oligonucleotide via a cleavable linker having the structure of Formula (I). The conjugate was administered to the mice at a dose equivalent to 10 mg/kg oligonucleotide on day 0 and day 7. Mice were sacrificed on day 14 and different muscle tissues were collected and analyzed for mouse Dmpk mRNA level and oligonucleotide concentration in the tissue. The conjugate reduced mouse wild-type Dmpk in tibialis anterior by 79% (FIG. 1A), in gastrocnemius by 76% (FIG. 1B), in the heart by 70% (FIG. 1C), and in diaphragm by 88% (FIG. 1D). Oligonucleotide distributions in tibialis anterior, gastrocnemius, heart, and diaphragm are shown in FIGS. 1E-1H.

These data indicate that anti-TfR1 Fab 3M12-VH4/Vκ3 enabled cellular internalization of the conjugate into muscle tissues in an in vivo mouse model, thereby allowing the DMPK-targeting oligonucleotide to reduce expression of DMPK. Similarly, an anti-TfR1 antibody (e.g., anti-TfR1 Fab 3M12-VH4/Vκ3) can enable cellular internalization of a conjugate containing the anti-TfR1 antibody conjugated to another DMPK-targeting oligonucleotide (e.g., a DMPK-targeting oligonucleotide provided herein) for reducing expression of DMPK.

### Example 3. In Vitro Activity of Conjugates Containing Anti-TfR1 Fab Covalently Linked to DMPK-Targeting Antisense Oligonucleotides (ASOs)

In vitro experiments were conducted to determine the activities of DMPK-targeting antisense oligonucleotides (ASOs) listed in Table 9 in reducing DMPK mRNA expression in rhabdomyosarcoma cells (RD; ATCC, Manassas, VA) and DM1-32F primary cells expressing a mutant DMPK mRNA containing 380 CUG repeats (32F cells; Cook MyoSite, Pittsburgh, PA) and in correcting BIN1 Exon 11 splicing defect in DM1-32F cells. All ASOs were covalently linked to an anti-TfR1 Fab antibody (3M12-VH4/Vκ3) to form a complex comprising the structure of formula (E).

RD cells were expanded and seeded into 384-well plates at a density of 10,000 cells/well. Cells recovered overnight at 37° C. The next day, the media was changed, and cells were treated with 1,000 nM ASO equivalent of Fab-ASO complexes and allowed to incubate for 72 hours. After 72 hours, total RNA was extracted, and cDNA generated using a TaqMan Fast-Advanced Cells-to-Ct kit (ThermoFisher Scientific, Waltham, MA). cDNA was used to assess total DMPK knockdown using a specific TaqMan PCR assay (ThermoFisher Scientific). The data were normalized to PPIB expression and the 2^{-ΔΔCt} method was used to determine residual DMPK expression compared to vehicle-treated control cells (Table 12).

DM1 32F primary cells were thawed, allowed to recover, and then seeded at a density of 10,000 cells/well in 384-well plates in growth medium. The following day, the growth medium was changed to a low-serum differentiation medium and the cells were treated with either 10, 100, or 1,000 nM ASO equivalent of Fab-ASO complexes. The cells were incubated with the complexes for ten days, then total RNA was extracted, and cDNA generated using a TaqMan Fast-Advanced Cells-to-Ct kit.

cDNA was used to assess total DMPK knockdown using a specific TaqMan PCR assay. The data was normalized to PPIB expression and the 2^{-ΔΔct} method was used to determine DMPK knockdown compared to a vehicle only control. Data are presented as residual DMPK expression compared to a vehicle-treated control cell (Table 12). Additionally, modification of DM1-mediated aberrant splicing was evaluated using a multiplex TaqMan qPCR assay (ThermoFisher Scientific) to evaluate the aberrantly spliced and normal transcript. BIN1 transcripts which include exon 11 were measured because exclusion of exon 11 from BIN1 is associated with DM1. These data are presented as a mean ratio of aberrantly spliced to normal compared to vehicle-treated cells (Table 13). A ratio of 1 indicates that no change in aberrant splicing was observed when compared to DM1 patient myotubes treated with vehicle control. A ratio greater than 1 indicates that more transcripts had the wild-type splicing pattern. A ratio less than 1 indicates that more transcripts had the DM1-associated splicing pattern.

These data indicate that anti-TfR1 Fab 3M12-VH4/Vκ3 enabled cellular internalization of the Fab-ASO complex into cells, thereby allowing the DMPK-targeting ASO to reduce expression of DMPK mRNA and to facilitate correction of BIN1 Exon 11 splicing defect. Similarly, an anti-TfR1 antibody (e.g., anti-TfR1 Fab 3M12-VH4/Vκ3) can enable cellular internalization of a conjugate containing the anti-TfR1 antibody conjugated to another DMPK-targeting oligonucleotide (e.g., a DMPK-targeting oligonucleotide provided herein) for reducing expression of DMPK and facilitating downstream effects thereof (e.g., correction of DM1-associated splicing defects).

TABLE 12
DMPK mRNA levels in RD and 32F cells treated with
anti-TfR1 Fab-ASO complexes or vehicle control

| | RD Cells | | | |
|---|---|---|---|---|
| | DMPK expression | 32F Cells DMPK expression vs. Vehicle | | |
| | vs. Vehicle | 1000 nM | 100 nM | 10 nM |
| ASO^{†} | 1000 nM ASO | ASO | ASO | ASO |
| ASO1 | 0.72 | 0.18 | 0.76 | 1.12 |
| ASO2 | 0.86 | 0.23 | 0.70 | 0.80 |
| ASO3 | 0.77 | 0.18 | 0.53 | 0.82 |
| ASO4 | 0.84 | 0.20 | 0.78 | 0.77 |
| ASO5 | 0.63 | 0.27 | 0.74 | 0.84 |
| ASO6 | 0.56 | 0.40 | 0.58 | 0.58 |
| ASO7 | 0.56 | 0.26 | 0.63 | 0.69 |
| ASO8 | 0.59 | 0.19 | 0.47 | 0.67 |
| ASO9 | 0.61 | 0.32 | 0.61 | 0.76 |
| ASO10 | 0.64 | 0.21 | 0.47 | 0.55 |
| ASO11 | 0.87 | 0.40 | 0.69 | 0.74 |
| ASO12 | 0.75 | 0.23 | 0.72 | 0.76 |
| ASO13 | 0.79 | 0.30 | 0.67 | 0.76 |
| ASO14 | 0.75 | 0.16 | 0.55 | 0.59 |
| ASO15 | 0.73 | 0.14 | 0.55 | 0.64 |
| ASO16 | 0.67 | 0.18 | 0.45 | 0.61 |
| ASO17 | 0.83 | 0.32 | 0.54 | 0.76 |
| ASO18 | 0.77 | 0.59 | 0.56 | 0.65 |
| ASO19 | 0.74 | 0.24 | 0.66 | 0.77 |
| ASO20 | 0.69 | 0.14 | 0.53 | 0.62 |
| ASO21 | 0.86 | 0.15 | 0.54 | 0.74 |
| ASO22 | 0.72 | 0.17 | 0.47 | 0.56 |
| ASO23 | 0.67 | 0.11 | 0.58 | 0.69 |
| ASO24 | 0.68 | 0.10 | 0.44 | 0.62 |
| ASO25 | 0.77 | 0.16 | 0.56 | 0.75 |
| ASO26 | 0.76 | 0.51 | 0.60 | 0.67 |
| ASO27 | 0.68 | 0.29 | 0.73 | 0.76 |
| ASO28 | 0.71 | 0.24 | 0.62 | 0.72 |
| ASO29 | 0.77 | 0.23 | 0.75 | 0.78 |
| ASO30 | 0.76 | 0.37 | 0.68 | 0.74 |
| ASO31 | 0.76 | 0.19 | 0.58 | 0.74 |
| ASO32 | 0.71 | 0.17 | 0.49 | 0.69 |
| ASO33 | 0.87 | 0.10 | 0.71 | 0.83 |
| ASO34 | 0.57 | 0.12 | 0.37 | 0.56 |
| ASO35 | 0.63 | 0.25 | 0.56 | 0.67 |
| ASO36 | 0.64 | 0.15 | 0.57 | 0.67 |
| ASO37 | 0.84 | 0.21 | 0.68 | 0.84 |
| ASO38 | 0.64 | 0.21 | 0.57 | 0.72 |
| ASO39 | 0.77 | 0.12 | 0.65 | 0.69 |
| ASO40 | 0.81 | 0.50 | 0.65 | 0.89 |
| ASO41 | 0.97 | 0.42 | 0.82 | 0.89 |
| ASO42 | 0.82 | 0.15 | 0.69 | 0.84 |
| ASO43 | 0.82 | 0.25 | 0.70 | 1.02 |
| ASO44 | 0.86 | 0.12 | 0.68 | 0.82 |

TABLE 13
Correction of BIN1 splicing defect in 32F cells
treated with anti-TfR1 Fab-ASO complexes

| | 32F Cells BIN1 Exon 11 inclusion vs. Vehicle | | |
|---|---|---|---|
| ASO^{†} | 1000 nM ASO | 100 nM ASO | 10 nM ASO |
| ASO1 | 2.11 | 1.44 | 1.05 |
| ASO2 | 2.19 | 1.63 | 1.42 |
| ASO3 | 1.92 | 1.17 | 1.20 |
| ASO4 | 1.63 | 1.48 | 1.15 |
| ASO5 | 2.10 | 1.36 | 1.15 |
| ASO6 | 1.38 | 1.58 | 1.40 |
| ASO7 | 1.55 | 1.24 | 1.31 |
| ASO8 | 1.60 | 1.56 | 1.34 |
| ASO9 | 1.90 | 1.37 | 1.19 |
| ASO10 | 1.47 | 1.86 | 1.49 |
| ASO11 | 2.00 | 1.18 | 1.43 |
| ASO12 | 1.47 | 1.51 | 1.33 |
| ASO13 | 2.06 | 1.31 | 1.13 |
| ASO14 | 1.62 | 1.61 | 1.43 |
| ASO15 | 1.99 | 1.39 | 1.42 |
| ASO16 | 1.86 | 1.81 | 1.40 |
| ASO17 | 1.89 | 1.46 | 1.26 |
| ASO18 | 1.83 | 1.66 | 1.37 |
| ASO19 | 2.33 | 1.22 | 1.35 |
| ASO20 | 2.06 | 1.66 | 1.37 |
| ASO21 | 1.77 | 1.36 | 1.26 |
| ASO22 | 1.95 | 1.73 | 1.41 |
| ASO23 | 2.10 | 1.48 | 1.43 |
| ASO24 | 3.00 | 1.97 | 1.34 |
| ASO25 | 2.41 | 1.50 | 1.14 |
| ASO26 | 1.63 | 1.41 | 1.20 |
| ASO27 | 2.01 | 1.17 | 1.36 |
| ASO28 | 1.96 | 1.58 | 1.40 |
| ASO29 | 1.44 | 1.31 | 1.18 |
| ASO30 | 1.66 | 1.41 | 1.22 |
| ASO31 | 2.10 | 1.30 | 1.34 |
| ASO32 | 2.11 | 1.64 | 1.31 |
| ASO33 | 1.84 | 1.15 | 1.18 |
| ASO34 | 2.25 | 1.76 | 1.34 |
| ASO35 | 2.06 | 1.31 | 1.37 |
| ASO36 | 2.75 | 1.60 | 1.35 |
| ASO37 | 2.59 | 1.29 | 1.12 |
| ASO38 | 2.56 | 1.64 | 1.22 |
| ASO39 | 2.97 | 1.87 | 1.55 |
| ASO40 | 1.42 | 1.23 | 1.14 |
| ASO41 | 1.52 | 1.12 | 1.15 |
| ASO42 | 2.53 | 1.38 | 1.19 |
| ASO43 | 1.96 | 1.26 | 0.96 |
| ASO44 | 1.20 | 1.57 | 1.01 |

^{†}ASOs in Tables 12 and 13 have the structures as shown in Table 9.

### Example 4. In Vitro Activity of Conjugates Containing Anti-TfR1 Fab Covalently Linked to DMPK-Targeting Antisense Oligonucleotides (ASOs)

In vitro experiments were conducted to determine the activities of conjugates containing DMPK-targeting antisense oligonucleotides (ASOs) listed in Table 10 covalently linked to an anti-TfR1 Fab (3M12-VH4/Vκ3) in reducing DMPK mRNA expression in rhabdomyosarcoma cells (RD; ATCC, Manassas, VA) and DM1-32F primary cells expressing a mutant DMPK mRNA containing 380 CUG repeats (32F cells; Cook MyoSite, Pittsburgh, PA) and in correcting BIN1 Exon 11 splicing defect in DM1-32F cells. All ASOs were covalently linked to an anti-TfR1 Fab antibody (3M12-VH4/Vκ3) to form a complex comprising the structure of formula (E).

RD cells were expanded and seeded into 384-well plates at a density of 10,000 cells/well. Cells recovered overnight at 37° C. The next day, the media was changed, and cells were treated with 100 nM ASO equivalent of Fab-ASO complexes and allowed to incubate for 72 hours. After 72 hours, total RNA was extracted, and cDNA generated using a TaqMan Fast-Advanced Cells-to-Ct kit (ThermoFisher Scientific, Waltham, MA). cDNA was used to assess total DMPK knockdown using a specific TaqMan PCR assay (ThermoFisher Scientific). The data were normalized to PPIB expression and the 2^{-ΔΔCt} method was used to determine DMPK expression in conjugate-treated cells relative to vehicle-treated control cells (Table 14). Data are presented as knockdown percentages, where a higher positive value indicates greater knockdown of DMPK expression.

DM1 32F primary cells were thawed, allowed to recover, and then seeded at a density of 10,000 cells/well in 384-well plates in growth medium. The following day, the growth medium was changed to a low-serum differentiation medium and the cells were treated with either 10, 100, or 1,000 nM ASO equivalent of Fab-ASO complexes. The cells were incubated with the complexes for ten days, then total RNA was extracted, and cDNA generated using a TaqMan Fast-Advanced Cells-to-Ct kit.

cDNA was used to assess total DMPK knockdown using a specific TaqMan PCR assay. The data was normalized to PPIB expression and the 2^{-ΔΔCt} method was used to determine DMPK expression in conjugate-treated cells relative to vehicle-treated control cells. (Table 14). Data are presented as knockdown percentages, where a higher positive value indicates greater knockdown of DMPK expression, and negative values indicate no DMPK knockdown was detected in the conjugate-treated cells relative to the corresponding vehicle-treated control cells.

Additionally, modification of DM1-mediated aberrant splicing was evaluated using a multiplex TaqMan qPCR assay (ThermoFisher Scientific) to evaluate the aberrantly spliced and normal BIN1 transcript in DM1 32F primary cells treated with 100 nM ASO equivalent of Fab-ASO complexes. These data are presented as a mean ratio of aberrantly spliced to normal BIN1 in Fab-ASO complex-treated cells compared to vehicle-treated cells (Table 14). A ratio of 1 indicates that no change in aberrant splicing was observed when compared to DM1 patient myotubes treated with vehicle control. A ratio greater than 1 indicates that more transcripts had the wild-type splicing pattern in the cells treated with Fab-ASO complexes relative to cells treated with vehicle control. A ratio less than 1 would indicate that more transcripts had the DM1-associated splicing pattern.

All of the complexes tested achieved DMPK knockdown in at least one of the cell types tested, and all facilitated correction of DM1-mediated aberrant splicing to some extent. The complexes comprising ASO47, ASO55, ASO58, ASO61, ASO66, ASO71, ASO76, and ASO81 were among the best performing.

These data indicate that anti-TfR1 Fab 3M12-VH4/Vκ3 enabled cellular internalization of the Fab-ASO complex into cells, thereby allowing the DMPK-targeting ASO to reduce expression of DMPK mRNA and to facilitate correction of BIN1 Exon 11 splicing defect. Similarly, an anti-TfR1 antibody (e.g., anti-TfR1 Fab 3M12-VH4/Vκ3) can enable cellular internalization of a conjugate containing the anti-TfR1 antibody conjugated to another DMPK-targeting oligonucleotide (e.g., a DMPK-targeting oligonucleotide provided herein) for reducing expression of DMPK and facilitating downstream effects thereof (e.g., correction of DM1-associated splicing defects).

TABLE 14
DMPK knockdown (KD) and correction of BIN1 splicing
defect in RD and 32F cells treated with anti-
TfR1 Fab-ASO complexes or vehicle control

| | | | | | 32F Cells |
|---|---|---|---|---|---|
| | RD Cells | 32F Cells | | | BIN1 Exon 11 |
| | % DMPK KD | % DMPK KD vs. Vehicle | | | inclusion |
| | vs. Vehicle | 1000 nM | 100 nM | 10 nM | vs. Vehicle |
| ASO^{‡} | 100 nM ASO | ASO | ASO | ASO | 100 nM ASO |
| ASO46 | 41.60 | 89.87 | 65.36 | 11.78 | 3.76 |
| ASO47 | 34.78 | 80.33 | 54.14 | 15.81 | 2.89 |
| ASO51 | 37.60 | 43.40 | −28.00 | −62.20 | 2.10 |
| ASO52 | 40.80 | 65.29 | 24.40 | −29.40 | 1.88 |
| ASO53 | 34.47 | 70.43 | −32.10 | −49.60 | 2.67 |
| ASO55 | 33.98 | 83.69 | 23.04 | −7.90 | 2.64 |
| ASO56 | 34.74 | 66.30 | 8.88 | −59.90 | 1.99 |
| ASO57 | 36.61 | 44.69 | −49.70 | −56.90 | 1.81 |
| ASO58 | 34.49 | 88.17 | 31.39 | −8.70 | 2.84 |
| ASO59 | 36.95 | 13.34 | −21.70 | −23.20 | 2.20 |
| ASO60 | 46.90 | 94.91 | 46.75 | −6.40 | 2.58 |
| ASO61 | 24.95 | 62.54 | −9.30 | −24.40 | 2.59 |
| ASO62 | 34.70 | 55.34 | −12.50 | −41.00 | 3.14 |
| ASO63 | 25.03 | −12.70 | −40.40 | −45.20 | 2.04 |
| ASO64 | 46.09 | 62.62 | 24.94 | 4.31 | 2.24 |
| ASO65 | 24.39 | 43.68 | −36.10 | −39.00 | 1.98 |
| ASO66 | 33.09 | 77.42 | 10.42 | −14.20 | 3.91 |
| ASO67 | 23.93 | −8.10 | −40.10 | −23.40 | 1.78 |
| ASO68 | 45.84 | 51.87 | 29.95 | −24.70 | 2.16 |
| ASO69 | 26.02 | −0.90 | −13.30 | −20.00 | 2.15 |
| ASO71 | 34.49 | 16.07 | −9.80 | −14.20 | 2.56 |
| ASO72 | 40.07 | 74.84 | 38.06 | 6.43 | 2.19 |
| ASO73 | 31.34 | 19.53 | −1.70 | −7.60 | 2.61 |
| ASO74 | 33.57 | 43.95 | −16.60 | 0.00 | 2.11 |
| ASO75 | 30.30 | −17.40 | −48.50 | −42.50 | 1.91 |
| ASO76 | 44.42 | 61.32 | 30.99 | −3.70 | 2.25 |
| ASO77 | 29.74 | 12.56 | −23.00 | −46.40 | 1.92 |
| ASO78 | 27.06 | 68.05 | 1.06 | −7.40 | 2.75 |
| ASO79 | 27.89 | 53.43 | −5.80 | −15.00 | 2.60 |
| ASO80 | 32.07 | 28.15 | −11.30 | −16.40 | 2.13 |
| ASO81 | 24.22 | 77.30 | 16.34 | −48.30 | 3.36 |
| ASO82 | 21.60 | 60.64 | 2.16 | −42.50 | 2.14 |
| ASO83 | 21.66 | 41.81 | −45.90 | −66.20 | 1.93 |
| ASO84 | 28.64 | 9.13 | −23.10 | −49.20 | 1.82 |
| ASO85 | 20.62 | 53.14 | 24.08 | −26.50 | 2.26 |
| ASO86 | 27.95 | 47.30 | 23.21 | −9.50 | 2.21 |

^{‡}ASOs in Table 14 have the structures as shown in Table 10. ASOs that are listed in Table 10 and not shown in Table 14 were not tested in this experiment.

### Example 5. Knockdown Activity of DMPK-Targeting Oligonucleotides (ASOs) in hTfR1/DMSXL Hemizygous Mice

Conjugates containing anti-TfR1 Fab 3M12-VH4/Vκ3 covalently linked to a DMPK-targeting oligonucleotide (ASO58, ASO47, ASO61, or ASO66) were tested in a mouse that expresses both human TfR1 and a mutant human DMPK transgene that harbors expanded CTG repeats (hTfR1/DMSXL mice). The anti-TfR1 Fab was covalently linked to each ASO via a cleavable linker having the structure of Formula (I). Mice were administered either vehicle control (PBS) or 7.5 mg/kg (ASO58 conjugates), 8.8 mg/kg (ASO47 conjugates), 8.1 mg/kg (ASO61 conjugates), or 5.6 mg/kg (ASO66 conjugates) ASO-equivalent doses of anti-TfR1 Fab-ASO conjugates on days 0 and 7. Mice were sacrificed at day 14 (two weeks following administration of the first dose of conjugates), and tissues were collected. RNA was extracted and reverse transcription-quantitative polymerase chain reaction (RT-qPCR) of the RNA samples was performed to measure human DMPK and mouse Ppib (peptidylprolyl isomerase) as an internal control.

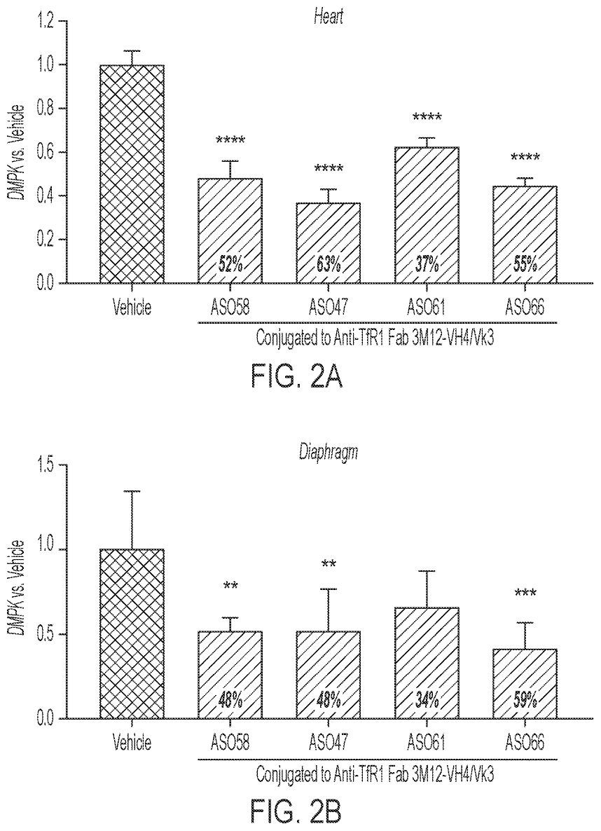

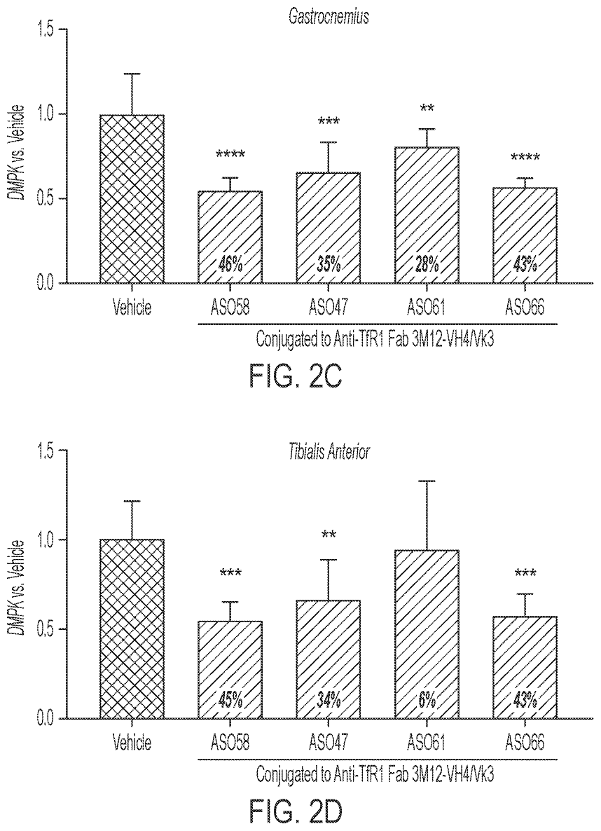

The conjugates reduced toxic human DMPK in heart by 37-63% (FIG. 2A), in diaphragm by 34-59% (FIG. 2B), in gastrocnemius by 28-46% (FIG. 2C), and in tibialis anterior by 6-45% (FIG. 2D).

These data indicate that anti-TfR1 Fab 3M12-VH4/Vκ3 enabled cellular internalization of the conjugate into muscle tissues in an in vivo mouse model, thereby allowing several DMPK-targeting oligonucleotides to reduce expression of toxic human DMPK.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides or nucleosides (e.g., an RNA counterpart of a DNA nucleoside or a DNA counterpart of an RNA nucleoside) and/or (e.g., and) one or more modified nucleotides/nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. A complex comprising an anti-transferrin receptor 1 (TfR1) antibody covalently linked to an oligonucleotide configured for reducing expression or activity of DMPK, wherein the anti-TfR1 antibody comprises a heavy chain complementarity determining region 1 (CDR-H1), a heavy chain complementarity determining region 2 (CDR-H2), and a heavy chain complementarity determining region 3 (CDR-H3) of a heavy chain variable region (VH) comprising the amino acid sequence SEQ ID NO: 72, and a light chain complementarity determining region 1 (CDR-L1), a light chain complementarity determining region 2 (CDR-L2), and a light chain complementarity determining region 3 (CDR-L3) of a light chain variable region (VL) comprising the amino acid sequence SEQ ID NO: 70,
and wherein the oligonucleotide comprises a 5′-X-Y-Z-3′ configuration, wherein
X comprises 3-7 linked nucleosides, wherein at least one of the nucleosides in X is a 2′-modified nucleoside;
Y comprises 6-15 linked 2′-deoxyribonucleosides, wherein each cytosine in Y is optionally and independently a 5-methyl-cytosine; and
Z comprises 3-7 linked nucleosides, wherein at least one of the nucleosides in Z is a 2′-modified nucleoside; and
wherein the oligonucleotide comprises a region of complementarity to at least 15 consecutive nucleosides of any one of SEQ ID NOs: 205, 214, 222, 217, 211, 215, 220, 225, 160-204, 206-210, 212, 213, 216, 218, 219, 221, 223, 224, and 226-230.

2. The complex of claim 1, wherein
X comprises 3-5 linked nucleosides, wherein at least one of the nucleosides in X is a 2′-modified nucleoside;
Y comprises 6-10 linked 2′-deoxyribonucleosides, wherein each cytosine in Y is optionally and independently a 5-methyl-cytosine; and
Z comprises 3-5 linked nucleosides, wherein at least one of the nucleosides in Z is a 2′-modified nucleoside.

3.-4. (canceled)

5. The complex of claim 1, wherein the antibody and the oligonucleotide are covalently linked via a cleavable linker, optionally wherein the cleavable linker comprises a valine-citrulline sequence.

6. The complex of claim 1, wherein the oligonucleotide is 15 to 25 nucleosides in length, optionally wherein the oligonucleotide is 15 to 20 nucleosides in length.

7. The complex of claim 1, wherein the oligonucleotide comprises at least 15 consecutive nucleosides of any one of SEQ ID NOs: 276, 348, 354, 350, 345, 286, 352, 357, 231-275, 277-285, 287-344, 346, 347, 349, 351, 353, 355, 356, and 358-362, wherein each thymine base (T) may independently and optionally be replaced with a uracil base (U), and each U may independently and optionally be replaced with a T.

8. The complex of claim 1, wherein each nucleoside in X is a 2′-modified nucleoside and/or each nucleoside in Z is a 2′-modified nucleoside, optionally wherein each 2′-modified nucleoside is independently a 2′-4′ bicyclic nucleoside or a non-bicyclic 2′-modified nucleoside.

9. The complex of claim 1, wherein the oligonucleotide comprises a 5′-X-Y-Z-3′ configuration of:

| X | Y | Z |
|---|---|---|
| EEEEE | (D)₁₀ | EEEEE, |
| EEF | (D)₁₀ | EEE, |
| EEEEE | (D)₁₀ | EEEE, |
| EEEEE | (D)₁₀ | EE, |
| LLL | (D)₁₀ | LLL, |
| EELL | (D)₈ | LLEE, |
| LLEE | (D)₈ | EELL, or |
| LLEEE | (D)₁₀ | EEELL, |

wherein “E” is a 2′-MOE modified ribonucleoside; “L” is LNA; “D” is 2′-deoxyribonucleoside; and “10” or “8” is the number of the 2′-deoxyribonucleosides in Y.

10. The complex of claim 1, wherein the oligonucleotide comprises one or more phosphorothioate internucleoside linkages, optionally wherein each internucleoside linkage in the oligonucleotide is a phosphorothioate internucleoside linkage.

11. (canceled)

12. The complex of claim 1, wherein the oligonucleotide comprises one or more phosphodiester internucleoside linkages, optionally wherein the one or more phosphodiester internucleoside linkages are in X and/or Z.

13. The complex of claim 1, wherein the oligonucleotide comprises a structure selected from:
(SEQ ID NO: 302)
oC*oA*oU*oG*oG*dC*dA*dT*dA*dC*dA*dC*dC*dT*dG*oG*oC*oC*oC*oG,
(SEQ ID NO: 303)
oC*oA*oC*oC*oA*dA*dC*dA*xdC*dG*dT*dC*dC*dC*dT*oC*oU*oC*C*oU,
(SEQ ID NO: 304)
oU*oC*oA*oC*oC*dA*dA*dC*dA*xdC*dG*dT*dC*dC*dC*oU*oC*oU*oC*oC,
(SEQ ID NO: 305)
oC*oC*oA*oU*oU*dC*dA*dC*dC*dA*dA*dC*dA*xdC*dG*oU*oC*C*oC*oU,
(SEQ ID NO: 306)
oU*oA*oC*oA*oG*dG*dT*dA*dG*dT*dT*dC*dT*dC*dA*oU*oC*oC*oU*oG,
(SEQ ID NO: 307)
oG*oU*oA*oC*oA*dG*dG*dT*dA*dG*dT*dT*dC*dT*dC*oA*oU*oC*oC*oU,
(SEQ ID NO: 308)
oA*oC*oC*oA*oG*dG*dT*dA*dC*dA*dG*dG*dT*dA*dG*oU*oU*oC*oU*oC,
(SEQ ID NO: 309)
oG*oA*oC*oC*oA*dG*dG*dT*dA*dC*dA*dG*dG*dT*dA*oG*oU*oU*oC*oU,
(SEQ ID NO: 310)
oU*oG*oA*C*oC*dA*dG*dG*dT*dA*dC*dA*dG*dG*dT*oA*xoG*oU*oU*oC,
(SEQ ID NO: 311)
oC*oC*oC*oA*oA*dA*dC*dT*dT*dG*dC*dT*dC*dA*dG*oC*oA*oG*oU*oG,
(SEQ ID NO: 312)
oU*oG*oA*oC*oA*dA*dT*dC*dT*dC*xdC*dG*dC*dC*dA*oG*oG*oU*oA*oG,
(SEQ ID NO: 313)
oA*oU*oG*oA*oC*dA*dA*dT*dC*dT*dC*xdC*dG*dC*dC*oA*oG*oG*oU*oA,
(SEQ ID NO: 314)
oC*oA*oU*oG*oA*dC*dA*dA*dT*dC*dT*dC*xdC*dG*dC*oC*oA*oG*oG*oU,
(SEQ ID NO: 315)
oC*oC*oA*oU*oG*dA*dC*dA*dA*dT*dC*dT*dC*xdC*dG*oC*oC*oA*oG*oG,
(SEQ ID NO: 316)
oG*oC*oC*oA*oU*dG*dA*dC*dA*dA*dT*dC*dT*dC*xdC*oG*oC*oC*oA*oG,
(SEQ ID NO: 246)
oG*oG*oC*oC*oA*dT*dG*dA*dC*dA*dA*dT*dC*dT*dC*oC*oG*oC*oC*oA,
(SEQ ID NO: 317)
oU*oG*oG*oC*oC*dA*dT*dG*dA*dC*dA*dA*dT*dC*dT*oC*oC*oG*oC*oC,
(SEQ ID NO: 318)
oU*oG*oU*oG*oC*dA*xdC*dG*dT*dA*dG*dC*dC*dA*dA*oG*oC*oC*oG*oG,
(SEQ ID NO: 319)
oC*oU*oG*oU*oG*dC*dA*xdC*dG*dT*dA*dG*dC*dC*dA*oA*oG*oC*oC*oG,
(SEQ ID NO: 320)
oC*oA*oC*oA*oG*xdC*dG*dG*dT*dC*dC*dA*dG*dC*dA*oG*oG*oA*oU*oG,
(SEQ ID NO: 321)
oU*oG*oG*oC*oC*dA*dC*dA*dG*xdC*dG*dG*dT*dC*dC*oA*oG*oC*oA*oG,
(SEQ ID NO: 322)
oA*oG*oC*oG*oC*dC*dC*dA*dC*dC*dA*dG*dT*dC*dA*oC*oA*oC*oU*oC,
(SEQ ID NO: 323)
oC*oA*oG*oC*oG*dC*dC*dC*dA*dC*dC*dA*dG*dT*dC*oA*oC*oA*oC*oU,
(SEQ ID NO: 254)
oC*oC*oA*oG*oC*dG*dC*dC*dC*dA*dC*dC*dA*dG*dT*oC*oA*oC*oA*oC,
(SEQ ID NO: 255)
oG*oC*oG*oA*oA*dT*dA*dC*dA*dC*dC*dC*dA*dG*xdC*oG*oC*oC*oC*oA,
(SEQ ID NO: 256)
oG*oG*oC*oG*oA*dA*dT*dA*dC*dA*dC*dC*dC*dA*dG*oC*oG*oC*oC*oC,
(SEQ ID NO: 324)
oU*oU*oG*oU*oA*dG*dT*dG*dG*dA*xdC*dG*dA*dT*dC*oU*oU*oG*oC*oC,
(SEQ ID NO: 325)
oC*oU*oU*oG*oU*dA*dG*dT*dG*dG*dA*xdC*dG*dA*dT*oC*oU*oU*oG*oC,
(SEQ ID NO: 326)
oC*oC*oU*oU*oG*dT*dA*dG*dT*dG*dG*dA*xdC*dG*dA*oU*oC*oU*oU*oG,
(SEQ ID NO: 327)
oC*oG*oG*oA*oG*dA*dC*dC*dA*dT*dC*dC*dC*dA*dG*oU*oC*oG*oA*oG,
(SEQ ID NO: 328)
oG*oA*oA*oU*oG*dT*dC*xdC*dG*dA*dC*dA*dG*dT*dG*oU*oC*oU*oC*oC,
(SEQ ID NO: 329)
*oG*oA*oA*oU*dG*dT*dC*xdC*dG*dA*dC*dA*dG*dT*oG*oU*oC*oU*oC,
(SEQ ID NO: 330)
oG*oG*oG*oC*oC*dT*dG*dG*dG*dA*dC*dC*dT*dC*dA*oC*oU*oG*oU*oC,
(SEQ ID NO: 331)
oU*oG*oC*oA*oC*dG*dT*dG*dT*dG*dG*dC*dT*dC*dA*oA*oG*oC*oA*oG,
(SEQ ID NO: 332)
oC*oC*oA*oC*oU*dT*dC*dA*dG*dC*dT*dG*dT*dT*dT*oC*oA*oU*oC*oC,
(SEQ ID NO: 333)
oG*oC*oG*oU*oC*dA*dC*dC*dT*xdC*dG*dG*dC*dC*dT*oC*oA*oG*oC*oC,
(SEQ ID NO: 334)
oA*oG*oC*oG*oU*dC*dA*dC*dC*dT*xdC*dG*dG*dC*dC*oU*oC*oA*oG*oC,
(SEQ ID NO: 335)
oC*oG*oU*oA*oG*dT*dT*dG*dA*dC*dT*dG*dG*xdC*dG*oA*oA*oG*oU*oU,
(SEQ ID NO: 336)
oG*oG*oG*oC*C*xdC*dG*dG*dA*dT*dC*dA*dC*dA*dG*oG*oA*oC*oU*oG,
(SEQ ID NO: 337)
-oU*oU*oG*oC*oC*dC*dA*dT*dC*dC*dA*xdC*dG*dT*dC*oA*oG*oG*oG*oC,
(SEQ ID NO: 338)
oG*oG*oA*oC*oG*dG*dC*dC*xdC*dG*dG*dC*dT*dT*dG*oC*oU*oG*oC*oC,
(SEQ ID NO: 339)
oU*oG*oG*oA*oA*dC*dA*xdC*dG*dG*dA*xdC*dG*dG*dC*oC*oC*oG*oG*oC,
(SEQ ID NO: 340)
oC*oA*oU*oC*oC*dA*dA*dA*dA*xdC*dG*dT*dG*dG*dA*oU*oU*oG*oG*oG,
and
(SEQ ID NO: 341)
oG*oC*oA*oU*oC*dC*dA*dA*dA*dA*xdC*dG*dT*dG*dG*oA*oU*oU*oG*oG,
wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

14. The complex of claim 13, wherein the oligonucleotide is conjugated to an amine group at its 5′-end and comprises a structure selected from:
(SEQ ID NO: 302)
NH₂-(CH₂)₆-oC*oA*oU*oG*oG*dC*dA*dT*dA*dC*dA*dC*dC*dT*dG*oG*oC*oC*oC*oG,
(SEQ ID NO: 303)
NH₂-(CH₂)₆-oC*oA*oC*oC*oA*dA*dC*dA*xdC*dG*dT*dC*dC*dC*dT*oC*oU*oC*C*oU,
(SEQ ID NO: 304)
NH₂-(CH₂)₆-oU*oC*oA*oC*oC*dA*dA*dC*dA*xdC*dG*dT*dC*dC*dC*oU*oC*oU*oC*oC,
(SEQ ID NO: 305)
NH₂-(CH₂)₆-oC*oC*oA*oU*oU*dC*dA*dC*dC*dA*dA*dC*dA*xdC*dG*oU*oC*C*oC*oU,
(SEQ ID NO: 306)
NH₂-(CH₂)₆-oU*oA*oC*oA*oG*dG*dT*dA*dG*dT*dT*dC*dT*dC*dA*oU*oC*oC*oU*oG,
(SEQ ID NO: 307)
NH₂-(CH₂)₆-oG*oU*oA*oC*oA*dG*dG*dT*dA*dG*dT*dT*dC*dT*dC*oA*oU*oC*oC*oU,
(SEQ ID NO: 308)
NH₂-(CH₂)₆-oA*oC*oC*oA*oG*dG*dT*dA*dC*dA*dG*dG*dT*dA*dG*oU*oU*oC*oU*oC,
(SEQ ID NO: 309)
NH₂-(CH₂)₆-oG*oA*oC*oC*oA*dG*dG*dT*dA*dC*dA*dG*dG*dT*dA*oG*oU*oU*oC*oU,
(SEQ ID NO: 310)
NH₂-(CH₂)₆-oU*oG*oA*C*oC*dA*dG*dG*dT*dA*dC*dA*dG*dG*dT*oA*xoG*oU*oU*oC,
(SEQ ID NO: 311)
NH₂-(CH₂)₆-oC*oC*oC*oA*oA*dA*dC*dT*dT*dG*dC*dT*dC*dA*dG*oC*oA*oG*oU*oG,
(SEQ ID NO: 312)
NH₂-(CH₂)₆-oU*oG*oA*oC*oA*dA*dT*dC*dT*dC*xdC*dG*dC*dC*dA*oG*oG*oU*oA*oG,
(SEQ ID NO: 313)
NH₂-(CH₂)₆-oA*oU*oG*oA*oC*dA*dA*dT*dC*dT*dC*xdC*dG*dC*dC*oA*oG*oG*oU*oA,
(SEQ ID NO: 314)
NH₂-(CH₂)₆-oC*oA*oU*oG*oA*dC*dA*dA*dT*dC*dT*dC*xdC*dG*dC*oC*oA*oG*oG*oU,
(SEQ ID NO: 315)
NH₂-(CH₂)₆-oC*oC*oA*oU*oG*dA*dC*dA*dA*dT*dC*dT*dC*xdC*dG*oC*oC*oA*oG*oG,
(SEQ ID NO: 316)
NH₂-(CH₂)₆-oG*oC*oC*oA*oU*dG*dA*dC*dA*dA*dT*dC*dT*dC*xdC*oG*oC*oC*oA*oG,
(SEQ ID NO: 246)
NH₂-(CH₂)₆-oG*oG*oC*oC*oA*dT*dG*dA*dC*dA*dA*dT*dC*dT*dC*oC*oG*oC*oC*oA,
(SEQ ID NO: 317)
NH₂-(CH₂)₆-oU*oG*oG*oC*oC*dA*dT*dG*dA*dC*dA*dA*dT*dC*dT*oC*oC*oG*oC*oC,
(SEQ ID NO: 318)
NH₂-(CH₂)₆-oU*oG*oU*oG*oC*dA*xdC*dG*dT*dA*dG*dC*dC*dA*dA*oG*oC*oC*oG*oG,
(SEQ ID NO: 319)
NH₂-(CH₂)₆-oC*oU*oG*oU*oG*dC*dA*xdC*dG*dT*dA*dG*dC*dC*dA*oA*oG*oC*oC*oG,
(SEQ ID NO: 320)
NH₂-(CH₂)₆-oC*oA*oC*oA*oG*xdC*dG*dG*dT*dC*dC*dA*dG*dC*dA*oG*oG*oA*oU*oG,
(SEQ ID NO: 321)
NH₂-(CH₂)₆-oU*oG*oG*oC*oC*dA*dC*dA*dG*xdC*dG*dG*dT*dC*dC*oA*oG*oC*oA*oG,
(SEQ ID NO: 322)
NH₂-(CH₂)₆-oA*oG*oC*oG*oC*dC*dC*dA*dC*dC*dA*dG*dT*dC*dA*oC*oA*oC*oU*oC,
(SEQ ID NO: 323)
NH₂-(CH₂)₆-oC*oA*oG*oC*oG*dC*dC*dC*dA*dC*dC*dA*dG*dT*dC*oA*oC*oA*oC*oU,
(SEQ ID NO: 254)
NH₂-(CH₂)₆-oC*oC*oA*oG*oC*dG*dC*dC*dC*dA*dC*dC*dA*dG*dT*oC*oA*oC*oA*oC,
(SEQ ID NO: 255)
NH₂-(CH₂)₆-oG*oC*oG*oA*oA*dT*dA*dC*dA*dC*dC*dC*dA*dG*xdC*oG*oC*oC*oC*oA,
(SEQ ID NO: 256)
NH₂-(CH₂)₆-oG*oG*oC*oG*oA*dA*dT*dA*dC*dA*dC*dC*dC*dA*dG*oC*oG*oC*oC*oC,
(SEQ ID NO: 324)
NH₂-(CH₂)₆-oU*oU*oG*oU*oA*dG*dT*dG*dG*dA*xdC*dG*dA*dT*dC*oU*oU*oG*oC*oC,
(SEQ ID NO: 325)
NH₂-(CH₂)₆-oC*oU*oU*oG*oU*dA*dG*dT*dG*dG*dA*xdC*dG*dA*dT*oC*oU*oU*oG*oC,
(SEQ ID NO: 326)
NH₂-(CH₂)₆-oC*oC*oU*oU*oG*dT*dA*dG*dT*dG*dG*dA*xdC*dG*dA*oU*oC*oU*oU*oG,
(SEQ ID NO: 327)
NH₂-(CH₂)₆-oC*oG*oG*oA*oG*dA*dC*dC*dA*dT*dC*dC*dC*dA*dG*oU*oC*oG*oA*oG,
(SEQ ID NO: 328)
NH₂-(CH₂)₆-oG*oA*oA*oU*oG*dT*dC*xdC*dG*dA*dC*dA*dG*dT*dG*oU*oC*oU*oC*oC,
(SEQ ID NO: 329)
NH₂-(CH₂)₆-oC*oG*oA*oA*oU*dG*dT*dC*xdC*dG*dA*dC*dA*dG*dT*oG*oU*oC*oU*oC,
(SEQ ID NO: 330)
NH₂-(CH₂)₆-oG*oG*oG*oC*oC*dT*dG*dG*dG*dA*dC*dC*dT*dC*dA*oC*oU*oG*oU*oC,
(SEQ ID NO: 331)
NH₂-(CH₂)₆-oU*oG*oC*oA*oC*dG*dT*dG*dT*dG*dG*dC*dT*dC*dA*oA*oG*oC*oA*oG,
(SEQ ID NO: 332)
NH₂-(CH₂)₆-oC*oC*oA*oC*oU*dT*dC*dA*dG*dC*dT*dG*dT*dT*dT*oC*oA*oU*oC*oC,
(SEQ ID NO: 333)
NH₂-(CH₂)₆-oG*oC*oG*oU*oC*dA*dC*dC*dT*xdC*dG*dG*dC*dC*dT*oC*oA*oG*oC*oC,
(SEQ ID NO: 334)
NH₂-(CH₂)₆-oA*oG*oC*oG*oU*dC*dA*dC*dC*dT*xdC*dG*dG*dC*dC*oU*oC*oA*oG*oC,
(SEQ ID NO: 335)
NH₂-(CH₂)₆-oC*oG*oU*oA*oG*dT*dT*dG*dA*dC*dT*dG*dG*xdC*dG*oA*oA*oG*oU*oU,
(SEQ ID NO: 336)
NH₂-(CH₂)₆-oG*oG*oG*oC*C*xdC*dG*dG*dA*dT*dC*dA*dC*dA*dG*oG*oA*oC*oU*oG,
(SEQ ID NO: 337)
NH₂-(CH₂)₆-oU*oU*oG*oC*oC*dC*dA*dT*dC*dC*dA*xdC*dG*dT*dC*oA*oG*oG*oG*oC,
(SEQ ID NO: 338)
NH₂-(CH₂)₆-oG*oG*oA*oC*oG*dG*dC*dC*xdC*dG*dG*dC*dT*dT*dG*oC*oU*oG*oC*oC,
(SEQ ID NO: 339)
NH₂-(CH₂)₆-oU*oG*oG*oA*oA*dC*dA*xdC*dG*dG*dA*xdC*dG*dG*dC*oC*oC*oG*oG*oC,
(SEQ ID NO: 340)
NH₂-(CH₂)₆-oC*oA*oU*oC*oC*dA*dA*dA*dA*xdC*dG*dT*dG*dG*dA*oU*oU*oG*oG*oG,
and
(SEQ ID NO: 341)
NH₂-(CH₂)₆-oG*oC*oA*oU*oC*dC*dA*dA*dA*dA*xdC*dG*dT*dG*dG*oA*oU*oU*oG*oG,
wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage,
and optionally wherein a phosphodiester linkage or other moiety is present between the 5′-NH₂—(CH₂)₆— and the oligonucleotide.

15. The complex of claim 1, wherein the oligonucleotide comprises a structure selected from:
(SEQ ID NO: 276)
+G*x+C*oA*xoC*dG*dT*dG*dT*dG*dG*xdC*dT*xoC*oA*+A*+G,
(SEQ ID NO: 348)
+A*x+C*xoC*oA*dA*xdC*dA*xdC*dG*dT*xdC*xdC*xoC*oU*x+C*+U,
(SEQ ID NO: 354)
x+C*x+C*xoC*oG*dG*dA*dT*xdC*dA*xdC*dA*dG*oG*oA*x+C*+U,
(SEQ ID NO: 350)
+G*+U*oA*oG*dT*dT*dG*dA*xdC*dT*dG*dG*xoC*oG*+A*+A,
(SEQ ID NO: 345)
x+C*+A*oU*oG*dA*xdC*dA*dA*dT*xdC*dT*xdC*xoC*oG*x+C*x+C,
(SEQ ID NO: 286)
xoC*xoC*+A*+A*dA*xdC*dT*dT*dG*xdC*dT*xdC*+A*+G*xoC*oA,
(SEQ ID NO: 352)
xoC*oU*+U*x+C*dA*dG*xdC*dT*dG*dT*dT*dT*x+C*+A*oU*xoC,
(SEQ ID NO: 357)
x+C*+G*oU*xoC*dA*xdC*xdC*dT*xdC*dG*dG*xdC*xoC*oU*x+C*+A,
(SEQ ID NO: 275)
x+C*+A*xoC*oG*dT*dG*dT*dG*dG*xdC*dT*xdC*oA*oA*+G*x+C,
(SEQ ID NO: 275)
xoC*oA*x+C*+G*dT*dG*dT*dG*dG*xdC*dT*xdC*+A*+A*oG*xoC,
(SEQ ID NO: 276)
oG*xoC*+A*x+C*dG*dT*dG*dT*dG*dG*xdC*dT*x+C*+A*oA*oG,
(SEQ ID NO: 342)
+A*x+C*oG*oU*dG*dT*dG*dG*xdC*dT*xdC*dA*oA*oG*x+C*+A,
(SEQ ID NO: 342)
oA*xoC*+G*+U*dG*dT*dG*dG*xdC*dT*xdC*dA*+A*+G*xoC*oA,
(SEQ ID NO: 278)
x+C*+A*oA*oA*xdC*dT*dT*dG*xdC*dT*xdC*dA*oG*xoC*+A*+G,
(SEQ ID NO: 343)
+A*x+C*oU*oU*xdC*dA*dG*xdC*dT*dG*dT*dT*oU*xoC*+A*+U,
(SEQ ID NO: 344)
+U*+A*oG*oU*dT*dG*dA*xdC*dT*dG*dG*xdC*oG*A*+A*+G,
(SEQ ID NO: 281)
+G*x+C*xoC*xoC*dG*dG*dA*dT*dC*dA*xdC*dA*oG*oG*+A*x+C,
(SEQ ID NO: 346)
+G*+U*xoC*oA*xdC*xdC*dT*xdC*dG*dG*xdC*dC*oU*xoC*+A*+G,
(SEQ ID NO: 347)
x+C*x+C*A*oG*dG*dT*dA*xdC*dA*dG*dG*dT*oA*oG*+U*+U,
(SEQ ID NO: 286)
x+C*x+C*oA*oA*dA*xdC*dT*dT*dG*xdC*dT*xdC*oA*oG*x+C*+A,
(SEQ ID NO: 349)
x+C*+A*xoC*oU*dT*xdC*dA*dG*xdC*dT*dG*dT*oU*oU*x+C*+A,
(SEQ ID NO: 289)
+G*+G*xoC*xoC*xdC*dG*dG*dA*dT*xdC*dA*xdC*oA*oG*+G*+A,
(SEQ ID NO: 351)
+A*+A*oA*xoC*dT*dT*dG*xdC*dT*xdC*dA*dG*xoC*oA*+G*+U,
(SEQ ID NO: 352)
x+C*+U*oU*xoC*dA*dG*xdC*dT*dG*dT*dT*dT*xoC*oA*+U*x+C,
(SEQ ID NO: 353)
+A*+G*oU*oU*dG*dA*xdC*dT*dG*dG*xdC*dG*oA*oA*+G*+U,
(SEQ ID NO: 278)
XoC*oA*+A*+A*xdC*dT*dT*dG*xdC*dT*xdC*dA*+G*x+C*oA*oG,
(SEQ ID NO: 343)
oA*xoC*+U*+U*xdC*dA*dG*xdC*dT*dG*dT*dT*+U*x+C*oA*oU,
(SEQ ID NO: 344)
oU*oA*+G*+U*dT*dG*dA*xdC*dT*dG*dG*xdC*+G*+A*oA*oG,
(SEQ ID NO: 281)
oG*xoC*x+C*x+C*dG*dG*dA*dT*dC*dA*dC*dA*+G*+G*oA*xoC,
(SEQ ID NO: 349)
xoC*oA*x+C*+U*dT*xdC*dA*dG*xdC*dT*dG*dT*+U*+U*xoC*oA,
(SEQ ID NO: 350)
oG*oU*+A*+G*dT*dT*dG*dA*xdC*dT*dG*dG*x+C*+G*oA*oA,
(SEQ ID NO: 289)
oG*oG*x+C*x+C*xdC*G*dG*dA*dT*xdC*dA*xdC*+A*+G*oG*oA,
(SEQ ID NO: 351)
oA*oA*+A*x+C*dT*dT*dG*xdC*dT*xdC*dA*dG*x+C*+A*oG*oU,
(SEQ ID NO: 353)
oA*oG*+U*+U*dG*dA*xdC*dT*dG*dG*xdC*dG*+A*+A*oG*oU,
(SEQ ID NO: 354)
xoC*xoC*x+C*+G*dG*dA*dT*xdC*dA*xdC*dA*dG*+G*+A*xoC*oU,
(SEQ ID NO: 355)
x+C*x+C*A*oU*dG*dA*xdC*dA*dA*dT*xdC*dT*xoC*xoC*+G*x+C,
(SEQ ID NO: 356)
+A*+U*G*oA*xdC*dA*dA*dT*xdC*dT*xdC*xdC*oG*xoC*x+C*+A,
(SEQ ID NO: 358)
+U*x+C*oA*xoC*xdC*dT*xdC*dG*dG*xdC*xdC*dT*xoC*oA*+G*x+C,
(SEQ ID NO: 359)
+A*x+C*xoC*oA*dG*dG*dT*dA*xdC*dA*dG*dG*oU*oA*+G*+U,
(SEQ ID NO: 360)
x+C*+A*oG*oG*dT*dA*xdC*dA*dG*dG*dT*dA*oG*oU*+U*x+C,
(SEQ ID NO: 361)
x+C*+A*xoC*xoC*dA*dA*xdC*dA*xdC*dG*dT*xdC*xoC*xoC*+U*x+C,
and
(SEQ ID NO: 362)
x+C*x+C*oA*oA*xdC*dA*xdC*dG*dT*xdC*xdC*xdC*oU*xoC*+U*x+C,
wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; and “*” indicates a phosphorothioate (PS) internucleoside linkage.

16. The complex of claim 15, wherein the oligonucleotide is conjugated to an amine group at its 5′-end and comprises a structure selected from:
(SEQ ID NO: 276)
NH₂-(CH₂)₆-+G*x+C*oA*xoC*dG*dT*dG*dT*dG*dG*xdC*dT*xoC*oA*+A*+G,
(SEQ ID NO: 348)
NH₂-(CH₂)₆-+A*x+C*xoC*oA*dA*xdC*dA*xdC*dG*dT*xdC*xdC*xoC*oU*x+C*+U,
(SEQ ID NO: 354)
NH₂-(CH₂)₆-x+C*x+C*xoC*oG*dG*dA*dT*xdC*dA*xdC*dA*dG*oG*oA*x+C*+U,
(SEQ ID NO: 350)
NH₂-(CH₂)₆-+G*+U*oA*oG*dT*dT*dG*dA*xdC*dT*dG*dG*xoC*oG*+A*+A,
(SEQ ID NO: 345)
NH₂-(CH₂)₆-x+C*+A*oU*oG*dA*xdC*dA*dA*dT*xdC*dT*xdC*xoC*oG*x+C*x+C,
(SEQ ID NO: 286)
NH₂-(CH₂)₆-xoC*xoC*+A*+A*dA*xdC*dT*dT*dG*xdC*dT*xdC*+A*+G*xoC*oA,
(SEQ ID NO: 352)
NH₂-(CH₂)₆-xoC*oU*+U*x+C*dA*dG*xdC*dT*dG*dT*dT*dT*x+C*+A*oU*xoC,
(SEQ ID NO: 357)
NH₂-(CH₂)₆-x+C*+G*oU*xoC*dA*xdC*xdC*dT*xdC*dG*dG*xdC*xoC*oU*x+C*+A,
(SEQ ID NO: 275)
NH₂-(CH₂)₆-x+C*+A*xoC*oG*dT*dG*dT*dG*dG*xdC*dT*xdC*oA*oA*+G*x+C,
(SEQ ID NO: 275)
NH₂-(CH₂)₆-xoC*oA*x+C*+G*dT*dG*dT*dG*dG*xdC*dT*xdC*+A*+A*oG*xoC,
(SEQ ID NO: 276)
NH₂-(CH₂)₆-oG*xoC*+A*x+C*dG*dT*dG*dT*dG*dG*xdC*dT*x+C*+A*oA*oG,
(SEQ ID NO: 342)
NH₂-(CH₂)₆-+A*x+C*oG*oU*dG*dT*dG*dG*xdC*dT*xdC*dA*oA*oG*x+C*+A,
(SEQ ID NO: 342)
NH₂-(CH₂)₆-oA*xoC*+G*+U*dG*dT*dG*dG*xdC*dT*xdC*dA*+A*+G*xoC*oA,
(SEQ ID NO: 278)
NH₂-(CH₂)₆-x+C*+A*oA*oA*xdC*dT*dT*dG*xdC*dT*xdC*dA*oG*xoC*+A*+G,
(SEQ ID NO: 343)
NH₂-(CH₂)₆-+A*x+C*oU*oU*xdC*dA*dG*xdC*dT*dG*dT*dT*oU*xoC*+A*+U,
(SEQ ID NO: 344)
NH₂-(CH₂)₆-+U*+A*oG*oU*dT*dG*dA*xdC*dT*dG*dG*xdC*oG*A*+A*+G,
(SEQ ID NO: 281)
NH₂-(CH₂)₆-+G*x+C*xoC*xoC*dG*dG*dA*dT*dC*dA*xdC*dA*oG*oG*+A*x+C,
(SEQ ID NO: 346)
NH₂-(CH₂)₆-+G*+U*xoC*oA*xdC*xdC*dT*xdC*dG*dG*xdC*dC*oU*xoC*+A*+G,
(SEQ ID NO: 347)
NH₂-(CH₂)₆-x+C*x+C*A*oG*dG*dT*dA*xdC*dA*dG*dG*dT*oA*oG*+U*+U,
(SEQ ID NO: 286)
NH₂-(CH₂)₆-x+C*x+C*oA*oA*dA*xdC*dT*dT*dG*xdC*dT*xdC*oA*oG*x+C*+A,
(SEQ ID NO: 349)
NH₂-(CH₂)₆-x+C*+A*xoC*oU*dT*xdC*dA*dG*xdC*dT*dG*dT*oU*oU*x+C*+A,
(SEQ ID NO: 289)
NH₂-(CH₂)₆-+G*+G*xoC*xoC*xdC*dG*dG*dA*dT*xdC*dA*xdC*oA*oG*+G*+A,
(SEQ ID NO: 351)
NH₂-(CH₂)₆-+A*+A*oA*xoC*dT*dT*dG*xdC*dT*xdC*dA*dG*xoC*oA*+G*+U,
(SEQ ID NO: 352)
NH₂-(CH₂)₆-x+C*+U*oU*xoC*dA*dG*xdC*dT*dG*dT*dT*dT*xoC*oA*+U*x+C,
(SEQ ID NO: 353)
NH₂-(CH₂)₆-+A*+G*oU*oU*dG*dA*xdC*dT*dG*dG*xdC*dG*oA*oA*+G*+U,
(SEQ ID NO: 278)
NH₂-(CH₂)₆-XoC*oA*+A*+A*xdC*dT*dT*dG*xdC*dT*xdC*dA*+G*x+C*oA*oG,
(SEQ ID NO: 343)
NH₂-(CH₂)₆-oA*xoC*+U*+U*xdC*dA*dG*xdC*dT*dG*dT*dT*+U*x+C*oA*oU,
(SEQ ID NO: 344)
NH₂-(CH₂)₆-oU*oA*+G*+U*dT*dG*dA*xdC*dT*dG*dG*xdC*+G*+A*oA*oG,
(SEQ ID NO: 281)
NH₂-(CH₂)₆-oG*xoC*x+C*x+C*dG*dG*dA*dT*dC*dA*dC*dA*+G*+G*oA*xoC,
(SEQ ID NO: 349)
NH₂-(CH₂)₆-xoC*oA*x+C*+U*dT*xdC*dA*dG*xdC*dT*dG*dT*+U*+U*xoC*oA,
(SEQ ID NO: 350)
NH₂-(CH₂)₆-oG*oU*+A*+G*dT*dT*dG*dA*xdC*dT*dG*dG*x+C*+G*oA*oA,
(SEQ ID NO: 289)
NH₂-(CH₂)₆-oG*oG*x+C*x+C*xdC*G*dG*dA*dT*xdC*dA*xdC*+A*+G*oG*oA,
(SEQ ID NO: 351)
NH₂-(CH₂)₆-oA*oA*+A*x+C*dT*dT*dG*xdC*dT*xdC*dA*dG*x+C*+A*oG*oU,
(SEQ ID NO: 353)
NH₂-(CH₂)₆-oA*oG*+U*+U*dG*dA*xdC*dT*dG*dG*xdC*dG*+A*+A*oG*oU,
(SEQ ID NO: 354)
NH₂-(CH₂)₆-xoC*xoC*x+C*+G*dG*dA*dT*xdC*dA*xdC*dA*dG*+G*+A*xoC*oU,
(SEQ ID NO: 355)
NH₂-(CH₂)₆-x+C*x+C*A*oU*dG*dA*xdC*dA*dA*dT*xdC*dT*xoC*xoC*+G*x+C,
(SEQ ID NO: 356)
NH₂-(CH₂)₆-+A*+U*G*oA*xdC*dA*dA*dT*xdC*dT*xdC*xdC*oG*xoC*x+C*+A,
(SEQ ID NO: 358)
NH₂-(CH₂)₆-+U*x+C*oA*xoC*xdC*dT*xdC*dG*dG*xdC*xdC*dT*xoC*oA*+G*x+C,
(SEQ ID NO: 359)
NH₂-(CH₂)₆-+A*x+C*xoC*oA*dG*dG*dT*dA*xdC*dA*dG*dG*oU*oA*+G*+U,
(SEQ ID NO: 360)
NH₂-(CH₂)₆-x+C*+A*oG*oG*dT*dA*xdC*dA*dG*dG*dT*dA*oG*oU*+U*x+C,
(SEQ ID NO: 361)
NH₂-(CH₂)₆-x+C*+A*xoC*xoC*dA*dA*xdC*dA*xdC*dG*dT*xdC*xoC*xoC*+U*x+C,
and
(SEQ ID NO: 362)
NH₂-(CH₂)₆-x+C*x+C*oA*oA*xdC*dA*xdC*dG*dT*xdC*xdC*xdC*oU*xoC*+U*x+C,
wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; and “*” indicates a phosphorothioate (PS) internucleoside linkage,
and optionally wherein a phosphodiester linkage or other moiety is present between the 5′-NH₂—(CH₂)₆— and the oligonucleotide.

17. A method of reducing DMPK expression in a muscle cell, the method comprising contacting the muscle cell with an effective amount of the complex of claim 1 to reduce DMPK expression in the muscle cell.

18.-19. (canceled)

20. A method of treating myotonic dystrophy type 1 (DM1), the method comprising administering to a subject in need thereof an effective amount of the complex of claim 1.

21.-22. (canceled)

23. An oligonucleotide comprising a structure selected from:
(SEQ ID NO: 302)
oC*oA*oU*oG*oG*dC*dA*dT*dA*dC*dA*dC*dC*dT*dG*oG*oC*oC*oC*oG,
(SEQ ID NO: 303)
oC*oA*oC*oC*oA*dA*dC*dA*xdC*dG*dT*dC*dC*dC*dT*oC*oU*oC*C*oU,
(SEQ ID NO: 304)
oU*oC*oA*oC*oC*dA*dA*dC*dA*xdC*dG*dT*dC*dC*dC*oU*oC*oU*oC*oC,
(SEQ ID NO: 305)
oC*oC*oA*oU*oU*dC*dA*dC*dC*dA*dA*dC*dA*xdC*dG*oU*oC*C*oC*oU,
(SEQ ID NO: 306)
oU*oA*oC*oA*oG*dG*dT*dA*dG*dT*dT*dC*dT*dC*dA*oU*oC*oC*oU*oG,
(SEQ ID NO: 307)
oG*oU*oA*oC*oA*dG*dG*dT*dA*dG*dT*dT*dC*dT*dC*oA*oU*oC*oC*oU,
(SEQ ID NO: 308)
oA*oC*oC*oA*oG*dG*dT*dA*dC*dA*dG*dG*dT*dA*dG*oU*oU*oC*oU*oC,
(SEQ ID NO: 309)
oG*oA*oC*oC*oA*dG*dG*dT*dA*dC*dA*dG*dG*dT*dA*oG*oU*oU*oC*oU,
(SEQ ID NO: 310)
oU*oG*oA*C*oC*dA*dG*dG*dT*dA*dC*dA*dG*dG*dT*oA*xoG*oU*oU*oC,
(SEQ ID NO: 311)
oC*oC*oC*oA*oA*dA*dC*dT*dT*dG*dC*dT*dC*dA*dG*oC*oA*oG*oU*oG,
(SEQ ID NO: 312)
oU*oG*oA*oC*oA*dA*dT*dC*dT*dC*xdC*dG*dC*dC*dA*oG*oG*oU*oA*oG,
(SEQ ID NO: 313)
oA*oU*oG*oA*oC*dA*dA*dT*dC*dT*dC*xdC*dG*dC*dC*oA*oG*oG*oU*oA,
(SEQ ID NO: 314)
oC*oA*oU*oG*oA*dC*dA*dA*dT*dC*dT*dC*xdC*dG*dC*oC*oA*oG*oG*oU,
(SEQ ID NO: 315)
oC*oC*oA*oU*oG*dA*dC*dA*dA*dT*dC*dT*dC*xdC*dG*oC*oC*oA*oG*oG,
(SEQ ID NO: 316)
oG*oC*oC*oA*oU*dG*dA*dC*dA*dA*dT*dC*dT*dC*xdC*oG*oC*oC*oA*oG,
(SEQ ID NO: 246)
oG*oG*oC*oC*oA*dT*dG*dA*dC*dA*dA*dT*dC*dT*dC*oC*oG*oC*oC*oA,
(SEQ ID NO: 317)
oU*oG*oG*oC*oC*dA*dT*dG*dA*dC*dA*dA*dT*dC*dT*oC*oC*oG*oC*oC,
(SEQ ID NO: 318)
oU*oG*oU*oG*oC*dA*xdC*dG*dT*dA*dG*dC*dC*dA*dA*oG*oC*oC*oG*oG,
(SEQ ID NO: 319)
oC*oU*oG*oU*oG*dC*dA*xdC*dG*dT*dA*dG*dC*dC*dA*oA*oG*oC*oC*oG,
(SEQ ID NO: 320)
oC*oA*oC*oA*oG*xdC*dG*dG*dT*dC*dC*dA*dG*dC*dA*oG*oG*oA*oU*oG,
(SEQ ID NO: 321)
oU*oG*oG*oC*oC*dA*dC*dA*dG*xdC*dG*dG*dT*dC*dC*oA*oG*oC*oA*oG,
(SEQ ID NO: 322)
oA*oG*oC*oG*oC*dC*dC*dA*dC*dC*dA*dG*dT*dC*dA*oC*oA*oC*oU*oC,
(SEQ ID NO: 323)
oC*oA*oG*oC*oG*dC*dC*dC*dA*dC*dC*dA*dG*dT*dC*oA*oC*oA*oC*oU,
(SEQ ID NO: 254)
oC*oC*oA*oG*oC*dG*dC*dC*dC*dA*dC*dC*dA*dG*dT*oC*oA*oC*oA*oC,
(SEQ ID NO: 255)
oG*oC*oG*oA*oA*dT*dA*dC*dA*dC*dC*dC*dA*dG*xdC*oG*oC*oC*oC*oA,
(SEQ ID NO: 256)
oG*oG*oC*oG*oA*dA*dT*dA*dC*dA*dC*dC*dC*dA*dG*oC*oG*oC*oC*oC,
(SEQ ID NO: 324)
oU*oU*oG*oU*oA*dG*dT*dG*dG*dA*xdC*dG*dA*dT*dC*oU*oU*oG*oC*oC,
(SEQ ID NO: 325)
oC*oU*oU*oG*oU*dA*dG*dT*dG*dG*dA*xdC*dG*dA*dT*oC*oU*oU*oG*oC,
(SEQ ID NO: 326)
oC*oC*oU*oU*oG*dT*dA*dG*dT*dG*dG*dA*xdC*dG*dA*oU*oC*oU*oU*oG,
(SEQ ID NO: 327)
oC*oG*oG*oA*oG*dA*dC*dC*dA*dT*dC*dC*dC*dA*dG*oU*oC*oG*oA*oG,
(SEQ ID NO: 328)
oG*oA*oA*oU*oG*dT*dC*xdC*dG*dA*dC*dA*dG*dT*dG*oU*oC*oU*oC*oC,
(SEQ ID NO: 329)
*oG*oA*oA*oU*dG*dT*dC*xdC*dG*dA*dC*dA*dG*dT*oG*oU*oC*oU*oC,
(SEQ ID NO: 330)
oG*oG*oG*oC*oC*dT*dG*dG*dG*dA*dC*dC*dT*dC*dA*oC*oU*oG*oU*oC,
(SEQ ID NO: 331)
oU*oG*oC*oA*oC*dG*dT*dG*dT*dG*dG*dC*dT*dC*dA*oA*oG*oC*oA*oG,
(SEQ ID NO: 332)
oC*oC*oA*oC*oU*dT*dC*dA*dG*dC*dT*dG*dT*dT*dT*oC*oA*oU*oC*oC,
(SEQ ID NO: 333)
oG*oC*oG*oU*oC*dA*dC*dC*dT*xdC*dG*dG*dC*dC*dT*oC*oA*oG*oC*oC,
(SEQ ID NO: 334)
oA*oG*oC*oG*oU*dC*dA*dC*dC*dT*xdC*dG*dG*dC*dC*oU*oC*oA*oG*oC,
(SEQ ID NO: 335)
oC*oG*oU*oA*oG*dT*dT*dG*dA*dC*dT*dG*dG*xdC*dG*oA*oA*oG*oU*oU,
(SEQ ID NO: 336)
oG*oG*oG*oC*C*xdC*dG*dG*dA*dT*dC*dA*dC*dA*dG*oG*oA*oC*oU*oG,
(SEQ ID NO: 337)
-oU*oU*oG*oC*oC*dC*dA*dT*dC*dC*dA*xdC*dG*dT*dC*oA*oG*oG*oG*oC,
(SEQ ID NO: 338)
oG*oG*oA*oC*oG*dG*dC*dC*xdC*dG*dG*dC*dT*dT*dG*oC*oU*oG*oC*oC,
(SEQ ID NO: 339)
oU*oG*oG*oA*oA*dC*dA*xdC*dG*dG*dA*xdC*dG*dG*dC*oC*oC*oG*oG*oC,
(SEQ ID NO: 340)
oC*oA*oU*oC*oC*dA*dA*dA*dA*xdC*dG*dT*dG*dG*dA*oU*oU*oG*oG*oG,
and
(SEQ ID NO: 341)
oG*oC*oA*oU*oC*dC*dA*dA*dA*dA*xdC*dG*dT*dG*dG*oA*oU*oU*oG*oG,
wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage.

24. The oligonucleotide of claim 23, wherein the oligonucleotide is conjugated to an amine group at its 5′-end and comprises a structure selected from:
(SEQ ID NO: 302)
NH₂-(CH₂)₆-oC*oA*oU*oG*oG*dC*dA*dT*dA*dC*dA*dC*dC*dT*dG*oG*oC*oC*oC*oG,
(SEQ ID NO: 303)
NH₂-(CH₂)₆-oC*oA*oC*oC*oA*dA*dC*dA*xdC*dG*dT*dC*dC*dC*dT*oC*oU*oC*C*oU,
(SEQ ID NO: 304)
NH₂-(CH₂)₆-oU*oC*oA*oC*oC*dA*dA*dC*dA*xdC*dG*dT*dC*dC*dC*oU*oC*oU*oC*oC,
(SEQ ID NO: 305)
NH₂-(CH₂)₆-oC*oC*oA*oU*oU*dC*dA*dC*dC*dA*dA*dC*dA*xdC*dG*oU*oC*C*oC*oU,
(SEQ ID NO: 306)
NH₂-(CH₂)₆-oU*oA*oC*oA*oG*dG*dT*dA*dG*dT*dT*dC*dT*dC*dA*oU*oC*oC*oU*oG,
(SEQ ID NO: 307)
NH₂-(CH₂)₆-oG*oU*oA*oC*oA*dG*dG*dT*dA*dG*dT*dT*dC*dT*dC*oA*oU*oC*oC*oU,
(SEQ ID NO: 308)
NH₂-(CH₂)₆-oA*oC*oC*oA*oG*dG*dT*dA*dC*dA*dG*dG*dT*dA*dG*oU*oU*oC*oU*oC,
(SEQ ID NO: 309)
NH₂-(CH₂)₆-oG*oA*oC*oC*oA*dG*dG*dT*dA*dC*dA*dG*dG*dT*dA*oG*oU*oU*oC*oU,
(SEQ ID NO: 310)
NH₂-(CH₂)₆-oU*oG*oA*C*oC*dA*dG*dG*dT*dA*dC*dA*dG*dG*dT*oA*xoG*oU*oU*oC,
(SEQ ID NO: 311)
NH₂-(CH₂)₆-oC*oC*oC*oA*oA*dA*dC*dT*dT*dG*dC*dT*dC*dA*dG*oC*oA*oG*oU*oG,
(SEQ ID NO: 312)
NH₂-(CH₂)₆-oU*oG*oA*oC*oA*dA*dT*dC*dT*dC*xdC*dG*dC*dC*dA*oG*oG*oU*oA*oG,
(SEQ ID NO: 313)
NH₂-(CH₂)₆-oA*oU*oG*oA*oC*dA*dA*dT*dC*dT*dC*xdC*dG*dC*dC*oA*oG*oG*oU*oA,
(SEQ ID NO: 314)
NH₂-(CH₂)₆-oC*oA*oU*oG*oA*dC*dA*dA*dT*dC*dT*dC*xdC*dG*dC*oC*oA*oG*oG*oU,
(SEQ ID NO: 315)
NH₂-(CH₂)₆-oC*oC*oA*oU*oG*dA*dC*dA*dA*dT*dC*dT*dC*xdC*dG*oC*oC*oA*oG*oG,
(SEQ ID NO: 316)
NH₂-(CH₂)₆-oG*oC*oC*oA*oU*dG*dA*dC*dA*dA*dT*dC*dT*dC*xdC*oG*oC*oC*oA*oG,
(SEQ ID NO: 246)
NH₂-(CH₂)₆-oG*oG*oC*oC*oA*dT*dG*dA*dC*dA*dA*dT*dC*dT*dC*oC*oG*oC*oC*oA,
(SEQ ID NO: 317)
NH₂-(CH₂)₆-oU*oG*oG*oC*oC*dA*dT*dG*dA*dC*dA*dA*dT*dC*dT*oC*oC*oG*oC*oC,
(SEQ ID NO: 318)
NH₂-(CH₂)₆-oU*oG*oU*oG*oC*dA*xdC*dG*dT*dA*dG*dC*dC*dA*dA*oG*oC*oC*oG*oG,
(SEQ ID NO: 319)
NH₂-(CH₂)₆-oC*oU*oG*oU*oG*dC*dA*xdC*dG*dT*dA*dG*dC*dC*dA*oA*oG*oC*oC*oG,
(SEQ ID NO: 320)
NH₂-(CH₂)₆-oC*oA*oC*oA*oG*xdC*dG*dG*dT*dC*dC*dA*dG*dC*dA*oG*oG*oA*oU*oG,
(SEQ ID NO: 321)
NH₂-(CH₂)₆-oU*oG*oG*oC*oC*dA*dC*dA*dG*xdC*dG*dG*dT*dC*dC*oA*oG*oC*oA*oG,
(SEQ ID NO: 322)
NH₂-(CH₂)₆-oA*oG*oC*oG*oC*dC*dC*dA*dC*dC*dA*dG*dT*dC*dA*oC*oA*oC*oU*oC,
(SEQ ID NO: 323)
NH₂-(CH₂)₆-oC*oA*oG*oC*oG*dC*dC*dC*dA*dC*dC*dA*dG*dT*dC*oA*oC*oA*oC*oU,
(SEQ ID NO: 254)
NH₂-(CH₂)₆-oC*oC*oA*oG*oC*dG*dC*dC*dC*dA*dC*dC*dA*dG*dT*oC*oA*oC*oA*oC,
(SEQ ID NO: 255)
NH₂-(CH₂)₆-oG*oC*oG*oA*oA*dT*dA*dC*dA*dC*dC*dC*dA*dG*xdC*oG*oC*oC*oC*oA,
(SEQ ID NO: 256)
NH₂-(CH₂)₆-oG*oG*oC*oG*oA*dA*dT*dA*dC*dA*dC*dC*dC*dA*dG*oC*oG*oC*oC*oC,
(SEQ ID NO: 324)
NH₂-(CH₂)₆-oU*oU*oG*oU*oA*dG*dT*dG*dG*dA*xdC*dG*dA*dT*dC*oU*oU*oG*oC*oC,
(SEQ ID NO: 325)
NH₂-(CH₂)₆-oC*oU*oU*oG*oU*dA*dG*dT*dG*dG*dA*xdC*dG*dA*dT*oC*oU*oU*oG*oC,
(SEQ ID NO: 326)
NH₂-(CH₂)₆-oC*oC*oU*oU*oG*dT*dA*dG*dT*dG*dG*dA*xdC*dG*dA*oU*oC*oU*oU*oG,
(SEQ ID NO: 327)
NH₂-(CH₂)₆-oC*oG*oG*oA*oG*dA*dC*dC*dA*dT*dC*dC*dC*dA*dG*oU*oC*oG*oA*oG,
(SEQ ID NO: 328)
NH₂-(CH₂)₆-oG*oA*oA*oU*oG*dT*dC*xdC*dG*dA*dC*dA*dG*dT*dG*oU*oC*oU*oC*oC,
(SEQ ID NO: 329)
NH₂-(CH₂)₆-oC*oG*oA*oA*oU*dG*dT*dC*xdC*dG*dA*dC*dA*dG*dT*oG*oU*oC*oU*oC,
(SEQ ID NO: 330)
NH₂-(CH₂)₆-oG*oG*oG*oC*oC*dT*dG*dG*dG*dA*dC*dC*dT*dC*dA*oC*oU*oG*oU*oC,
(SEQ ID NO: 331)
NH₂-(CH₂)₆-oU*oG*oC*oA*oC*dG*dT*dG*dT*dG*dG*dC*dT*dC*dA*oA*oG*oC*oA*oG,
(SEQ ID NO: 332)
NH₂-(CH₂)₆-oC*oC*oA*oC*oU*dT*dC*dA*dG*dC*dT*dG*dT*dT*dT*oC*oA*oU*oC*oC,
(SEQ ID NO: 333)
NH₂-(CH₂)₆-oG*oC*oG*oU*oC*dA*dC*dC*dT*xdC*dG*dG*dC*dC*dT*oC*oA*oG*oC*oC,
(SEQ ID NO: 334)
NH₂-(CH₂)₆-oA*oG*oC*oG*oU*dC*dA*dC*dC*dT*xdC*dG*dG*dC*dC*oU*oC*oA*oG*oC,
(SEQ ID NO: 335)
NH₂-(CH₂)₆-oC*oG*oU*oA*oG*dT*dT*dG*dA*dC*dT*dG*dG*xdC*dG*oA*oA*oG*oU*oU,
(SEQ ID NO: 336)
NH₂-(CH₂)₆-oG*oG*oG*oC*C*xdC*dG*dG*dA*dT*dC*dA*dC*dA*dG*oG*oA*oC*oU*oG,
(SEQ ID NO: 337)
NH₂-(CH₂)₆-oU*oU*oG*oC*oC*dC*dA*dT*dC*dC*dA*xdC*dG*dT*dC*oA*oG*oG*oG*oC,
(SEQ ID NO: 338)
NH₂-(CH₂)₆-oG*oG*oA*oC*oG*dG*dC*dC*xdC*dG*dG*dC*dT*dT*dG*oC*oU*oG*oC*oC,
(SEQ ID NO: 339)
NH₂-(CH₂)₆-oU*oG*oG*oA*oA*dC*dA*xdC*dG*dG*dA*xdC*dG*dG*dC*oC*oC*oG*oG*oC,
(SEQ ID NO: 340)
NH₂-(CH₂)₆-oC*oA*oU*oC*oC*dA*dA*dA*dA*xdC*dG*dT*dG*dG*dA*oU*oU*oG*oG*oG,
and
(SEQ ID NO: 341)
NH₂-(CH₂)₆-oG*oC*oA*oU*oC*dC*dA*dA*dA*dA*xdC*dG*dT*dG*dG*oA*oU*oU*oG*oG,
wherein “xdC” is 5-methyl-deoxycytidine; “dN” is 2′-deoxyribonucleoside; “oN” is 2′-MOE modified ribonucleoside; “oC” is 5-methyl-2′-MOE-cytidine; “oU” is 5-methyl-2′-MOE-uridine; “xoG” is 7-methyl-2′-MOE-guanosine; and “*” indicates a phosphorothioate (PS) internucleoside linkage,
and optionally wherein a phosphodiester linkage or other moiety is present between the 5′-NH₂—(CH₂)₆— and the oligonucleotide.

25. An oligonucleotide comprising a structure selected from:
(SEQ ID NO: 276)
+G*x+C*oA*xoC*dG*dT*dG*dT*dG*dG*xdC*dT*xoC*oA*+A*+G,
(SEQ ID NO: 348)
+A*x+C*xoC*oA*dA*xdC*dA*xdC*dG*dT*xdC*xdC*xoC*oU*x+C*+U,
(SEQ ID NO: 354)
x+C*x+C*xoC*oG*dG*dA*dT*xdC*dA*xdC*dA*dG*oG*oA*x+C*+U,
(SEQ ID NO: 350)
+G*+U*oA*oG*dT*dT*dG*dA*xdC*dT*dG*dG*xoC*oG*+A*+A,
(SEQ ID NO: 345)
x+C*+A*oU*oG*dA*xdC*dA*dA*dT*xdC*dT*xdC*xoC*oG*x+C*x+C,
(SEQ ID NO: 286)
xoC*xoC*+A*+A*dA*xdC*dT*dT*dG*xdC*dT*xdC*+A*+G*xoC*oA,
(SEQ ID NO: 352)
xoC*oU*+U*x+C*dA*dG*xdC*dT*dG*dT*dT*dT*x+C*+A*oU*xoC,
(SEQ ID NO: 357)
x+C*+G*oU*xoC*dA*xdC*xdC*dT*xdC*dG*dG*xdC*xoC*oU*x+C*+A,
(SEQ ID NO: 275)
x+C*+A*xoC*oG*dT*dG*dT*dG*dG*xdC*dT*xdC*oA*oA*+G*x+C,
(SEQ ID NO: 275)
xoC*oA*x+C*+G*dT*dG*dT*dG*dG*xdC*dT*xdC*+A*+A*oG*xoC,
(SEQ ID NO: 276)
oG*xoC*+A*x+C*dG*dT*dG*dT*dG*dG*xdC*dT*x+C*+A*oA*oG,
(SEQ ID NO: 342)
+A*x+C*oG*oU*dG*dT*dG*dG*xdC*dT*xdC*dA*oA*oG*x+C*+A,
(SEQ ID NO: 342)
oA*xoC*+G*+U*dG*dT*dG*dG*xdC*dT*xdC*dA*+A*+G*xoC*oA,
(SEQ ID NO: 278)
x+C*+A*oA*oA*xdC*dT*dT*dG*xdC*dT*xdC*dA*oG*xoC*+A*+G,
(SEQ ID NO: 343)
+A*x+C*oU*oU*xdC*dA*dG*xdC*dT*dG*dT*dT*oU*xoC*+A*+U,
(SEQ ID NO: 344)
+U*+A*oG*oU*dT*dG*dA*xdC*dT*dG*dG*xdC*oG*A*+A*+G,
(SEQ ID NO: 281)
+G*x+C*xoC*xoC*dG*dG*dA*dT*dC*dA*xdC*dA*oG*oG*+A*x+C,
(SEQ ID NO: 346)
+G*+U*xoC*oA*xdC*xdC*dT*xdC*dG*dG*xdC*dC*oU*xoC*+A*+G,
(SEQ ID NO: 347)
x+C*x+C*A*oG*dG*dT*dA*xdC*dA*dG*dG*dT*oA*oG*+U*+U,
(SEQ ID NO: 286)
x+C*x+C*oA*oA*dA*xdC*dT*dT*dG*xdC*dT*xdC*oA*oG*x+C*+A,
(SEQ ID NO: 349)
x+C*+A*xoC*oU*dT*xdC*dA*dG*xdC*dT*dG*dT*oU*oU*x+C*+A,
(SEQ ID NO: 289)
+G*+G*xoC*xoC*xdC*dG*dG*dA*dT*xdC*dA*xdC*oA*oG*+G*+A,
(SEQ ID NO: 351)
+A*+A*oA*xoC*dT*dT*dG*xdC*dT*xdC*dA*dG*xoC*oA*+G*+U,
(SEQ ID NO: 352)
x+C*+U*oU*xoC*dA*dG*xdC*dT*dG*dT*dT*dT*xoC*oA*+U*x+C,
(SEQ ID NO: 353)
+A*+G*oU*oU*dG*dA*xdC*dT*dG*dG*xdC*dG*oA*oA*+G*+U,
(SEQ ID NO: 278)
XoC*oA*+A*+A*xdC*dT*dT*dG*xdC*dT*xdC*dA*+G*x+C*oA*oG,
(SEQ ID NO: 343)
oA*xoC*+U*+U*xdC*dA*dG*xdC*dT*dG*dT*dT*+U*x+C*oA*oU,
(SEQ ID NO: 344)
oU*oA*+G*+U*dT*dG*dA*xdC*dT*dG*dG*xdC*+G*+A*oA*oG,
(SEQ ID NO: 281)
oG*xoC*x+C*x+C*dG*dG*dA*dT*dC*dA*dC*dA*+G*+G*oA*xoC,
(SEQ ID NO: 349)
xoC*oA*x+C*+U*dT*xdC*dA*dG*xdC*dT*dG*dT*+U*+U*xoC*oA,
(SEQ ID NO: 350)
oG*oU*+A*+G*dT*dT*dG*dA*xdC*dT*dG*dG*x+C*+G*oA*oA,
(SEQ ID NO: 289)
oG*oG*x+C*x+C*xdC*G*dG*dA*dT*xdC*dA*xdC*+A*+G*oG*oA,
(SEQ ID NO: 351)
oA*oA*+A*x+C*dT*dT*dG*xdC*dT*xdC*dA*dG*x+C*+A*oG*oU,
(SEQ ID NO: 353)
oA*oG*+U*+U*dG*dA*xdC*dT*dG*dG*xdC*dG*+A*+A*oG*oU,
(SEQ ID NO: 354)
xoC*xoC*x+C*+G*dG*dA*dT*xdC*dA*xdC*dA*dG*+G*+A*xoC*oU,
(SEQ ID NO: 355)
x+C*x+C*A*oU*dG*dA*xdC*dA*dA*dT*xdC*dT*xoC*xoC*+G*x+C,
(SEQ ID NO: 356)
+A*+U*G*oA*xdC*dA*dA*dT*xdC*dT*xdC*xdC*oG*xoC*x+C*+A,
(SEQ ID NO: 358)
+U*x+C*oA*xoC*xdC*dT*xdC*dG*dG*xdC*xdC*dT*xoC*oA*+G*x+C,
(SEQ ID NO: 359)
+A*x+C*xoC*oA*dG*dG*dT*dA*xdC*dA*dG*dG*oU*oA*+G*+U,
(SEQ ID NO: 360)
x+C*+A*oG*oG*dT*dA*xdC*dA*dG*dG*dT*dA*oG*oU*+U*x+C,
(SEQ ID NO: 361)
x+C*+A*xoC*xoC*dA*dA*xdC*dA*xdC*dG*dT*xdC*xoC*xoC*+U*x+C,
and
(SEQ ID NO: 362)
x+C*x+C*oA*oA*xdC*dA*xdC*dG*dT*xdC*xdC*xdC*oU*xoC*+U*x+C,
wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; “*” indicates a phosphorothioate (PS) internucleoside linkage.

26. The oligonucleotide of claim 25, wherein the oligonucleotide is conjugated to an amine group at its 5′-end and comprises a structure selected from:
(SEQ ID NO: 276)
NH₂-(CH₂)₆-+G*x+C*oA*xoC*dG*dT*dG*dT*dG*dG*xdC*dT*xoC*oA*+A*+G,
(SEQ ID NO: 348)
NH₂-(CH₂)₆-+A*x+C*xoC*oA*dA*xdC*dA*xdC*dG*dT*xdC*xdC*xoC*oU*x+C*+U,
(SEQ ID NO: 354)
NH₂-(CH₂)₆-x+C*x+C*xoC*oG*dG*dA*dT*xdC*dA*xdC*dA*dG*oG*oA*x+C*+U,
(SEQ ID NO: 350)
NH₂-(CH₂)₆-+G*+U*oA*oG*dT*dT*dG*dA*xdC*dT*dG*dG*xoC*oG*+A*+A,
(SEQ ID NO: 345)
NH₂-(CH₂)₆-x+C*+A*oU*oG*dA*xdC*dA*dA*dT*xdC*dT*xdC*xoC*oG*x+C*x+C,
(SEQ ID NO: 286)
NH₂-(CH₂)₆-xoC*xoC*+A*+A*dA*xdC*dT*dT*dG*xdC*dT*xdC*+A*+G*xoC*oA,
(SEQ ID NO: 352)
NH₂-(CH₂)₆-xoC*oU*+U*x+C*dA*dG*xdC*dT*dG*dT*dT*dT*x+C*+A*oU*xoC,
(SEQ ID NO: 357)
NH₂-(CH₂)₆-x+C*+G*oU*xoC*dA*xdC*xdC*dT*xdC*dG*dG*xdC*xoC*oU*x+C*+A,
(SEQ ID NO: 275)
NH₂-(CH₂)₆-x+C*+A*xoC*oG*dT*dG*dT*dG*dG*xdC*dT*xdC*oA*oA*+G*x+C,
(SEQ ID NO: 275)
NH₂-(CH₂)₆-xoC*oA*x+C*+G*dT*dG*dT*dG*dG*xdC*dT*xdC*+A*+A*oG*xoC,
(SEQ ID NO: 276)
NH₂-(CH₂)₆-oG*xoC*+A*x+C*dG*dT*dG*dT*dG*dG*xdC*dT*x+C*+A*oA*oG,
(SEQ ID NO: 342)
NH₂-(CH₂)₆-+A*x+C*oG*oU*dG*dT*dG*dG*xdC*dT*xdC*dA*oA*oG*x+C*+A,
(SEQ ID NO: 342)
NH₂-(CH₂)₆-oA*xoC*+G*+U*dG*dT*dG*dG*xdC*dT*xdC*dA*+A*+G*xoC*oA,
(SEQ ID NO: 278)
NH₂-(CH₂)₆-x+C*+A*oA*oA*xdC*dT*dT*dG*xdC*dT*xdC*dA*oG*xoC*+A*+G,
(SEQ ID NO: 343)
NH₂-(CH₂)₆-+A*x+C*oU*oU*xdC*dA*dG*xdC*dT*dG*dT*dT*oU*xoC*+A*+U,
(SEQ ID NO: 344)
NH₂-(CH₂)₆-+U*+A*oG*oU*dT*dG*dA*xdC*dT*dG*dG*xdC*oG*A*+A*+G,
(SEQ ID NO: 281)
NH₂-(CH₂)₆-+G*x+C*xoC*xoC*dG*dG*dA*dT*dC*dA*xdC*dA*oG*oG*+A*x+C,
(SEQ ID NO: 346)
NH₂-(CH₂)₆-+G*+U*xoC*oA*xdC*xdC*dT*xdC*dG*dG*xdC*dC*oU*xoC*+A*+G,
(SEQ ID NO: 347)
NH₂-(CH₂)₆-x+C*x+C*A*oG*dG*dT*dA*xdC*dA*dG*dG*dT*oA*oG*+U*+U,
(SEQ ID NO: 286)
NH₂-(CH₂)₆-x+C*x+C*oA*oA*dA*xdC*dT*dT*dG*xdC*dT*xdC*oA*oG*x+C*+A,
(SEQ ID NO: 349)
NH₂-(CH₂)₆-x+C*+A*xoC*oU*dT*xdC*dA*dG*xdC*dT*dG*dT*oU*oU*x+C*+A,
(SEQ ID NO: 289)
NH₂-(CH₂)₆-+G*+G*xoC*xoC*xdC*dG*dG*dA*dT*xdC*dA*xdC*oA*oG*+G*+A,
(SEQ ID NO: 351)
NH₂-(CH₂)₆-+A*+A*oA*xoC*dT*dT*dG*xdC*dT*xdC*dA*dG*xoC*oA*+G*+U,
(SEQ ID NO: 352)
NH₂-(CH₂)₆-x+C*+U*oU*xoC*dA*dG*xdC*dT*dG*dT*dT*dT*xoC*oA*+U*x+C,
(SEQ ID NO: 353)
NH₂-(CH₂)₆-+A*+G*oU*oU*dG*dA*xdC*dT*dG*dG*xdC*dG*oA*oA*+G*+U,
(SEQ ID NO: 278)
NH₂-(CH₂)₆-XoC*oA*+A*+A*xdC*dT*dT*dG*xdC*dT*xdC*dA*+G*x+C*oA*oG,
(SEQ ID NO: 343)
NH₂-(CH₂)₆-oA*xoC*+U*+U*xdC*dA*dG*xdC*dT*dG*dT*dT*+U*x+C*oA*oU,
(SEQ ID NO: 344)
NH₂-(CH₂)₆-oU*oA*+G*+U*dT*dG*dA*xdC*dT*dG*dG*xdC*+G*+A*oA*oG,
(SEQ ID NO: 281)
NH₂-(CH₂)₆-oG*xoC*x+C*x+C*dG*dG*dA*dT*dC*dA*dC*dA*+G*+G*oA*xoC,
(SEQ ID NO: 349)
NH₂-(CH₂)₆-xoC*oA*x+C*+U*dT*xdC*dA*dG*xdC*dT*dG*dT*+U*+U*xoC*oA,
(SEQ ID NO: 350)
NH₂-(CH₂)₆-oG*oU*+A*+G*dT*dT*dG*dA*xdC*dT*dG*dG*x+C*+G*oA*oA,
(SEQ ID NO: 289)
NH₂-(CH₂)₆-oG*oG*x+C*x+C*xdC*G*dG*dA*dT*xdC*dA*xdC*+A*+G*oG*oA,
(SEQ ID NO: 351)
NH₂-(CH₂)₆-oA*oA*+A*x+C*dT*dT*dG*xdC*dT*xdC*dA*dG*x+C*+A*oG*oU,
(SEQ ID NO: 353)
NH₂-(CH₂)₆-oA*oG*+U*+U*dG*dA*xdC*dT*dG*dG*xdC*dG*+A*+A*oG*oU,
(SEQ ID NO: 354)
NH₂-(CH₂)₆-xoC*xoC*x+C*+G*dG*dA*dT*xdC*dA*xdC*dA*dG*+G*+A*xoC*oU,
(SEQ ID NO: 355)
NH₂-(CH₂)₆-x+C*x+C*A*oU*dG*dA*xdC*dA*dA*dT*xdC*dT*xoC*xoC*+G*x+C,
(SEQ ID NO: 356)
NH₂-(CH₂)₆-+A*+U*G*oA*xdC*dA*dA*dT*xdC*dT*xdC*xdC*oG*xoC*x+C*+A,
(SEQ ID NO: 358)
NH₂-(CH₂)₆-+U*x+C*oA*xoC*xdC*dT*xdC*dG*dG*xdC*xdC*dT*xoC*oA*+G*x+C,
(SEQ ID NO: 359)
NH₂-(CH₂)₆-+A*x+C*xoC*oA*dG*dG*dT*dA*xdC*dA*dG*dG*oU*oA*+G*+U,
(SEQ ID NO: 360)
NH₂-(CH₂)₆-x+C*+A*oG*oG*dT*dA*xdC*dA*dG*dG*dT*dA*oG*oU*+U*x+C,
(SEQ ID NO: 361)
NH₂-(CH₂)₆-x+C*+A*xoC*xoC*dA*dA*xdC*dA*xdC*dG*dT*xdC*xoC*xoC*+U*x+C,
and
(SEQ ID NO: 362)
NH₂-(CH₂)₆-x+C*x+C*oA*oA*xdC*dA*xdC*dG*dT*xdC*xdC*xdC*oU*xoC*+U*x+C,
wherein “xdC” is 5-methyl-deoxycytidine; “dN” is 2′-deoxyribonucleoside; “oN” is 2′-MOE modified ribonucleoside; “xoC” is 5-methyl-2′-MOE-cytidine; “x+C” is 5-methyl LNA cytidine; “+N” is an LNA nucleoside; “oU” is 5-methyl-2′-MOE-uridine; “+U” is 5-methyl LNA uridine; “*” indicates a phosphorothioate (PS) internucleoside linkage,
and optionally wherein a phosphodiester linkage or other moiety is present between the 5′-NH₂—(CH₂)₆— and the oligonucleotide.

27. A composition comprising the oligonucleotide of claim 23 in sodium salt form.


SEQUENCE LISTING (395 sequences)

SEQ ID NO: 1 - protein, 8 aa, synthetic construct
  REGION 1..8: note = Synthetic
GFNIKDDY

SEQ ID NO: 2 - protein, 8 aa, synthetic construct
  REGION 1..8: note = Synthetic
IDPENGDT

SEQ ID NO: 3 - protein, 10 aa, synthetic construct
  REGION 1..10: note = Synthetic
TLWLRRGLDY

SEQ ID NO: 4 - protein, 11 aa, synthetic construct
  REGION 1..11: note = Synthetic
KSLLHSNGYTY

SEQ ID NO: 5 - skipped

SEQ ID NO: 6 - protein, 9 aa, synthetic construct
  REGION 1..9: note = Synthetic
MQHLEYPFT

SEQ ID NO: 7 - protein, 5 aa, synthetic construct
  REGION 1..5: note = Synthetic
DDYMY

SEQ ID NO: 8 - protein, 17 aa, synthetic construct
  REGION 1..17: note = Synthetic
WIDPENGDTEYASKFQD

SEQ ID NO: 9 - protein, 8 aa, synthetic construct
  REGION 1..8: note = Synthetic
WLRRGLDY

SEQ ID NO: 10 - protein, 16 aa, synthetic construct
  REGION 1..16: note = Synthetic
RSSKSLLHSNGYTYLF

SEQ ID NO: 11 - protein, 7 aa, synthetic construct
  REGION 1..7: note = Synthetic
RMSNLAS

SEQ ID NO: 12 - protein, 7 aa, synthetic construct
  REGION 1..7: note = Synthetic
GFNIKDD

SEQ ID NO: 13 - skipped

SEQ ID NO: 14 - protein, 6 aa, synthetic construct
  REGION 1..6: note = Synthetic
LRRGLD

SEQ ID NO: 15 - protein, 12 aa, synthetic construct
  REGION 1..12: note = Synthetic
SKSLLHSNGYTY

SEQ ID NO: 16 - protein, 6 aa, synthetic construct
  REGION 1..6: note = Synthetic
HLEYPF

SEQ ID NO: 17 - protein, 117 aa, synthetic construct
  REGION 1..117: note = Synthetic
EVQLQQSGAELVRPGASVKLSCTASGFNIKDDYMYWVKQRPEQGLEWIGWIDPENGDTEYASKFQDKATVTADTSSNTAYLQLSSLTSEDTAVYYCTLWLRRGLDYWGQGTSVTVSS

SEQ ID NO: 18 - protein, 112 aa, synthetic construct
  REGION 1..112: note = Synthetic
DIVMTQAAPSVPVTPGESVSISCRSSKSLLHSNGYTYLFWFLQRPGQSPQLLIYRMSNLASGVPDRFSGSGSGTAFTLRISRVEAEDVGVYYCMQHLEYPFTFGGGTKLEIK

SEQ ID NO: 19 - protein, 8 aa, synthetic construct
  REGION 1..8: note = Synthetic
IDPETGDT

SEQ ID NO: 20 - protein, 17 aa, synthetic construct
  REGION 1..17: note = Synthetic
WIDPETGDTEYASKFQD

SEQ ID NO: 21 - skipped

SEQ ID NO: 22 - protein, 117 aa, synthetic construct
  REGION 1..117: note = Synthetic
EVQLQQSGAELVRPGASVKLSCTASGFNIKDDYMYWVKQRPEQGLEWIGWIDPETGDTEYASKFQDKATVTADTSSNTAYLQLSSLTSEDTAVYYCTLWLRRGLDYWGQGTSVTVSS

SEQ ID NO: 23 - protein, 8 aa, synthetic construct
  REGION 1..8: note = Synthetic
IDPESGDT

SEQ ID NO: 24 - protein, 17 aa, synthetic construct
  REGION 1..17: note = Synthetic
WIDPESGDTEYASKFQD

SEQ ID NO: 25 - skipped

SEQ ID NO: 26 - protein, 117 aa, synthetic construct
  REGION 1..117: note = Synthetic
EVQLQQSGAELVRPGASVKLSCTASGFNIKDDYMYWVKQRPEQGLEWIGWIDPESGDTEYASKFQDKATVTADTSSNTAYLQLSSLTSEDTAVYYCTLWLRRGLDYWGQGTSVTVSS

SEQ ID NO: 27 - protein, 9 aa, synthetic construct
  REGION 1..9: note = Synthetic
GYSITSGYY

SEQ ID NO: 28 - protein, 7 aa, synthetic construct
  REGION 1..7: note = Synthetic
ITFDGAN

SEQ ID NO: 29 - protein, 12 aa, synthetic construct
  REGION 1..12: note = Synthetic
TRSSYDYDVLDY

SEQ ID NO: 30 - protein, 6 aa, synthetic construct
  REGION 1..6: note = Synthetic
QDISNF

SEQ ID NO: 31 - skipped

SEQ ID NO: 32 - protein, 9 aa, synthetic construct
  REGION 1..9: note = Synthetic
QQGHTLPYT

SEQ ID NO: 33 - protein, 6 aa, synthetic construct
  REGION 1..6: note = Synthetic
SGYYWN

SEQ ID NO: 34 - protein, 16 aa, synthetic construct
  REGION 1..16: note = Synthetic
YITFDGANNYNPSLKN

SEQ ID NO: 35 - protein, 10 aa, synthetic construct
  REGION 1..10: note = Synthetic
SSYDYDVLDY

SEQ ID NO: 36 - protein, 11 aa, synthetic construct
  REGION 1..11: note = Synthetic
RASQDISNFLN

SEQ ID NO: 37 - protein, 7 aa, synthetic construct
  REGION 1..7: note = Synthetic
YTSRLHS

SEQ ID NO: 38 - protein, 8 aa, synthetic construct
  REGION 1..8: note = Synthetic
GYSITSGY

SEQ ID NO: 39 - skipped

SEQ ID NO: 40 - protein, 8 aa, synthetic construct
  REGION 1..8: note = Synthetic
SYDYDVLD

SEQ ID NO: 41 - protein, 7 aa, synthetic construct
  REGION 1..7: note = Synthetic
SQDISNF

SEQ ID NO: 42 - protein, 6 aa, synthetic construct
  REGION 1..6: note = Synthetic
GHTLPY

SEQ ID NO: 43 - protein, 119 aa, synthetic construct
  REGION 1..119: note = Synthetic
DVQLQESGPGLVKPSQSLSLTCSVTGYSITSGYYWNWIRQFPGNKLEWMGYITFDGANNYNPSLKNRISITRDTSKNQFFLKLTSVTTEDTATYYCTRSSYDYDVLDYWGQGTTLTVSS

SEQ ID NO: 44 - protein, 107 aa, synthetic construct
  REGION 1..107: note = Synthetic
DIQMTQTTSSLSASLGDRVTISCRASQDISNFLNWYQQRPDGTVKLLIYYTSRLHSGVPSRFSGSGSGTDFSLTVSNLEQEDIATYFCQQGHTLPYTFGGGTKLEIK

SEQ ID NO: 45 - protein, 8 aa, synthetic construct
  REGION 1..8: note = Synthetic
GYSFTDYC

SEQ ID NO: 46 - protein, 8 aa, synthetic construct
  REGION 1..8: note = Synthetic
IYPGSGNT

SEQ ID NO: 47 - protein, 13 aa, synthetic construct
  REGION 1..13: note = Synthetic
AREDYYPYHGMDY

SEQ ID NO: 48 - protein, 10 aa, synthetic construct
  REGION 1..10: note = Synthetic
ESVDGYDNSF

SEQ ID NO: 49 - skipped

SEQ ID NO: 50 - protein, 9 aa, synthetic construct
  REGION 1..9: note = Synthetic
QQSSEDPWT

SEQ ID NO: 51 - protein, 5 aa, synthetic construct
  REGION 1..5: note = Synthetic
DYCIN

SEQ ID NO: 52 - protein, 17 aa, synthetic construct
  REGION 1..17: note = Synthetic
WIYPGSGNTRYSERFKG

SEQ ID NO: 53 - protein, 11 aa, synthetic construct
  REGION 1..11: note = Synthetic
EDYYPYHGMDY

SEQ ID NO: 54 - protein, 15 aa, synthetic construct
  REGION 1..15: note = Synthetic
RASESVDGYDNSFMH

SEQ ID NO: 55 - protein, 7 aa, synthetic construct
  REGION 1..7: note = Synthetic
RASNLES

SEQ ID NO: 56 - protein, 7 aa, synthetic construct
  REGION 1..7: note = Synthetic
GYSFTDY

SEQ ID NO: 57 - skipped

SEQ ID NO: 58 - protein, 9 aa, synthetic construct
  REGION 1..9: note = Synthetic
DYYPYHGMD

SEQ ID NO: 59 - protein, 11 aa, synthetic construct
  REGION 1..11: note = Synthetic
SESVDGYDNSF

SEQ ID NO: 60 - protein, 6 aa, synthetic construct
  REGION 1..6: note = Synthetic
SSEDPW

SEQ ID NO: 61 - protein, 120 aa, synthetic construct
  REGION 1..120: note = Synthetic
QIQLQQSGPELVRPGASVKISCKASGYSFTDYCINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 62 - protein, 111 aa, synthetic construct
  REGION 1..111: note = Synthetic
DIVLTQSPTSLAVSLGQRATISCRASESVDGYDNSFMHWYQQKPGQPPKLLIFRASNLESGIPARFSGSGSRTDFTLTINPVEAADVATYYCQQSSEDPWTFGGGTKLEIK

SEQ ID NO: 63 - protein, 8 aa, synthetic construct
  REGION 1..8: note = Synthetic
GYSFTDYY

SEQ ID NO: 64 - protein, 5 aa, synthetic construct
  REGION 1..5: note = Synthetic
DYYIN

SEQ ID NO: 65 - protein, 120 aa, synthetic construct
  REGION 1..120: note = Synthetic
QIQLQQSGPELVRPGASVKISCKASGYSFTDYYINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 66 - protein, 8 aa, synthetic construct
  REGION 1..8: note = Synthetic
GYSFTDYD

SEQ ID NO: 67 - protein, 5 aa, synthetic construct
  REGION 1..5: note = Synthetic
DYDIN

SEQ ID NO: 68 - protein, 120 aa, synthetic construct
  REGION 1..120: note = Synthetic
QIQLQQSGPELVRPGASVKISCKASGYSFTDYDINWVNQRPGQGLEWIGWIYPGSGNTRYSERFKGKATLTVDTSSNTAYMQLSSLTSEDSAVYFCAREDYYPYHGMDYWGQGTSVTVSS

SEQ ID NO: 69 - protein, 117 aa, synthetic construct
  REGION 1..117: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSS

SEQ ID NO: 70 - protein, 112 aa, synthetic construct
  REGION 1..112: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIK

SEQ ID NO: 71 - protein, 117 aa, synthetic construct
  REGION 1..117: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSS

SEQ ID NO: 72 - protein, 117 aa, synthetic construct
  REGION 1..117: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSS

SEQ ID NO: 73 - protein, 119 aa, synthetic construct
  REGION 1..119: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSS

SEQ ID NO: 74 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIK

SEQ ID NO: 75 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 76 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 77 - protein, 120 aa, synthetic construct
  REGION 1..120: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 78 - protein, 111 aa, synthetic construct
  REGION 1..111: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIK

SEQ ID NO: 79 - protein, 120 aa, synthetic construct
  REGION 1..120: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSS

SEQ ID NO: 80 - protein, 111 aa, synthetic construct
  REGION 1..111: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIK

SEQ ID NO: 81 - protein, 330 aa, Homo sapiens [sequence omitted]

SEQ ID NO: 82 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 107 aa, synthetic construct [sequence omitted]

SEQ ID NO: 84 - protein, 447 aa, synthetic construct
  REGION 1..447: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 219 aa, synthetic construct
  REGION 1..219: note = Synthetic
DIVMTQSPLSLPVTPGEPASISCRSSKSLLHSNGYTYLFWFQQRPGQSPRLLIYRMSNLASGVPDRFSGSGSGTDFTLKISRVEAEDVGVYYCMQHLEYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 447 aa, synthetic construct
  REGION 1..447: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 447 aa, synthetic construct
  REGION 1..447: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 449 aa, synthetic construct
  REGION 1..449: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYFCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 449 aa, synthetic construct
  REGION 1..449: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 450 aa, synthetic construct
  REGION 1..450: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 218 aa, synthetic construct
  REGION 1..218: note = Synthetic
DIVLTQSPDSLAVSLGERATINCRASESVDGYDNSFMHWYQQKPGQPPKLLIFRASNLESGVPDRFSGSGSRTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 450 aa, synthetic construct
  REGION 1..450: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 218 aa, synthetic construct
  REGION 1..218: note = Synthetic
DIVMTQSPDSLAVSLGERATINCRASESVDGYDNSFMHWYQQKPGQPPKLLIFRASNLESGVPDRFSGSGSGTDFTLTISSLQAEDVAVYYCQQSSEDPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 108 aa, synthetic construct
  REGION 1..108: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 225 aa, synthetic construct
  REGION 1..225: note = Synthetic
EVQLVQSGSELKKPGASVKVSCTASGFNIKDDYMYWVRQPPGKGLEWIGWIDPETGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 98 - protein, 225 aa, synthetic construct
  REGION 1..225: note = Synthetic
EVQLVQSGSELKKPGASVKVSCTASGFNIKDDYMYWVRQPPGKGLEWIGWIDPESGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 99 - protein, 225 aa, synthetic construct
  REGION 1..225: note = Synthetic
EVQLVQSGSELKKPGASVKVSCTASGFNIKDDYMYWVRQPPGKGLEWIGWIDPENGDTEYASKFQDRVTVTADTSTNTAYMELSSLRSEDTAVYYCTLWLRRGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 100 - protein, 227 aa, synthetic construct
  REGION 1..227: note = Synthetic
QVQLQESGPGLVKPSQTLSLTCSVTGYSITSGYYWNWIRQPPGKGLEWMGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 101 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 102 - protein, 228 aa, synthetic construct
  REGION 1..228: note = Synthetic
QVQLVQSGAEVKKPGASVKVSCKASGYSFTDYYINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 103 - protein, 228 aa, synthetic construct
  REGION 1..228: note = Synthetic
QVQLVQSGAEVKKPGASVKVSCKASGYSFTDYDINWVRQAPGQGLEWMGWIYPGSGNTRYSERFKGRVTITRDTSASTAYMELSSLRSEDTAVYYCAREDYYPYHGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 104 - protein, 19 aa, synthetic construct
  REGION 1..19: note = Synthetic
MGWSCIILFLVATATGVHS

SEQ ID NO: 105 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 106 - protein, 760 aa, Macaca mulatta
MMDQARSAFSNLFGGEPLSYTRFSLARQVDGDNSHVEMKLGVDEEENTDNNTKPNGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 107 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 108 - protein, 763 aa, Mus musculus
MMDQARSAFSNLFGGEPLSYTRFSLARQVDGDNSHVEMKLAADEEENADNNMKASVRKPKRFNGRLCFAAIALVIFFLIGFMSGYLGYCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 109 - protein, 197 aa, synthetic construct
  REGION 1..197: note = Synthetic
FVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKE

SEQ ID NO: 110 - protein, 5 aa, synthetic construct
  REGION 1..5: note = Synthetic
SYWMH

SEQ ID NO: 111 - protein, 17 aa, synthetic construct
  REGION 1..17: note = Synthetic
EINPTNGRTNYIEKFKS

SEQ ID NO: 112 - protein, 7 aa, synthetic construct
  REGION 1..7: note = Synthetic
GTRAYHY

SEQ ID NO: 113 - protein, 11 aa, synthetic construct
  REGION 1..11: note = Synthetic
RASDNLYSNLA

SEQ ID NO: 114 - protein, 7 aa, synthetic construct
  REGION 1..7: note = Synthetic
DATNLAD

SEQ ID NO: 115 - protein, 9 aa, synthetic construct
  REGION 1..9: note = Synthetic
QHFWGTPLT

SEQ ID NO: 116 - protein, 7 aa, synthetic construct
  REGION 1..7: note = Synthetic
GYTFTSY

SEQ ID NO: 117 - protein, 6 aa, synthetic construct
  REGION 1..6: note = Synthetic
NPTNGR

SEQ ID NO: 118 - protein, 6 aa, synthetic construct
  REGION 1..6: note = Synthetic
TSYWMH

SEQ ID NO: 119 - protein, 13 aa, synthetic construct
  REGION 1..13: note = Synthetic
WIGEINPTNGRTN

SEQ ID NO: 120 - protein, 6 aa, synthetic construct
  REGION 1..6: note = Synthetic
ARGTRA

SEQ ID NO: 121 - protein, 7 aa, synthetic construct
  REGION 1..7: note = Synthetic
YSNLAWY

SEQ ID NO: 122 - protein, 10 aa, synthetic construct
  REGION 1..10: note = Synthetic
LLVYDATNLA

SEQ ID NO: 123 - protein, 8 aa, synthetic construct
  REGION 1..8: note = Synthetic
QHFWGTPL

SEQ ID NO: 124 - protein, 116 aa, Mus musculus
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSS

SEQ ID NO: 125 - protein, 107 aa, Mus musculus
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELK

SEQ ID NO: 126 - protein, 9 aa, synthetic construct
  REGION 1..9: note = Synthetic
QHFAGTPLT

SEQ ID NO: 127 - protein, 8 aa, synthetic construct
  REGION 1..8: note = Synthetic
QHFAGTPL

SEQ ID NO: 128 - protein, 116 aa, synthetic construct
  REGION 1..116: note = Synthetic
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSS

SEQ ID NO: 129 - protein, 107 aa, synthetic construct
  REGION 1..107: note = Synthetic
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIK

SEQ ID NO: 130 - protein, 2859 aa, Homo sapiens
AGGGGGGCTGGACCAAGGGGTGGGGAGAAGGGGAGGAGGCCTCGGCCGGCCGCAGAGAGAAGTGGCCAGAGAGGCCCAGGGGACAGCCAGGGACAGGCAGACATGCAGCCAGGGCTCCAGGGCCTGGACAGGGGCTGCCAGGCCCTGTGACAGGAGGACCCCGAGCCCCCGGCCCGGGGAGGGGCCATGGTGCTGCCTGTCCAACATGTCAGCCGAGGTGCGGCTGAGGCGGCTCCAGCAGCTGGTGTTGGACCCGGGCTTCCTGGGGCTGGAGCCCCTGCTCGACCTTCTCCTGGGCGTCCACCAGGAGCTGGGCGCCTCCGAACTGGCCCAGGACAAGTACGTGGCCGACTTCTTGCAGTGGGCGGAGCCCATCGTGGTGAGGCTTAAGGAGGTCCGACTGCAGAGGGACGACTTCGAGATTCTGAAGGTGATCGGACGCGGGGCGTTCAGCGAGGTAGCGGTAGTGAAGATGAAGCAGACGGGCCAGGTGTATGCCATGAAGATCATGAACAAGTGGGACATGCTGAAGAGGGGCGAGGTGTCGTGCTTCCGTGAGGAGAGGGACGTGTTGGTGAATGGGGACCGGCGGTGGATCACGCAGCTGCACTTCGCCTTCCAGGATGAGAACTACCTGTACCTGGTCATGGAGTATTACGTGGGCGGGGACCTGCTGACACTGCTGAGCAAGTTTGGGGAGCGGATTCCGGCCGAGATGGCGCGCTTCTACCTGGCGGAGATTGTCATGGCCATAGACTCGGTGCACCGGCTTGGCTACGTGCACAGGGACATCAAACCCGACAACATCCTGCTGGACCGCTGTGGCCACATCCGCCTGGCCGACTTCGGCTCTTGCCTCAAGCTGCGGGCAGATGGAACGGTGCGGTCGCTGGTGGCTGTGGGCACCCCAGACTACCTGTCCCCCGAGATCCTGCAGGCTGTGGGCGGTGGGCCTGGGACAGGCAGCTACGGGCCCGAGTGTGACTGGTGGGCGCTGGGTGTATTCGCCTATGAAATGTTCTATGGGCAGACGCCCTTCTACGCGGATTCCACGGCGGAGACCTATGGCAAGATCGTCCACTACAAGGAGCACCTCTCTCTGCCGCTGGTGGACGAAGGGGTCCCTGAGGAGGCTCGAGACTTCATTCAGCGGTTGCTGTGTCCCCCGGAGACACGGCTGGGCCGGGGTGGAGCAGGCGACTTCCGGACACATCCCTTCTTCTTTGGCCTCGACTGGGATGGTCTCCGGGACAGCGTGCCCCCCTTTACACCGGATTTCGAAGGTGCCACCGACACATGCAACTTCGACTTGGTGGAGGACGGGCTCACTGCCATGGAGACACTGTCGGACATTCGGGAAGGTGCGCCGCTAGGGGTCCACCTGCCTTTTGTGGGCTACTCCTACTCCTGCATGGCCCTCAGGGACAGTGAGGTCCCAGGCCCCACACCCATGGAACTGGAGGCCGAGCAGCTGCTTGAGCCACACGTGCAAGCGCCCAGCCTGGAGCCCTCGGTGTCCCCACAGGATGAAACAGCTGAAGTGGCAGTTCCAGCGGCTGTCCCTGCGGCAGAGGCTGAGGCCGAGGTGACGCTGCGGGAGCTCCAGGAAGCCCTGGAGGAGGAGGTGCTCACCCGGCAGAGCCTGAGCCGGGAGATGGAGGCCATCCGCACGGACAACCAGAACTTCGCCAGTCAACTACGCGAGGCAGAGGCTCGGAACCGGGACCTAGAGGCACACGTCCGGCAGTTGCAGGAGCGGATGGAGTTGCTGCAGGCAGAGGGAGCCACAGCTGTCACGGGGGTCCCCAGTCCCCGGGCCACGGATCCACCTTCCCATCTAGATGGCCCCCCGGCCGTGGCTGTGGGCCAGTGCCCGCTGGTGGGGCCAGGCCCCATGCACCGCCGCCACCTGCTGCTCCCTGCCAGGGTCCCTAGGCCTGGCCTATCGGAGGCGCTTTCCCTGCTCCTGTTCGCCGTTGTTCTGTCTCGTGCCGCCGCCCTGGGCTGCATTGGGTTGGTGGCCCACGCCGGCCAACTCACCGCAGTCTGGCGCCGCCCAGGAGCCGCCCGCGCTCCCTGAACCCTAGAACTGTCTTCGACTCCGGGGCCCCGTTGGAAGACTGAGTGCCCGGGGCACGGCACAGAAGCCGCGCCCACCGCCTGCCAGTTCACAACCGCTCCGAGCGTGGGTCTCCGCCCAGCTCCAGTCCTGTGATCCGGGCCCGCCCCCTAGCGGCCGGGGAGGGAGGGGCCGGGTCCGCGGCCGGCGAACGGGGCTCGAAGGGTCCTTGTAGCCGGGAATGCTGCTGCTGCTGCTGCTGCTGCTGCTGCTGCTGCTGCTGCTGCTGCTGCTGCTGCTGCTGGGGGGATCACAGACCATTTCTTTCTTTCGGCCAGGCTGAGGCCCTGACGTGGATGGGCAAACTGCAGGCCTGGGAAGGCAGCAAGCCGGGCCGTCCGTGTTCCATCCTCCACGCACCCCCACCTATCGTTGGTTCGCAAAGTGCAAAGCTTTCTTGTGCATGACGCCCTGCTCTGGGGAGCGTCTGGCGCGATCTCTGCCTGCTTACTCGGGAAATTTGCTTTTGCCAAACCCGCTTTTTCGGGGATCCCGCGCCCCCCTCCTCACTTGCGCTGCTCTCGGAGCCCCAGCCGGCTCCGCCCGCTTCGGCGGTTTGGATATTTATTGACCTCGTCCTCCGACTCGCTGACAGGCTACAGGACCCCCAACAACCCCAATCCACGTTTTGGATGCACTGAGACCCCGACATTCCTCGGTATTTATTGTCTGTCCCCACCTAGGACCCCCACCCCCGACCCTCGCGAATAAAAGGCCCTCCATCTGCCCAAAGCTCTGGA

SEQ ID NO: 131 - protein, 2683 aa, Mus musculus
GAACTGGCCAGAGAGACCCAAGGGATAGTCAGGGACGGGCAGACATGCAGCTAGGGTTCTGGGGCCTGGACAGGGGCAGCCAGGCCCTGTGACGGGAAGACCCCGAGCTCCGGCCCGGGGAGGGGCCATGGTGTTGCCTGCCCAACATGTCAGCCGAAGTGCGGCTGAGGCAGCTCCAGCAGCTGGTGCTGGACCCAGGCTTCCTGGGACTGGAGCCCCTGCTCGACCTTCTCCTGGGCGTCCACCAGGAGCTGGGTGCCTCTCACCTAGCCCAGGACAAGTATGTGGCCGACTTCTTGCAGTGGGTGGAGCCCATTGCAGCAAGGCTTAAGGAGGTCCGACTGCAGAGGGATGATTTTGAGATTTTGAAGGTGATCGGGCGTGGGGCGTTCAGCGAGGTAGCGGTGGTGAAGATGAAACAGACGGGCCAAGTGTATGCCATGAAGATTATGAATAAGTGGGACATGCTGAAGAGAGGCGAGGTGTCGTGCTTCCGGGAAGAAAGGGATGTATTAGTGAAAGGGGACCGGCGCTGGATCACACAGCTGCACTTTGCCTTCCAGGATGAGAACTACCTGTACCTGGTCATGGAATACTACGTGGGCGGGGACCTGCTAACGCTGCTGAGCAAGTTTGGGGAGCGGATCCCCGCCGAGATGGCTCGCTTCTACCTGGCCGAGATTGTCATGGCCATAGACTCCGTGCACCGGCTGGGCTACGTGCACAGGGACATCAAACCAGATAACATTCTGCTGGACCGATGTGGGCACATTCGCCTGGCAGACTTCGGCTCCTGCCTCAAACTGCAGCCTGATGGAATGGTGAGGTCGCTGGTGGCTGTGGGCACCCCGGACTACCTGTCTCCTGAGATTCTGCAGGCCGTTGGTGGAGGGCCTGGGGCAGGCAGCTACGGGCCAGAGTGTGACTGGTGGGCACTGGGCGTGTTCGCCTATGAGATGTTCTATGGGCAGACCCCCTTCTACGCGGACTCCACAGCCGAGACATATGCCAAGATTGTGCACTACAGGGAACACTTGTCGCTGCCGCTGGCAGACACAGTTGTCCCCGAGGAAGCTCAGGACCTCATTCGTGGGCTGCTGTGTCCTGCTGAGATAAGGCTAGGTCGAGGTGGGGCAGACTTCGAGGGTGCCACGGACACATGCAATTTCGATGTGGTGGAGGACCGGCTCACTGCCATGGTGAGCGGGGGCGGGGAGACGCTGTCAGACATGCAGGAAGACATGCCCCTTGGGGTGCGCCTGCCCTTCGTGGGCTACTCCTACTGCTGCATGGCCTTCAGAGACAATCAGGTCCCGGACCCCACCCCTATGGAACTAGAGGCCCTGCAGTTGCCTGTGTCAGACTTGCAAGGGCTTGACTTGCAGCCCCCAGTGTCCCCACCGGATCAAGTGGCTGAAGAGGCTGACCTAGTGGCTGTCCCTGCCCCTGTGGCTGAGGCAGAGACCACGGTAACGCTGCAGCAGCTCCAGGAAGCCCTGGAAGAAGAGGTTCTCACCCGGCAGAGCCTGAGCCGCGAGCTGGAGGCCATCCGGACCGCCAACCAGAACTTCTCCAGCCAACTACAGGAGGCCGAGGTCCGAAACCGAGACCTGGAGGCGCATGTTCGGCAGCTACAGGAACGGATGGAGATGCTGCAGGCCCCAGGAGCCGCAGCCATCACGGGGGTCCCCAGTCCCCGGGCCACGGATCCACCTTCCCATCTAGATGGCCCCCCGGCCGTGGCTGTGGGCCAGTGCCCGCTGGTGGGGCCAGGCCCCATGCACCGCCGTCACCTGCTGCTCCCTGCCAGGATCCCTAGGCCTGGCCTATCCGAGGCGCGTTGCCTGCTCCTGTTCGCCGCTGCTCTGGCTGCTGCCGCCACACTGGGCTGCACTGGGTTGGTGGCCTATACCGGCGGTCTCACCCCAGTCTGGTGTTTCCCGGGAGCCACCTTCGCCCCCTGAACCCTAAGACTCCAAGCCATCTTTCATTTAGGCCTCCTAGGAAGGTCGAGCGACCAGGGAGCGACCCAAAGCGTCTCTGTGCCCATCGCGCCCCCCCCCCCCCCCCACCGCTCCGCTCCACACTTCTGTGAGCCTGGGTCCCCACCCAGCTCCGCTCCTGTGATCCAGGCCTGCCACCTGGCGGCCGGGGAGGGAGGAACAGGGCTCGTGCCCAGCACCCCTGGTTCCTGCAGAGCTGGTAGCCACCGCTGCTGCAGCAGCTGGGCATTCGCCGACCTTGCTTTACTCAGCCCCGACGTGGATGGGCAAACTGCTCAGCTCATCCGATTTCACTTTTTCACTCTCCCAGCCATCAGTTACAAGCCATAAGCATGAGCCCCCTATTTCCAGGGACATCCCATTCCCATAGTGATGGATCAGCAAGACCTCTGCCAGCACACACGGAGTCTTTGGCTTCGGACAGCCTCACTCCTGGGGGTTGCTGCAACTCCTTCCCCGTGTACACGTCTGCACTCTAACAACGGAGCCACAGCTGCACTCCCCCCTCCCCCAAAGCAGTGTGGGTATTTATTGATCTTGTTATCTGACTCACTGACAGACTCCGGGACCCACGTTTTAGATGCATTGAGACTCGACATTCCTCGGTATTTATTGTCTGTCCCCACCTACGACCTCCACTCCCGACCCTTGCGAATAAAATACTTCTGGTCTGCCCTAAA

SEQ ID NO: 132 - protein, 446 aa, synthetic construct
  REGION 1..446: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 214 aa, synthetic construct
  REGION 1..214: note = Synthetic
DIQMTQSPASLSVSVGETVTITCRASDNLYSNLAWYQQKQGKSPQLLVYDATNLADGVPSRFSGSGSGTQYSLKINSLQSEDFGTYYCQHFWGTPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 446 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, Homo sapiens
DIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 226 aa, synthetic construct
  REGION 1..226: note = Synthetic
QVQLQQPGAELVKPGASVKLSCKASGYTFTSYWMHWVKQRPGQGLEWIGEINPTNGRTNYIEKFKSKATLTVDKSSSTAYMQLSSLTSEDSAVYYCARGTRAYHYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 137 - protein, 226 aa, Homo sapiens
EVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 138 - protein, 8 aa, synthetic construct
  REGION 1..8: note = Synthetic
GYSFTSYW

SEQ ID NO: 139 - protein, 8 aa, synthetic construct
  REGION 1..8: note = Synthetic
IYPGDSDT

SEQ ID NO: 140 - protein, 15 aa, synthetic construct
  REGION 1..15: note = Synthetic
ARFPYDSSGYYSFDY

SEQ ID NO: 141 - protein, 6 aa, synthetic construct
  REGION 1..6: note = Synthetic
QSISSY

SEQ ID NO: 142 - skipped

SEQ ID NO: 143 - protein, 9 aa, synthetic construct
  REGION 1..9: note = Synthetic
QQSYSTPLT

SEQ ID NO: 144 - protein, 5 aa, synthetic construct
  REGION 1..5: note = Synthetic
SYWIG

SEQ ID NO: 145 - protein, 18 aa, synthetic construct
  REGION 1..18: note = Synthetic
IIYPGDSDTRYSPSFQGQ

SEQ ID NO: 146 - protein, 13 aa, synthetic construct
  REGION 1..13: note = Synthetic
FPYDSSGYYSFDY

SEQ ID NO: 147 - protein, 11 aa, synthetic construct
  REGION 1..11: note = Synthetic
RASQSISSYLN

SEQ ID NO: 148 - protein, 7 aa, synthetic construct
  REGION 1..7: note = Synthetic
AASSLQS

SEQ ID NO: 149 - protein, 7 aa, synthetic construct
  REGION 1..7: note = Synthetic
GYSFTSY

SEQ ID NO: 150 - skipped

SEQ ID NO: 151 - protein, 11 aa, synthetic construct
  REGION 1..11: note = Synthetic
PYDSSGYYSFD

SEQ ID NO: 152 - protein, 7 aa, synthetic construct
  REGION 1..7: note = Synthetic
SQSISSY

SEQ ID NO: 153 - protein, 6 aa, synthetic construct
  REGION 1..6: note = Synthetic
SYSTPL

SEQ ID NO: 154 - protein, 122 aa, synthetic construct
  REGION 1..122: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSS

SEQ ID NO: 155 - protein, 107 aa, synthetic construct [sequence omitted]

SEQ ID NO: 156 - protein, 452 aa, synthetic construct
  REGION 1..452: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, synthetic construct
  REGION 1..214: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 232 aa, synthetic construct
  REGION 1..232: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCP

SEQ ID NO: 159 - protein, 230 aa, synthetic construct
  REGION 1..230: note = Synthetic
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARFPYDSSGYYSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 160 - DNA, 20 bp, synthetic construct
  misc_feature 1..20: note = Synthetic
  source 1..20: mol_type = other DNA
cgggccaggtgtatgccatg

SEQ ID NO: 161 - DNA, 20 bp, synthetic construct
  misc_feature 1..20: note = Synthetic
  source 1..20: mol_type = other DNA
aggagagggacgtgttggtg

SEQ ID NO: 162 - DNA, 20 bp, synthetic construct
  misc_feature 1..20: note = Synthetic
  source 1..20: mol_type = other DNA
ggagagggacgtgttggtga

SEQ ID NO: 163 - DNA, 20 bp, synthetic construct
  misc_feature 1..20: note = Synthetic
  source 1..20: mol_type = other DNA
agggacgtgttggtgaatgg

SEQ ID NO: 164 - DNA, 20 bp, synthetic construct
  misc_feature 1..20: note = Synthetic
  source 1..20: mol_type = other DNA
caggatgagaactacctgta

SEQ ID NO: 165 - DNA, 20 bp, synthetic construct
  misc_feature 1..20: note = Synthetic
  source 1..20: mol_type = other DNA
aggatgagaactacctgtac

SEQ ID NO: 166 - DNA, 20 bp, synthetic construct
  misc_feature 1..20: note = Synthetic
  source 1..20: mol_type = other DNA
gagaactacctgtacctggt

SEQ ID NO: 167 - DNA, 20 bp, synthetic construct
  misc_feature 1..20: note = Synthetic
  source 1..20: mol_type = other DNA
agaactacctgtacctggtc

SEQ ID NO: 168 - DNA, 20 bp, synthetic construct
  misc_feature 1..20: note = Synthetic
  source 1..20: mol_type = other DNA
gaactacctgtacctggtca

SEQ ID NO: 169 - DNA, 20 bp, synthetic construct
  misc_feature 1..20: note = Synthetic
  source 1..20: mol_type = other DNA
cactgctgagcaagtttggg

SEQ ID NO: 170 - DNA, 20 bp, synthetic construct
  misc_feature 1..20: note = Synthetic
  source 1..20: mol_type = other DNA
ctacctggcggagattgtca

SEQ ID NO: 171 - DNA, 20 bp, synthetic construct
  misc_feature 1..20: note = Synthetic
  source 1..20: mol_type = other DNA
tacctggcggagattgtcat

SEQ ID NO: 172 - DNA, 20 bp, synthetic construct
  misc_feature 1..20: note = Synthetic
  source 1..20: mol_type = other DNA
acctggcggagattgtcatg

SEQ ID NO: 173 - DNA, 20 bp, synthetic construct
  misc_feature 1..20: note = Synthetic
  source 1..20: mol_type = other DNA
cctggcggagattgtcatgg

SEQ ID NO: 174 - DNA, 20 bp, synthetic construct
  misc_feature 1..20: note = Synthetic
  source 1..20: mol_type = other DNA
ctggcggagattgtcatggc

SEQ ID NO: 175 - DNA, 20 bp, synthetic construct
  misc_feature 1..20: note = Synthetic
  source 1..20: mol_type = other DNA
tggcggagattgtcatggcc

SEQ ID NO: 176 - DNA, 20 bp, synthetic construct
  misc_feature 1..20: note = Synthetic
  source 1..20: mol_type = other DNA
ggcggagattgtcatggcca

SEQ ID NO: 177 - DNA, 20 bp, synthetic construct
  misc_feature 1..20: note = Synthetic
  source 1..20: mol_type = other DNA
ccggcttggctacgtgcaca

SEQ ID NO: 178 - DNA, 20 bp, synthetic construct
  misc_feature 1..20: note = Synthetic
  source 1..20: mol_type = other DNA
cggcttggctacgtgcacag

SEQ ID NO: 179 - DNA, 20 bp, synthetic construct
  misc_feature 1..20: note = Synthetic
  source 1..20: mol_type = other DNA
catcctgctggaccgctgtg

SEQ ID NO: 180 - DNA, 20 bp, synthetic construct
  misc_feature 1..20: note = Synthetic
  source 1..20: mol_type = other DNA
ctgctggaccgctgtggcca

SEQ ID NO: 181 - DNA, 20 bp, synthetic construct
  misc_feature 1..20: note = Synthetic
  source 1..20: mol_type = other DNA
gagtgtgactggtgggcgct

SEQ ID NO: 182 - DNA, 20 bp, synthetic construct
  misc_feature 1..20: note = Synthetic
  source 1..20: mol_type = other DNA
agtgtgactggtgggcgctg

SEQ ID NO: 183 - DNA, 20 bp, synthetic construct
  misc_feature 1..20: note = Synthetic
  source 1..20: mol_type = other DNA
gtgtgactggtgggcgctgg

SEQ ID NO: 184 - DNA, 20 bp, synthetic construct
  misc_feature 1..20: note = Synthetic
  source 1..20: mol_type = other DNA
tgggcgctgggtgtattcgc

SEQ ID NO: 185 - DNA, 20 bp, synthetic construct
  misc_feature 1..20: note = Synthetic
  source 1..20: mol_type = other DNA
gggcgctgggtgtattcgcc

SEQ ID NO: 186 - DNA, 20 bp, synthetic construct
  misc_feature 1..20: note = Synthetic
  source 1..20: mol_type = other DNA
ggcaagatcgtccactacaa

SEQ ID NO: 187 - DNA, 20 bp, synthetic construct
  misc_feature 1..20: note = Synthetic
  source 1..20: mol_type = other DNA
gcaagatcgtccactacaag

SEQ ID NO: 188 - DNA, 20 bp, synthetic construct
  misc_feature 1..20: note = Synthetic
  source 1..20: mol_type = other DNA
caagatcgtccactacaagg

SEQ ID NO: 189 - DNA, 20 bp, synthetic construct
  misc_feature 1..20: note = Synthetic
  source 1..20: mol_type = other DNA
ctcgactgggatggtctccg

SEQ ID NO: 190 - DNA, 20 bp, synthetic construct
  misc_feature 1..20: note = Synthetic
  source 1..20: mol_type = other DNA
ggagacactgtcggacattc

SEQ ID NO: 191 - DNA, 20 bp, synthetic construct
  misc_feature 1..20: note = Synthetic
  source 1..20: mol_type = other DNA
gagacactgtcggacattcg

SEQ ID NO: 192 - DNA, 20 bp, synthetic construct
  misc_feature 1..20: note = Synthetic
  source 1..20: mol_type = other DNA
gacagtgaggtcccaggccc

SEQ ID NO: 193 - DNA, 20 bp, synthetic construct
  misc_feature 1..20: note = Synthetic
  source 1..20: mol_type = other DNA
ctgcttgagccacacgtgca

SEQ ID NO: 194 - DNA, 20 bp, synthetic construct
  misc_feature 1..20: note = Synthetic
  source 1..20: mol_type = other DNA
ggatgaaacagctgaagtgg

SEQ ID NO: 195 - DNA, 20 bp, synthetic construct
  misc_feature 1..20: note = Synthetic
  source 1..20: mol_type = other DNA
ggctgaggccgaggtgacgc

SEQ ID NO: 196 - DNA, 20 bp, synthetic construct
  misc_feature 1..20: note = Synthetic
  source 1..20: mol_type = other DNA
gctgaggccgaggtgacgct

SEQ ID NO: 197 - DNA, 20 bp, synthetic construct
  misc_feature 1..20: note = Synthetic
  source 1..20: mol_type = other DNA
aacttcgccagtcaactacg

SEQ ID NO: 198 - DNA, 20 bp, synthetic construct
  misc_feature 1..20: note = Synthetic
  source 1..20: mol_type = other DNA
cagtcctgtgatccgggccc

SEQ ID NO: 199 - DNA, 20 bp, synthetic construct
  misc_feature 1..20: note = Synthetic
  source 1..20: mol_type = other DNA
gccctgacgtggatgggcaa

SEQ ID NO: 200 - DNA, 20 bp, synthetic construct
  misc_feature 1..20: note = Synthetic
  source 1..20: mol_type = other DNA
ggcagcaagccgggccgtcc

SEQ ID NO: 201 - DNA, 20 bp, synthetic construct
  misc_feature 1..20: note = Synthetic
  source 1..20: mol_type = other DNA
gccgggccgtccgtgttcca

SEQ ID NO: 202 - DNA, 20 bp, synthetic construct
  misc_feature 1..20: note = Synthetic
  source 1..20: mol_type = other DNA
cccaatccacgttttggatg

SEQ ID NO: 203 - DNA, 20 bp, synthetic construct
  misc_feature 1..20: note = Synthetic
  source 1..20: mol_type = other DNA
ccaatccacgttttggatgc

SEQ ID NO: 204 - DNA, 16 bp, synthetic construct
  misc_feature 1..16: note = Synthetic
  source 1..16: mol_type = other DNA
gcttgagccacacgtg

SEQ ID NO: 205 - DNA, 16 bp, synthetic construct
  misc_feature 1..16: note = Synthetic
  source 1..16: mol_type = other DNA
cttgagccacacgtgc

SEQ ID NO: 206 - DNA, 16 bp, synthetic construct
  misc_feature 1..16: note = Synthetic
  source 1..16: mol_type = other DNA
tgcttgagccacacgt

SEQ ID NO: 207 - DNA, 16 bp, synthetic construct
  misc_feature 1..16: note = Synthetic
  source 1..16: mol_type = other DNA
ctgctgagcaagtttg

SEQ ID NO: 208 - DNA, 16 bp, synthetic construct
  misc_feature 1..16: note = Synthetic
  source 1..16: mol_type = other DNA
atgaaacagctgaagt

SEQ ID NO: 209 - DNA, 16 bp, synthetic construct
  misc_feature 1..16: note = Synthetic
  source 1..16: mol_type = other DNA
cttcgccagtcaacta

SEQ ID NO: 210 - DNA, 16 bp, synthetic construct
  misc_feature 1..16: note = Synthetic
  source 1..16: mol_type = other DNA
gtcctgtgatccgggc

SEQ ID NO: 211 - DNA, 16 bp, synthetic construct
  misc_feature 1..16: note = Synthetic
  source 1..16: mol_type = other DNA
ggcggagattgtcatg

SEQ ID NO: 212 - DNA, 16 bp, synthetic construct
  misc_feature 1..16: note = Synthetic
  source 1..16: mol_type = other DNA
ctgaggccgaggtgac

SEQ ID NO: 213 - DNA, 16 bp, synthetic construct
  misc_feature 1..16: note = Synthetic
  source 1..16: mol_type = other DNA
aactacctgtacctgg

SEQ ID NO: 214 - DNA, 16 bp, synthetic construct
  misc_feature 1..16: note = Synthetic
  source 1..16: mol_type = other DNA
agagggacgtgttggt

SEQ ID NO: 215 - DNA, 16 bp, synthetic construct
  misc_feature 1..16: note = Synthetic
  source 1..16: mol_type = other DNA
tgctgagcaagtttgg

SEQ ID NO: 216 - DNA, 16 bp, synthetic construct
  misc_feature 1..16: note = Synthetic
  source 1..16: mol_type = other DNA
tgaaacagctgaagtg

SEQ ID NO: 217 - DNA, 16 bp, synthetic construct
  misc_feature 1..16: note = Synthetic
  source 1..16: mol_type = other DNA
ttcgccagtcaactac

SEQ ID NO: 218 - DNA, 16 bp, synthetic construct
  misc_feature 1..16: note = Synthetic
  source 1..16: mol_type = other DNA
tcctgtgatccgggcc

SEQ ID NO: 219 - DNA, 16 bp, synthetic construct
  misc_feature 1..16: note = Synthetic
  source 1..16: mol_type = other DNA
actgctgagcaagttt

SEQ ID NO: 220 - DNA, 16 bp, synthetic construct
  misc_feature 1..16: note = Synthetic
  source 1..16: mol_type = other DNA
gatgaaacagctgaag

SEQ ID NO: 221 - DNA, 16 bp, synthetic construct
  misc_feature 1..16: note = Synthetic
  source 1..16: mol_type = other DNA
acttcgccagtcaact

SEQ ID NO: 222 - DNA, 16 bp, synthetic construct
  misc_feature 1..16: note = Synthetic
  source 1..16: mol_type = other DNA
agtcctgtgatccggg

SEQ ID NO: 223 - DNA, 16 bp, synthetic construct
  misc_feature 1..16: note = Synthetic
  source 1..16: mol_type = other DNA
gcggagattgtcatgg

SEQ ID NO: 224 - DNA, 16 bp, synthetic construct
  misc_feature 1..16: note = Synthetic
  source 1..16: mol_type = other DNA
tggcggagattgtcat

SEQ ID NO: 225 - DNA, 16 bp, synthetic construct
  misc_feature 1..16: note = Synthetic
  source 1..16: mol_type = other DNA
tgaggccgaggtgacg

SEQ ID NO: 226 - DNA, 16 bp, synthetic construct
  misc_feature 1..16: note = Synthetic
  source 1..16: mol_type = other DNA
gctgaggccgaggtga

SEQ ID NO: 227 - DNA, 16 bp, synthetic construct
  misc_feature 1..16: note = Synthetic
  source 1..16: mol_type = other DNA
actacctgtacctggt

SEQ ID NO: 228 - DNA, 16 bp, synthetic construct
  misc_feature 1..16: note = Synthetic
  source 1..16: mol_type = other DNA
gaactacctgtacctg

SEQ ID NO: 229 - DNA, 16 bp, synthetic construct
  misc_feature 1..16: note = Synthetic
  source 1..16: mol_type = other DNA
gagggacgtgttggtg

SEQ ID NO: 230 - DNA, 16 bp, synthetic construct
  misc_feature 1..16: note = Synthetic
  source 1..16: mol_type = other DNA
gagagggacgtgttgg

SEQ ID NO: 231 - DNA, 20 bp, synthetic construct
  misc_feature 1..20: note = Synthetic
  source 1..20: mol_type = other DNA
catggcatacacctggcccg

SEQ ID NO: 232 - DNA, 20 bp, synthetic construct
  misc_feature 1..20: note = Synthetic
  source 1..20: mol_type = other DNA
caccaacacgtccctctcct

SEQ ID NO: 233 - DNA, 20 bp, synthetic construct
  misc_feature 1..20: note = Synthetic
  source 1..20: mol_type = other DNA
tcaccaacacgtccctctcc

SEQ ID NO: 234 - DNA, 20 bp, synthetic construct
  misc_feature 1..20: note = Synthetic
  source 1..20: mol_type = other DNA
ccattcaccaacacgtccct

SEQ ID NO: 235 - DNA, 20 bp, synthetic construct
  misc_feature 1..20: note = Synthetic
  source 1..20: mol_type = other DNA
tacaggtagttctcatcctg

SEQ ID NO: 236 - DNA, 20 bp, synthetic construct
  misc_feature 1..20: note = Synthetic
  source 1..20: mol_type = other DNA
gtacaggtagttctcatcct

SEQ ID NO: 237 - DNA, 20 bp, synthetic construct
  misc_feature 1..20: note = Synthetic
  source 1..20: mol_type = other DNA
accaggtacaggtagttctc

SEQ ID NO: 238 - DNA, 20 bp, synthetic construct
  misc_feature 1..20: note = Synthetic
  source 1..20: mol_type = other DNA
gaccaggtacaggtagttct

SEQ ID NO: 239 - DNA, 20 bp, synthetic construct
  misc_feature 1..20: note = Synthetic
  source 1..20: mol_type = other DNA
tgaccaggtacaggtagttc

SEQ ID NO: 240 - DNA, 20 bp, synthetic construct
  misc_feature 1..20: note = Synthetic
  source 1..20: mol_type = other DNA
cccaaacttgctcagcagtg

SEQ ID NO: 241 - DNA, 20 bp, synthetic construct
  misc_feature 1..20: note = Synthetic
  source 1..20: mol_type = other DNA
tgacaatctccgccaggtag

SEQ ID NO: 242 - DNA, 20 bp, synthetic construct
  misc_feature 1..20: note = Synthetic
  source 1..20: mol_type = other DNA
atgacaatctccgccaggta

SEQ ID NO: 243 - DNA, 20 bp, synthetic construct
  misc_feature 1..20: note = Synthetic
  source 1..20: mol_type = other DNA
catgacaatctccgccaggt

SEQ ID NO: 244 - DNA, 20 bp, synthetic construct
  misc_feature 1..20: note = Synthetic
  source 1..20: mol_type = other DNA
ccatgacaatctccgccagg

SEQ ID NO: 245 - DNA, 20 bp, synthetic construct
  misc_feature 1..20: note = Synthetic
  source 1..20: mol_type = other DNA
gccatgacaatctccgccag

SEQ ID NO: 246 - DNA, 20 bp, synthetic construct
  misc_feature 1..20: note = Synthetic
  source 1..20: mol_type = other DNA
ggccatgacaatctccgcca

SEQ ID NO: 247 - DNA, 20 bp, synthetic construct
  misc_feature 1..20: note = Synthetic
  source 1..20: mol_type = other DNA
tggccatgacaatctccgcc

SEQ ID NO: 248 - DNA, 20 bp, synthetic construct
  misc_feature 1..20: note = Synthetic
  source 1..20: mol_type = other DNA
tgtgcacgtagccaagccgg

SEQ ID NO: 249 - DNA, 20 bp, synthetic construct
  misc_feature 1..20: note = Synthetic
  source 1..20: mol_type = other DNA
ctgtgcacgtagccaagccg

SEQ ID NO: 250 - DNA, 20 bp, synthetic construct
  misc_feature 1..20: note = Synthetic
  source 1..20: mol_type = other DNA
cacagcggtccagcaggatg

SEQ ID NO: 251 - DNA, 20 bp, synthetic construct
  misc_feature 1..20: note = Synthetic
  source 1..20: mol_type = other DNA
tggccacagcggtccagcag

SEQ ID NO: 252 - DNA, 20 bp, synthetic construct
  misc_feature 1..20: note = Synthetic
  source 1..20: mol_type = other DNA
agcgcccaccagtcacactc

SEQ ID NO: 253 - DNA, 20 bp, synthetic construct
  misc_feature 1..20: note = Synthetic
  source 1..20: mol_type = other DNA
cagcgcccaccagtcacact

SEQ ID NO: 254 - DNA, 20 bp, synthetic construct
  misc_feature 1..20: note = Synthetic
  source 1..20: mol_type = other DNA
ccagcgcccaccagtcacac

SEQ ID NO: 255 - DNA, 20 bp, synthetic construct
  misc_feature 1..20: note = Synthetic
  source 1..20: mol_type = other DNA
gcgaatacacccagcgccca

SEQ ID NO: 256 - DNA, 20 bp, synthetic construct
  misc_feature 1..20: note = Synthetic
  source 1..20: mol_type = other DNA
ggcgaatacacccagcgccc

SEQ ID NO: 257 - DNA, 20 bp, synthetic construct
  misc_feature 1..20: note = Synthetic
  source 1..20: mol_type = other DNA
ttgtagtggacgatcttgcc

SEQ ID NO: 258 - DNA, 20 bp, synthetic construct
  misc_feature 1..20: note = Synthetic
  source 1..20: mol_type = other DNA
cttgtagtggacgatcttgc

SEQ ID NO: 259 - DNA, 20 bp, synthetic construct
  misc_feature 1..20: note = Synthetic
  source 1..20: mol_type = other DNA
ccttgtagtggacgatcttg

SEQ ID NO: 260 - DNA, 20 bp, synthetic construct
  misc_feature 1..20: note = Synthetic
  source 1..20: mol_type = other DNA
cggagaccatcccagtcgag

SEQ ID NO: 261 - DNA, 20 bp, synthetic construct
  misc_feature 1..20: note = Synthetic
  source 1..20: mol_type = other DNA
gaatgtccgacagtgtctcc

SEQ ID NO: 262 - DNA, 20 bp, synthetic construct
  misc_feature 1..20: note = Synthetic
  source 1..20: mol_type = other DNA
cgaatgtccgacagtgtctc

SEQ ID NO: 263 - DNA, 20 bp, synthetic construct
  misc_feature 1..20: note = Synthetic
  source 1..20: mol_type = other DNA
gggcctgggacctcactgtc

SEQ ID NO: 264 - DNA, 20 bp, synthetic construct
  misc_feature 1..20: note = Synthetic
  source 1..20: mol_type = other DNA
tgcacgtgtggctcaagcag

SEQ ID NO: 265 - DNA, 20 bp, synthetic construct
  misc_feature 1..20: note = Synthetic
  source 1..20: mol_type = other DNA
ccacttcagctgtttcatcc

SEQ ID NO: 266 - DNA, 20 bp, synthetic construct
  misc_feature 1..20: note = Synthetic
  source 1..20: mol_type = other DNA
gcgtcacctcggcctcagcc

SEQ ID NO: 267 - DNA, 20 bp, synthetic construct
  misc_feature 1..20: note = Synthetic
  source 1..20: mol_type = other DNA
agcgtcacctcggcctcagc

SEQ ID NO: 268 - DNA, 20 bp, synthetic construct
  misc_feature 1..20: note = Synthetic
  source 1..20: mol_type = other DNA
cgtagttgactggcgaagtt

SEQ ID NO: 269 - DNA, 20 bp, synthetic construct
  misc_feature 1..20: note = Synthetic
  source 1..20: mol_type = other DNA
gggcccggatcacaggactg

SEQ ID NO: 270 - DNA, 20 bp, synthetic construct
  misc_feature 1..20: note = Synthetic
  source 1..20: mol_type = other DNA
ttgcccatccacgtcagggc

SEQ ID NO: 271 - DNA, 20 bp, synthetic construct
  misc_feature 1..20: note = Synthetic
  source 1..20: mol_type = other DNA
ggacggcccggcttgctgcc

SEQ ID NO: 272 - DNA, 20 bp, synthetic construct
  misc_feature 1..20: note = Synthetic
  source 1..20: mol_type = other DNA
tggaacacggacggcccggc

SEQ ID NO: 273 - DNA, 20 bp, synthetic construct
  misc_feature 1..20: note = Synthetic
  source 1..20: mol_type = other DNA
catccaaaacgtggattggg

SEQ ID NO: 274 - DNA, 20 bp, synthetic construct
  misc_feature 1..20: note = Synthetic
  source 1..20: mol_type = other DNA
gcatccaaaacgtggattgg

SEQ ID NO: 275 - DNA, 16 bp, synthetic construct
  misc_feature 1..16: note = Synthetic
  source 1..16: mol_type = other DNA
cacgtgtggctcaagc

SEQ ID NO: 276 - DNA, 16 bp, synthetic construct
  misc_feature 1..16: note = Synthetic
  source 1..16: mol_type = other DNA
gcacgtgtggctcaag

SEQ ID NO: 277 - DNA, 16 bp, synthetic construct
  misc_feature 1..16: note = Synthetic
  source 1..16: mol_type = other DNA
acgtgtggctcaagca

SEQ ID NO: 278 - DNA, 16 bp, synthetic construct
  misc_feature 1..16: note = Synthetic
  source 1..16: mol_type = other DNA
caaacttgctcagcag

SEQ ID NO: 279 - DNA, 16 bp, synthetic construct
  misc_feature 1..16: note = Synthetic
  source 1..16: mol_type = other DNA
acttcagctgtttcat

SEQ ID NO: 280 - DNA, 16 bp, synthetic construct
  misc_feature 1..16: note = Synthetic
  source 1..16: mol_type = other DNA
tagttgactggcgaag

SEQ ID NO: 281 - DNA, 16 bp, synthetic construct
  misc_feature 1..16: note = Synthetic
  source 1..16: mol_type = other DNA
gcccggatcacaggac

SEQ ID NO: 282 - DNA, 16 bp, synthetic construct
  misc_feature 1..16: note = Synthetic
  source 1..16: mol_type = other DNA
catgacaatctccgcc

SEQ ID NO: 283 - DNA, 16 bp, synthetic construct
  misc_feature 1..16: note = Synthetic
  source 1..16: mol_type = other DNA
gtcacctcggcctcag

SEQ ID NO: 284 - DNA, 16 bp, synthetic construct
  misc_feature 1..16: note = Synthetic
  source 1..16: mol_type = other DNA
ccaggtacaggtagtt

SEQ ID NO: 285 - DNA, 16 bp, synthetic construct
  misc_feature 1..16: note = Synthetic
  source 1..16: mol_type = other DNA
accaacacgtccctct

SEQ ID NO: 286 - DNA, 16 bp, synthetic construct
  misc_feature 1..16: note = Synthetic
  source 1..16: mol_type = other DNA
ccaaacttgctcagca

SEQ ID NO: 287 - DNA, 16 bp, synthetic construct
  misc_feature 1..16: note = Synthetic
  source 1..16: mol_type = other DNA
cacttcagctgtttca

SEQ ID NO: 288 - DNA, 16 bp, synthetic construct
  misc_feature 1..16: note = Synthetic
  source 1..16: mol_type = other DNA
gtagttgactggcgaa

SEQ ID NO: 289 - DNA, 16 bp, synthetic construct
  misc_feature 1..16: note = Synthetic
  source 1..16: mol_type = other DNA
ggcccggatcacagga

SEQ ID NO: 290 - DNA, 16 bp, synthetic construct
  misc_feature 1..16: note = Synthetic
  source 1..16: mol_type = other DNA
aaacttgctcagcagt

SEQ ID NO: 291 - DNA, 16 bp, synthetic construct
  misc_feature 1..16: note = Synthetic
  source 1..16: mol_type = other DNA
cttcagctgtttcatc

SEQ ID NO: 292 - DNA, 16 bp, synthetic construct
  misc_feature 1..16: note = Synthetic
  source 1..16: mol_type = other DNA
agttgactggcgaagt

SEQ ID NO: 293 - DNA, 16 bp, synthetic construct
  misc_feature 1..16: note = Synthetic
  source 1..16: mol_type = other DNA
cccggatcacaggact

SEQ ID NO: 294 - DNA, 16 bp, synthetic construct
  misc_feature 1..16: note = Synthetic
  source 1..16: mol_type = other DNA
ccatgacaatctccgc

SEQ ID NO: 295 - DNA, 16 bp, synthetic construct
  misc_feature 1..16: note = Synthetic
  source 1..16: mol_type = other DNA
atgacaatctccgcca

SEQ ID NO: 296 - DNA, 16 bp, synthetic construct
  misc_feature 1..16: note = Synthetic
  source 1..16: mol_type = other DNA
cgtcacctcggcctca

SEQ ID NO: 297 - DNA, 16 bp, synthetic construct
  misc_feature 1..16: note = Synthetic
  source 1..16: mol_type = other DNA
tcacctcggcctcagc

SEQ ID NO: 298 - DNA, 16 bp, synthetic construct
  misc_feature 1..16: note = Synthetic
  source 1..16: mol_type = other DNA
accaggtacaggtagt

SEQ ID NO: 299 - DNA, 16 bp, synthetic construct
  misc_feature 1..16: note = Synthetic
  source 1..16: mol_type = other DNA
caggtacaggtagttc

SEQ ID NO: 300 - DNA, 16 bp, synthetic construct
  misc_feature 1..16: note = Synthetic
  source 1..16: mol_type = other DNA
caccaacacgtccctc

SEQ ID NO: 301 - DNA, 16 bp, synthetic construct
  misc_feature 1..16: note = Synthetic
  source 1..16: mol_type = other DNA
ccaacacgtccctctc

SEQ ID NO: 302 - DNA, 20 bp, synthetic construct
  misc_feature 1..20: note = Synthetic
  source 1..20: mol_type = other DNA
  modified_base 3: mod_base = OTHER; note = Uracil
catggcatacacctggcccg

SEQ ID NO: 303 - DNA, 20 bp, synthetic construct
  misc_feature 1..20: note = Synthetic
  source 1..20: mol_type = other DNA
  modified_base 17: mod_base = OTHER; note = Uracil
  modified_base 20: mod_base = OTHER; note = Uracil
caccaacacgtccctctcct

SEQ ID NO: 304 - DNA, 20 bp, synthetic construct
  misc_feature 1..20: note = Synthetic
  source 1..20: mol_type = other DNA
  modified_base 1: mod_base = OTHER; note = Uracil
  modified_base 16: mod_base = OTHER; note = Uracil
  modified_base 18: mod_base = OTHER; note = Uracil
tcaccaacacgtccctctcc

SEQ ID NO: 305 - DNA, 20 bp, synthetic construct
  misc_feature 1..20: note = Synthetic
  source 1..20: mol_type = other DNA
  modified_base 4..5: mod_base = OTHER; note = Uracil
  modified_base 16: mod_base = OTHER; note = Uracil
  modified_base 20: mod_base = OTHER; note = Uracil
ccattcaccaacacgtccct

SEQ ID NO: 306 - DNA, 20 bp, synthetic construct
  misc_feature 1..20: note = Synthetic
  source 1..20: mol_type = other DNA
  modified_base 1: mod_base = OTHER; note = Uracil
  modified_base 16: mod_base = OTHER; note = Uracil
  modified_base 19: mod_base = OTHER; note = Uracil
tacaggtagttctcatcctg

SEQ ID NO: 307 - DNA, 20 bp, synthetic construct
  misc_feature 1..20: note = Synthetic
  source 1..20: mol_type = other DNA
  modified_base 2: mod_base = OTHER; note = Uracil
  modified_base 17: mod_base = OTHER; note = Uracil
  modified_base 20: mod_base = OTHER; note = Uracil
gtacaggtagttctcatcct

SEQ ID NO: 308 - DNA, 20 bp, synthetic construct
  misc_feature 1..20: note = Synthetic
  source 1..20: mol_type = other DNA
  modified_base 16..17: mod_base = OTHER; note = Uracil
  modified_base 19: mod_base = OTHER; note = Uracil
accaggtacaggtagttctc

SEQ ID NO: 309 - DNA, 20 bp, synthetic construct
  misc_feature 1..20: note = Synthetic
  source 1..20: mol_type = other DNA
  modified_base 17..18: mod_base = OTHER; note = Uracil
  modified_base 20: mod_base = OTHER; note = Uracil
gaccaggtacaggtagttct

SEQ ID NO: 310 - DNA, 20 bp, synthetic construct
  misc_feature 1..20: note = Synthetic
  source 1..20: mol_type = other DNA
  modified_base 18..19: mod_base = OTHER; note = Uracil
  modified_base 1: mod_base = OTHER; note = Uracil
tgaccaggtacaggtagttc

SEQ ID NO: 311 - DNA, 20 bp, synthetic construct
  misc_feature 1..20: note = Synthetic
  source 1..20: mol_type = other DNA
  modified_base 19: mod_base = OTHER; note = Uracil
cccaaacttgctcagcagtg

SEQ ID NO: 312 - DNA, 20 bp, synthetic construct
  misc_feature 1..20: note = Synthetic
  source 1..20: mol_type = other DNA
  modified_base 1: mod_base = OTHER; note = Uracil
  modified_base 18: mod_base = OTHER; note = Uracil
tgacaatctccgccaggtag

SEQ ID NO: 313 - DNA, 20 bp, synthetic construct
  misc_feature 1..20: note = Synthetic
  source 1..20: mol_type = other DNA
  modified_base 2: mod_base = OTHER; note = Uracil
  modified_base 19: mod_base = OTHER; note = Uracil
atgacaatctccgccaggta

SEQ ID NO: 314 - DNA, 20 bp, synthetic construct
  misc_feature 1..20: note = Synthetic
  source 1..20: mol_type = other DNA
  modified_base 3: mod_base = OTHER; note = Uracil
  modified_base 20: mod_base = OTHER; note = Uracil
catgacaatctccgccaggt

SEQ ID NO: 315 - DNA, 20 bp, synthetic construct
  misc_feature 1..20: note = Synthetic
  source 1..20: mol_type = other DNA
  modified_base 4: mod_base = OTHER; note = Uracil
ccatgacaatctccgccagg

SEQ ID NO: 316 - DNA, 20 bp, synthetic construct
  misc_feature 1..20: note = Synthetic
  source 1..20: mol_type = other DNA
  modified_base 5: mod_base = OTHER; note = Uracil
gccatgacaatctccgccag

SEQ ID NO: 317 - DNA, 20 bp, synthetic construct
  misc_feature 1..20: note = Synthetic
  source 1..20: mol_type = other DNA
  modified_base 1: mod_base = OTHER; note = Uracil
tggccatgacaatctccgcc

SEQ ID NO: 318 - DNA, 20 bp, synthetic construct
  misc_feature 1..20: note = Synthetic
  source 1..20: mol_type = other DNA
  modified_base 1: mod_base = OTHER; note = Uracil
  modified_base 3: mod_base = OTHER; note = Uracil
tgtgcacgtagccaagccgg

SEQ ID NO: 319 - DNA, 20 bp, synthetic construct
  misc_feature 1..20: note = Synthetic
  source 1..20: mol_type = other DNA
  modified_base 2: mod_base = OTHER; note = Uracil
  modified_base 4: mod_base = OTHER; note = Uracil
ctgtgcacgtagccaagccg

SEQ ID NO: 320 - DNA, 20 bp, synthetic construct
  misc_feature 1..20: note = Synthetic
  source 1..20: mol_type = other DNA
  modified_base 19: mod_base = OTHER; note = Uracil
cacagcggtccagcaggatg

SEQ ID NO: 321 - DNA, 20 bp, synthetic construct
  misc_feature 1..20: note = Synthetic
  source 1..20: mol_type = other DNA
  modified_base 1: mod_base = OTHER; note = Uracil
tggccacagcggtccagcag

SEQ ID NO: 322 - DNA, 20 bp, synthetic construct
  misc_feature 1..20: note = Synthetic
  source 1..20: mol_type = other DNA
  modified_base 19: mod_base = OTHER; note = Uracil
agcgcccaccagtcacactc

SEQ ID NO: 323 - DNA, 20 bp, synthetic construct
  misc_feature 1..20: note = Synthetic
  source 1..20: mol_type = other DNA
  modified_base 20: mod_base = OTHER; note = Uracil
cagcgcccaccagtcacact

SEQ ID NO: 324 - DNA, 20 bp, synthetic construct
  misc_feature 1..20: note = Synthetic
  source 1..20: mol_type = other DNA
  modified_base 1..2: mod_base = OTHER; note = Uracil
  modified_base 4: mod_base = OTHER; note = Uracil
  modified_base 16..17: mod_base = OTHER; note = Uracil
ttgtagtggacgatcttgcc

SEQ ID NO: 325 - DNA, 20 bp, synthetic construct
  misc_feature 1..20: note = Synthetic
  source 1..20: mol_type = other DNA
  modified_base 2..3: mod_base = OTHER; note = Uracil
  modified_base 5: mod_base = OTHER; note = Uracil
  modified_base 17..18: mod_base = OTHER; note = Uracil
cttgtagtggacgatcttgc

SEQ ID NO: 326 - DNA, 20 bp, synthetic construct
  misc_feature 1..20: note = Synthetic
  source 1..20: mol_type = other DNA
  modified_base 3..4: mod_base = OTHER; note = Uracil
  modified_base 16: mod_base = OTHER; note = Uracil
  modified_base 18..19: mod_base = OTHER; note = Uracil
ccttgtagtggacgatcttg

SEQ ID NO: 327 - DNA, 20 bp, synthetic construct
  misc_feature 1..20: note = Synthetic
  source 1..20: mol_type = other DNA
  modified_base 16: mod_base = OTHER; note = Uracil
cggagaccatcccagtcgag

SEQ ID NO: 328 - DNA, 20 bp, synthetic construct
  misc_feature 1..20: note = Synthetic
  source 1..20: mol_type = other DNA
  modified_base 4: mod_base = OTHER; note = uracil
  modified_base 16: mod_base = OTHER; note = Uracil
  modified_base 18: mod_base = OTHER; note = Uracil
gaatgtccgacagtgtctcc

SEQ ID NO: 329 - DNA, 20 bp, synthetic construct
  misc_feature 1..20: note = Synthetic
  source 1..20: mol_type = other DNA
  modified_base 5: mod_base = OTHER; note = Uracil
  modified_base 17: mod_base = OTHER; note = Uracil
  modified_base 19: mod_base = OTHER; note = Uracil
cgaatgtccgacagtgtctc

SEQ ID NO: 330 - DNA, 20 bp, synthetic construct
  misc_feature 1..20: note = Synthetic
  source 1..20: mol_type = other DNA
  modified_base 17: mod_base = OTHER; note = Uracil
  modified_base 19: mod_base = OTHER; note = Uracil
gggcctgggacctcactgtc

SEQ ID NO: 331 - DNA, 20 bp, synthetic construct
  misc_feature 1..20: note = Synthetic
  source 1..20: mol_type = other DNA
  modified_base 1: mod_base = OTHER; note = Uracil
tgcacgtgtggctcaagcag

SEQ ID NO: 332 - DNA, 20 bp, synthetic construct
  misc_feature 1..20: note = Synthetic
  source 1..20: mol_type = other DNA
  modified_base 5: mod_base = OTHER; note = Uracil
  modified_base 18: mod_base = OTHER; note = Uracil
ccacttcagctgtttcatcc

SEQ ID NO: 333 - DNA, 20 bp, synthetic construct
  misc_feature 1..20: note = Synthetic
  source 1..20: mol_type = other DNA
  modified_base 4: mod_base = OTHER; note = Uracil
gcgtcacctcggcctcagcc

SEQ ID NO: 334 - DNA, 20 bp, synthetic construct
  misc_feature 1..20: note = Synthetic
  source 1..20: mol_type = other DNA
  modified_base 5: mod_base = OTHER; note = Uracil
  modified_base 16: mod_base = OTHER; note = Uracil
agcgtcacctcggcctcagc

SEQ ID NO: 335 - DNA, 20 bp, synthetic construct
  misc_feature 1..20: note = Synthetic
  source 1..20: mol_type = other DNA
  modified_base 3: mod_base = OTHER; note = Uracil
  modified_base 19..20: mod_base = OTHER; note = Uracil
cgtagttgactggcgaagtt

SEQ ID NO: 336 - DNA, 20 bp, synthetic construct
  misc_feature 1..20: note = Synthetic
  source 1..20: mol_type = other DNA
  modified_base 19: mod_base = OTHER; note = Uracil
gggcccggatcacaggactg

SEQ ID NO: 337 - DNA, 20 bp, synthetic construct
  misc_feature 1..20: note = Synthetic
  source 1..20: mol_type = other DNA
  modified_base 1..2: mod_base = OTHER; note = Uracil
ttgcccatccacgtcagggc

SEQ ID NO: 338 - DNA, 20 bp, synthetic construct
  misc_feature 1..20: note = Synthetic
  source 1..20: mol_type = other DNA
  modified_base 17: mod_base = OTHER; note = Uracil
ggacggcccggcttgctgcc

SEQ ID NO: 339 - RNA, 20 nt, synthetic construct
  misc_feature 1..20: note = Synthetic
  source 1..20: mol_type = other RNA
tggaacacggacggcccggc

SEQ ID NO: 340 - DNA, 20 bp, synthetic construct
  misc_feature 1..20: note = Synthetic
  source 1..20: mol_type = other DNA
  modified_base 3: mod_base = OTHER; note = Uracil
  modified_base 16..17: mod_base = OTHER; note = Uracil
catccaaaacgtggattggg

SEQ ID NO: 341 - DNA, 20 bp, synthetic construct
  misc_feature 1..20: note = Synthetic
  source 1..20: mol_type = other DNA
  modified_base 4: mod_base = OTHER; note = Uracil
  modified_base 17..18: mod_base = OTHER; note = Uracil
gcatccaaaacgtggattgg

SEQ ID NO: 342 - DNA, 16 bp, synthetic construct
  misc_feature 1..16: note = Synthetic
  source 1..16: mol_type = other DNA
  modified_base 4: mod_base = OTHER; note = Uracil
acgtgtggctcaagca

SEQ ID NO: 343 - DNA, 16 bp, synthetic construct
  misc_feature 1..16: note = Synthetic
  source 1..16: mol_type = other DNA
  modified_base 3..4: mod_base = OTHER; note = Uracil
  modified_base 13: mod_base = OTHER; note = Uracil
  modified_base 16: mod_base = OTHER; note = Uracil
acttcagctgtttcat

SEQ ID NO: 344 - DNA, 16 bp, synthetic construct
  misc_feature 1..16: note = Synthetic
  source 1..16: mol_type = other DNA
  modified_base 1: mod_base = OTHER; note = Uracil
  modified_base 4: mod_base = OTHER; note = Uracil
tagttgactggcgaag

SEQ ID NO: 345 - DNA, 16 bp, synthetic construct
  misc_feature 1..16: note = Synthetic
  source 1..16: mol_type = other DNA
  modified_base 3: mod_base = OTHER; note = Uracil
catgacaatctccgcc

SEQ ID NO: 346 - DNA, 16 bp, synthetic construct
  misc_feature 1..16: note = Synthetic
  source 1..16: mol_type = other DNA
  modified_base 2: mod_base = OTHER; note = Uracil
  modified_base 13: mod_base = OTHER; note = Uracil
gtcacctcggcctcag

SEQ ID NO: 347 - DNA, 16 bp, synthetic construct
  misc_feature 1..16: note = Synthetic
  source 1..16: mol_type = other DNA
  modified_base 15..16: mod_base = OTHER; note = Uracil
ccaggtacaggtagtt

SEQ ID NO: 348 - DNA, 16 bp, synthetic construct
  misc_feature 1..16: note = Synthetic
  source 1..16: mol_type = other DNA
  modified_base 14: mod_base = OTHER; note = Uracil
  modified_base 16: mod_base = OTHER; note = Uracil
accaacacgtccctct

SEQ ID NO: 349 - DNA, 16 bp, synthetic construct
  misc_feature 1..16: note = Synthetic
  source 1..16: mol_type = other DNA
  modified_base 4: mod_base = OTHER; note = Uracil
  modified_base 13..14: mod_base = OTHER; note = Uracil
cacttcagctgtttca

SEQ ID NO: 350 - DNA, 16 bp, synthetic construct
  misc_feature 1..16: note = Synthetic
  source 1..16: mol_type = other DNA
  modified_base 2: mod_base = OTHER; note = Uracil
gtagttgactggcgaa

SEQ ID NO: 351 - DNA, 16 bp, synthetic construct
  misc_feature 1..16: note = Synthetic
  source 1..16: mol_type = other DNA
  modified_base 16: mod_base = OTHER; note = Uracil
aaacttgctcagcagt

SEQ ID NO: 352 - DNA, 16 bp, synthetic construct
  misc_feature 1..16: note = Synthetic
  source 1..16: mol_type = other DNA
  modified_base 2..3: mod_base = OTHER; note = Uracil
  modified_base 15: mod_base = OTHER; note = Uracil
cttcagctgtttcatc

SEQ ID NO: 353 - DNA, 16 bp, synthetic construct
  misc_feature 1..16: note = Synthetic
  source 1..16: mol_type = other DNA
  modified_base 3..4: mod_base = OTHER; note = Uracil
  modified_base 16: mod_base = OTHER; note = Uracil
agttgactggcgaagt

SEQ ID NO: 354 - DNA, 16 bp, synthetic construct
  misc_feature 1..16: note = Synthetic
  source 1..16: mol_type = other DNA
  modified_base 16: mod_base = OTHER; note = Uracil
cccggatcacaggact

SEQ ID NO: 355 - DNA, 16 bp, synthetic construct
  misc_feature 1..16: note = Synthetic
  source 1..16: mol_type = other DNA
  modified_base 4: mod_base = OTHER; note = Uracil
ccatgacaatctccgc

SEQ ID NO: 356 - DNA, 16 bp, synthetic construct
  misc_feature 1..16: note = Synthetic
  source 1..16: mol_type = other DNA
  modified_base 2: mod_base = OTHER; note = Uracil
atgacaatctccgcca

SEQ ID NO: 357 - DNA, 16 bp, synthetic construct
  misc_feature 1..16: note = Synthetic
  source 1..16: mol_type = other DNA
  modified_base 3: mod_base = OTHER; note = Uracil
  modified_base 14: mod_base = OTHER; note = Uracil
cgtcacctcggcctca

SEQ ID NO: 358 - DNA, 16 bp, synthetic construct
  misc_feature 1..16: note = Synthetic
  source 1..16: mol_type = other DNA
  modified_base 1: mod_base = OTHER; note = Uracil
tcacctcggcctcagc

SEQ ID NO: 359 - DNA, 16 bp, synthetic construct
  misc_feature 1..16: note = Synthetic
  source 1..16: mol_type = other DNA
  modified_base 13: mod_base = OTHER; note = Uracil
  modified_base 16: mod_base = OTHER; note = Uracil
accaggtacaggtagt

SEQ ID NO: 360 - DNA, 16 bp, synthetic construct
  misc_feature 1..16: note = Synthetic
  source 1..16: mol_type = other DNA
  modified_base 14..15: mod_base = OTHER; note = Uracil
caggtacaggtagttc

SEQ ID NO: 361 - DNA, 16 bp, synthetic construct
  misc_feature 1..16: note = Synthetic
  source 1..16: mol_type = other DNA
  modified_base 15: mod_base = OTHER; note = Uracil
caccaacacgtccctc

SEQ ID NO: 362 - DNA, 16 bp, synthetic construct
  misc_feature 1..16: note = Synthetic
  source 1..16: mol_type = other DNA
  modified_base 13: mod_base = OTHER; note = Uracil
  modified_base 15: mod_base = OTHER; note = Uracil
ccaacacgtccctctc

SEQ ID NO: 363 - protein, 7 aa, synthetic construct
  REGION 1..7: note = Synthetic
ASSLNIA

SEQ ID NO: 364 - protein, 12 aa, synthetic construct
  REGION 1..12: note = Synthetic
SKTFNTHPQSTP

SEQ ID NO: 365 - protein, 12 aa, synthetic construct
  REGION 1..12: note = Synthetic
TARGEHKEEELI

SEQ ID NO: 366 - protein, 9 aa, synthetic construct
  REGION 1..9: note = Synthetic
CQAQGQLVC

SEQ ID NO: 367 - protein, 9 aa, synthetic construct
  REGION 1..9: note = Synthetic
CSERSMNFC

SEQ ID NO: 368 - protein, 9 aa, synthetic construct
  REGION 1..9: note = Synthetic
CPKTRRVPC

SEQ ID NO: 369 - protein, 20 aa, synthetic construct
  REGION 1..20: note = Synthetic
WLSEAGPVVTVRALRGTGSW

SEQ ID NO: 370 - protein, 9 aa, synthetic construct
  REGION 1..9: note = Synthetic
CMQHSMRVC

SEQ ID NO: 371 - protein, 7 aa, synthetic construct
  REGION 1..7: note = Synthetic
DDTRHWG

SEQ ID NO: 372 - protein, 10 aa, synthetic construct
  REGION 1..10: note = Synthetic
GYTFTNYWMH

SEQ ID NO: 373 - protein, 17 aa, synthetic construct
  REGION 1..17: note = Synthetic
EINPINGRSNYGERFKT

SEQ ID NO: 374 - protein, 7 aa, synthetic construct
  REGION 1..7: note = Synthetic
GTRAMHY

SEQ ID NO: 375 - protein, 11 aa, synthetic construct
  REGION 1..11: note = Synthetic
RTSENIYNNLA

SEQ ID NO: 376 - protein, 7 aa, synthetic construct
  REGION 1..7: note = Synthetic
AATNLAD

SEQ ID NO: 377 - protein, 10 aa, synthetic construct
  REGION 1..10: note = Synthetic
QHFWGTPLTF

SEQ ID NO: 378 - protein, 116 aa, synthetic construct
  REGION 1..116: note = Synthetic
QVQLQQPGAELVKPGASVKLSCKASGYTFTNYWMHWVKQRPGQGLEWIGEINPINGRSNYGERFKTKATLTVDKSSSTAYMQLSSLTSEDSAVYYCARGTRAMHYWGQGTSVTVSS

SEQ ID NO: 379 - protein, 107 aa, synthetic construct
  REGION 1..107: note = Synthetic
DIQMTQSPASLSVSVGETVTITCRTSENIYNNLAWYQQKQGKSPQLLVYAATNLADGVPSRFSGSGSGTQYSLKINSLQSEDFGNYYCQHFWGTPLTFGAGTKLELK

SEQ ID NO: 380 - protein, 9 aa, synthetic construct
  REGION 1..9: note = Synthetic
YTFTNYWMH

SEQ ID NO: 381 - protein, 17 aa, synthetic construct
  REGION 1..17: note = Synthetic
EINPINGRSNYAQKFQG

SEQ ID NO: 382 - protein, 17 aa, synthetic construct
  REGION 1..17: note = Synthetic
EINPINGRSNYAEKFQG

SEQ ID NO: 383 - protein, 17 aa, synthetic construct
  REGION 1..17: note = Synthetic
EINPIQGRSNYAEKFQG

SEQ ID NO: 384 - protein, 7 aa, synthetic construct
  REGION 1..7: note = Synthetic
AATNLAE

SEQ ID NO: 385 - protein, 11 aa, synthetic construct
  REGION 1..11: note = Synthetic
RTSENIYSNLA

SEQ ID NO: 386 - protein, 7 aa, synthetic construct
  REGION 1..7: note = Synthetic
AGTNLAD

SEQ ID NO: 387 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAQKFQGRVTLTVDTSISTAYMELSRLRSDDTAVYYCARGTRAMHYWGQGTLVTVSS

SEQ ID NO: 388 - protein, 116 aa, synthetic construct
  REGION 1..116: note = Synthetic
QVQLVQSGAEVKKPGASVKVSCKASGYTFTNYWMHWVRQAPGQGLEWIGEINPINGRSNYAEKFQGRVTLTVDTSSSTAYMELSRLRSDDTAVYYCARGTRAMHYWGQGTLVTVSS

SEQ ID NO: 389 - protein, 116 aa, synthetic construct
  REGION 1..116: note = Synthetic
QVQLVQSGAEVKKPGASVKVSCKASGYTFTNYWMHWVRQAPGQGLEWMGEINPIQGRSNYAEKFQGRVTLTVDTSSSTAYMELSSLRSEDTATYYCARGTRAMHYWGQGTLVTVSS

SEQ ID NO: 390 - protein, 116 aa, synthetic construct
  REGION 1..116: note = Synthetic
QVQLVQSGAEVKKPGASVKVSCKASGYTFTNYWMHWVRQAPGQGLEWMGEINPINGRSNYAEKFQGRVTLTVDTSSSTAYMELSSLRSEDTATYYCARGTRAMHYWGQGTLVTVSS

SEQ ID NO: 391 - protein, 107 aa, synthetic construct
  REGION 1..107: note = Synthetic
DIQMTQSPSSLSASVGDRVTITCRTSENIYNNLAWYQQKPGKSPKLLIYAATNLADGVPSRFSGSGSGTDYTLTISSLQPEDFATYYCQHFWGTPLTFGGGTKVEIK

SEQ ID NO: 392 - protein, 107 aa, synthetic construct
  REGION 1..107: note = Synthetic
DIQMTQSPSSLSASVGDRVTITCRTSENIYNNLAWYQQKPGKAPKLLIYAATNLADGVPSRFSGSGSGTDYTLTISSLQPEDFATYYCQHFWGTPLTFGGGTKVEIK

SEQ ID NO: 393 - protein, 107 aa, synthetic construct
  REGION 1..107: note = Synthetic
DIQMTQSPSSLSASVGDRVTITCRTSENIYNNLAWYQQKPGKAPKLLIYAATNLAEGVPSRFSGSGSGTDYTLTISSLQPEDFATYYCQHFWGTPLTFGGGTKVEIK

SEQ ID NO: 394 - protein, 107 aa, synthetic construct
  REGION 1..107: note = Synthetic
DIQMTQSPSSLSASVGDRVTITCRTSENIYSNLAWYQQKPGKAPKLLIYAGTNLADGVPSRFSGSGSGTDYTLTISSLQPEDFANYYCQHFWGTPLTFGGGTKVEIK

SEQ ID NO: 395 - DNA, 19841 bp, Homo sapiens
  source 1..19841: mol_type = genomic DNA
tttcctacttgaagcctgacgtagtaaagatcgggggagggttagacagatacagtggtccccaaccattttggcaccagggactggtcttatggaagacagtttttccacagactgttggggggatggttttgggatgaaaccgttctgcctctgatcatcaggtgttagattctaataaggagcgcacacctagatccctcgcatgcatagttcatggtggggttcgcactcttacgaggattgaatggtgcgctgctccggtaggaggctgggctcaggctgtaatgcctgctcgcccaccactcacctcctgctgcatggcctggttcctaacaggccacggaccactactgttccatggcccggaggttgaggaccccgagatacaggacaattctgtggcaagcaggactgtcccctcgccaaagatgggacatcgaggctccttggagcaccctgtggccaccttgcagcagcctctgtttccccatgtttccatgacctggtgtccatctgtcttccccagtttgggagcttctctccgaggaggacctgggcctggtgtggcacctgctgtgtgagcggggccatgtccaacggccttcttggggactttgggtcggggagaagttctgcctgggttttactgccttctcccaaccccacactgtctcccctggcagcggttgatcgacaagaccaaggtgacatatctgaagtggctgcctgagtcggagagcctgttcctggcatcacacgccagtggccacctgtacctgtacaacgtcagccacccctgcgcctcggccccgccccagtacagcctgctgaagcagggcgagggcttctctgtctatgctgccaagagcaaggcaccccgcaacccgctggccaagtgggcggtgggtgaggggcccctcaacgagttcgccttctcgcccgatggccggcacctggcctgtgtgagccaggatggctgcctgcgcgtcttccacttcgactccatgctcctgcgtgggctcatgaagagctactttgggggcctgctgtgtgtgtgctggagccctgacggccgctacgtggtgacgggtggcgaagatgacctggtcaccgtgtggtccttcaccgagggccgcgtggtggctcgaggccatggccacaagtcctgggtcaacgctgtggcctttgacccctacaccacaagggcagaggaggcggcgacagcagccggtgctgatggggagcggagcggcgaagaggaggaggaggagcccgaggctgcgggcacaggctcggccgggggcgccccgctctctccactgcccaaggctggctccattacttaccgctttggctcggcgggccaggacacgcagttctgcctgtgggacctcactgaagacgtgctctacccgcacccccccctggcccgcacccgcaccctccctggcacacctggcaccacgccaccggccgccagcagctcgaggggtggcgagcctggcccaggccccctgcctcgctcgctgtcccgctccaacagtctcccgcacccagctggcgggggcaaggcgggcggcccgggtgtggcggcagagcctggcacaccattcagcattggccgcttcgccacgctcacactgcaggagcggcgggaccggggggcagagaaggagcacaagcgctaccacagcctgggcaacatcagccggggtggcagtggcggcagtggcagtggtggggagaagcccagcggccctgttccccgcagccgcctggaccccgccaaggtgctgggcactgcgctgtgcccgcgcatccacgaggtgcccctgctggagccccttgtgtgcaagaagatcgcccaggagcggctcacagtcctcctgttcctggaggactgcatcatcactgcctgccaggagggcctcatctgcacctgggcccggccgggcaaggcggtgagtggccccacaccagcctgccggggacctggcaggacctttcgtgggaagaggcaggcattggcagagagagggctttgttgctgtcacagcctctggctccgtggggtgaggggaagccagggaaatcttagtgtctcagtacaagacctctcagatccttagagtgagggggtctagccctaggcagcaggcagcagaaagaggggtgggtgtgagagccagctaggaattggggcatccaaggctggccgtctgaagggcagcagatgggccccacatggccaggtcttactgcctgtcactcgaaccagaatctatttctgttgaacatctgttttttaaatcgtgaaacttttttgagtacttcaggccaaaactaggggcgagctcaagcctgtgggcatggctgccagcctgggtctgggactcaggatctgagcctcctgctgaaggcacaggctgggaatcccaggcctgggttccagtcccactccctctgtgaccctggacaagtcactgccccctctgacctccaactcatcacctcttagaacagagcctgtaggatgggcagtgggtggatgtgcttgcctcctgggtgggctgtggcgttgggaaggtcatagtaggcgaatcaggcctggcatcttgtaagttcggagctcgtcttgggtgtctcagcttcttagggcttggactcagttgcccagggtcctggaggccgtggcttggttcctcagatcctcagttttggaatcgtagagtcctgagtccctagaacttgagagcacagtctgagtgactcagaggcaagagtggtgggatttggggagtctggttgagtcctaaaagagacccctctgtctccgtagttcacagacgaggagaccgaggcccagacaggggaaggaagttggcccaggtcacccagcaagtcagtggtagaggtaggactgtccctgagttctttccccagcacctcagggtccctcccaagttagaagggagctccagtttccccctcccctcccacccttacccttaccccatggtctcactcaggatccgccaaggactttgattattgcgtgaaagtgctgactgccaggacaggaagctagctaagatgcaagttcccagcctagagcagtggcctctggggggtctagggcggacccaagggcaaggccagggtggcagcagctttggggactctgggctggctccctccccttgacactggctgaagcccaggtggtctctaacccctcccatctctccctctcatcttccccagggcatctcctcccaaccaggcaactccccgagtggcacagtggtgtgaagccatggatatcgggcccccccaaccccatgcccccagcctcctagccataaccctccctgctgacctcacagatcaacgtattaacaagactaaccatgatggatggactgctccagtccccccacctgcacaaaatttgggggccccccagactggcccggacacgggcgatgtaatagcccttgtggcctcagccttgtcccccacccactgccaagtacaatgacctcttcctctgaaacatcagtgttaccctcatccctgtccccagcatgtgactggtcactcctggggagagactccccgcccctgccacaagagccccaggtctgcagtgtgcccctcagttgagtgggcagggccgggggtggtccagccctcgcccggcccccaccccagctgcccttgctattgtctgtgcttttgaagagtgttaaattatggaagcccctcaggttcctccctgtcccgcaggacctcttatttatactaaagttccctgttttctcagcgggtctgtccccttcggaggagatgatgtagaggacctgtgtgtgtactctgtggttctaggcagtccgctttccccagaggaggagtgcaggcctgctcccagcccagcgcctcccaccccttttcatagcaggaaaagccggagcccagggagggaacggacctgcgagtcacacaactggtgacccacaccagcggctggagcaggaccctcttggggagaagagcatcctgcccgcagccagggcccctcatcaaagtcctcggtgttttttaaattatcagaactgcccaggaccacgtttcccaggccctgcccagctgggactcctcggtccttgcctcctagtttctcaggcctggccctctcaaggcccaggcaccccaggccggttggaggccccgacttccactctggagaaccgtccaccctggaaagaagagctcagattcctcttggctctcggagccgcagggagtgtgtcttcccgcgccaccctccaccccccgaaatgtttctgtttctaatcccagcctgggcaggaatgtggctccccggccaggggccaaggagctattttggggtctcgtttgcccagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaaagctggtataaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgtttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtcctcctggacggcccgttaccttctgcgttgtcccgatggggaaactgaggccctgagccagaagcacacgctggggggaggcagaaagcgcggccagaggcggagggaaaacaaagggagaatcacagacagacgggagggggacggacacacacaaggggacagagacccgagtggagagctggatctcgccttcccggcgtggggcgcagggtcggccagaaagaagatcgagaagagcggggagtgggggcgaaaaggggggacaggtgggggaggaggctggggaaagcccgagggaggaagagagggagggaggaacttcccaaagttgcaaaacatggctaccttgcctgcggagccgagcgcggggccggcggctgggggggaggcggtggcggcggcggcggcgaccgaagaggaggaggaggaagcgcgccagctcttgcagactttgcaggcggccgagggtgaggcggcggcggcggccggggccggggcgggcgcagcggctgcgggagctgagggcccgggatccccgggcgtccccgggtcgccccccgaggccgcttccgaaccgcccacgggcctccgcttctcgcccgagcaggtggcgtgcgtctgcgaggcgctgctccaggcgggccacgccggccgcttgagccgcttcctgggcgcactgcccccggccgagcgcctacgtggcagcgacccggtgttgcgcgcgcgggccctggtggccttccagcggggcgagtacgccgagctctaccggctactcgagagccgccccttccccgccgcccaccacgccttcctgcaggacctctacctgcgcgcgcgctaccatgaggccgagcgggcccgcggccgcgcgcttggcgcagtggacaagtatcgactgcgcaagaagttcccgctgcccaagaccatctgggacggcgaggagacagtctactgcttcaaggagcgctcccgcgcagcgctcaaggcctgctaccgcggcaaccgctaccccacgccggacgagaagcgccgcctggccacactcaccggcctgtcgctcacgcaggtcagcaactggttcaagaaccggcgacagcgcgaccggaccggggccggaggcggcgcgccctgcaagaggtgaggggcctcgggcggcgcaagtccagctctcccggggacatcccgtccaccagccctcttcccccgtgcccactgctggggccggcgcgccgaggtcctcggacatctcccgggaccagctcacaatctcaggcgcccgcggggcgcggggactaagtgtggacgggacaggcacccgcccgggccctctccccgcacgcgtctcctcttccagcggctccattccgagctccttcccaaatcccatcggtgttggggaatcacactgcggggggcactagagggactgaggaaaaaggacagggcctgtggccactccact